(19) United States
(12) Patent Application Publication
(10) Pub. No.: US 2015/0246907 A1
(43) Pub. Date: Sep. 3, 2015

(54) PYRIDOMYCIN BASED COMPOUNDS EXHIBITING AN ANTITUBERCULAR ACTIVITY

(71) Applicant: ETH Zurich, Zurich (CH)

(72) Inventors: Karl-Heinz Altmann, Reinach BL (CH); Oliver Horlacher, Zurich (CH); Ruben Hartkoorn, Ecublens (CH); Stewart Cole, Ecublens (CH)

(21) Appl. No.: 14/427,406

(22) PCT Filed: Sep. 4, 2013
(86) PCT No.: PCT/EP2013/002657

§ 371 Date: Mar. 11, 2015

(30) Foreign Application Priority Data
Sep. 11, 2012 (EP) 12405095.6

(51) Int. Cl. C07D 413/14; C07D 413/12; C07D 401/12; C07D 413/06; C07D 405/04; C07D 273/01; C07D 273/02; C07D 255/02; C07D 405/14; C07D 413/04; C07D 401/14; C07D 401/06

(52) U.S. Cl. CPC C07D 413/14; C07D 401/14; C07D 413/12; C07D 401/12; C07D 413/06; C07D 401/06; C07D 273/01; C07D 273/02; C07D 255/02; C07D 405/14; C07D 413/04; C07D 405/04

(57) ABSTRACT

Formula (1), X₁ represents O or NR₆, X₂ represents O or NR₆, X₃ represents O or NR₁, R₁ represents H or C₁ to C₃ alkyl, R₂ represents H, or linear or branched C₁-C₈ alkyl optionally including one or more heteroatoms, cyclopropyl, cyclobutyl, cyclohexyl or oxetanyl or amino acid side chain or protected amino acid side chain, or R₁ and R₂ may form together a saturated, partly saturated or unsaturated 5 or 6 membered ring system, optionally substituted, R₃ represents H, cyclopentyl, cyclohexyl, aryl or hydroxyaryl, aryl or hydroxyaryl being optionally substituted by fluorine, or linear or branched C₁ to C₈ alkyl optionally including a hetero atom, R₄ represents phenyl or 5- or 6-membered heterocycles including one or more nitrogen or oxygen atoms optionally substituted with 1 to 4, respectively 5 fluorine atoms, and R₆ represents H, or linear or branched alkyl chain having 1 to 3 carbon atoms.

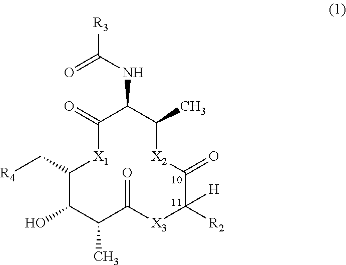

The present invention relates to new compounds, the method for preparation thereof as well as their use as medicaments for the treatment of tuberculosis.

Tuberculosis was considered eliminated in industrialized countries but global migration and immigration have led to an alarming number of multi- and extensively-drug-resistant strains of Mycobacterium tuberculosis. The situation is exacerbated by the fact that it has been more than 40 years since a novel antituberculotic was introduced and today's combination therapy is not sufficient to eliminate extensively-drug-resistant strains of Mycobacterium tuberculosis.

Pydridomycin is a bacterial natural product that was first isolated from the Streptomyces strain 6706 in 1953 (Maeda, K. et al, Journal of Antibiotics (Tokyo), 1953, 6(3), 140. Pydridomycin has the formula

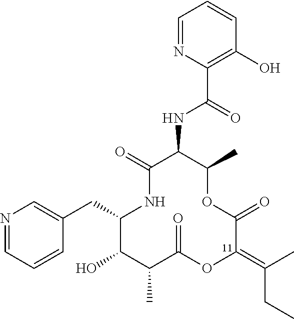 [SMILES: CC/C(C)=C1\OC(=O)[C@H](C)[C@H](O)[C@H](Cc2cccnc2)NC(=O)[C@@H](NC(=O)c2ncccc2O)[C@@H](C)OC1=O]

Later, it was shown, that said compound exhibits a significant in vitro antitubercular activity and low systemic toxicity in mice. To date there is one total synthesis of pyrodomycin which was published in Kinoshita, Tetrahedron Letters 52, 7419-7422 (1989)). However, the chemical synthesis is time consuming and does not include any structural modifications in terms of antitubercular activity.

The problem addressed in the present invention is to provide new compounds which exhibit antitubercular activity and a simple synthesis route for their preparation.

The problem is solved by the compounds according to claim 1. Further preferred embodiments are subject to the dependent claims.

The present invention provides compounds, which exhibit antibacterial activity, in particular antitubercular activity, said compounds having the general formula (1)

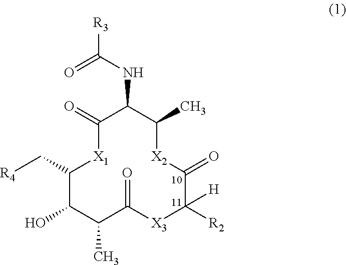

wherein
X₁ represents O or NR₆,
X₂ represents O or NR₆,
X₃ represents O or NR₁,
R₁ represents H or C₁ to C₃ alkyl,
R₂ represents H, or a linear or branched C₁-C₈ alkyl optionally comprising one or more heteroatoms, cyclopropyl, cyclobutyl, cyclopentyl, cyclohexyl, oxetanyl or an amino acid side chain or a protected amino acid side chain, or
R₁ and R₂ may form together a 5 or 6 membered ring system which may be saturated, partly saturated or unsaturated, said ring system being optionally substituted,
R₃ represents H, cyclopentyl, cyclohexyl, aryl or hydroxyaryl, said aryl or hydroxyaryl being optionally substituted by fluorine, or linear or branched C₁ to C₈ alkyl, optionally comprising a hetero atom,
R₄ represents 5 or 6 membered heterocycles comprising one or more nitrogen or oxygen atoms, in particular 2-, 3- or 4-pyridyl, or phenyl, optionally substituted with 1 to 4, respectively 5 fluorine atoms, and
R₆ represents H, or a linear or branched alkyl chain having 1 to 3 carbon atoms.

In all these compounds C11 is a sp³ carbon atom instead of a sp² atom as in the enol-ester moiety in the pyridomycin molecule. It is highly remarkable that, despite the significant change in the steric and electronic properties of the scaffold, the compounds according to the present invention retain the activity against mycobacteria.

The compounds according to the present invention or pharmaceutically acceptable salts thereof include the diastereomers of said compounds and also mixtures thereof in any mixing ratios. Furthermore, within the context of the invention the term “compounds or pharmaceutically acceptable salts thereof” is meant to include also hydrates and solvates of the compounds of formula I and their salts.

The compounds according to the present invention show potent antibacterial activity against pathogenic bacteria, in particular against tuberculosis bacteria, especially against multi- and extensively-drug-resistant strains of Mycobacterium tuberculosis.

Preferably, in the compound of the present invention R₂ represents H, or a linear or branched C₁-C₈ alkyl optionally comprising one or more heteroatoms, cyclopropyl, cyclobutyl or cyclopentyl, oxetanyl or an amino acid side chain or a protected amino acid side chain, or R₁ and R₂ may form together a 5- or 6-membered ring system which may be saturated, partly saturated or unsaturated, said ring system being optionally substituted.

In one embodiment of the present invention X₃ represents O, resulting in a compound having the formula (2)

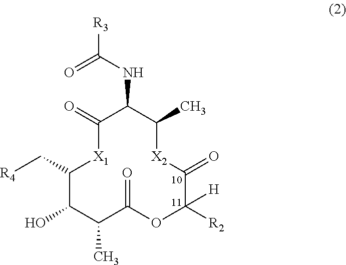

and
X₁, X₂, R₂, R₃, and R₄ have the same definition as above.

In a preferred embodiment of the present invention in the compound of formula (2) X₁ and X₂ are both oxygen, or X₁ is NR₆ and X₂ is oxygen or X₁ is oxygen and X₂ is NR₆, whereby R₆ is hydrogen or C₁ to C₃ alkyl, most preferred hydrogen or methyl.

In another embodiment of the present invention X₃ represents NR₁, resulting in a compound having the formula (3)

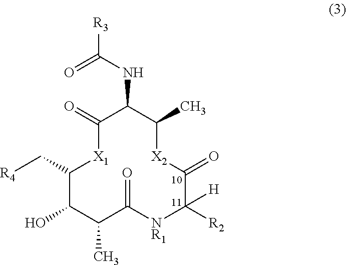

wherein R₁ is selected from the group of hydrogen, methyl, ethyl, propyl or isopropyl and X₁, X₂, R₂, R₃, and R₄ have the same definition as above. Preferably R₁ is hydrogen or methyl.

In a preferred embodiment of the present invention in the compound of formula (3) X₁ and X₂ are both oxygen, or X₁ is NR₆ and X₂ is oxygen, or X₁ is oxygen and X₂ is NR₆, whereby R₆ is hydrogen or C₁ to C₃ alkyl, preferably hydrogen or methyl, most preferably hydrogen.

In another embodiment of the present invention R₂ in the compound of formula (1), and in particular in the compounds of formula (2) and (3), is a side residue of an amino acid or a protected amino acid side chain. This includes the side chain of glycine, or of l- or d-enantiomers of alanine, valine, leucine, isoleucine, serine or a protected serine, threonine or a protected threonine, lysine or a protected lysine, phenylalanine, tyrosine or a protected tyrosine, tryptophan, cysteine or a protected cysteine, asparagine, glutamine, methionine aspartate, glutamate, lysine, arginine and histidine. Most preferred are valine, leucine, isoleucine, methionine, tryptophan and phenylalanine, in particular, valine, leucine and isoleucine. Such compounds are preferred, especially compounds of formula (2) and (3), wherein R₂ is a side residue of an amino acid or a protected amino acid side chain as defined above and X₁ and X₂ are both oxygen, or X₁ is NR₆ and X₂ is oxygen, or X₁ is oxygen and X₂ is NR₆, whereby R₆ is hydrogen or C₁ to C₃ alkyl, most preferred hydrogen or methyl.

In another embodiment of the present invention in the compound of formula (1), and in particular in the compounds of formula (2) and (3), wherein X₁ and X₂ are both oxygen, or X₁ is NR₆ and X₂ is oxygen, or X₁ is oxygen and X₂ is NR₆, whereby R₆ is hydrogen or C₁ to C₃ alkyl, most preferred hydrogen or methyl, R₂ is linear or branched C₁-C₈ alkyl optionally comprising one or more heteroatoms or cyclopropyl, cyclopentyl, cyclohexyl and 2-, or 3-oxetantyl. Especially preferred R₂ is ethyl, pentyl, isopentyl, hexyl, isohexyl, octyl, methoxy, ethoxy, (CH₂O)₂, cyclopropyl, cyclopentyl, cyclohexyl and 2-, or 3-oxetantyl.

In another preferred embodiment of the present invention R₄ is a 5 or 6 membered heterocycle comprising one or more nitrogen or oxygen atoms selected from the group of 2H-pyran, 4H-pyran, furan, pyrrole, 2-pyridine, 3-pyridine, 4-piridine, pyrazine, pyrimidine, pyridazine, furazan, piperidine, pyrrolidine, piperazine, 2-pyrroline, 3-pyrroline, imidazolidine, 2-imidazoline, 4-imidazoline, pyrazolidine, morpholine, 2-pyrazoline and 3-pyrazoline. Especially preferred are 2-pyridine, 3-pyridine and 4-pyridine, and in particular 3-pyridine resulting in a compound of formula (5)

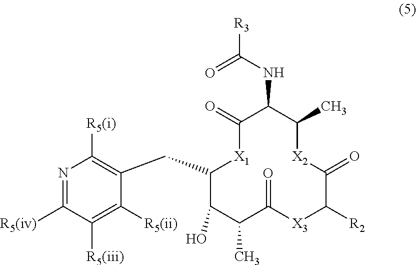

wherein R₅₍ᵢ₎, R₅₍ᵢᵢ₎, R₅₍ᵢᵢᵢ₎ and R₅₍ᵢᵥ₎ are independently from each other hydrogen or fluorine. Preferably R₅₍ᵢ₎, R₅₍ᵢᵢ₎, R₅₍ᵢᵢᵢ₎ and R₅₍ᵢᵥ₎ are all hydrogen or all fluorine.

Especially preferred are compounds of formula (1), and in particular compounds of formula (2) or (3), wherein R₄ is pyridine and all of R₅₍ᵢ₎, R₅₍ᵢᵢ₎, R₅₍ᵢᵢᵢ₎ and R₅₍ᵢᵥ₎ are hydrogen, R₂ is a side residue of an amino acid, or a protected amino acid side chain as defined above in particular the side residue of valine, leucine, isoleucine, methionine, tryptophan and phenylalanine, or is linear or branched C₁-C₈ alkyl optionally comprising one or more heteroatoms, or a cyclopropyl, cyclobutyl or oxetanyl. Especially preferred R₂ is ethyl, pentyl, isopentyl, hexyl, isohexyl, octyl, methoxy, ethoxy, (CH₂O)₂ or or a cyclopropyl, cyclobutyl or oxetanyl,

and wherein X₁ and X₂ are both oxygen, or X₁ is NR₆ and X₂ is oxygen, or X₁ is oxygen and X₂ is NR₆, whereby R₆ is hydrogen or C₁ to C₃ alkyl, most preferred hydrogen or methyl.

In another preferred embodiment of the present invention R₄ is phenyl, resulting in a compound of formula

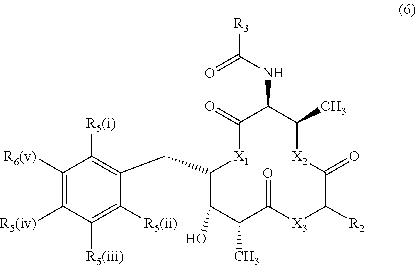

wherein R₅₍ᵢ₎, R₅₍ᵢᵢ₎, R₅₍ᵢᵢᵢ₎, R₅₍ᵢᵥ₎ and R₅₍ᵥ₎ are independently from each other hydrogen or fluorine. Preferably R₅₍ᵢ₎, R₅₍ᵢᵢ₎, R₅₍ᵢᵢᵢ₎, R₅₍ᵢᵥ₎ and R₅₍ᵥ₎ are all hydrogen or all fluorine.

Especially preferred are compounds of formula (1), and in particular compounds of formula (2) or (3), wherein R₄ is phenyl and all of R₅₍ᵢ₎, R₅₍ᵢᵢ₎, R₅₍ᵢᵢᵢ₎, R₅₍ᵢᵥ₎ and R₍ᵥ₎ are hydrogen, R₂ is a side residue of an amino acid or a protected amino acid side chain as defined above, in particular, the side residue of valine, leucine, isoleucine, methionine, tryptophan and phenylalanine, or is linear or branched C₁-C₈ alkyl optionally comprising one or more heteroatoms or a cyclopropyl, cyclobutyl or oxetanyl. Especially preferred R₂ is ethyl, pentyl, isopentyl, hexyl, isohexyl, octyl, methoxy, ethoxy, (CH₂O)₂ or a cyclopropyl, cyclobutyl or oxetanyl,

and wherein X₁ and X₂ are both oxygen, or X₁ is NR₆ and X₂ is oxygen, or X₁ is oxygen and X₂ is NR₆, whereby R₆ is hydrogen or C₁ to C₃ alkyl, most preferred hydrogen or methyl.

In one embodiment of the present invention X₂ represents O, resulting in a compound having the formula 7

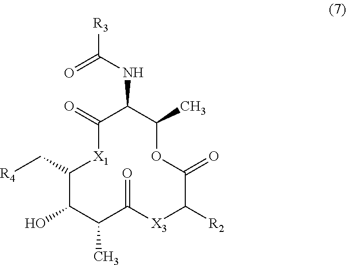

and X₁, X₃, R₂, R₃, R₄ have the same definition as above.

In a preferred embodiment of the present invention in the compound of formula (7) X₁ and X₃ are both oxygen, or X₁ is NR₆ and X₃ is oxygen or X₁ is oxygen and X₃ is NR₆, whereby R₆ is hydrogen or C₁ to C₃ alkyl, most preferred hydrogen or methyl.

In one embodiment of the present invention X₂ represents NR₆, resulting in a compound having the formula (8)

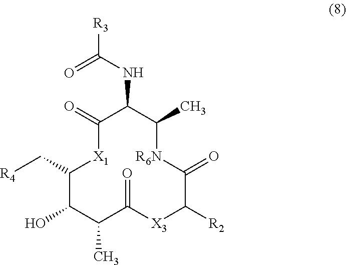

wherein R₆ is hydrogen or a linear or branched C₁ to C₃ alkyl, preferably hydrogen or methyl, most preferably hydrogen and X₁, X₃, R₂, R₃, and R₄ have the same definition as above. Preferably R6 is hydrogen or methyl, most preferably hydrogen.

In a preferred embodiment in the compound of formula (1), and in particular of formula (2) and (3), R₃ is phenyl, pyridyl, 3-hydroxypyridyl, 4-hydroxypyridyl or 5-hydroxypyridyl, whereas said residue may be substituted with fluorine atoms. Most preferred R₃ is a non fluorinated phenyl, a non fluorinated pyridyl or a non fluorinated 3-hydroxypyridyl residue or said residues are completely fluorinated, i.e. 2,3,4,5,6-pentafluorophenyl, 3,4,5,6-tetrapyridyl or 4,5,6-trifluoro-3-hydroxypyridyl.

Alternatively in the compound of formula (1), and in particular of formula (2) and (3), R₃ may be a linear or branched C₁ to C₈ alkyl, optionally comprising a heteroatom or cyclopentyl or cyclohexyl. Preferably R₃ is methyl, ethyl, propyl, isopropyl, pentyl, isopentyl, hexyl, isohexyl, heptyl or octyl, methoxy, ethoxy, (CH₂O)₂, cyclopentyl or cyclohexyl.

In a most preferred embodiment the compound of the present invention have an (R) configuration in position C11 resulting in a compound of formula (1b)

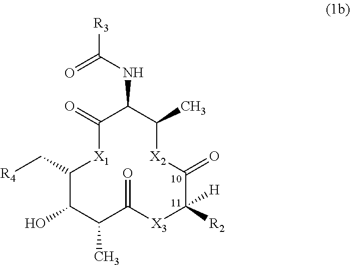

and X₁, X₃, R₂, R₃, R₄, and R₆ have the same definition as above.

In another embodiment the compound of the present invention have an (S) configuration in position C11 resulting in a compound of formula (1a)

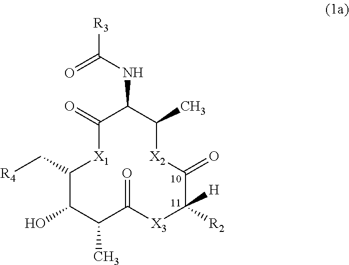

and X₁, X₃, R₂, R₃, R₄ and R₆ have the same definition as above.

For the compound of formula (100 b)

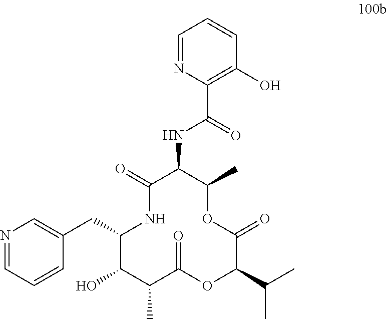 [SMILES: CC(C)[C@H]1OC(=O)[C@H](C)[C@H](O)[C@H](Cc2cccnc2)NC(=O)[C@@H](NC(=O)c2ncccc2O)[C@@H](C)OC1=O]

outstanding results regarding the activity against Mycobacterium tuberculosis (strain H37Rv) could be obtained. The activity pattern is in the same range of inhibitory activity as for pyridomycin. Therefore, the simplification of saturating the compound between C11 and C12 position does not jeopardize the potency of compound 100b in comparison with pyridomycin.

The synthesis of the compound of formula 1 is carried out by coupling of a first general building block X in the form of compound 60 and of a second general building block Y in the form of formula 74a or 74b.

The synthesis of the first general building block X in the form of compound 60 is preferably carried out according to the following reaction schemes:

### Preparation of Aldehyde 57:

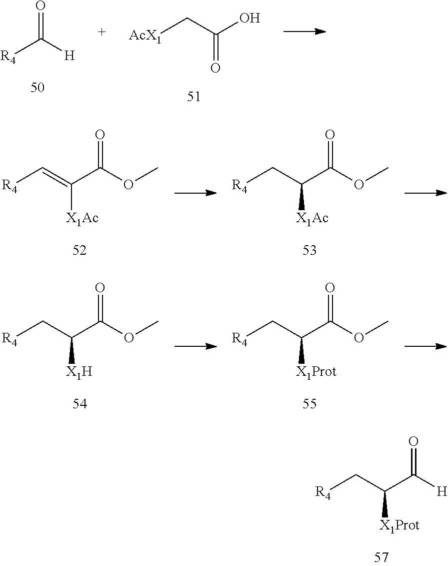

Starting from aldehyde 50, a Knoevenagel-type condensation with protected α-hetero carboxylic acid 51 is carried out. In structure 50, R₄ represents phenyl or 5- or 6-membered heterocycles comprising one or more nitrogen or oxygen atoms optionally substituted with 1 to 4, respectively 5 fluorine atoms, and in structure 51, X₁ is O or NH. The condensation reaction is typically performed by treatment with a suitable base and acetic anhydride at elevated temperature. Subsequent esterification under standard conditions affords α,β-unsaturated ester 52.

Enantioselective hydrogenation of 52, e.g. using [Rh(COD)(R,R-DIPAMP)]BF₄ as a catalyst and HBF₄ as a non-complexing acid, leads to the formation of saturated ester 53. Subsequent removal of the acetate protecting group to give alcohol/amine 54 and reprotection affords protected α-hetero ester 55. Both steps are performed under standard conditions well known to the person skilled in the art. Preferably, the benzyl or silyl ether or dibenzyl amine, respectively, is formed. Ester 55 is then converted to the corresponding aldehyde 57, setting the stage for the subsequent coupling reaction. Aldehyde 57 may be obtained by reduction to the alcohol 56 (not shown), e.g. with LiAlH₄, followed by oxidation to the aldehyde, e.g. with Dess-Martin periodinane. Preferably, aldehyde 57 is not isolated but directly used for the coupling reaction (see below), however the isolation is not sensible from a chemical point of view.

The subsequent coupling en route to the first general building block 60 may be brought about by (a) an anti-selective aldol reaction or (b) a diastereoselective crotylation reaction. The two different pathways are described separately below.

### (a) Via Anti-Aldol Reaction:

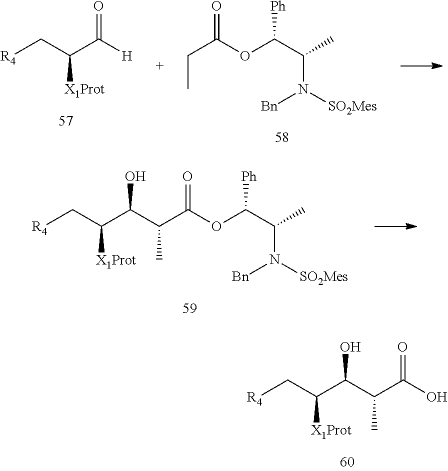

Aldehyde 57 is treated with Masamune auxiliary 58 in order to selectively install the C2-C3 anti-oriented stereocenters via a Masamune anti-aldol reaction (J. Am. Chem. Soc. 1986, 108 (26), 8279-8281). Subsequent removal of the auxiliary under basic conditions affords the desired first general building block 60.

### (b) Via Diastereoselective Crotylation Reaction:

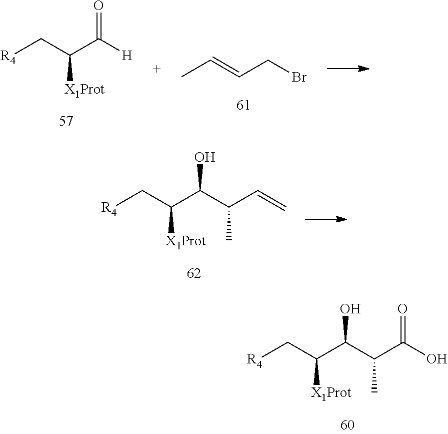

As an alternative to the aldol protocol, it is also possible to access building block 60 via a crotylation reaction. To this end, aldehyde 57 is subjected to standard crotylation conditions with allyl bromide 61 in the presence of a chromium salt, such as CrCl₂. CrCl₂ may be used catalytically with Mn/TMSCl as stoichiometric reducing agents. Crotyl alcohol 62 is thus obtained in high diastereoselectivity.

Subsequently, oxidative cleavage of the terminal double bond of 62 to the aldehyde and subsequent oxidation affords carboxylic acid 60. The oxidative cleavage may be achieved by standard reactions, such as ozonolysis or dihydroxylation and subsequent oxidation. Preferably, crotyl alcohol 61 is subjected to a Sharpless dihydroxylation, followed by double oxidation with NaIO₄ and NaClO₂.

In general, the crotylation pathway tends to be more selective, while the aldol alternative tends to be higher yielding.

The first general building block 60 is a stable intermediate product, which may be stored at room temperature for several weeks.

The synthesis of the second general building block Y in the form of compound 74 (whereas compound 74a is in the S-configuration and compound 74b (not shown) is in the R-configuration) is preferably carried out according to the following scheme:

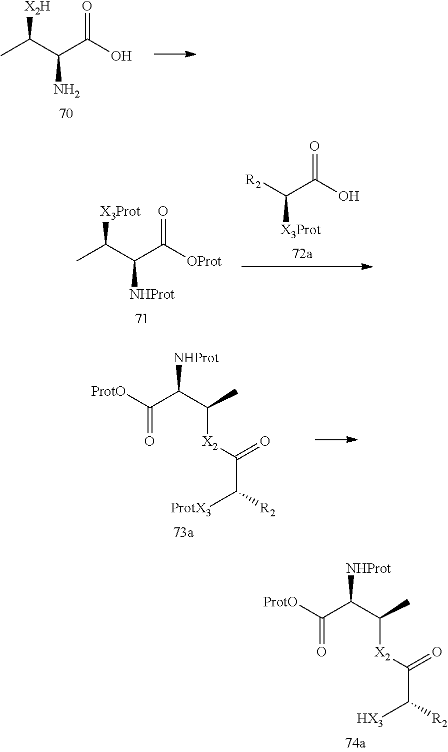

The synthesis of the second general building block 74a starts from α-amino β-hetero butanoic acid 70, wherein X₂ represents O or NR₆, with R₆ being H or a linear or branched alkyl chain having 1 to 3 carbon atoms. First of all, the α-amino group and the carboxyl group of 70 are protected using standard conditions to afford 71. Preferably, the carboxyl group is protected by a benzyl group (Bn), while the α-amino group is preferably protected by tert-butoxycarbonyl (Boc). Typical conditions for such protections are known to the person skilled in the art. If X₂ is nitrogen, the reaction starts with X₂ being an azide. First of all, the second (alpha) amine group is protected by tert-butoxycarbonyl (Boc) (see Angewandte Chemie, International Edition, 47(15), 2844-2848; 2008), followed by Staudinger reduction of the azide group to an amino group.

The protected compound 71 is then treated with carboxylic acid 72a (and for the R-configuration with the carboxylic acid 72b) in order to form the corresponding ester or amide 73a, respectively, depending on X₂.

In carboxylic acid 72a (or in carboxylic acid 72b), X₃ is O or NR₁, wherein R₁ represents hydrogen or C1 to C3 alkyl. 72a (or 72b) is prepared by protection of the corresponding α-amino or α-hydroxy acid. A suitable protecting group for X3=O is silyl and for X3=N a suitable protecting group is Fmoc. Typical conditions for this protection are known to the person skilled in the art. The α-hydroxy acid (i.e. X₃═O) is preferably protected with a silyl protecting group, such as tert-butyldimethylsilyl (TBS), while the α-amino acid is preferably protected with Fmoc.

The coupling reaction between 71 and 72a (or 72b) is performed under standard conditions, which are well known to the person skilled in the art for ester or amide formation, respectively. Preferably, the esterification (i.e. in case X₂═O) is performed using Yamaguchi conditions (B. Chem. Soc. Jpn. 1979, 52 (7), 1989-1993). In the case of an amide formation (i.e. for X₂═NR₆), the reaction is carried out with DCC (dicyclohexylcarboiimide).

Subsequent deprotection of X₃ affords the second general building block 74a (or 74b). Again, the removal of the protecting group is carried out under standard conditions.

The second general building block X in form of compound 74a or 74b is a stable intermediate product, which may be stored for several weeks at room temperature.

The coupling of the two general building blocks 60 and 74a, or 60 and 74b, respectively, is preferably carried out according to the following scheme:

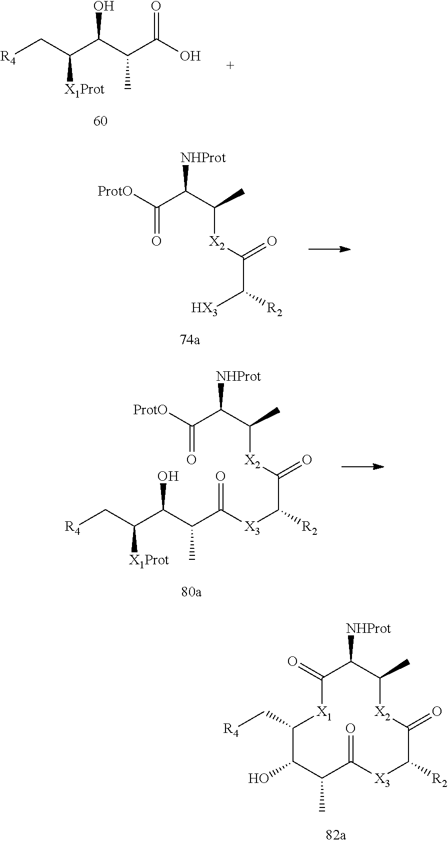

Carboxylic acid 60 is coupled with compound 74a (shown in S-configuration in the above scheme; of course the same procedure applies if compound 74b is in R-configuration) to give the corresponding ester or amide 80a, depending on X₃. Therefore, for X₃═O, an esterification reaction is performed, preferably using Yamaguchi conditions. In the case of X₃═NH, the amide formation is achieved, for instance, by using Yamaguchi conditions as well.

Deprotection of both the terminal ester moiety and X₁ sets the stage for the ring-closing macrolactonisation or macrolactamisation, respectively (depending on X₁), of intermediate 80a. Removal of the protecting groups is achieved by standard conditions. For X₁=0, the macrolactonisation is preferably performed using Yamaguchi macrolactonization (standard procedure), while the macrolatamisation for X₁═NH is preferably achieved by treatment with O-(7-azabenzotriazol-1-yl)-N,N,N′,N′-tetramethyluronium hexafluorophosphate (HATU). This affords macrocyclic compound 82a.

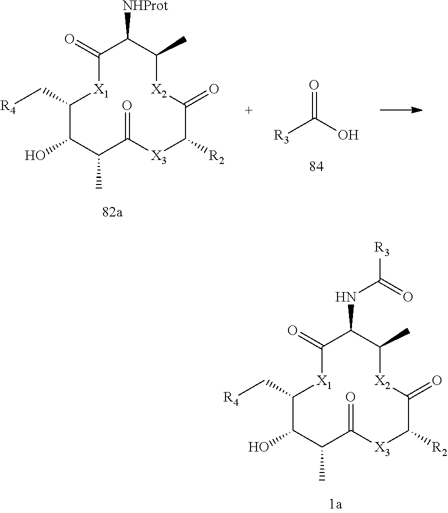

The synthesis of compound 1a is finalized by deprotection of the C7 amino group of 82a resulting in the amine 83a (or 83b) (not shown) and subsequent amide formation with carboxylic acid 84, wherein R₃ is H, cyclopentyl, cyclohexyl, aryl or hydroxyaryl, said aryl or hydroxyaryl being optionally substituted by fluorine, or linear or branched C₁ to C₈ alkyl optionally comprising a hetero atom.

According to a particularly preferred embodiment, X₁ is NH, X₂ is O, X₃ is O, R₂ is iso-propyl, R₃ is 2-(3-hydroxy pyridyl), and R₄ is 3-pyridyl. A preferred synthesis of this preferred product 100 is shown in the following reaction schemes:

The synthesis commences with the preparation of preferred aldehyde 957:

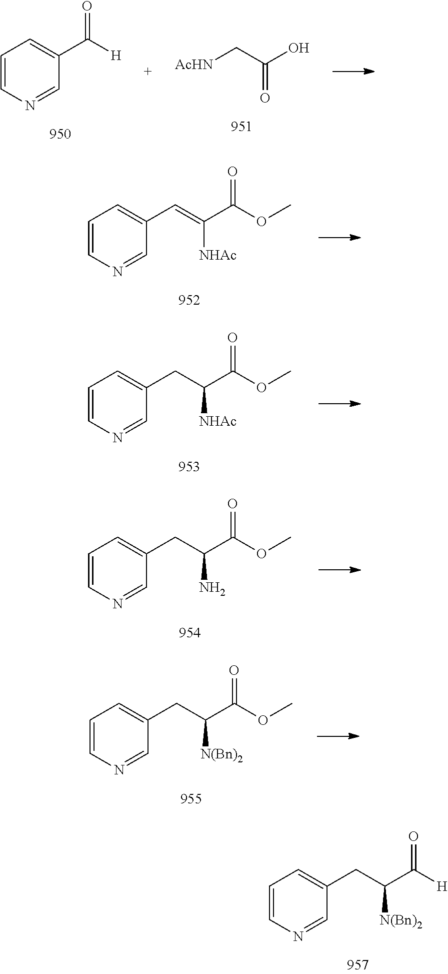

Starting from 3-pyridinecarbocaldehyde 950, a Knoevenagel-type condensation with N-acetylglycine 951 is carried out. Upon treatment with NaOAc and Ac₂O at elevated temperature, followed by esterification with NaOAc and MeOH, exclusively the Z-configured ester 952 is formed. Enantioselective hydrogenation of ester 952 using [Rh(COD)(R,R-DIPAMP)]BF₄ as a catalyst and HBF₄ as a non-complexing acid to protonate the pyridine nitrogen leads to the formation of saturated ester 953. Subsequent removal of the acetate protecting group with SOCl₂ in MeOH to give the free alcohol 954 and reprotection with benzaldehyde in the presence of NaCNBH₃ affords the dibenzyl α-amino ester 955.

Ester 955 is then converted to the corresponding aldehyde 957, setting the stage for the subsequent coupling reaction. Aldehyde 957 is preferably obtained by reduction to the corresponding alcohol using LiAlH₄, followed by oxidation to the aldehyde with Dess-Martin periodinane. Preferably, aldehyde 957 is directly used for the coupling reaction without purification (see below).

The subsequent coupling en route to the preferred first main building block 960 is preferably brought about by an anti-selective Masamune aldol reaction:

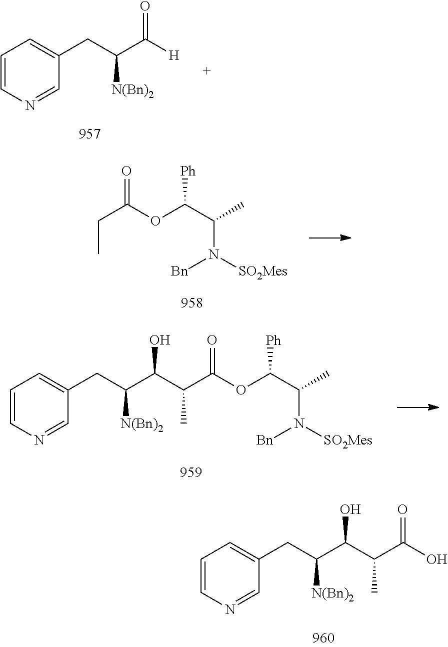

Thus, aldehyde 957 is treated with Masamune auxiliary 958 (J. Am. Chem. Soc. 1986, 108 (26), 8279-8281) in order to selectively install the C2-C3 anti-oriented stereocenters and afford β-hydroxy ester 959. In this reaction, c-Hex₂BOTf is preferably used as Lewis acid. Subsequent removal of the auxiliary under basic conditions using LiOH affords the desired first main building block 960.

Alternatively, it is also possible to prepare acid 960 via a diastereoselective crotylation reaction:

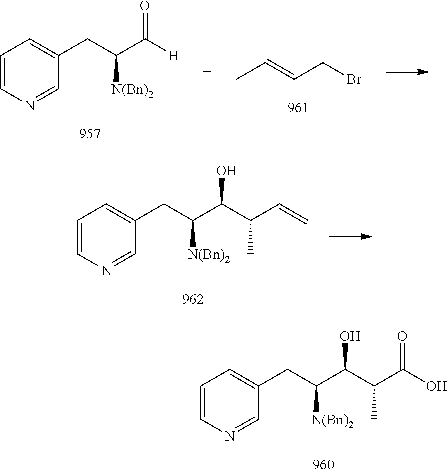

To this end, aldehyde 957 is treated with allyl bromide 961 in the presence of CrCl₂ to afford crotyl alcohol 962 in high diastereoselectivity.

Subsequently, oxidative cleavage of the terminal double bond of 962 to the aldehyde and subsequent oxidation affords carboxylic acid 960. The oxidative cleavage is preferably achieved by a Sharpless dihydroxylation with AD-mix, followed by double oxidation with NaIO₄ and NaClO₂.

The synthesis of the preferred second main building block 974 (whereas compound 974a is in the S-configuration and compound 974b is in the R-configuration (not shown)) is preferably carried out according to the following scheme:

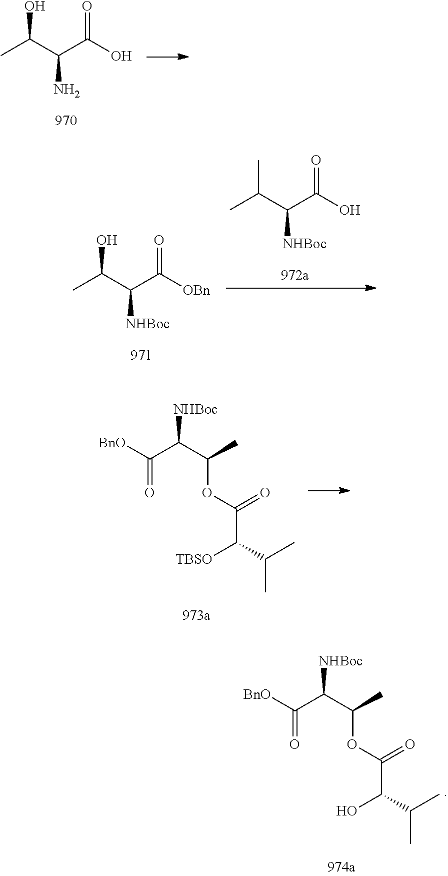

The synthesis of the preferred second general building block 974a starts with the protection of the α-amino group and the carboxyl group of L-threonine 970. Preferably, first the α-amino group is converted to the tert-butoxycarbonyl (Boc) protected amine, e.g. using Boc₂O and a suitable base, such as NaCO₃, followed by treatment with benzyl bromide (BnBr) and a suitable base, such as Cs₂CO₃, to afford benzyl ester 971.

The protected compound 971 is then treated with L-hydroxy isovaleric acid 972a (and for the R-configuration with the D-hydroxy isovaleric acid 972b), wherein the α-hydroxy group is preferably protect with tert-butyldimethylsilyl (TBS), in order to form the corresponding ester 973a (or 973b). The coupling reaction between alcohol 971 and carboxylic acid 972a (or 972b) is preferably achieved by using Yamaguchi conditions (B. Chem. Soc. Jpn. 1979, 52 (7), 1989-1993). Subsequent deprotection of the TBS ether, e.g. by treatment with HF•pyridine affords the preferred second main building block 974a (or 974b).

The coupling of the two preferred main building blocks carboxylic acid 960 and alcohol 974a or carbocylic acid 960 and alcohol 974b is preferably carried out according to the following scheme:

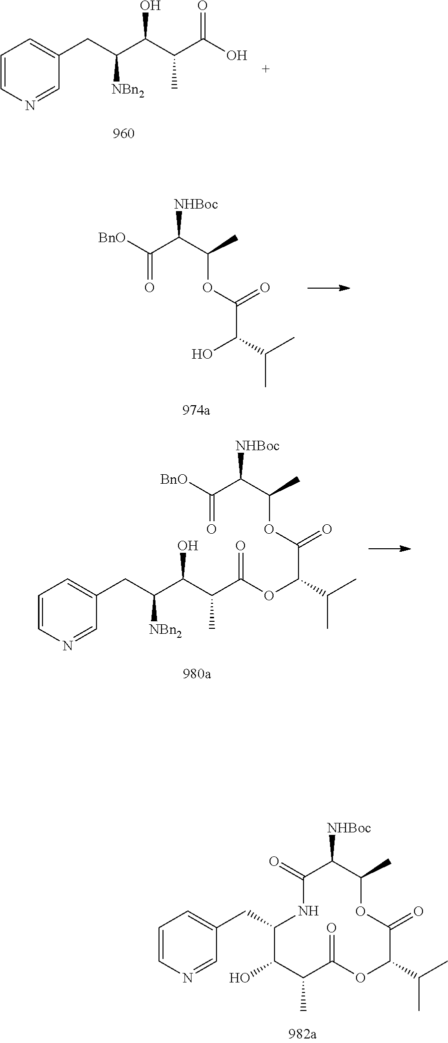

Carboxylic acid 960 is preferably esterified with alcohol 974a (shown is S-configuration in the above scheme; of course the same applies if alcohol 974b is in R-configuration) by treating the acid 960 with 2,4,6-trichlorobenzoyl chloride at low temperatures and subsequent simultaneous addition of the alcohol 974a (or 974b) and DMAP (4-dimethylamino-pyridine) to afford ester 980a (or 980b).

Simultaneous deprotection of both the terminal ester moiety and the C4-amino group sets the stage for the ring-closing macrolactamisation. Thus, intermediate 980a (or 980b) is treated with H₂ and Pd/C in order to remove the benzyl groups.

The key macrolactamisation is then preferably performed by treatment with HATU at high dilution, delivering depsipeptide 982a (or 982b) in high yield.

The synthesis of final compound 100a (or 100b) is finalized by deprotection of the C7 amino group of depsipeptide 982a (or 982b) resulting in the amine 983a (or 983b) (not shown), e.g. using TFA (trifluoroacetic acid) and subsequent coupling with 3-hydroxypyridine-2-carboxylic acid 984. This amide formation is preferably brought about by treatment with HATU and DIPEA (N,N-diisopropylethylamine).

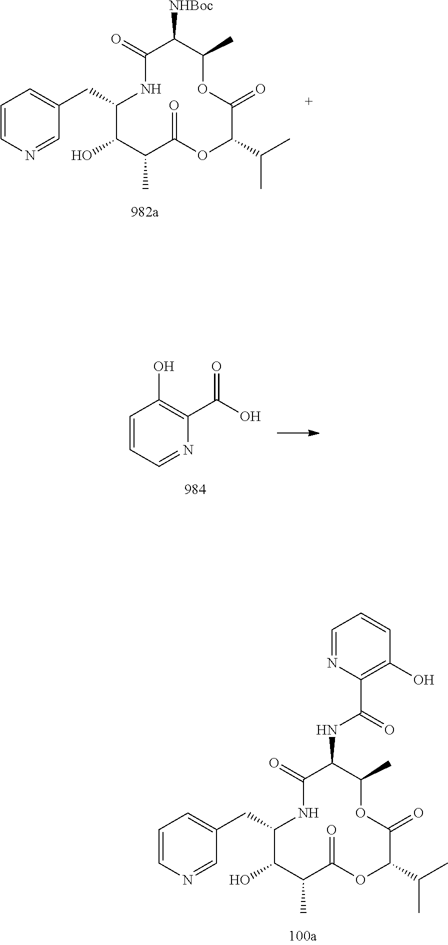 [SMILES: CC(C)[C@@H]1OC(=O)[C@H](C)[C@H](O)[C@H](Cc2cccnc2)NC(=O)[C@@H](NC(=O)OC(C)(C)C)[C@@H](C)OC1=O ; O=C(O)c1ncccc1O ; CC(C)[C@@H]1OC(=O)[C@H](C)[C@H](O)[C@H](Cc2cccnc2)NC(=O)[C@@H](NC(=O)c2ncccc2O)[C@@H](C)OC1=O]

Preferably the compound of the present invention is selected from the group of the compounds following below, with said list including the C11 (R) and (S) diastereomers of said compounds as well as mixtures thereof.

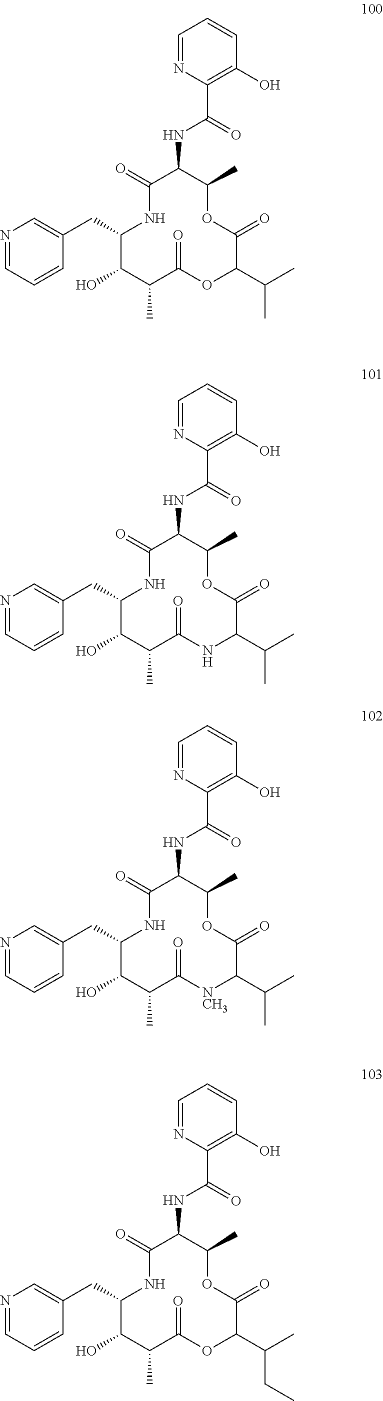 [SMILES: CC(C)C1OC(=O)[C@H](C)[C@H](O)[C@H](Cc2cccnc2)NC(=O)[C@@H](NC(=O)c2ncccc2O)[C@@H](C)OC1=O ; CC(C)C1NC(=O)[C@H](C)[C@H](O)[C@H](Cc2cccnc2)NC(=O)[C@@H](NC(=O)c2ncccc2O)[C@@H](C)OC1=O ; CC(C)C1C(=O)O[C@H](C)[C@H](NC(=O)c2ncccc2O)C(=O)N[C@@H](Cc2cccnc2)[C@@H](O)[C@@H](C)C(=O)N1C ; CCC(C)C1OC(=O)[C@H](C)[C@H](O)[C@H](Cc2cccnc2)NC(=O)[C@@H](NC(=O)c2ncccc2O)[C@@H](C)OC1=O]

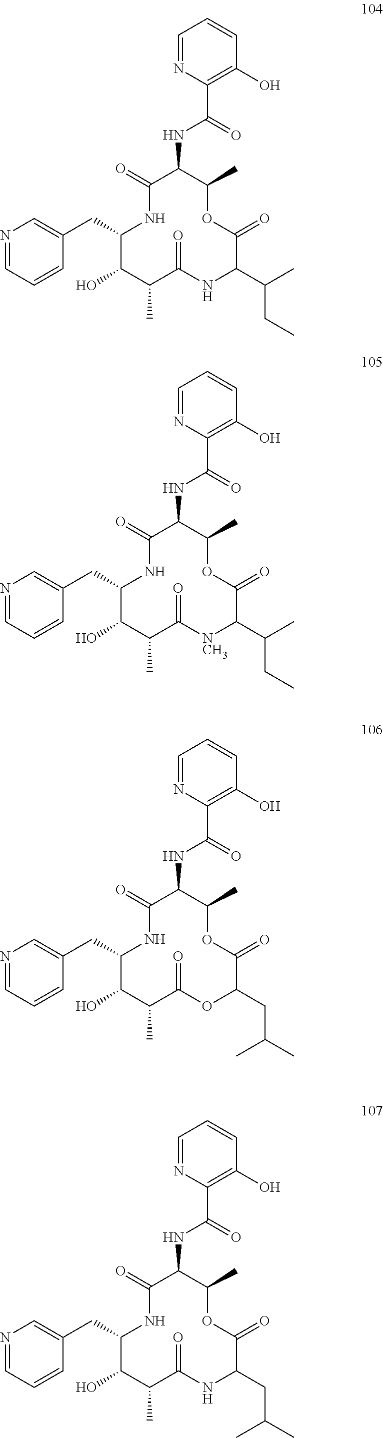 [SMILES: CCC(C)C1NC(=O)[C@H](C)[C@H](O)[C@H](Cc2cccnc2)NC(=O)[C@@H](NC(=O)c2ncccc2O)[C@@H](C)OC1=O ; CCC(C)C1C(=O)O[C@H](C)[C@H](NC(=O)c2ncccc2O)C(=O)N[C@@H](Cc2cccnc2)[C@@H](O)[C@@H](C)C(=O)N1C ; CC(C)CC1OC(=O)[C@H](C)[C@H](O)[C@H](Cc2cccnc2)NC(=O)[C@@H](NC(=O)c2ncccc2O)[C@@H](C)OC1=O ; CC(C)CC1NC(=O)[C@H](C)[C@H](O)[C@H](Cc2cccnc2)NC(=O)[C@@H](NC(=O)c2ncccc2O)[C@@H](C)OC1=O]

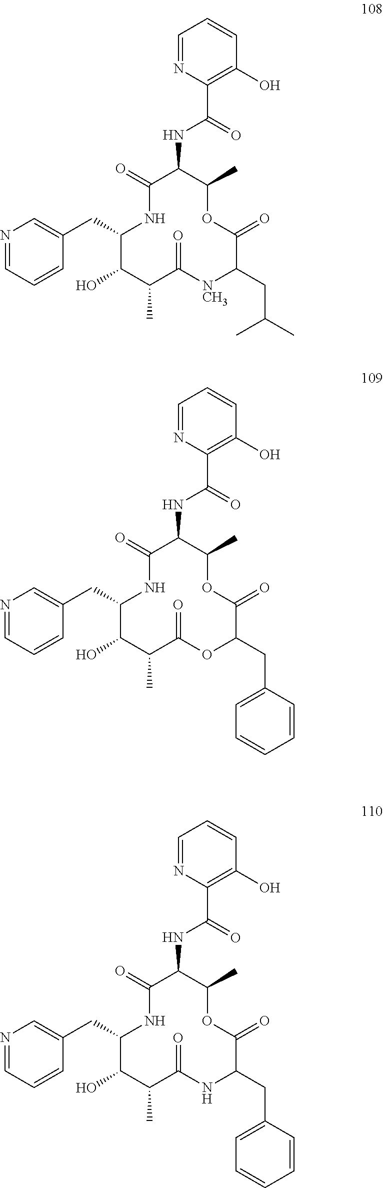 [SMILES: CC(C)CC1C(=O)O[C@H](C)[C@H](NC(=O)c2ncccc2O)C(=O)N[C@@H](Cc2cccnc2)[C@@H](O)[C@@H](C)C(=O)N1C ; C[C@H]1OC(=O)C(Cc2ccccc2)OC(=O)[C@H](C)[C@H](O)[C@H](Cc2cccnc2)NC(=O)[C@H]1NC(=O)c1ncccc1O ; C[C@H]1OC(=O)C(Cc2ccccc2)NC(=O)[C@H](C)[C@H](O)[C@H](Cc2cccnc2)NC(=O)[C@H]1NC(=O)c1ncccc1O]

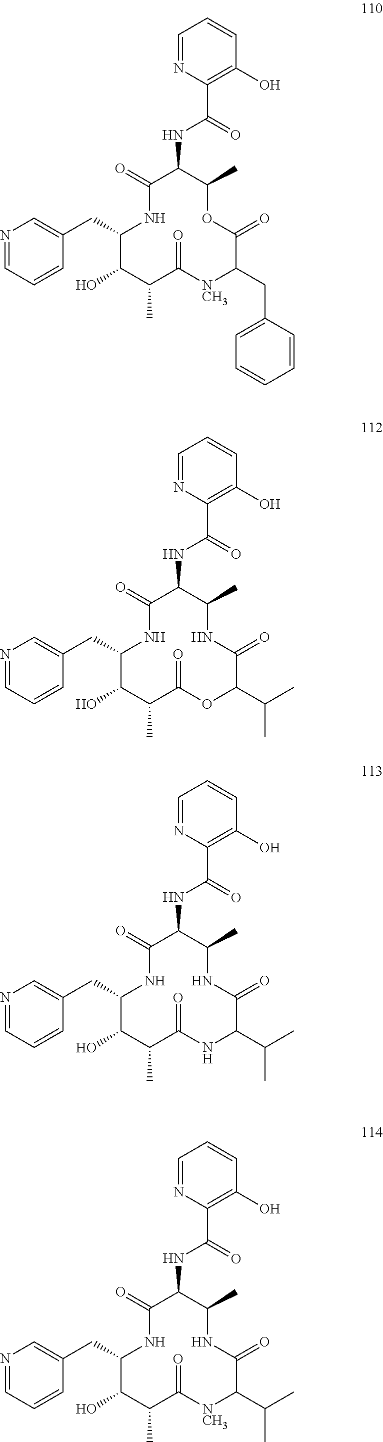 [SMILES: C[C@H]1OC(=O)C(Cc2ccccc2)N(C)C(=O)[C@H](C)[C@H](O)[C@H](Cc2cccnc2)NC(=O)[C@H]1NC(=O)c1ncccc1O ; CC(C)C1OC(=O)[C@H](C)[C@H](O)[C@H](Cc2cccnc2)NC(=O)[C@@H](NC(=O)c2ncccc2O)[C@@H](C)NC1=O ; CC(C)C1NC(=O)[C@H](C)[C@H](O)[C@H](Cc2cccnc2)NC(=O)[C@@H](NC(=O)c2ncccc2O)[C@@H](C)NC1=O ; CC(C)C1C(=O)N[C@H](C)[C@H](NC(=O)c2ncccc2O)C(=O)N[C@@H](Cc2cccnc2)[C@@H](O)[C@@H](C)C(=O)N1C]

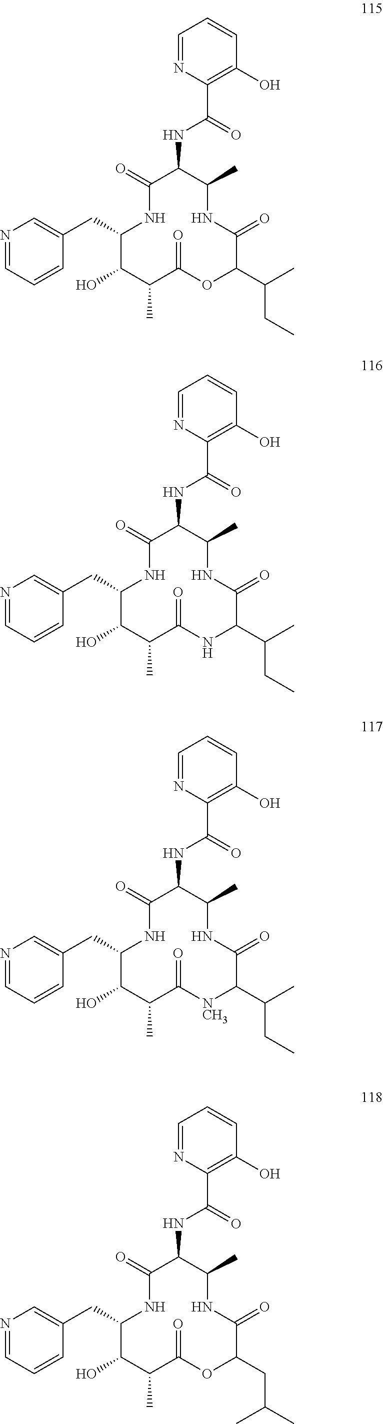 [SMILES: CCC(C)C1OC(=O)[C@H](C)[C@H](O)[C@H](Cc2cccnc2)NC(=O)[C@@H](NC(=O)c2ncccc2O)[C@@H](C)NC1=O ; CCC(C)C1NC(=O)[C@H](C)[C@H](O)[C@H](Cc2cccnc2)NC(=O)[C@@H](NC(=O)c2ncccc2O)[C@@H](C)NC1=O ; CCC(C)C1C(=O)N[C@H](C)[C@H](NC(=O)c2ncccc2O)C(=O)N[C@@H](Cc2cccnc2)[C@@H](O)[C@@H](C)C(=O)N1C ; CC(C)CC1OC(=O)[C@H](C)[C@H](O)[C@H](Cc2cccnc2)NC(=O)[C@@H](NC(=O)c2ncccc2O)[C@@H](C)NC1=O]

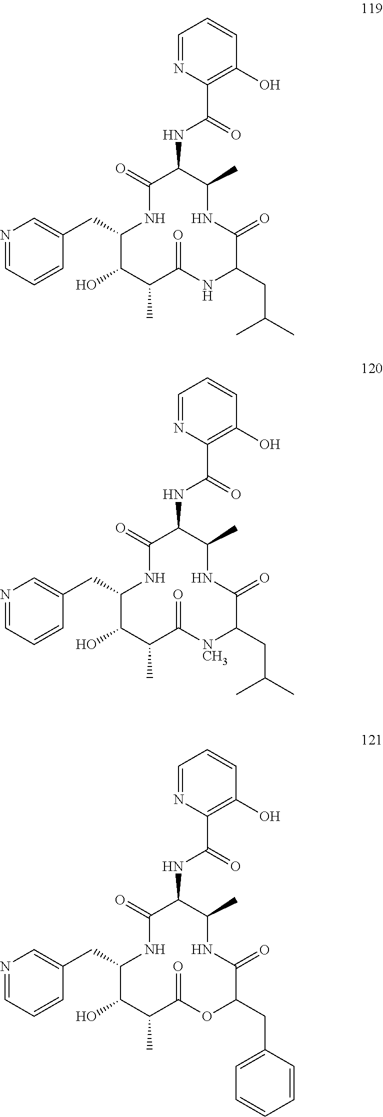 [SMILES: CC(C)CC1NC(=O)[C@H](C)[C@H](O)[C@H](Cc2cccnc2)NC(=O)[C@@H](NC(=O)c2ncccc2O)[C@@H](C)NC1=O ; CC(C)CC1C(=O)N[C@H](C)[C@H](NC(=O)c2ncccc2O)C(=O)N[C@@H](Cc2cccnc2)[C@@H](O)[C@@H](C)C(=O)N1C ; C[C@H]1NC(=O)C(Cc2ccccc2)OC(=O)[C@H](C)[C@H](O)[C@H](Cc2cccnc2)NC(=O)[C@H]1NC(=O)c1ncccc1O]

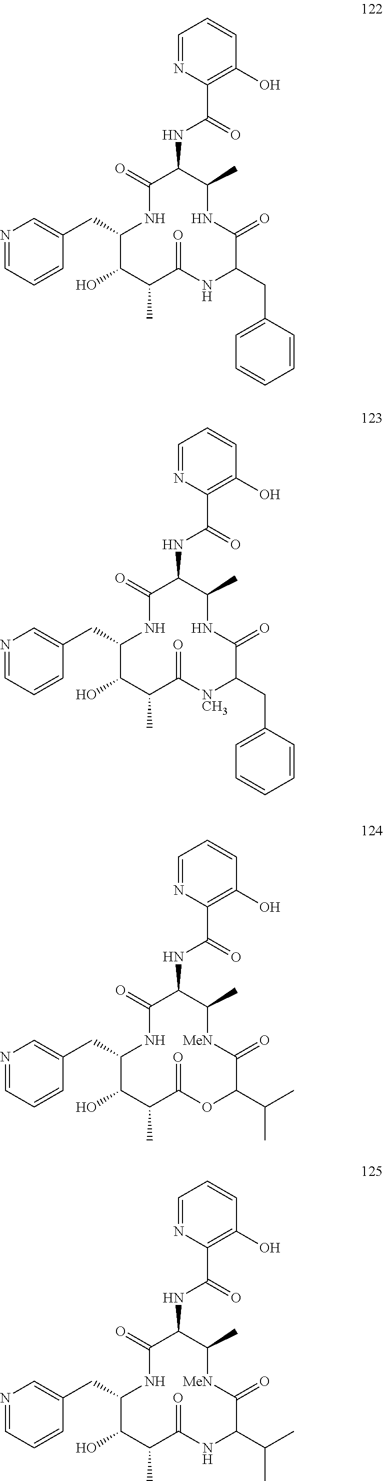 [SMILES: C[C@H]1NC(=O)C(Cc2ccccc2)NC(=O)[C@H](C)[C@H](O)[C@H](Cc2cccnc2)NC(=O)[C@H]1NC(=O)c1ncccc1O ; C[C@H]1NC(=O)C(Cc2ccccc2)N(C)C(=O)[C@H](C)[C@H](O)[C@H](Cc2cccnc2)NC(=O)[C@H]1NC(=O)c1ncccc1O ; CC(C)C1OC(=O)[C@H](C)[C@H](O)[C@H](Cc2cccnc2)NC(=O)[C@@H](NC(=O)c2ncccc2O)[C@@H](C)N(C)C1=O ; CC(C)C1NC(=O)[C@H](C)[C@H](O)[C@H](Cc2cccnc2)NC(=O)[C@@H](NC(=O)c2ncccc2O)[C@@H](C)N(C)C1=O]

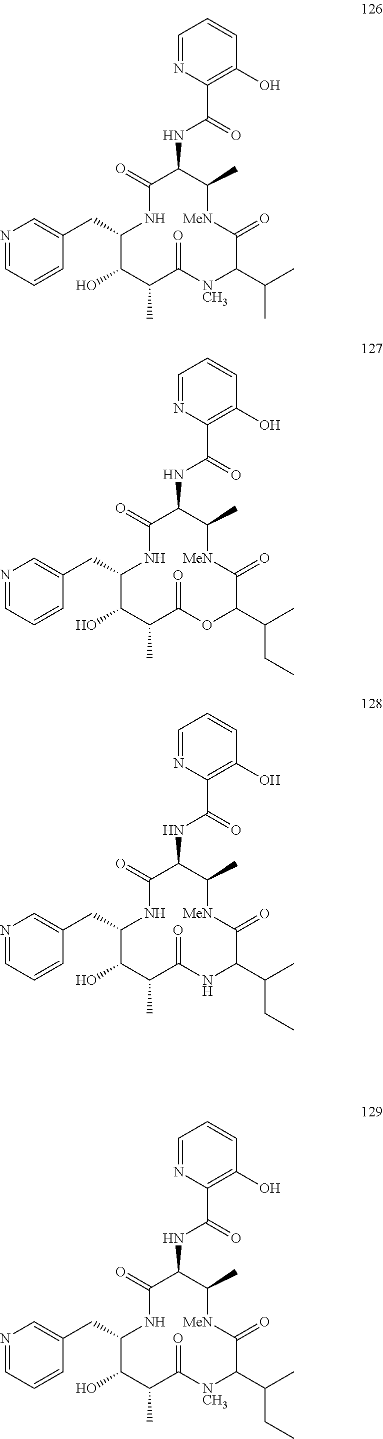 [SMILES: CC(C)C1C(=O)N(C)[C@H](C)[C@H](NC(=O)c2ncccc2O)C(=O)N[C@@H](Cc2cccnc2)[C@@H](O)[C@@H](C)C(=O)N1C ; CCC(C)C1OC(=O)[C@H](C)[C@H](O)[C@H](Cc2cccnc2)NC(=O)[C@@H](NC(=O)c2ncccc2O)[C@@H](C)N(C)C1=O ; CCC(C)C1NC(=O)[C@H](C)[C@H](O)[C@H](Cc2cccnc2)NC(=O)[C@@H](NC(=O)c2ncccc2O)[C@@H](C)N(C)C1=O ; CCC(C)C1C(=O)N(C)[C@H](C)[C@H](NC(=O)c2ncccc2O)C(=O)N[C@@H](Cc2cccnc2)[C@@H](O)[C@@H](C)C(=O)N1C]

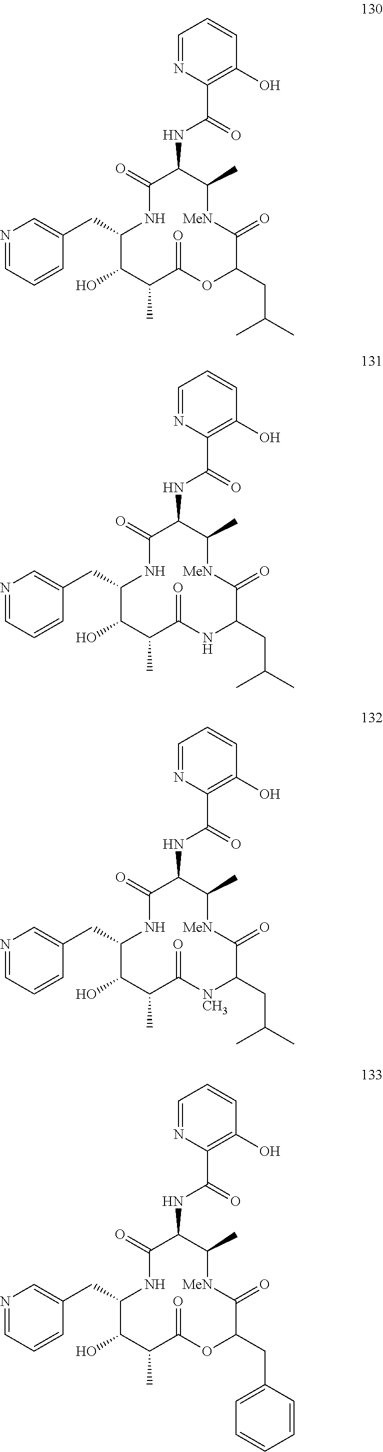

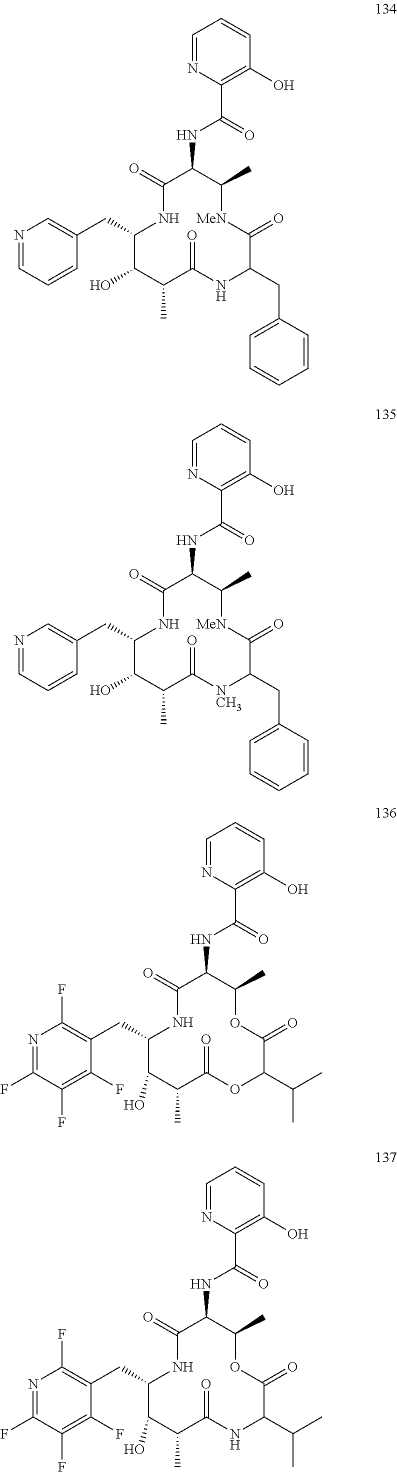 [SMILES: C[C@@H]1[C@H](NC(=O)c2ncccc2O)C(=O)N[C@@H](Cc2cccnc2)[C@@H](O)[C@@H](C)C(=O)NC(Cc2ccccc2)C(=O)N1C ; C[C@@H]1[C@H](NC(=O)c2ncccc2O)C(=O)N[C@@H](Cc2cccnc2)[C@@H](O)[C@@H](C)C(=O)N(C)C(Cc2ccccc2)C(=O)N1C ; CC(C)C1OC(=O)[C@H](C)[C@H](O)[C@H](Cc2c(F)nc(F)c(F)c2F)NC(=O)[C@@H](NC(=O)c2ncccc2O)[C@@H](C)OC1=O ; CC(C)C1NC(=O)[C@H](C)[C@H](O)[C@H](Cc2c(F)nc(F)c(F)c2F)NC(=O)[C@@H](NC(=O)c2ncccc2O)[C@@H](C)OC1=O]

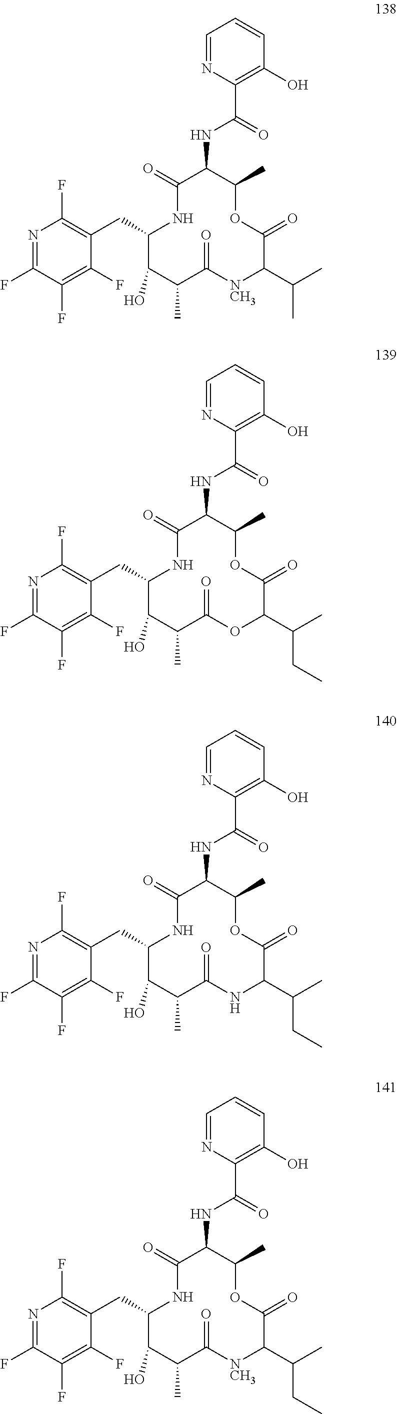 [SMILES: CC(C)C1C(=O)O[C@H](C)[C@H](NC(=O)c2ncccc2O)C(=O)N[C@@H](Cc2c(F)nc(F)c(F)c2F)[C@@H](O)[C@@H](C)C(=O)N1C ; CCC(C)C1OC(=O)[C@H](C)[C@H](O)[C@H](Cc2c(F)nc(F)c(F)c2F)NC(=O)[C@@H](NC(=O)c2ncccc2O)[C@@H](C)OC1=O ; CCC(C)C1NC(=O)[C@H](C)[C@H](O)[C@H](Cc2c(F)nc(F)c(F)c2F)NC(=O)[C@@H](NC(=O)c2ncccc2O)[C@@H](C)OC1=O ; CCC(C)C1C(=O)O[C@H](C)[C@H](NC(=O)c2ncccc2O)C(=O)N[C@@H](Cc2c(F)nc(F)c(F)c2F)[C@@H](O)[C@@H](C)C(=O)N1C]

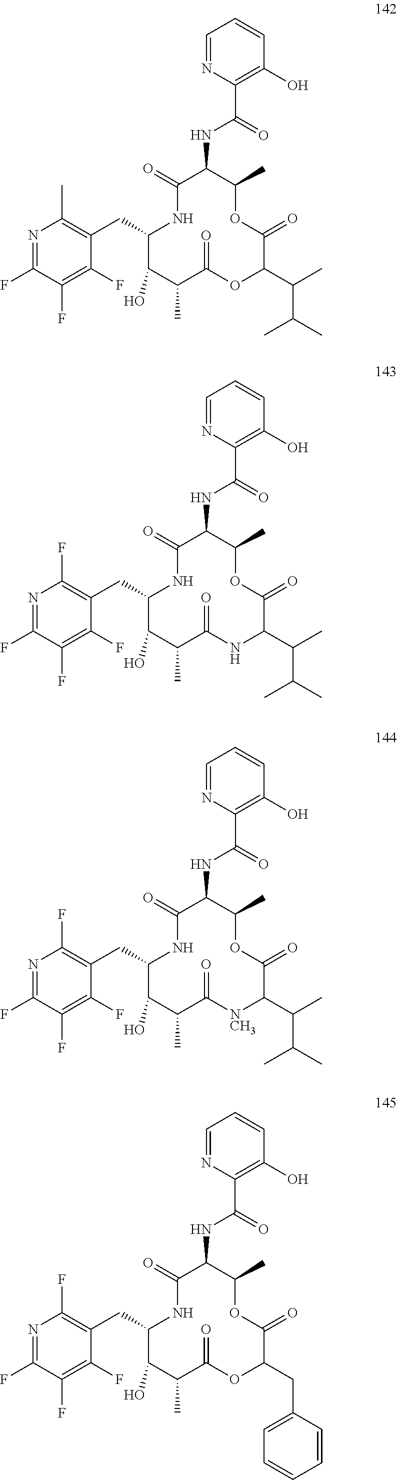 [SMILES: Cc1nc(F)c(F)c(F)c1C[C@@H]1NC(=O)[C@@H](NC(=O)c2ncccc2O)[C@@H](C)OC(=O)C(C(C)C(C)C)OC(=O)[C@H](C)[C@@H]1O ; CC(C)C(C)C1NC(=O)[C@H](C)[C@H](O)[C@H](Cc2c(F)nc(F)c(F)c2F)NC(=O)[C@@H](NC(=O)c2ncccc2O)[C@@H](C)OC1=O ; CC(C)C(C)C1C(=O)O[C@H](C)[C@H](NC(=O)c2ncccc2O)C(=O)N[C@@H](Cc2c(F)nc(F)c(F)c2F)[C@@H](O)[C@@H](C)C(=O)N1C ; C[C@H]1OC(=O)C(Cc2ccccc2)OC(=O)[C@H](C)[C@H](O)[C@H](Cc2c(F)nc(F)c(F)c2F)NC(=O)[C@H]1NC(=O)c1ncccc1O]

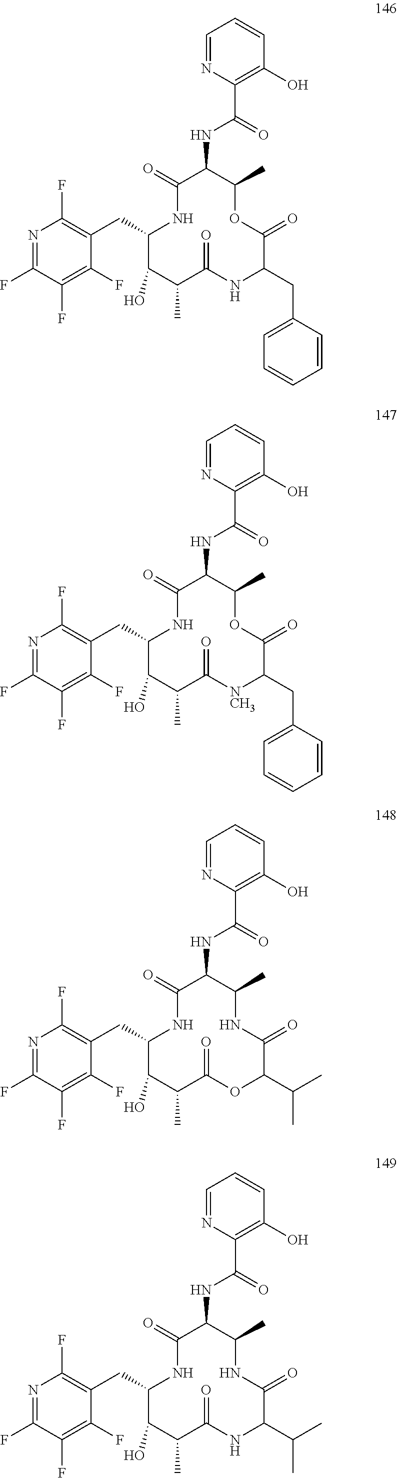 [SMILES: C[C@H]1OC(=O)C(Cc2ccccc2)NC(=O)[C@H](C)[C@H](O)[C@H](Cc2c(F)nc(F)c(F)c2F)NC(=O)[C@H]1NC(=O)c1ncccc1O ; C[C@H]1OC(=O)C(Cc2ccccc2)N(C)C(=O)[C@H](C)[C@H](O)[C@H](Cc2c(F)nc(F)c(F)c2F)NC(=O)[C@H]1NC(=O)c1ncccc1O ; CC(C)C1OC(=O)[C@H](C)[C@H](O)[C@H](Cc2c(F)nc(F)c(F)c2F)NC(=O)[C@@H](NC(=O)c2ncccc2O)[C@@H](C)NC1=O ; CC(C)C1NC(=O)[C@H](C)[C@H](O)[C@H](Cc2c(F)nc(F)c(F)c2F)NC(=O)[C@@H](NC(=O)c2ncccc2O)[C@@H](C)NC1=O]

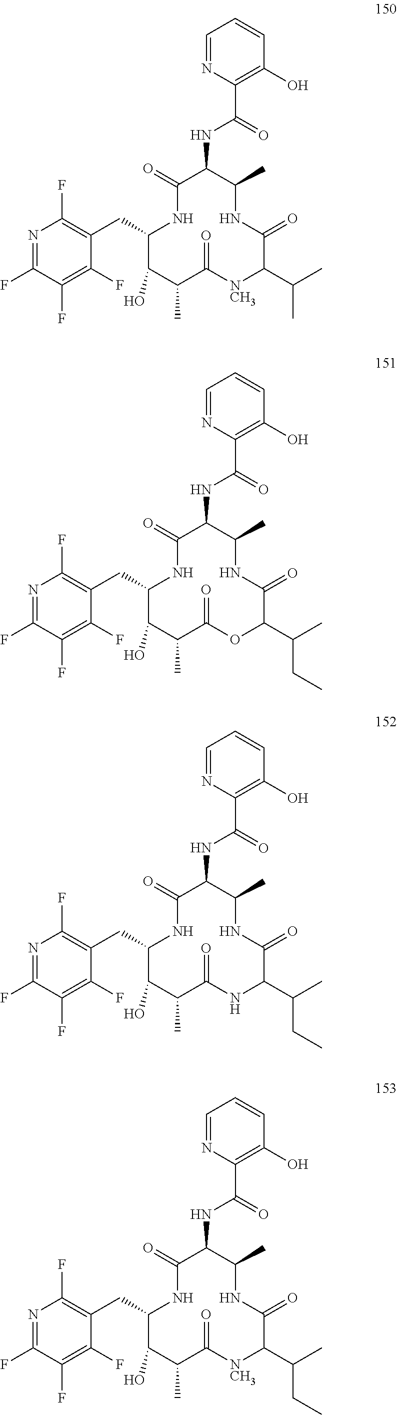 [SMILES: CC(C)C1C(=O)N[C@H](C)[C@H](NC(=O)c2ncccc2O)C(=O)N[C@@H](Cc2c(F)nc(F)c(F)c2F)[C@@H](O)[C@@H](C)C(=O)N1C ; CCC(C)C1OC(=O)[C@H](C)[C@H](O)[C@H](Cc2c(F)nc(F)c(F)c2F)NC(=O)[C@@H](NC(=O)c2ncccc2O)[C@@H](C)NC1=O ; CCC(C)C1NC(=O)[C@H](C)[C@H](O)[C@H](Cc2c(F)nc(F)c(F)c2F)NC(=O)[C@@H](NC(=O)c2ncccc2O)[C@@H](C)NC1=O ; CCC(C)C1C(=O)N[C@H](C)[C@H](NC(=O)c2ncccc2O)C(=O)N[C@@H](Cc2c(F)nc(F)c(F)c2F)[C@@H](O)[C@@H](C)C(=O)N1C]

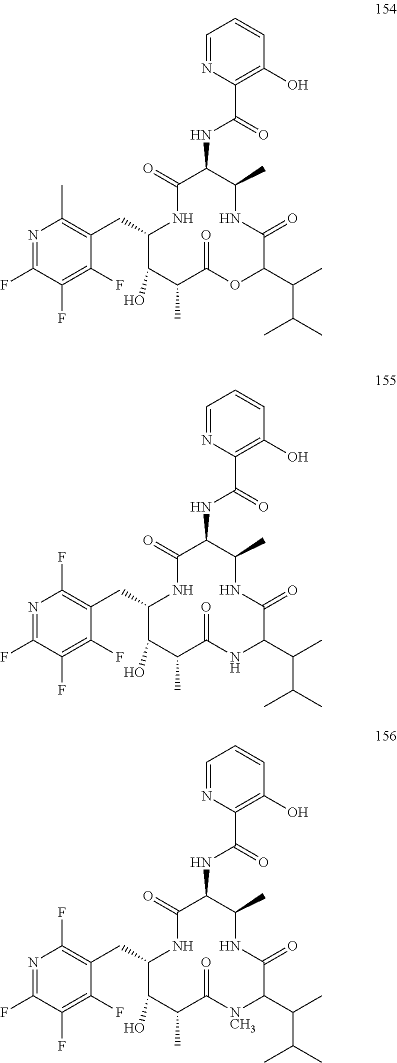 [SMILES: Cc1nc(F)c(F)c(F)c1C[C@@H]1NC(=O)[C@@H](NC(=O)c2ncccc2O)[C@@H](C)NC(=O)C(C(C)C(C)C)OC(=O)[C@H](C)[C@@H]1O ; CC(C)C(C)C1NC(=O)[C@H](C)[C@H](O)[C@H](Cc2c(F)nc(F)c(F)c2F)NC(=O)[C@@H](NC(=O)c2ncccc2O)[C@@H](C)NC1=O ; CC(C)C(C)C1C(=O)N[C@H](C)[C@H](NC(=O)c2ncccc2O)C(=O)N[C@@H](Cc2c(F)nc(F)c(F)c2F)[C@@H](O)[C@@H](C)C(=O)N1C]

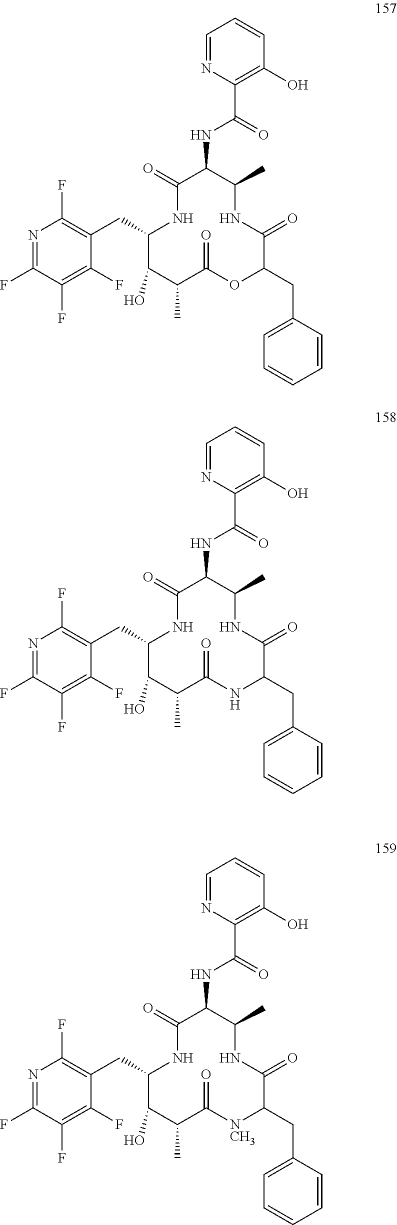 [SMILES: C[C@H]1NC(=O)C(Cc2ccccc2)OC(=O)[C@H](C)[C@H](O)[C@H](Cc2c(F)nc(F)c(F)c2F)NC(=O)[C@H]1NC(=O)c1ncccc1O ; C[C@H]1NC(=O)C(Cc2ccccc2)NC(=O)[C@H](C)[C@H](O)[C@H](Cc2c(F)nc(F)c(F)c2F)NC(=O)[C@H]1NC(=O)c1ncccc1O ; C[C@H]1NC(=O)C(Cc2ccccc2)N(C)C(=O)[C@H](C)[C@H](O)[C@H](Cc2c(F)nc(F)c(F)c2F)NC(=O)[C@H]1NC(=O)c1ncccc1O]

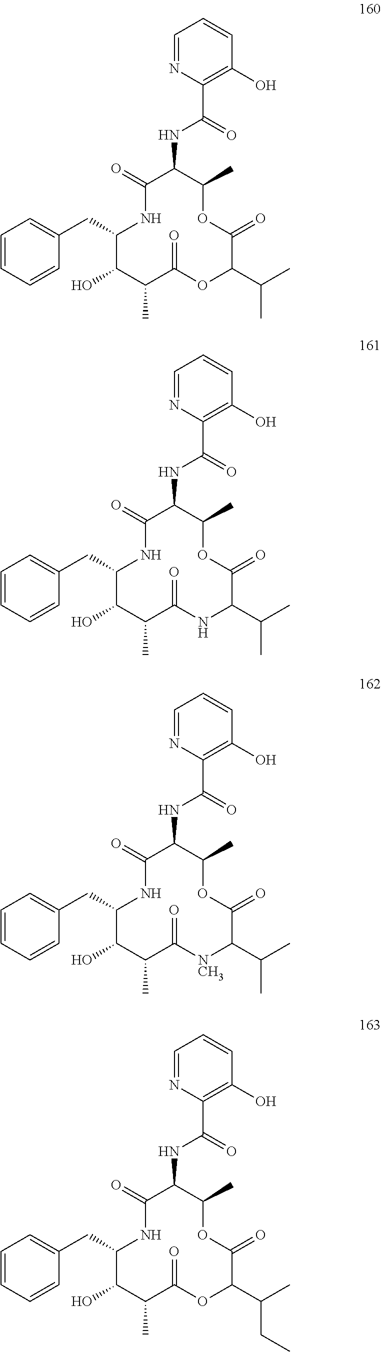 [SMILES: CC(C)C1OC(=O)[C@H](C)[C@H](O)[C@H](Cc2ccccc2)NC(=O)[C@@H](NC(=O)c2ncccc2O)[C@@H](C)OC1=O ; CC(C)C1NC(=O)[C@H](C)[C@H](O)[C@H](Cc2ccccc2)NC(=O)[C@@H](NC(=O)c2ncccc2O)[C@@H](C)OC1=O ; CC(C)C1C(=O)O[C@H](C)[C@H](NC(=O)c2ncccc2O)C(=O)N[C@@H](Cc2ccccc2)[C@@H](O)[C@@H](C)C(=O)N1C ; CCC(C)C1OC(=O)[C@H](C)[C@H](O)[C@H](Cc2ccccc2)NC(=O)[C@@H](NC(=O)c2ncccc2O)[C@@H](C)OC1=O]

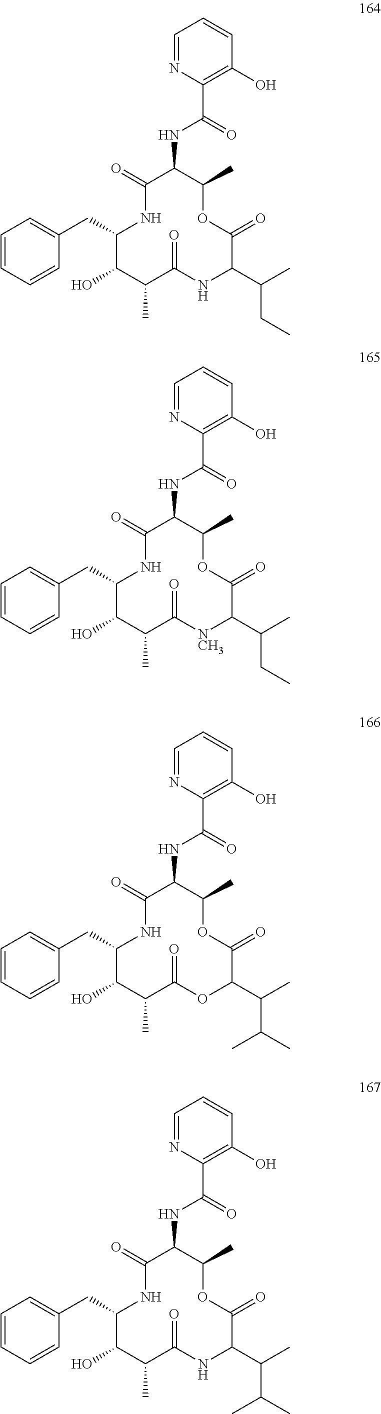 [SMILES: CCC(C)C1NC(=O)[C@H](C)[C@H](O)[C@H](Cc2ccccc2)NC(=O)[C@@H](NC(=O)c2ncccc2O)[C@@H](C)OC1=O ; CCC(C)C1C(=O)O[C@H](C)[C@H](NC(=O)c2ncccc2O)C(=O)N[C@@H](Cc2ccccc2)[C@@H](O)[C@@H](C)C(=O)N1C ; CC(C)C(C)C1OC(=O)[C@H](C)[C@H](O)[C@H](Cc2ccccc2)NC(=O)[C@@H](NC(=O)c2ncccc2O)[C@@H](C)OC1=O ; CC(C)C(C)C1NC(=O)[C@H](C)[C@H](O)[C@H](Cc2ccccc2)NC(=O)[C@@H](NC(=O)c2ncccc2O)[C@@H](C)OC1=O]

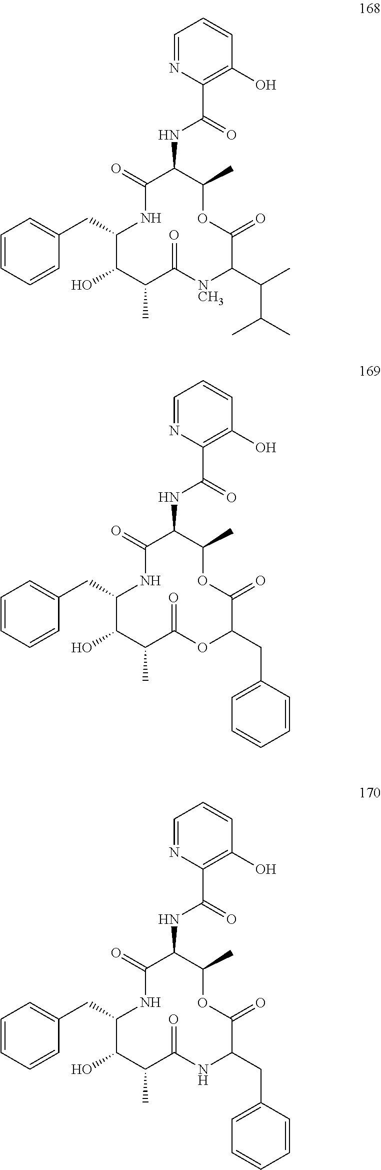 [SMILES: CC(C)C(C)C1C(=O)O[C@H](C)[C@H](NC(=O)c2ncccc2O)C(=O)N[C@@H](Cc2ccccc2)[C@@H](O)[C@@H](C)C(=O)N1C ; C[C@H]1OC(=O)C(Cc2ccccc2)OC(=O)[C@H](C)[C@H](O)[C@H](Cc2ccccc2)NC(=O)[C@H]1NC(=O)c1ncccc1O ; C[C@H]1OC(=O)C(Cc2ccccc2)NC(=O)[C@H](C)[C@H](O)[C@H](Cc2ccccc2)NC(=O)[C@H]1NC(=O)c1ncccc1O]

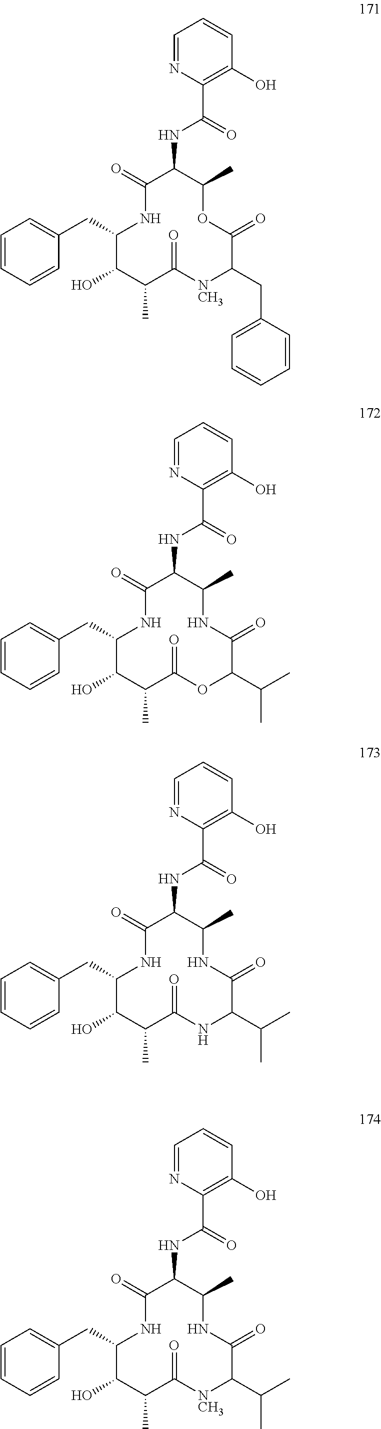 [SMILES: C[C@H]1OC(=O)C(Cc2ccccc2)N(C)C(=O)[C@H](C)[C@H](O)[C@H](Cc2ccccc2)NC(=O)[C@H]1NC(=O)c1ncccc1O ; CC(C)C1OC(=O)[C@H](C)[C@H](O)[C@H](Cc2ccccc2)NC(=O)[C@@H](NC(=O)c2ncccc2O)[C@@H](C)NC1=O ; CC(C)C1NC(=O)[C@H](C)[C@H](O)[C@H](Cc2ccccc2)NC(=O)[C@@H](NC(=O)c2ncccc2O)[C@@H](C)NC1=O ; CC(C)C1C(=O)N[C@H](C)[C@H](NC(=O)c2ncccc2O)C(=O)N[C@@H](Cc2ccccc2)[C@@H](O)[C@@H](C)C(=O)N1C]

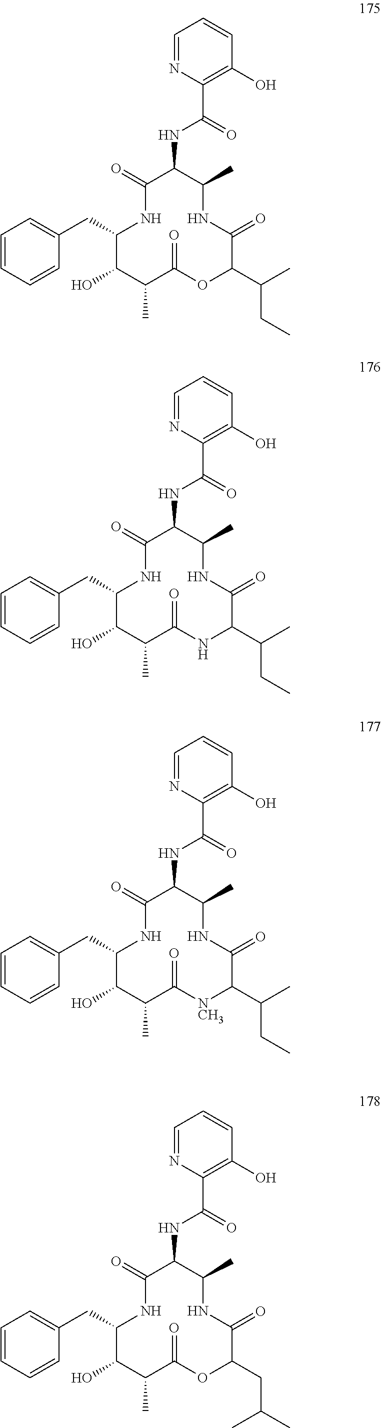 [SMILES: CCC(C)C1OC(=O)[C@H](C)[C@H](O)[C@H](Cc2ccccc2)NC(=O)[C@@H](NC(=O)c2ncccc2O)[C@@H](C)NC1=O ; CCC(C)C1NC(=O)[C@H](C)[C@H](O)[C@H](Cc2ccccc2)NC(=O)[C@@H](NC(=O)c2ncccc2O)[C@@H](C)NC1=O ; CCC(C)C1C(=O)N[C@H](C)[C@H](NC(=O)c2ncccc2O)C(=O)N[C@@H](Cc2ccccc2)[C@@H](O)[C@@H](C)C(=O)N1C ; CC(C)CC1OC(=O)[C@H](C)[C@H](O)[C@H](Cc2ccccc2)NC(=O)[C@@H](NC(=O)c2ncccc2O)[C@@H](C)NC1=O]

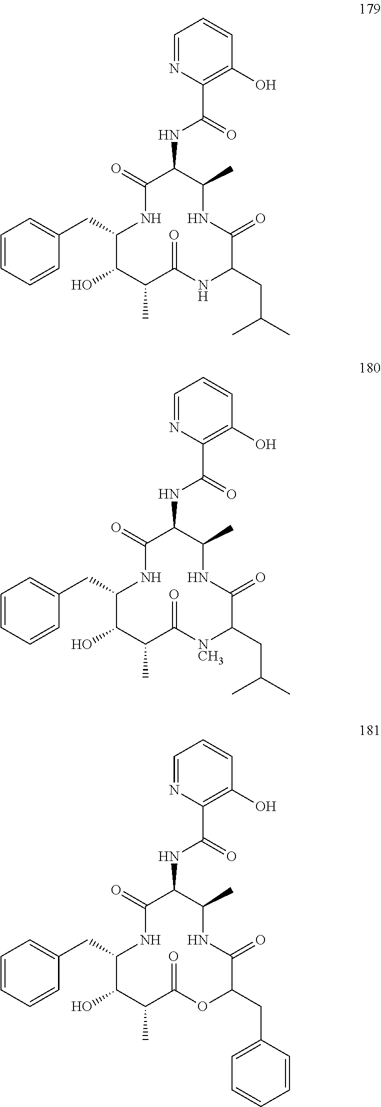 [SMILES: CC(C)CC1NC(=O)[C@H](C)[C@H](O)[C@H](Cc2ccccc2)NC(=O)[C@@H](NC(=O)c2ncccc2O)[C@@H](C)NC1=O ; CC(C)CC1C(=O)N[C@H](C)[C@H](NC(=O)c2ncccc2O)C(=O)N[C@@H](Cc2ccccc2)[C@@H](O)[C@@H](C)C(=O)N1C ; C[C@H]1NC(=O)C(Cc2ccccc2)OC(=O)[C@H](C)[C@H](O)[C@H](Cc2ccccc2)NC(=O)[C@H]1NC(=O)c1ncccc1O]

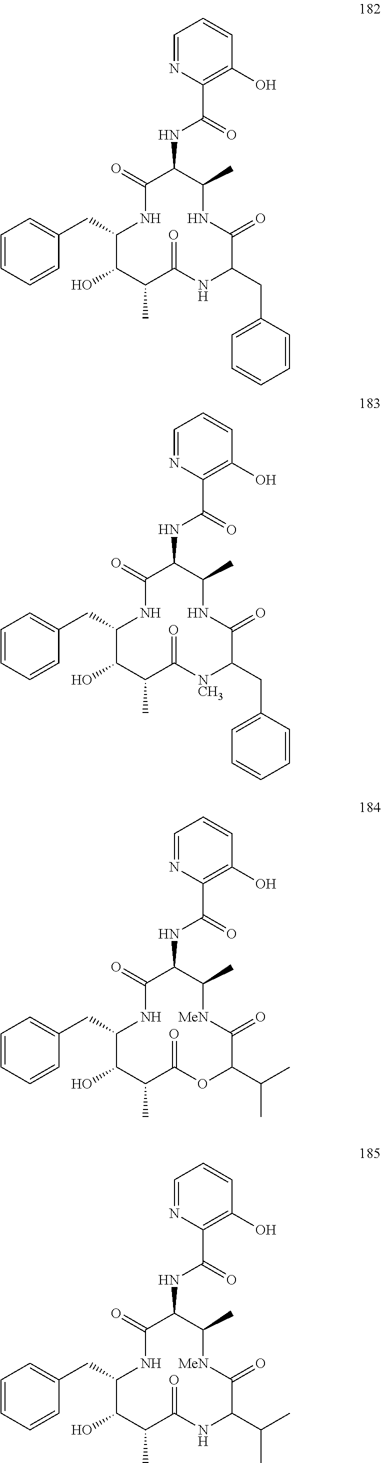 [SMILES: C[C@H]1NC(=O)C(Cc2ccccc2)NC(=O)[C@H](C)[C@H](O)[C@H](Cc2ccccc2)NC(=O)[C@H]1NC(=O)c1ncccc1O ; C[C@H]1NC(=O)C(Cc2ccccc2)N(C)C(=O)[C@H](C)[C@H](O)[C@H](Cc2ccccc2)NC(=O)[C@H]1NC(=O)c1ncccc1O ; CC(C)C1OC(=O)[C@H](C)[C@H](O)[C@H](Cc2ccccc2)NC(=O)[C@@H](NC(=O)c2ncccc2O)[C@@H](C)N(C)C1=O ; CC(C)C1NC(=O)[C@H](C)[C@H](O)[C@H](Cc2ccccc2)NC(=O)[C@@H](NC(=O)c2ncccc2O)[C@@H](C)N(C)C1=O]

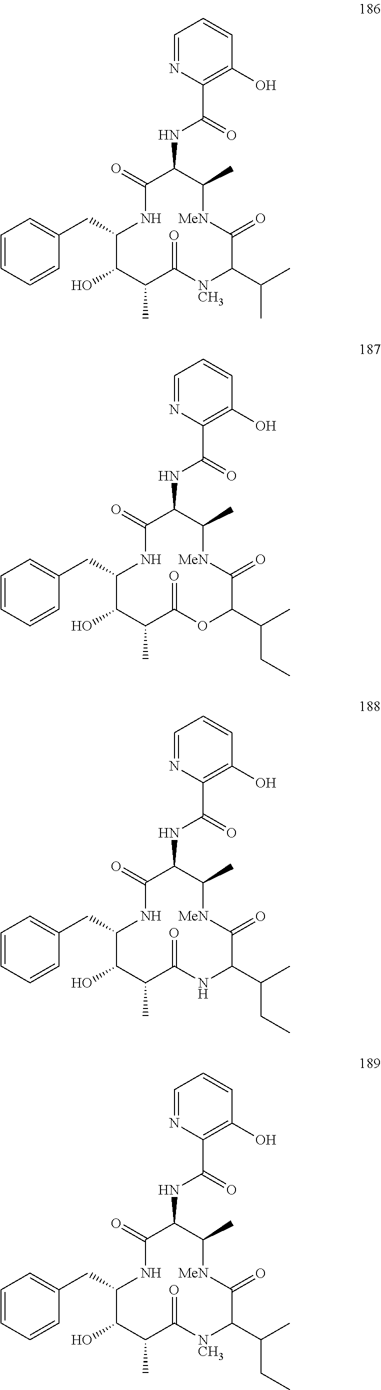 [SMILES: CC(C)C1C(=O)N(C)[C@H](C)[C@H](NC(=O)c2ncccc2O)C(=O)N[C@@H](Cc2ccccc2)[C@@H](O)[C@@H](C)C(=O)N1C ; CCC(C)C1OC(=O)[C@H](C)[C@H](O)[C@H](Cc2ccccc2)NC(=O)[C@@H](NC(=O)c2ncccc2O)[C@@H](C)N(C)C1=O ; CCC(C)C1NC(=O)[C@H](C)[C@H](O)[C@H](Cc2ccccc2)NC(=O)[C@@H](NC(=O)c2ncccc2O)[C@@H](C)N(C)C1=O ; CCC(C)C1C(=O)N(C)[C@H](C)[C@H](NC(=O)c2ncccc2O)C(=O)N[C@@H](Cc2ccccc2)[C@@H](O)[C@@H](C)C(=O)N1C]

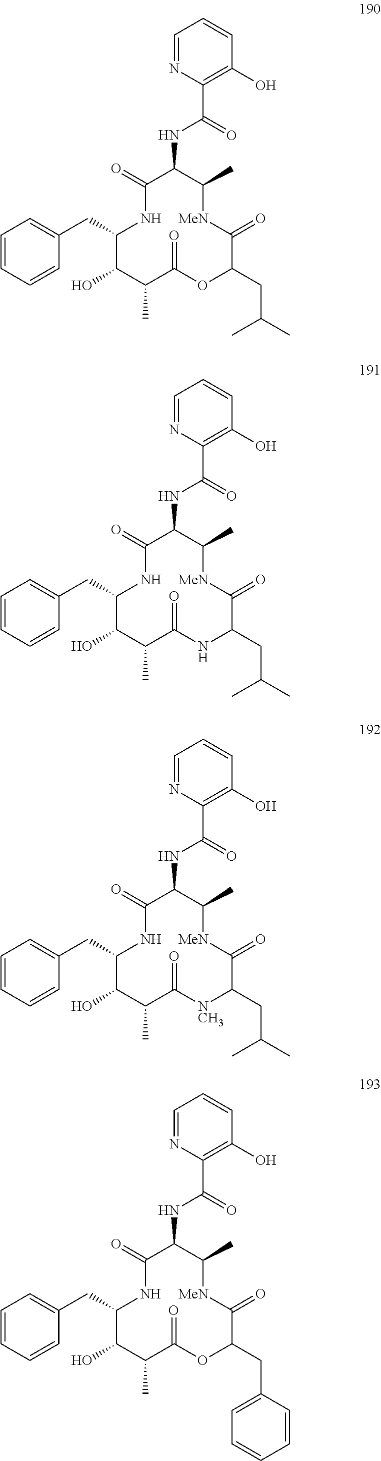 [SMILES: CC(C)CC1OC(=O)[C@H](C)[C@H](O)[C@H](Cc2ccccc2)NC(=O)[C@@H](NC(=O)c2ncccc2O)[C@@H](C)N(C)C1=O ; CC(C)CC1NC(=O)[C@H](C)[C@H](O)[C@H](Cc2ccccc2)NC(=O)[C@@H](NC(=O)c2ncccc2O)[C@@H](C)N(C)C1=O ; CC(C)CC1C(=O)N(C)[C@H](C)[C@H](NC(=O)c2ncccc2O)C(=O)N[C@@H](Cc2ccccc2)[C@@H](O)[C@@H](C)C(=O)N1C ; C[C@@H]1[C@H](NC(=O)c2ncccc2O)C(=O)N[C@@H](Cc2ccccc2)[C@@H](O)[C@@H](C)C(=O)OC(Cc2ccccc2)C(=O)N1C]

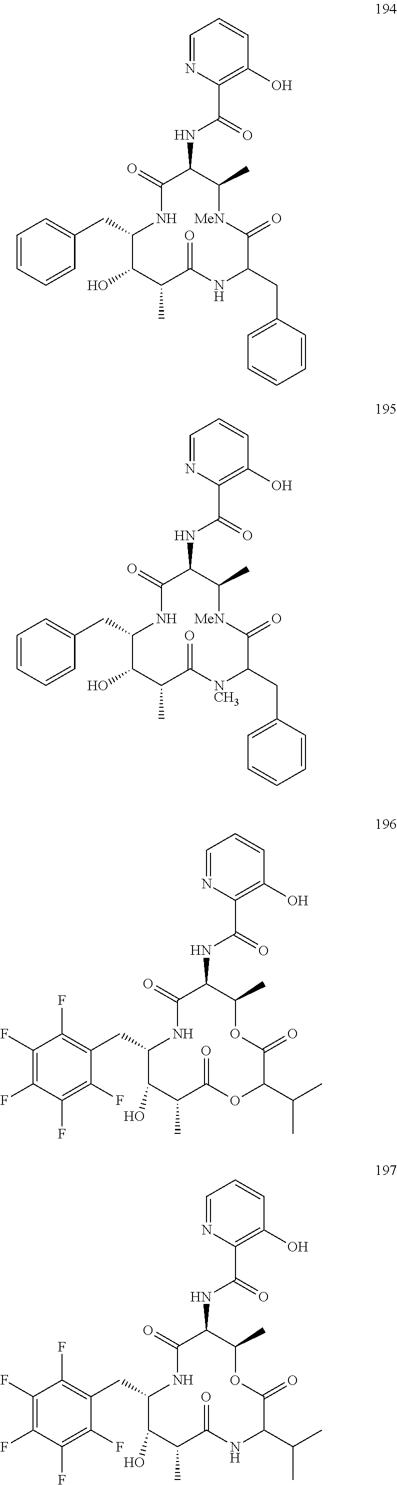 [SMILES: C[C@@H]1[C@H](NC(=O)c2ncccc2O)C(=O)N[C@@H](Cc2ccccc2)[C@@H](O)[C@@H](C)C(=O)NC(Cc2ccccc2)C(=O)N1C ; C[C@@H]1[C@H](NC(=O)c2ncccc2O)C(=O)N[C@@H](Cc2ccccc2)[C@@H](O)[C@@H](C)C(=O)N(C)C(Cc2ccccc2)C(=O)N1C ; CC(C)C1OC(=O)[C@H](C)[C@H](O)[C@H](Cc2c(F)c(F)c(F)c(F)c2F)NC(=O)[C@@H](NC(=O)c2ncccc2O)[C@@H](C)OC1=O ; CC(C)C1NC(=O)[C@H](C)[C@H](O)[C@H](Cc2c(F)c(F)c(F)c(F)c2F)NC(=O)[C@@H](NC(=O)c2ncccc2O)[C@@H](C)OC1=O]

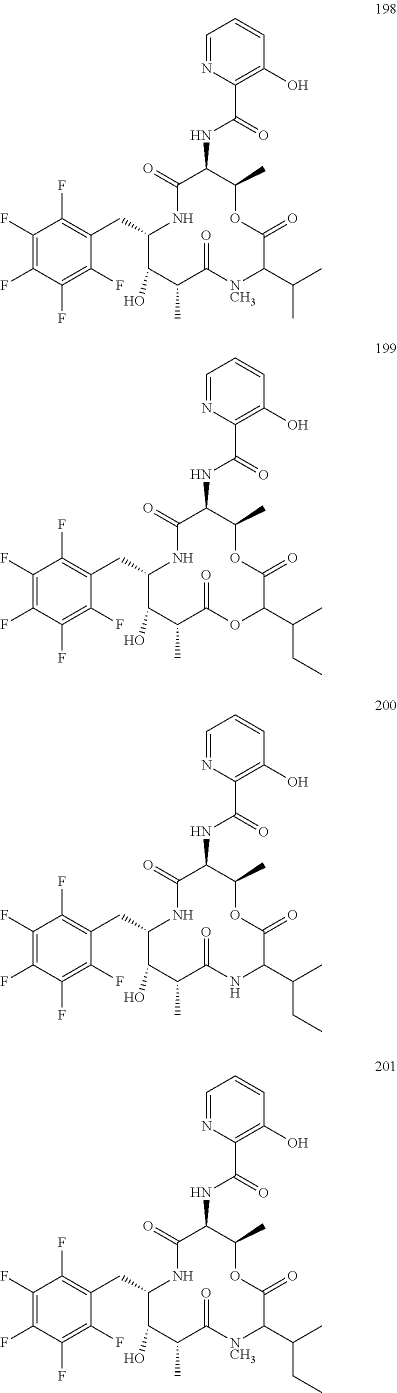 [SMILES: CC(C)C1C(=O)O[C@H](C)[C@H](NC(=O)c2ncccc2O)C(=O)N[C@@H](Cc2c(F)c(F)c(F)c(F)c2F)[C@@H](O)[C@@H](C)C(=O)N1C ; CCC(C)C1OC(=O)[C@H](C)[C@H](O)[C@H](Cc2c(F)c(F)c(F)c(F)c2F)NC(=O)[C@@H](NC(=O)c2ncccc2O)[C@@H](C)OC1=O ; CCC(C)C1NC(=O)[C@H](C)[C@H](O)[C@H](Cc2c(F)c(F)c(F)c(F)c2F)NC(=O)[C@@H](NC(=O)c2ncccc2O)[C@@H](C)OC1=O ; CCC(C)C1C(=O)O[C@H](C)[C@H](NC(=O)c2ncccc2O)C(=O)N[C@@H](Cc2c(F)c(F)c(F)c(F)c2F)[C@@H](O)[C@@H](C)C(=O)N1C]

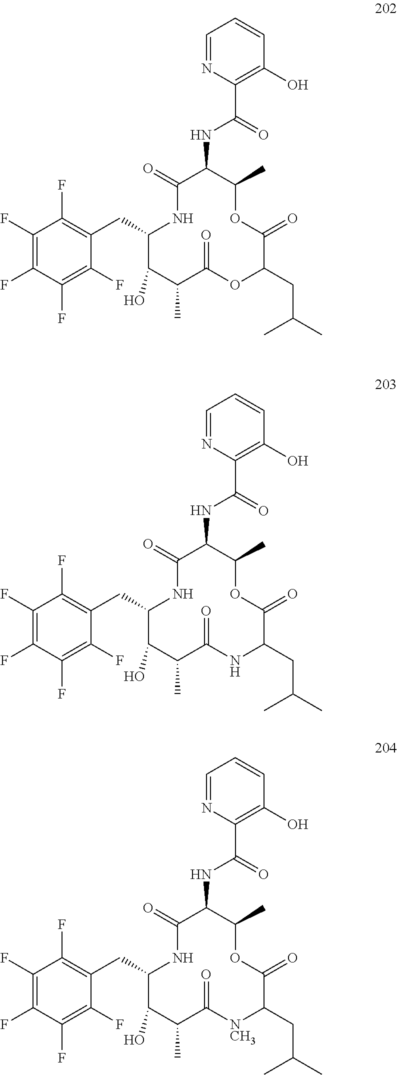 [SMILES: CC(C)CC1OC(=O)[C@H](C)[C@H](O)[C@H](Cc2c(F)c(F)c(F)c(F)c2F)NC(=O)[C@@H](NC(=O)c2ncccc2O)[C@@H](C)OC1=O ; CC(C)CC1NC(=O)[C@H](C)[C@H](O)[C@H](Cc2c(F)c(F)c(F)c(F)c2F)NC(=O)[C@@H](NC(=O)c2ncccc2O)[C@@H](C)OC1=O ; CC(C)CC1C(=O)O[C@H](C)[C@H](NC(=O)c2ncccc2O)C(=O)N[C@@H](Cc2c(F)c(F)c(F)c(F)c2F)[C@@H](O)[C@@H](C)C(=O)N1C]

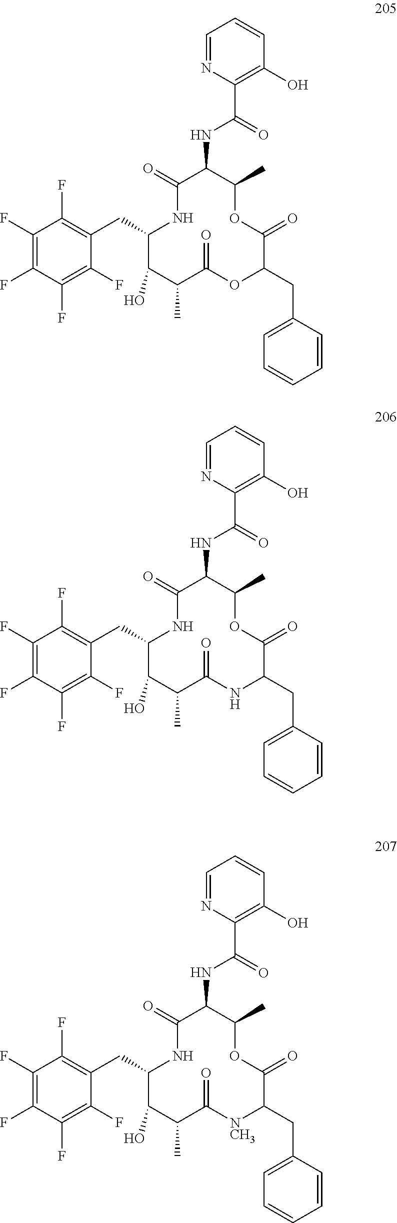 [SMILES: C[C@H]1OC(=O)C(Cc2ccccc2)OC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(Cc2ccccc2)NC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(Cc2ccccc2)N(C)C(=O)[C@H](C)[C@H](O)[C@H](Cc2c(F)c(F)c(F)c(F)c2F)NC(=O)[C@H]1NC(=O)c1ncccc1O]

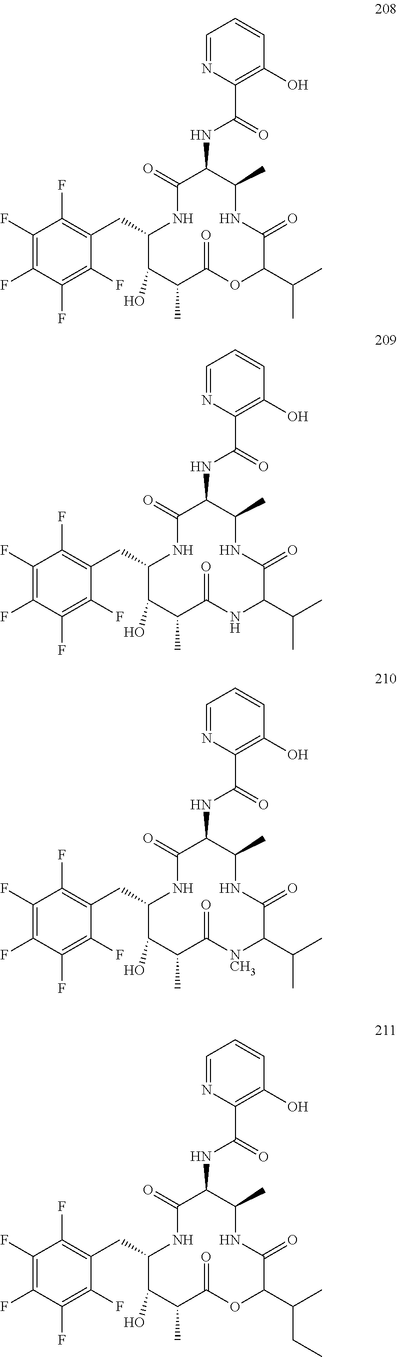 [SMILES: CC(C)C1OC(=O)[C@H](C)[C@H](O)[C@H](Cc2c(F)c(F)c(F)c(F)c2F)NC(=O)[C@@H](NC(=O)c2ncccc2O)[C@@H](C)NC1=O ; CC(C)C1NC(=O)[C@H](C)[C@H](O)[C@H](Cc2c(F)c(F)c(F)c(F)c2F)NC(=O)[C@@H](NC(=O)c2ncccc2O)[C@@H](C)NC1=O ; CC(C)C1C(=O)N[C@H](C)[C@H](NC(=O)c2ncccc2O)C(=O)N[C@@H](Cc2c(F)c(F)c(F)c(F)c2F)[C@@H](O)[C@@H](C)C(=O)N1C ; CCC(C)C1OC(=O)[C@H](C)[C@H](O)[C@H](Cc2c(F)c(F)c(F)c(F)c2F)NC(=O)[C@@H](NC(=O)c2ncccc2O)[C@@H](C)NC1=O]

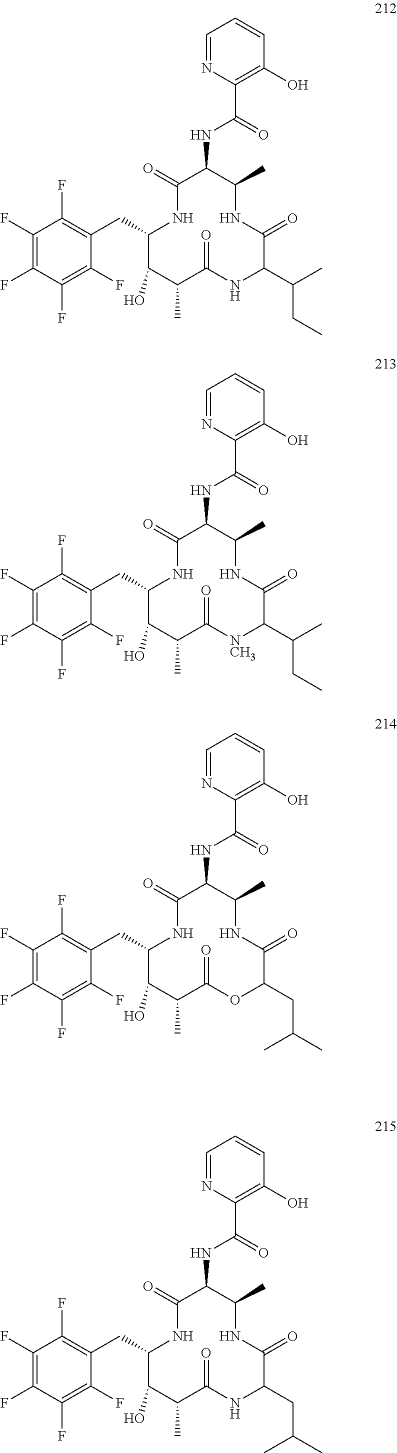 [SMILES: CCC(C)C1NC(=O)[C@H](C)[C@H](O)[C@H](Cc2c(F)c(F)c(F)c(F)c2F)NC(=O)[C@@H](NC(=O)c2ncccc2O)[C@@H](C)NC1=O ; CCC(C)C1C(=O)N[C@H](C)[C@H](NC(=O)c2ncccc2O)C(=O)N[C@@H](Cc2c(F)c(F)c(F)c(F)c2F)[C@@H](O)[C@@H](C)C(=O)N1C ; CC(C)CC1OC(=O)[C@H](C)[C@H](O)[C@H](Cc2c(F)c(F)c(F)c(F)c2F)NC(=O)[C@@H](NC(=O)c2ncccc2O)[C@@H](C)NC1=O ; CC(C)CC1NC(=O)[C@H](C)[C@H](O)[C@H](Cc2c(F)c(F)c(F)c(F)c2F)NC(=O)[C@@H](NC(=O)c2ncccc2O)[C@@H](C)NC1=O]

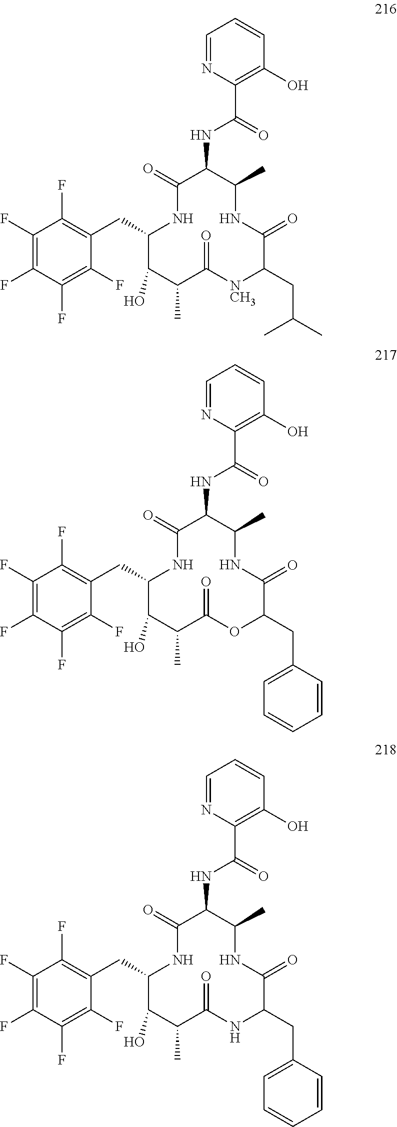

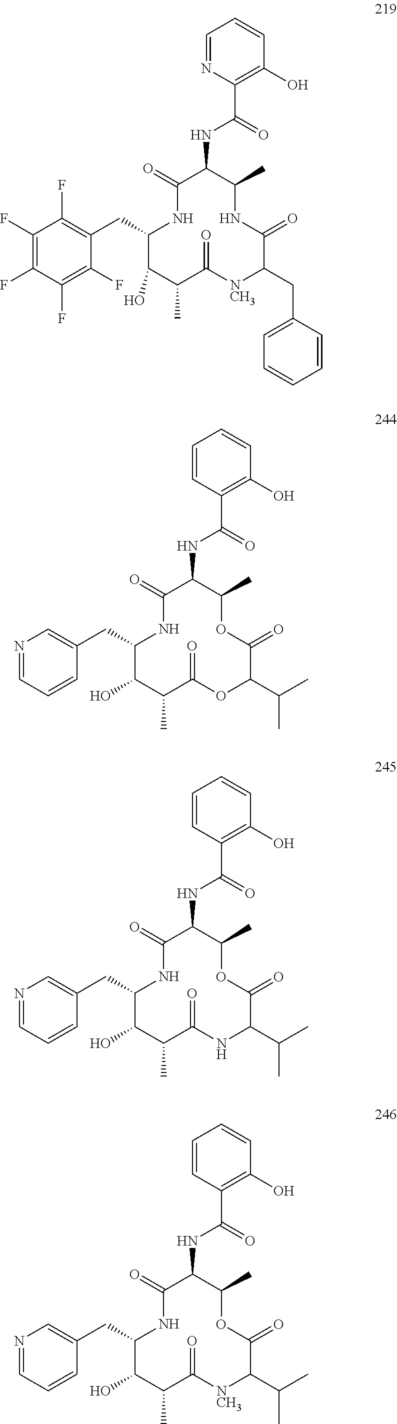 [SMILES: C[C@H]1NC(=O)C(Cc2ccccc2)N(C)C(=O)[C@H](C)[C@H](O)[C@H](Cc2c(F)c(F)c(F)c(F)c2F)NC(=O)[C@H]1NC(=O)c1ncccc1O ; CC(C)C1OC(=O)[C@H](C)[C@H](O)[C@H](Cc2cccnc2)NC(=O)[C@@H](NC(=O)c2ccccc2O)[C@@H](C)OC1=O ; CC(C)C1NC(=O)[C@H](C)[C@H](O)[C@H](Cc2cccnc2)NC(=O)[C@@H](NC(=O)c2ccccc2O)[C@@H](C)OC1=O ; CC(C)C1C(=O)O[C@H](C)[C@H](NC(=O)c2ccccc2O)C(=O)N[C@@H](Cc2cccnc2)[C@@H](O)[C@@H](C)C(=O)N1C]

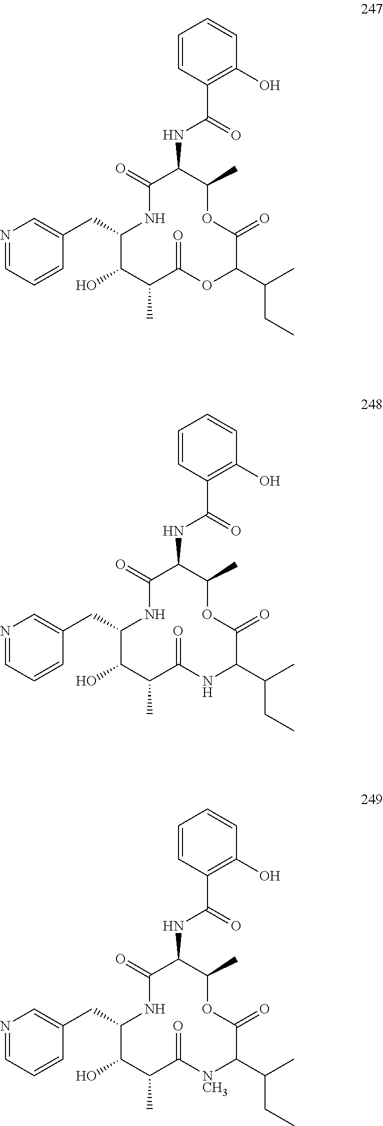 [SMILES: CCC(C)C1OC(=O)[C@H](C)[C@H](O)[C@H](Cc2cccnc2)NC(=O)[C@@H](NC(=O)c2ccccc2O)[C@@H](C)OC1=O ; CCC(C)C1NC(=O)[C@H](C)[C@H](O)[C@H](Cc2cccnc2)NC(=O)[C@@H](NC(=O)c2ccccc2O)[C@@H](C)OC1=O ; CCC(C)C1C(=O)O[C@H](C)[C@H](NC(=O)c2ccccc2O)C(=O)N[C@@H](Cc2cccnc2)[C@@H](O)[C@@H](C)C(=O)N1C]

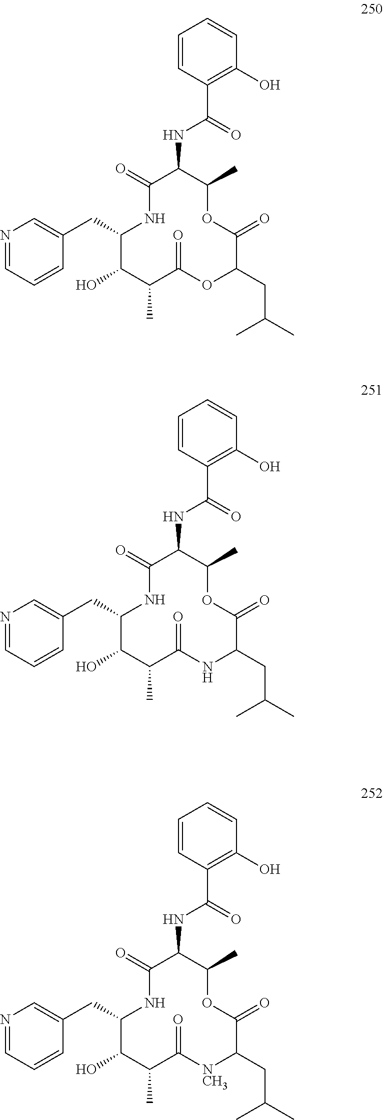 [SMILES: CC(C)CC1OC(=O)[C@H](C)[C@H](O)[C@H](Cc2cccnc2)NC(=O)[C@@H](NC(=O)c2ccccc2O)[C@@H](C)OC1=O ; CC(C)CC1NC(=O)[C@H](C)[C@H](O)[C@H](Cc2cccnc2)NC(=O)[C@@H](NC(=O)c2ccccc2O)[C@@H](C)OC1=O ; CC(C)CC1C(=O)O[C@H](C)[C@H](NC(=O)c2ccccc2O)C(=O)N[C@@H](Cc2cccnc2)[C@@H](O)[C@@H](C)C(=O)N1C]

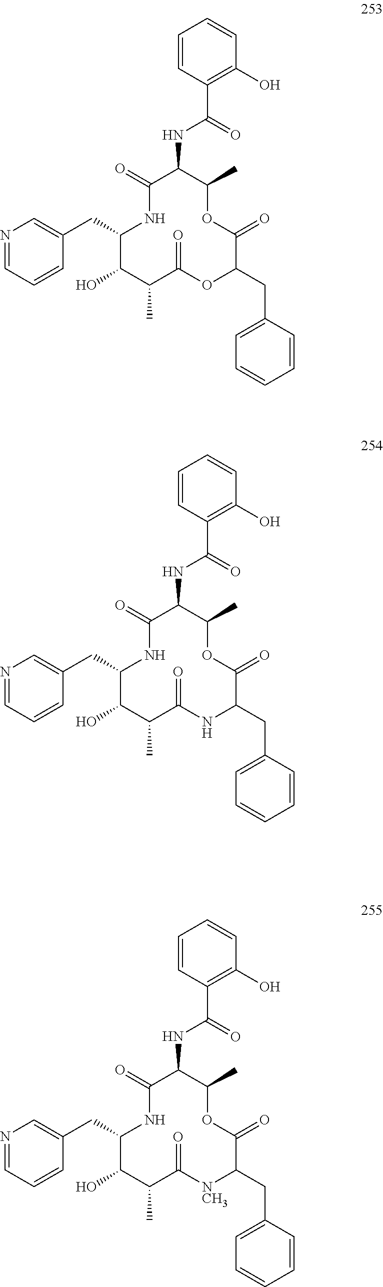 [SMILES: C[C@H]1OC(=O)C(Cc2ccccc2)OC(=O)[C@H](C)[C@H](O)[C@H](Cc2cccnc2)NC(=O)[C@H]1NC(=O)c1ccccc1O ; C[C@H]1OC(=O)C(Cc2ccccc2)NC(=O)[C@H](C)[C@H](O)[C@H](Cc2cccnc2)NC(=O)[C@H]1NC(=O)c1ccccc1O ; C[C@H]1OC(=O)C(Cc2ccccc2)N(C)C(=O)[C@H](C)[C@H](O)[C@H](Cc2cccnc2)NC(=O)[C@H]1NC(=O)c1ccccc1O]

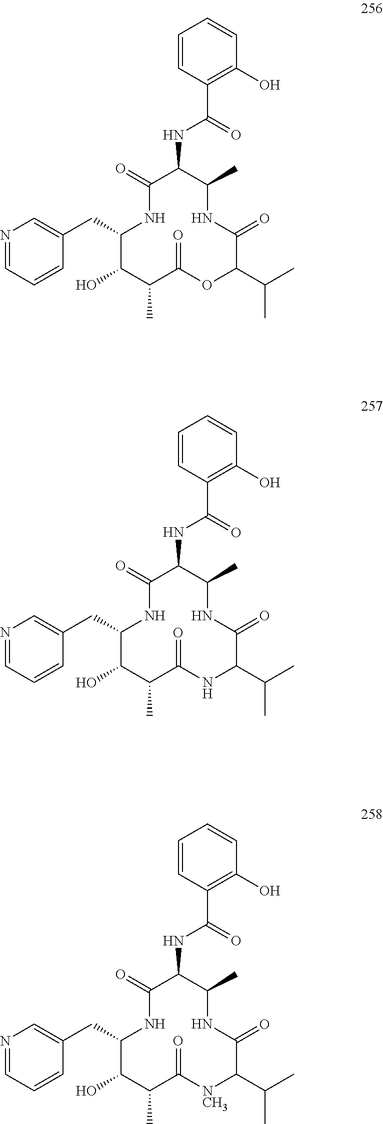 [SMILES: CC(C)C1OC(=O)[C@H](C)[C@H](O)[C@H](Cc2cccnc2)NC(=O)[C@@H](NC(=O)c2ccccc2O)[C@@H](C)NC1=O ; CC(C)C1NC(=O)[C@H](C)[C@H](O)[C@H](Cc2cccnc2)NC(=O)[C@@H](NC(=O)c2ccccc2O)[C@@H](C)NC1=O ; CC(C)C1C(=O)N[C@H](C)[C@H](NC(=O)c2ccccc2O)C(=O)N[C@@H](Cc2cccnc2)[C@@H](O)[C@@H](C)C(=O)N1C]

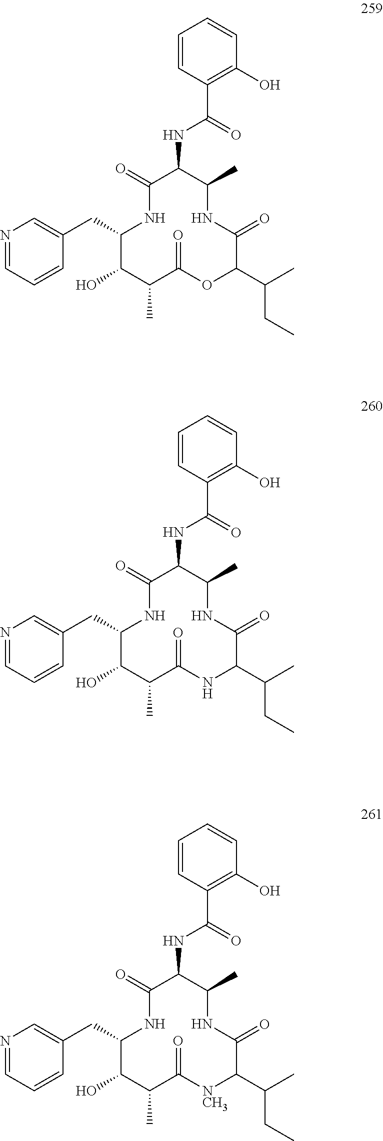 [SMILES: CCC(C)C1OC(=O)[C@H](C)[C@H](O)[C@H](Cc2cccnc2)NC(=O)[C@@H](NC(=O)c2ccccc2O)[C@@H](C)NC1=O ; CCC(C)C1NC(=O)[C@H](C)[C@H](O)[C@H](Cc2cccnc2)NC(=O)[C@@H](NC(=O)c2ccccc2O)[C@@H](C)NC1=O ; CCC(C)C1C(=O)N[C@H](C)[C@H](NC(=O)c2ccccc2O)C(=O)N[C@@H](Cc2cccnc2)[C@@H](O)[C@@H](C)C(=O)N1C]

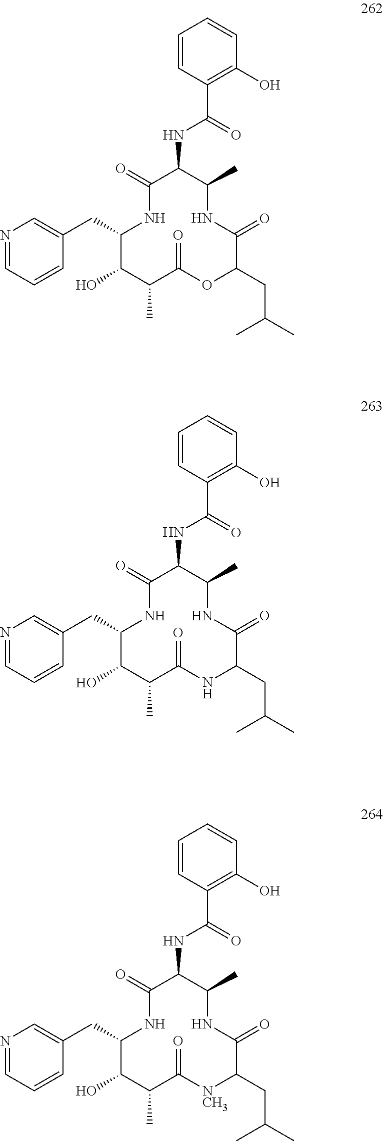 [SMILES: CC(C)CC1OC(=O)[C@H](C)[C@H](O)[C@H](Cc2cccnc2)NC(=O)[C@@H](NC(=O)c2ccccc2O)[C@@H](C)NC1=O ; CC(C)CC1NC(=O)[C@H](C)[C@H](O)[C@H](Cc2cccnc2)NC(=O)[C@@H](NC(=O)c2ccccc2O)[C@@H](C)NC1=O ; CC(C)CC1C(=O)N[C@H](C)[C@H](NC(=O)c2ccccc2O)C(=O)N[C@@H](Cc2cccnc2)[C@@H](O)[C@@H](C)C(=O)N1C]

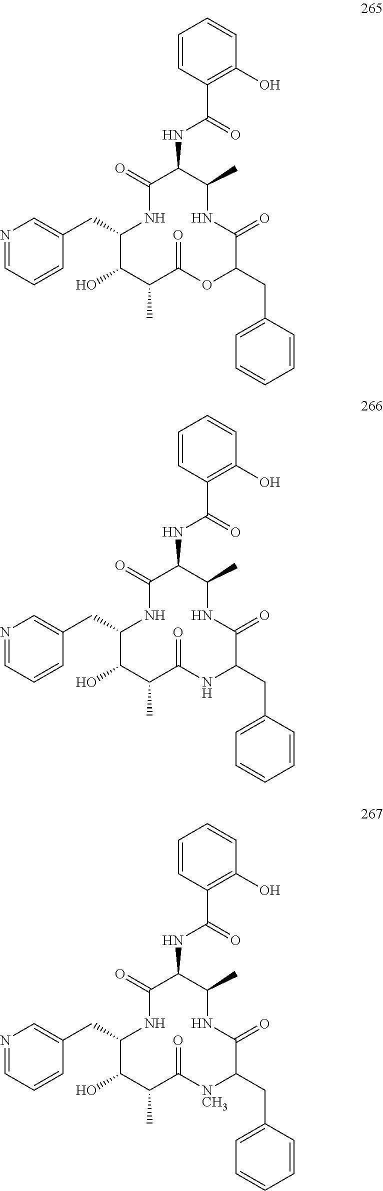 [SMILES: C[C@H]1NC(=O)C(Cc2ccccc2)OC(=O)[C@H](C)[C@H](O)[C@H](Cc2cccnc2)NC(=O)[C@H]1NC(=O)c1ccccc1O ; C[C@H]1NC(=O)C(Cc2ccccc2)NC(=O)[C@H](C)[C@H](O)[C@H](Cc2cccnc2)NC(=O)[C@H]1NC(=O)c1ccccc1O ; C[C@H]1NC(=O)C(Cc2ccccc2)N(C)C(=O)[C@H](C)[C@H](O)[C@H](Cc2cccnc2)NC(=O)[C@H]1NC(=O)c1ccccc1O]

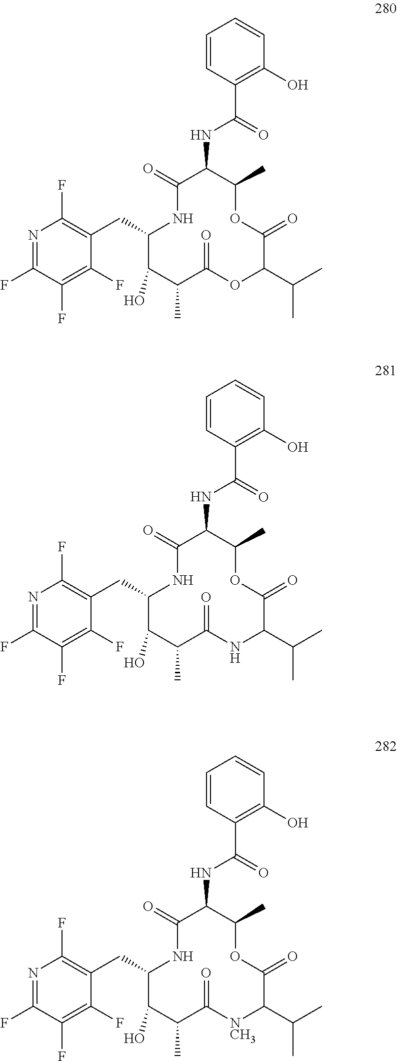 [SMILES: CC(C)C1OC(=O)[C@H](C)[C@H](O)[C@H](Cc2c(F)nc(F)c(F)c2F)NC(=O)[C@@H](NC(=O)c2ccccc2O)[C@@H](C)OC1=O ; CC(C)C1NC(=O)[C@H](C)[C@H](O)[C@H](Cc2c(F)nc(F)c(F)c2F)NC(=O)[C@@H](NC(=O)c2ccccc2O)[C@@H](C)OC1=O ; CC(C)C1C(=O)O[C@H](C)[C@H](NC(=O)c2ccccc2O)C(=O)N[C@@H](Cc2c(F)nc(F)c(F)c2F)[C@@H](O)[C@@H](C)C(=O)N1C]

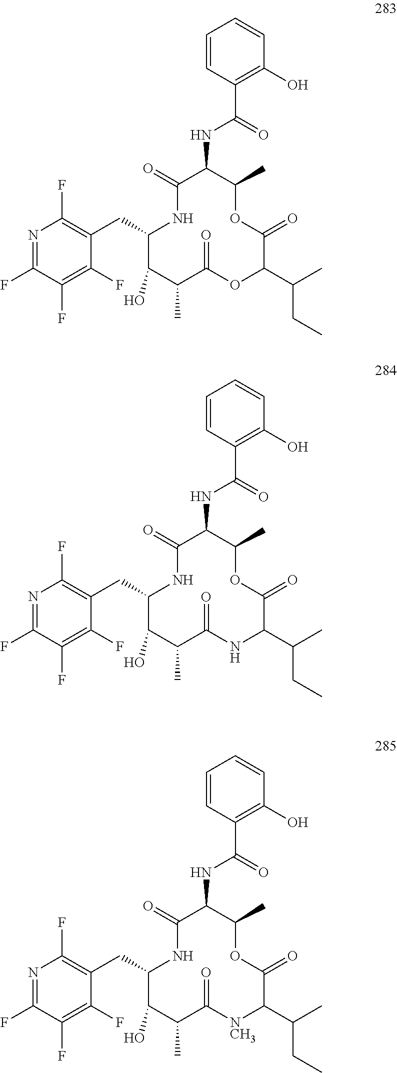 [SMILES: CCC(C)C1OC(=O)[C@H](C)[C@H](O)[C@H](Cc2c(F)nc(F)c(F)c2F)NC(=O)[C@@H](NC(=O)c2ccccc2O)[C@@H](C)OC1=O ; CCC(C)C1NC(=O)[C@H](C)[C@H](O)[C@H](Cc2c(F)nc(F)c(F)c2F)NC(=O)[C@@H](NC(=O)c2ccccc2O)[C@@H](C)OC1=O ; CCC(C)C1C(=O)O[C@H](C)[C@H](NC(=O)c2ccccc2O)C(=O)N[C@@H](Cc2c(F)nc(F)c(F)c2F)[C@@H](O)[C@@H](C)C(=O)N1C]

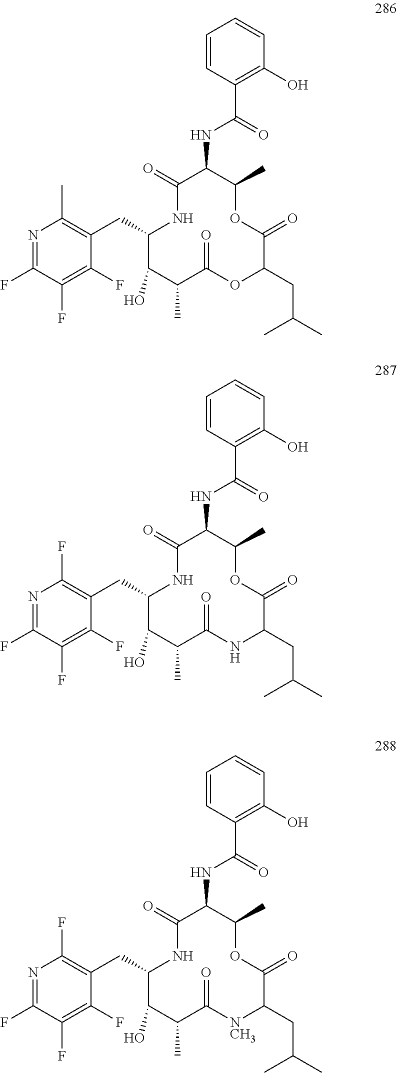 [SMILES: Cc1nc(F)c(F)c(F)c1C[C@@H]1NC(=O)[C@@H](NC(=O)c2ccccc2O)[C@@H](C)OC(=O)C(CC(C)C)OC(=O)[C@H](C)[C@@H]1O ; CC(C)CC1NC(=O)[C@H](C)[C@H](O)[C@H](Cc2c(F)nc(F)c(F)c2F)NC(=O)[C@@H](NC(=O)c2ccccc2O)[C@@H](C)OC1=O ; CC(C)CC1C(=O)O[C@H](C)[C@H](NC(=O)c2ccccc2O)C(=O)N[C@@H](Cc2c(F)nc(F)c(F)c2F)[C@@H](O)[C@@H](C)C(=O)N1C]

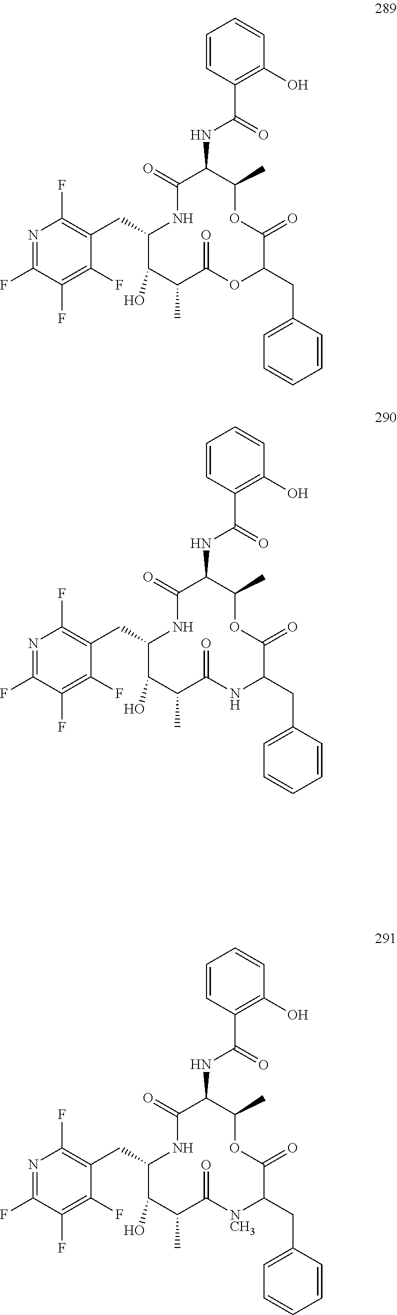 [SMILES: C[C@H]1OC(=O)C(Cc2ccccc2)OC(=O)[C@H](C)[C@H](O)[C@H](Cc2c(F)nc(F)c(F)c2F)NC(=O)[C@H]1NC(=O)c1ccccc1O ; C[C@H]1OC(=O)C(Cc2ccccc2)NC(=O)[C@H](C)[C@H](O)[C@H](Cc2c(F)nc(F)c(F)c2F)NC(=O)[C@H]1NC(=O)c1ccccc1O ; C[C@H]1OC(=O)C(Cc2ccccc2)N(C)C(=O)[C@H](C)[C@H](O)[C@H](Cc2c(F)nc(F)c(F)c2F)NC(=O)[C@H]1NC(=O)c1ccccc1O]

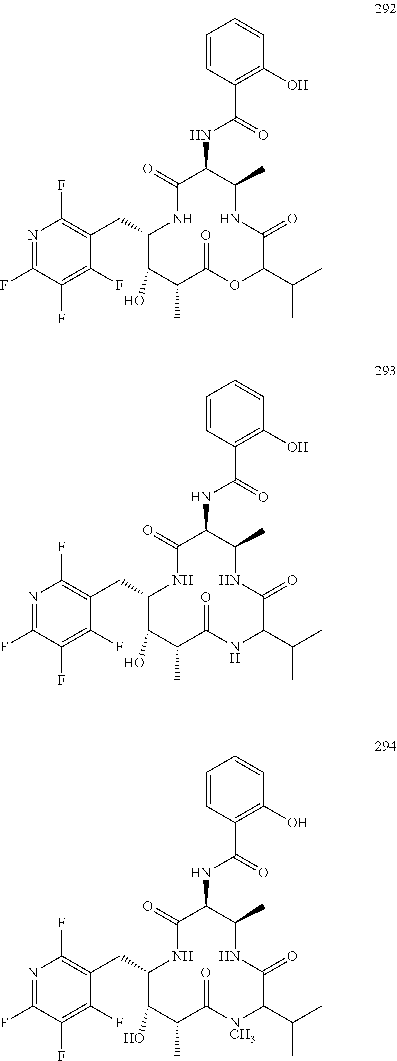 [SMILES: CC(C)C1OC(=O)[C@H](C)[C@H](O)[C@H](Cc2c(F)nc(F)c(F)c2F)NC(=O)[C@@H](NC(=O)c2ccccc2O)[C@@H](C)NC1=O ; CC(C)C1NC(=O)[C@H](C)[C@H](O)[C@H](Cc2c(F)nc(F)c(F)c2F)NC(=O)[C@@H](NC(=O)c2ccccc2O)[C@@H](C)NC1=O ; CC(C)C1C(=O)N[C@H](C)[C@H](NC(=O)c2ccccc2O)C(=O)N[C@@H](Cc2c(F)nc(F)c(F)c2F)[C@@H](O)[C@@H](C)C(=O)N1C]

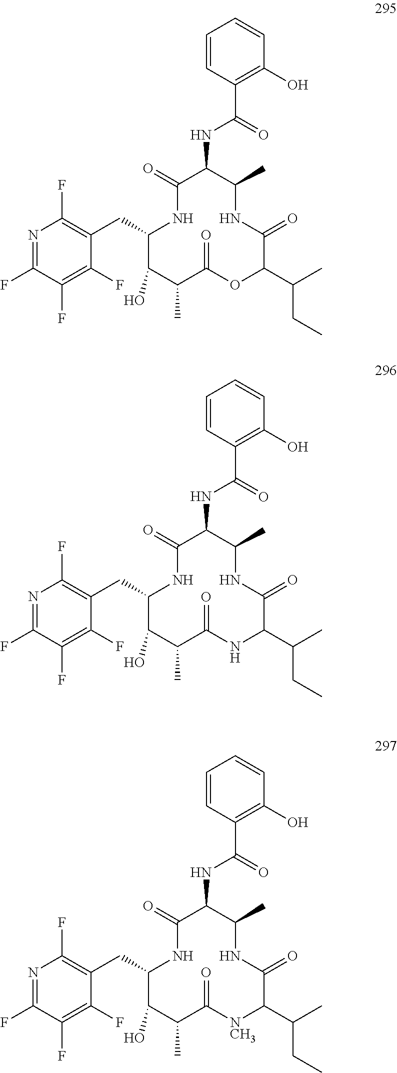 [SMILES: CCC(C)C1OC(=O)[C@H](C)[C@H](O)[C@H](Cc2c(F)nc(F)c(F)c2F)NC(=O)[C@@H](NC(=O)c2ccccc2O)[C@@H](C)NC1=O ; CCC(C)C1NC(=O)[C@H](C)[C@H](O)[C@H](Cc2c(F)nc(F)c(F)c2F)NC(=O)[C@@H](NC(=O)c2ccccc2O)[C@@H](C)NC1=O ; CCC(C)C1C(=O)N[C@H](C)[C@H](NC(=O)c2ccccc2O)C(=O)N[C@@H](Cc2c(F)nc(F)c(F)c2F)[C@@H](O)[C@@H](C)C(=O)N1C]

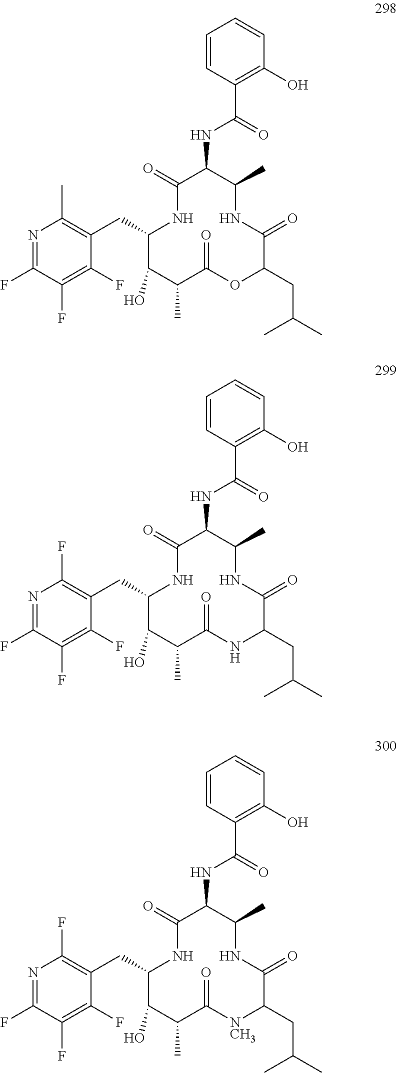 [SMILES: Cc1nc(F)c(F)c(F)c1C[C@@H]1NC(=O)[C@@H](NC(=O)c2ccccc2O)[C@@H](C)NC(=O)C(CC(C)C)OC(=O)[C@H](C)[C@@H]1O ; CC(C)CC1NC(=O)[C@H](C)[C@H](O)[C@H](Cc2c(F)nc(F)c(F)c2F)NC(=O)[C@@H](NC(=O)c2ccccc2O)[C@@H](C)NC1=O ; CC(C)CC1C(=O)N[C@H](C)[C@H](NC(=O)c2ccccc2O)C(=O)N[C@@H](Cc2c(F)nc(F)c(F)c2F)[C@@H](O)[C@@H](C)C(=O)N1C]

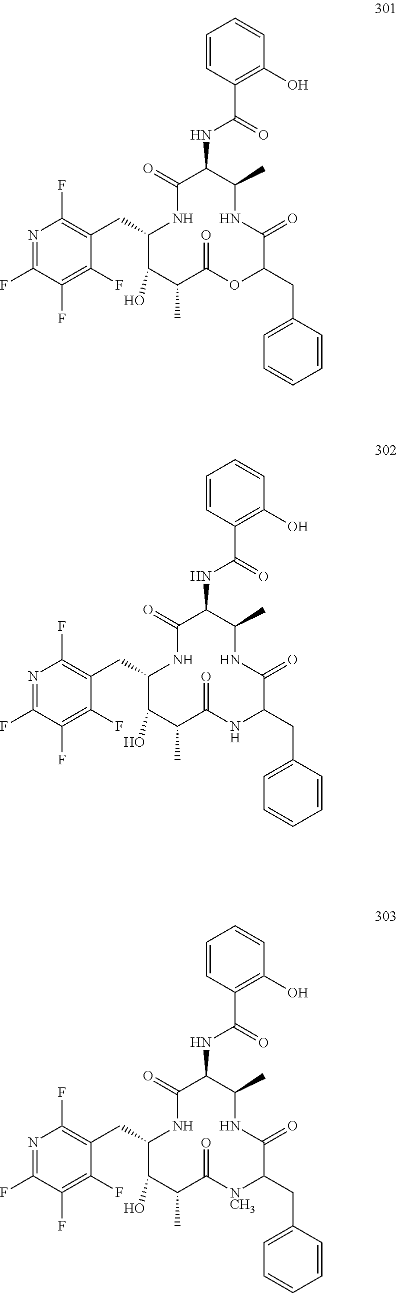 [SMILES: C[C@H]1NC(=O)C(Cc2ccccc2)OC(=O)[C@H](C)[C@H](O)[C@H](Cc2c(F)nc(F)c(F)c2F)NC(=O)[C@H]1NC(=O)c1ccccc1O ; C[C@H]1NC(=O)C(Cc2ccccc2)NC(=O)[C@H](C)[C@H](O)[C@H](Cc2c(F)nc(F)c(F)c2F)NC(=O)[C@H]1NC(=O)c1ccccc1O ; C[C@H]1NC(=O)C(Cc2ccccc2)N(C)C(=O)[C@H](C)[C@H](O)[C@H](Cc2c(F)nc(F)c(F)c2F)NC(=O)[C@H]1NC(=O)c1ccccc1O]

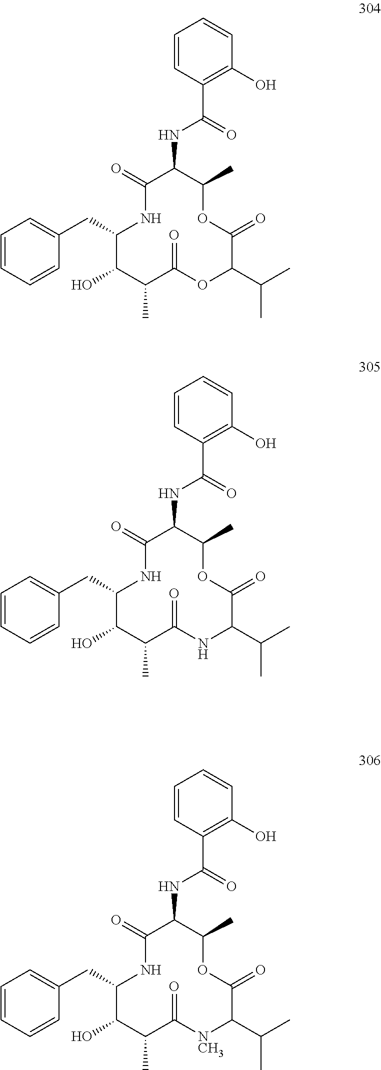 [SMILES: CC(C)C1OC(=O)[C@H](C)[C@H](O)[C@H](Cc2ccccc2)NC(=O)[C@@H](NC(=O)c2ccccc2O)[C@@H](C)OC1=O ; CC(C)C1NC(=O)[C@H](C)[C@H](O)[C@H](Cc2ccccc2)NC(=O)[C@@H](NC(=O)c2ccccc2O)[C@@H](C)OC1=O ; CC(C)C1C(=O)O[C@H](C)[C@H](NC(=O)c2ccccc2O)C(=O)N[C@@H](Cc2ccccc2)[C@@H](O)[C@@H](C)C(=O)N1C]

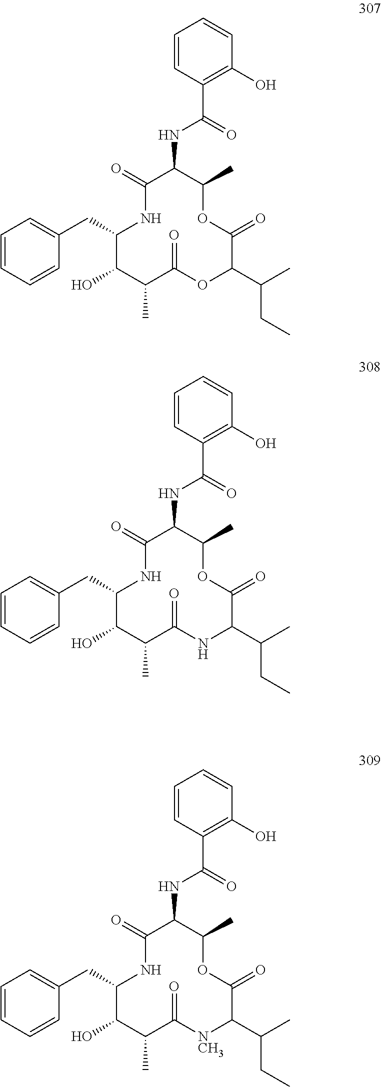 [SMILES: CCC(C)C1OC(=O)[C@H](C)[C@H](O)[C@H](Cc2ccccc2)NC(=O)[C@@H](NC(=O)c2ccccc2O)[C@@H](C)OC1=O ; CCC(C)C1NC(=O)[C@H](C)[C@H](O)[C@H](Cc2ccccc2)NC(=O)[C@@H](NC(=O)c2ccccc2O)[C@@H](C)OC1=O ; CCC(C)C1C(=O)O[C@H](C)[C@H](NC(=O)c2ccccc2O)C(=O)N[C@@H](Cc2ccccc2)[C@@H](O)[C@@H](C)C(=O)N1C]

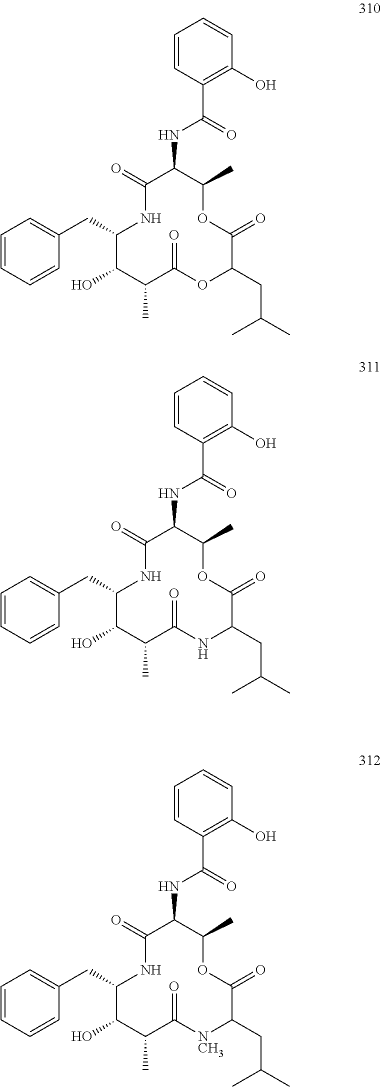 [SMILES: CC(C)CC1OC(=O)[C@H](C)[C@H](O)[C@H](Cc2ccccc2)NC(=O)[C@@H](NC(=O)c2ccccc2O)[C@@H](C)OC1=O ; CC(C)CC1NC(=O)[C@H](C)[C@H](O)[C@H](Cc2ccccc2)NC(=O)[C@@H](NC(=O)c2ccccc2O)[C@@H](C)OC1=O ; CC(C)CC1C(=O)O[C@H](C)[C@H](NC(=O)c2ccccc2O)C(=O)N[C@@H](Cc2ccccc2)[C@@H](O)[C@@H](C)C(=O)N1C]

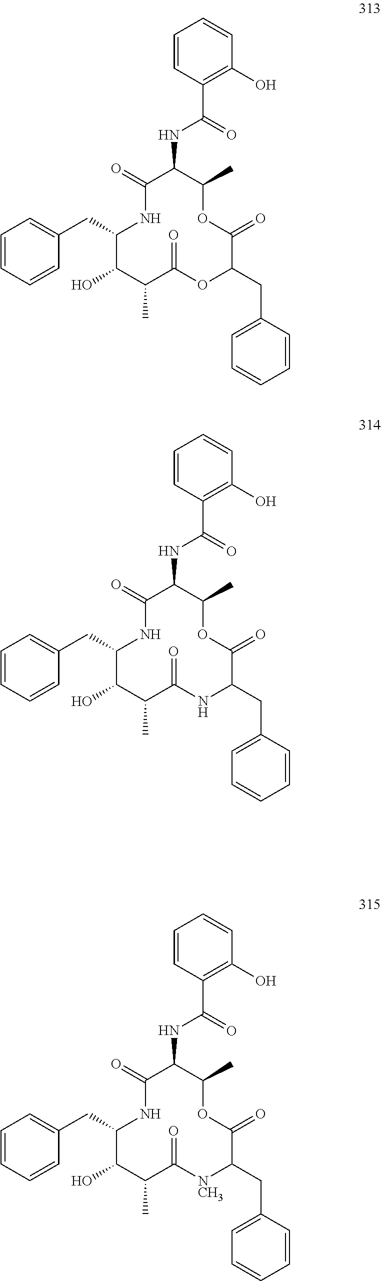 [SMILES: C[C@H]1OC(=O)C(Cc2ccccc2)OC(=O)[C@H](C)[C@H](O)[C@H](Cc2ccccc2)NC(=O)[C@H]1NC(=O)c1ccccc1O ; C[C@H]1OC(=O)C(Cc2ccccc2)NC(=O)[C@H](C)[C@H](O)[C@H](Cc2ccccc2)NC(=O)[C@H]1NC(=O)c1ccccc1O ; C[C@H]1OC(=O)C(Cc2ccccc2)N(C)C(=O)[C@H](C)[C@H](O)[C@H](Cc2ccccc2)NC(=O)[C@H]1NC(=O)c1ccccc1O]

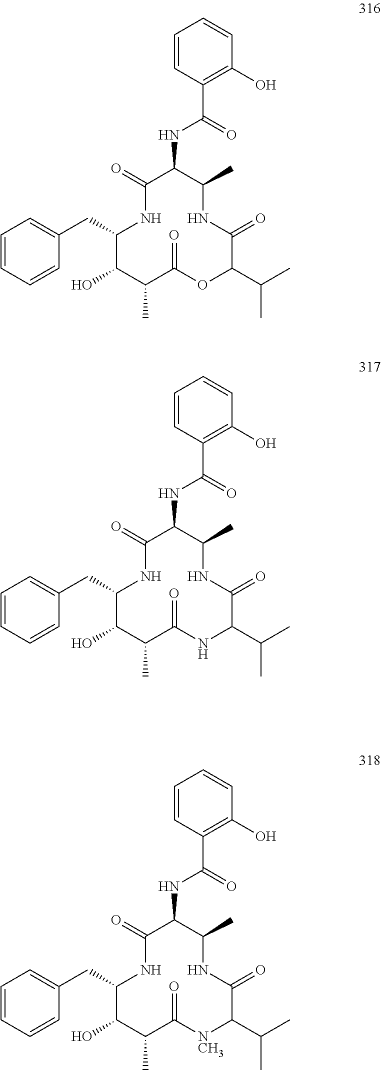 [SMILES: CC(C)C1OC(=O)[C@H](C)[C@H](O)[C@H](Cc2ccccc2)NC(=O)[C@@H](NC(=O)c2ccccc2O)[C@@H](C)NC1=O ; CC(C)C1NC(=O)[C@H](C)[C@H](O)[C@H](Cc2ccccc2)NC(=O)[C@@H](NC(=O)c2ccccc2O)[C@@H](C)NC1=O ; CC(C)C1C(=O)N[C@H](C)[C@H](NC(=O)c2ccccc2O)C(=O)N[C@@H](Cc2ccccc2)[C@@H](O)[C@@H](C)C(=O)N1C]

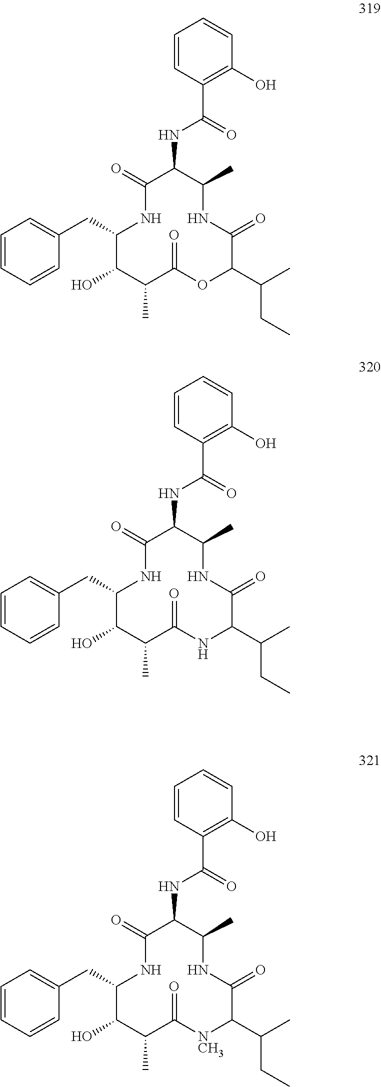 [SMILES: CCC(C)C1OC(=O)[C@H](C)[C@H](O)[C@H](Cc2ccccc2)NC(=O)[C@@H](NC(=O)c2ccccc2O)[C@@H](C)NC1=O ; CCC(C)C1NC(=O)[C@H](C)[C@H](O)[C@H](Cc2ccccc2)NC(=O)[C@@H](NC(=O)c2ccccc2O)[C@@H](C)NC1=O ; CCC(C)C1C(=O)N[C@H](C)[C@H](NC(=O)c2ccccc2O)C(=O)N[C@@H](Cc2ccccc2)[C@@H](O)[C@@H](C)C(=O)N1C]

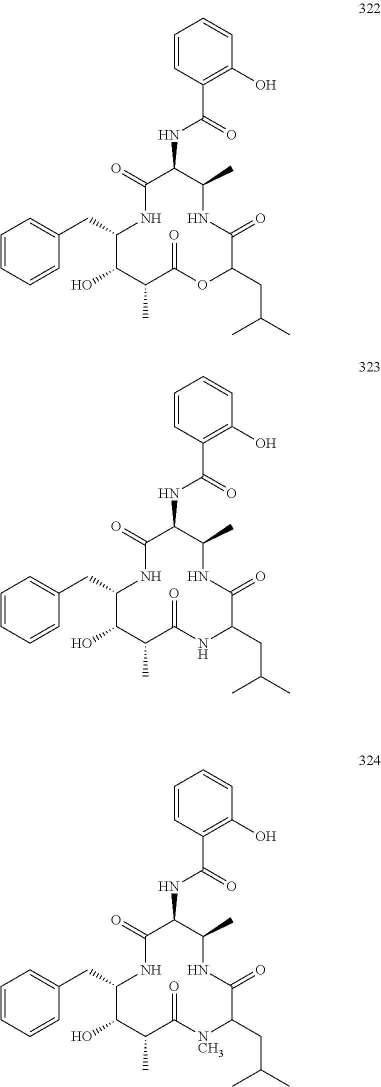 [SMILES: CC(C)CC1OC(=O)[C@H](C)[C@H](O)[C@H](Cc2ccccc2)NC(=O)[C@@H](NC(=O)c2ccccc2O)[C@@H](C)NC1=O ; CC(C)CC1NC(=O)[C@H](C)[C@H](O)[C@H](Cc2ccccc2)NC(=O)[C@@H](NC(=O)c2ccccc2O)[C@@H](C)NC1=O ; CC(C)CC1C(=O)N[C@H](C)[C@H](NC(=O)c2ccccc2O)C(=O)N[C@@H](Cc2ccccc2)[C@@H](O)[C@@H](C)C(=O)N1C]

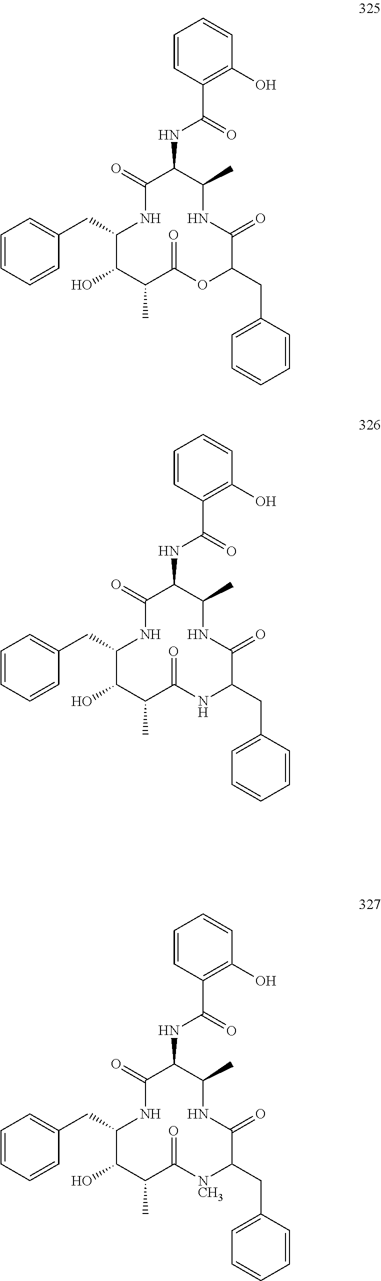 [SMILES: C[C@H]1NC(=O)C(Cc2ccccc2)OC(=O)[C@H](C)[C@H](O)[C@H](Cc2ccccc2)NC(=O)[C@H]1NC(=O)c1ccccc1O ; C[C@H]1NC(=O)C(Cc2ccccc2)NC(=O)[C@H](C)[C@H](O)[C@H](Cc2ccccc2)NC(=O)[C@H]1NC(=O)c1ccccc1O ; C[C@H]1NC(=O)C(Cc2ccccc2)N(C)C(=O)[C@H](C)[C@H](O)[C@H](Cc2ccccc2)NC(=O)[C@H]1NC(=O)c1ccccc1O]

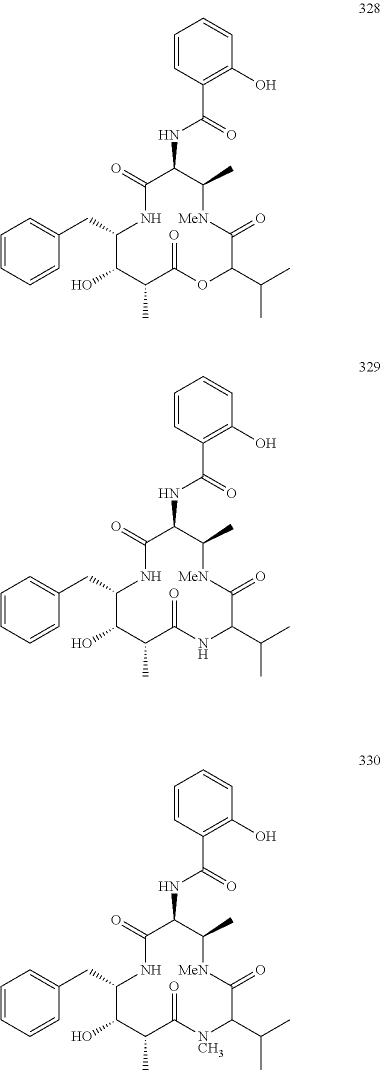 [SMILES: CC(C)C1OC(=O)[C@H](C)[C@H](O)[C@H](Cc2ccccc2)NC(=O)[C@@H](NC(=O)c2ccccc2O)[C@@H](C)N(C)C1=O ; CC(C)C1NC(=O)[C@H](C)[C@H](O)[C@H](Cc2ccccc2)NC(=O)[C@@H](NC(=O)c2ccccc2O)[C@@H](C)N(C)C1=O ; CC(C)C1C(=O)N(C)[C@H](C)[C@H](NC(=O)c2ccccc2O)C(=O)N[C@@H](Cc2ccccc2)[C@@H](O)[C@@H](C)C(=O)N1C]

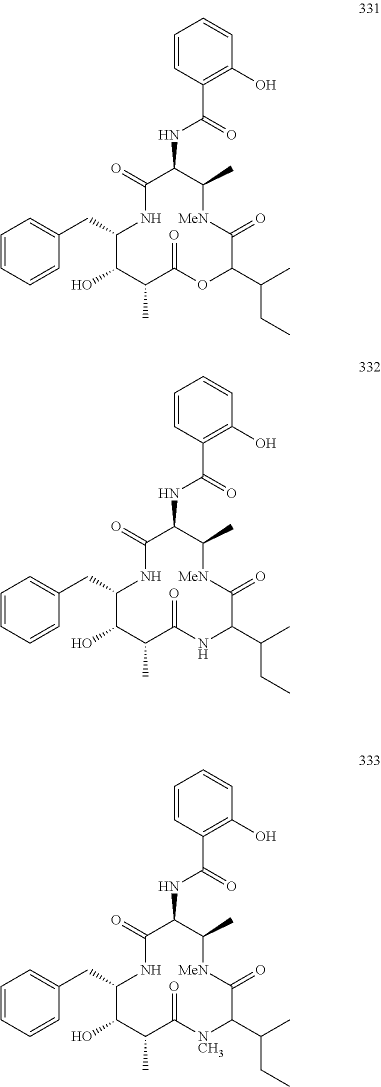 [SMILES: CCC(C)C1OC(=O)[C@H](C)[C@H](O)[C@H](Cc2ccccc2)NC(=O)[C@@H](NC(=O)c2ccccc2O)[C@@H](C)N(C)C1=O ; CCC(C)C1NC(=O)[C@H](C)[C@H](O)[C@H](Cc2ccccc2)NC(=O)[C@@H](NC(=O)c2ccccc2O)[C@@H](C)N(C)C1=O ; CCC(C)C1C(=O)N(C)[C@H](C)[C@H](NC(=O)c2ccccc2O)C(=O)N[C@@H](Cc2ccccc2)[C@@H](O)[C@@H](C)C(=O)N1C]

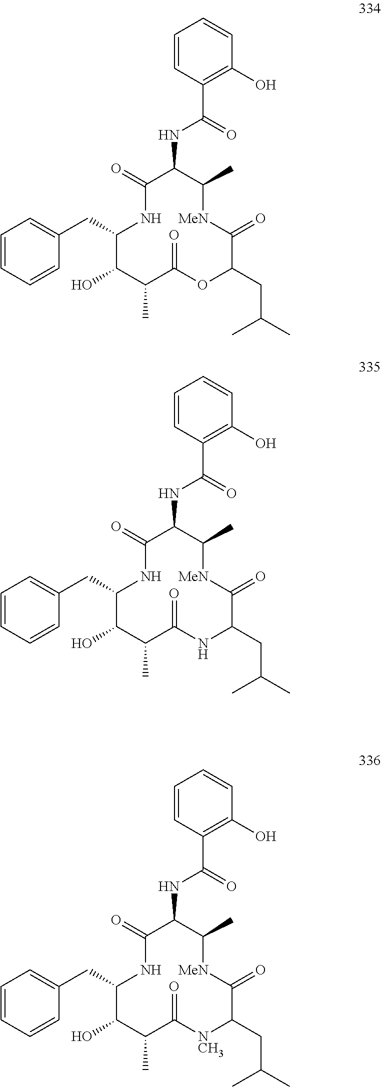 [SMILES: CC(C)CC1OC(=O)[C@H](C)[C@H](O)[C@H](Cc2ccccc2)NC(=O)[C@@H](NC(=O)c2ccccc2O)[C@@H](C)N(C)C1=O ; CC(C)CC1NC(=O)[C@H](C)[C@H](O)[C@H](Cc2ccccc2)NC(=O)[C@@H](NC(=O)c2ccccc2O)[C@@H](C)N(C)C1=O ; CC(C)CC1C(=O)N(C)[C@H](C)[C@H](NC(=O)c2ccccc2O)C(=O)N[C@@H](Cc2ccccc2)[C@@H](O)[C@@H](C)C(=O)N1C]

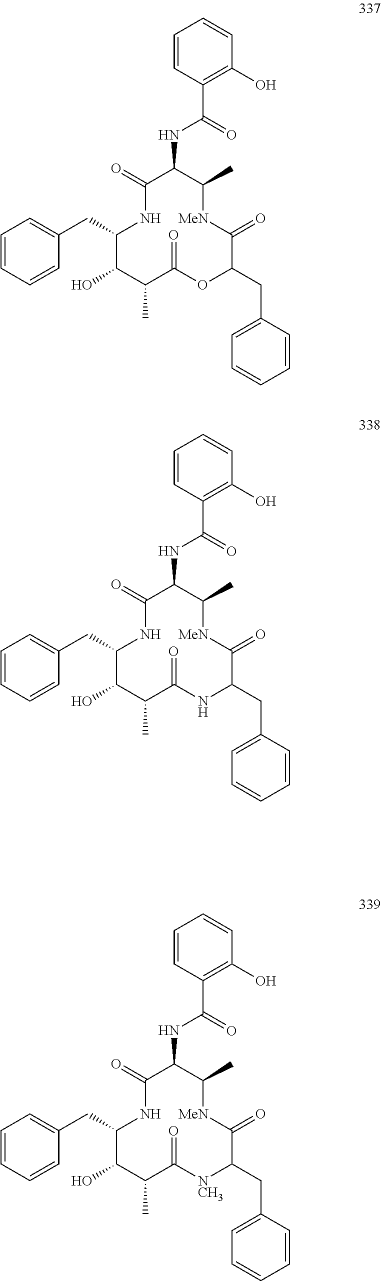 [SMILES: C[C@@H]1[C@H](NC(=O)c2ccccc2O)C(=O)N[C@@H](Cc2ccccc2)[C@@H](O)[C@@H](C)C(=O)OC(Cc2ccccc2)C(=O)N1C ; C[C@@H]1[C@H](NC(=O)c2ccccc2O)C(=O)N[C@@H](Cc2ccccc2)[C@@H](O)[C@@H](C)C(=O)NC(Cc2ccccc2)C(=O)N1C ; C[C@@H]1[C@H](NC(=O)c2ccccc2O)C(=O)N[C@@H](Cc2ccccc2)[C@@H](O)[C@@H](C)C(=O)N(C)C(Cc2ccccc2)C(=O)N1C]

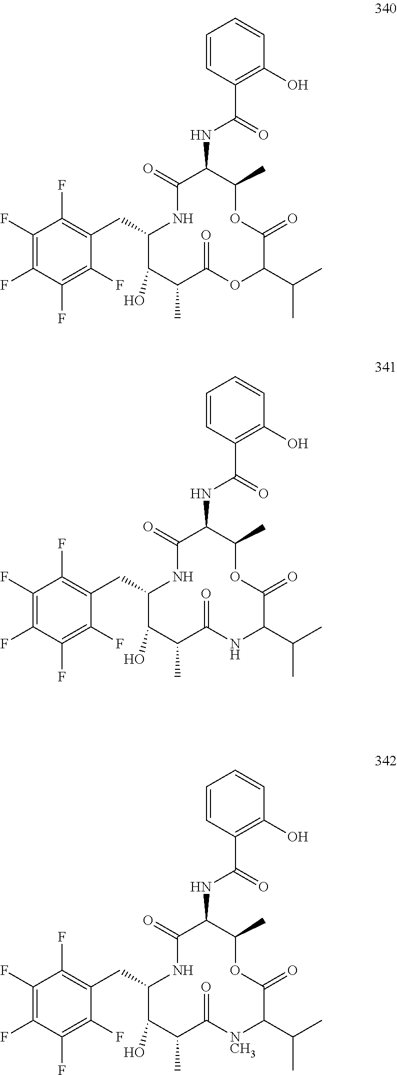 [SMILES: CC(C)C1OC(=O)[C@H](C)[C@H](O)[C@H](Cc2c(F)c(F)c(F)c(F)c2F)NC(=O)[C@@H](NC(=O)c2ccccc2O)[C@@H](C)OC1=O ; CC(C)C1NC(=O)[C@H](C)[C@H](O)[C@H](Cc2c(F)c(F)c(F)c(F)c2F)NC(=O)[C@@H](NC(=O)c2ccccc2O)[C@@H](C)OC1=O ; CC(C)C1C(=O)O[C@H](C)[C@H](NC(=O)c2ccccc2O)C(=O)N[C@@H](Cc2c(F)c(F)c(F)c(F)c2F)[C@@H](O)[C@@H](C)C(=O)N1C]

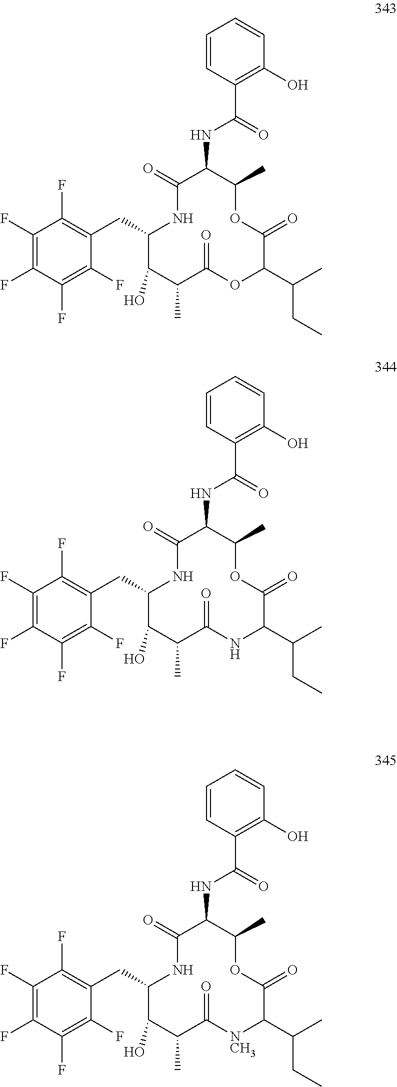 [SMILES: CCC(C)C1OC(=O)[C@H](C)[C@H](O)[C@H](Cc2c(F)c(F)c(F)c(F)c2F)NC(=O)[C@@H](NC(=O)c2ccccc2O)[C@@H](C)OC1=O ; CCC(C)C1NC(=O)[C@H](C)[C@H](O)[C@H](Cc2c(F)c(F)c(F)c(F)c2F)NC(=O)[C@@H](NC(=O)c2ccccc2O)[C@@H](C)OC1=O ; CCC(C)C1C(=O)O[C@H](C)[C@H](NC(=O)c2ccccc2O)C(=O)N[C@@H](Cc2c(F)c(F)c(F)c(F)c2F)[C@@H](O)[C@@H](C)C(=O)N1C]

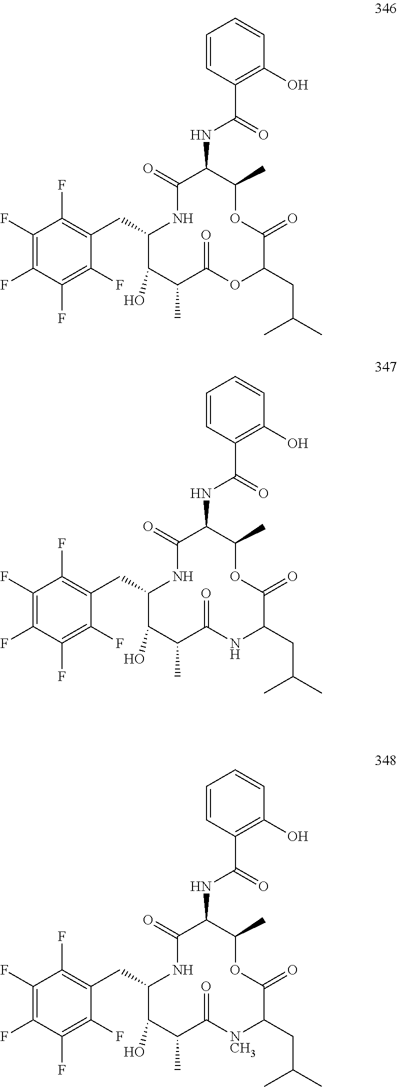 [SMILES: CC(C)CC1OC(=O)[C@H](C)[C@H](O)[C@H](Cc2c(F)c(F)c(F)c(F)c2F)NC(=O)[C@@H](NC(=O)c2ccccc2O)[C@@H](C)OC1=O ; CC(C)CC1NC(=O)[C@H](C)[C@H](O)[C@H](Cc2c(F)c(F)c(F)c(F)c2F)NC(=O)[C@@H](NC(=O)c2ccccc2O)[C@@H](C)OC1=O ; CC(C)CC1C(=O)O[C@H](C)[C@H](NC(=O)c2ccccc2O)C(=O)N[C@@H](Cc2c(F)c(F)c(F)c(F)c2F)[C@@H](O)[C@@H](C)C(=O)N1C]

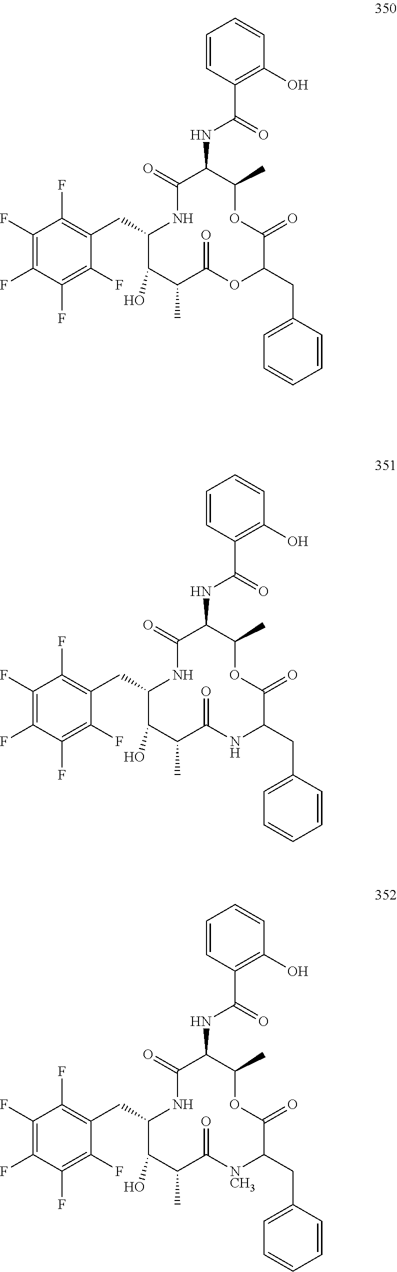 [SMILES: C[C@H]1OC(=O)C(Cc2ccccc2)OC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(Cc2ccccc2)NC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(Cc2ccccc2)N(C)C(=O)[C@H](C)[C@H](O)[C@H](Cc2c(F)c(F)c(F)c(F)c2F)NC(=O)[C@H]1NC(=O)c1ccccc1O]

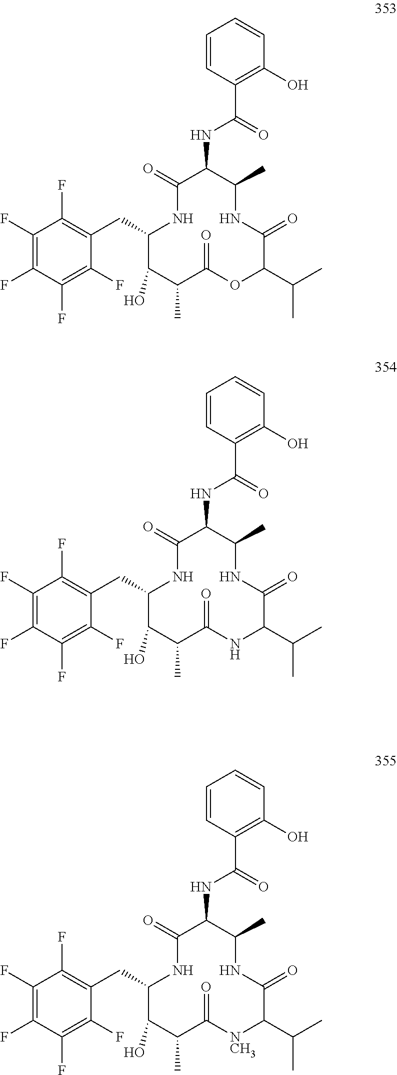 [SMILES: CC(C)C1OC(=O)[C@H](C)[C@H](O)[C@H](Cc2c(F)c(F)c(F)c(F)c2F)NC(=O)[C@@H](NC(=O)c2ccccc2O)[C@@H](C)NC1=O ; CC(C)C1NC(=O)[C@H](C)[C@H](O)[C@H](Cc2c(F)c(F)c(F)c(F)c2F)NC(=O)[C@@H](NC(=O)c2ccccc2O)[C@@H](C)NC1=O ; CC(C)C1C(=O)N[C@H](C)[C@H](NC(=O)c2ccccc2O)C(=O)N[C@@H](Cc2c(F)c(F)c(F)c(F)c2F)[C@@H](O)[C@@H](C)C(=O)N1C]

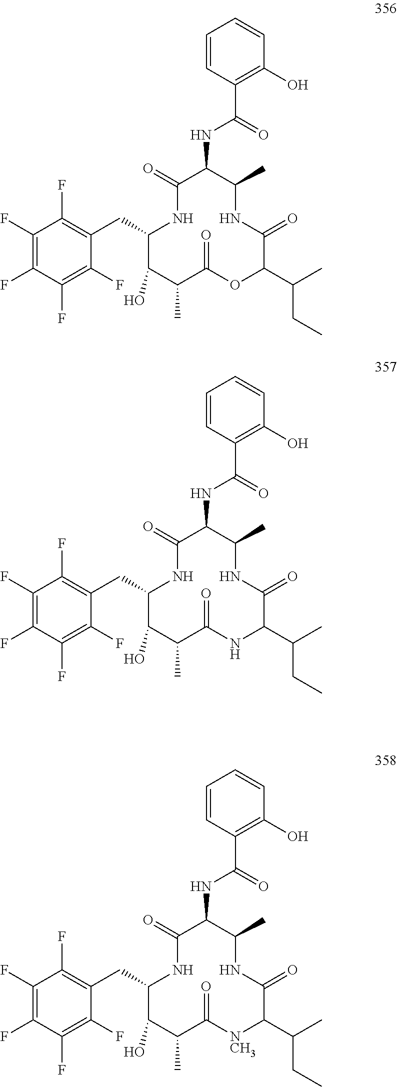 [SMILES: CCC(C)C1OC(=O)[C@H](C)[C@H](O)[C@H](Cc2c(F)c(F)c(F)c(F)c2F)NC(=O)[C@@H](NC(=O)c2ccccc2O)[C@@H](C)NC1=O ; CCC(C)C1NC(=O)[C@H](C)[C@H](O)[C@H](Cc2c(F)c(F)c(F)c(F)c2F)NC(=O)[C@@H](NC(=O)c2ccccc2O)[C@@H](C)NC1=O ; CCC(C)C1C(=O)N[C@H](C)[C@H](NC(=O)c2ccccc2O)C(=O)N[C@@H](Cc2c(F)c(F)c(F)c(F)c2F)[C@@H](O)[C@@H](C)C(=O)N1C]

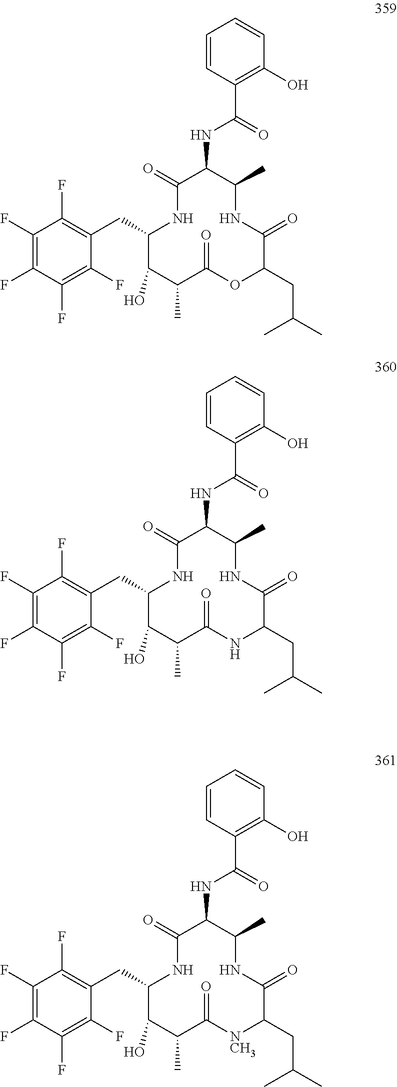 [SMILES: CC(C)CC1OC(=O)[C@H](C)[C@H](O)[C@H](Cc2c(F)c(F)c(F)c(F)c2F)NC(=O)[C@@H](NC(=O)c2ccccc2O)[C@@H](C)NC1=O ; CC(C)CC1NC(=O)[C@H](C)[C@H](O)[C@H](Cc2c(F)c(F)c(F)c(F)c2F)NC(=O)[C@@H](NC(=O)c2ccccc2O)[C@@H](C)NC1=O ; CC(C)CC1C(=O)N[C@H](C)[C@H](NC(=O)c2ccccc2O)C(=O)N[C@@H](Cc2c(F)c(F)c(F)c(F)c2F)[C@@H](O)[C@@H](C)C(=O)N1C]

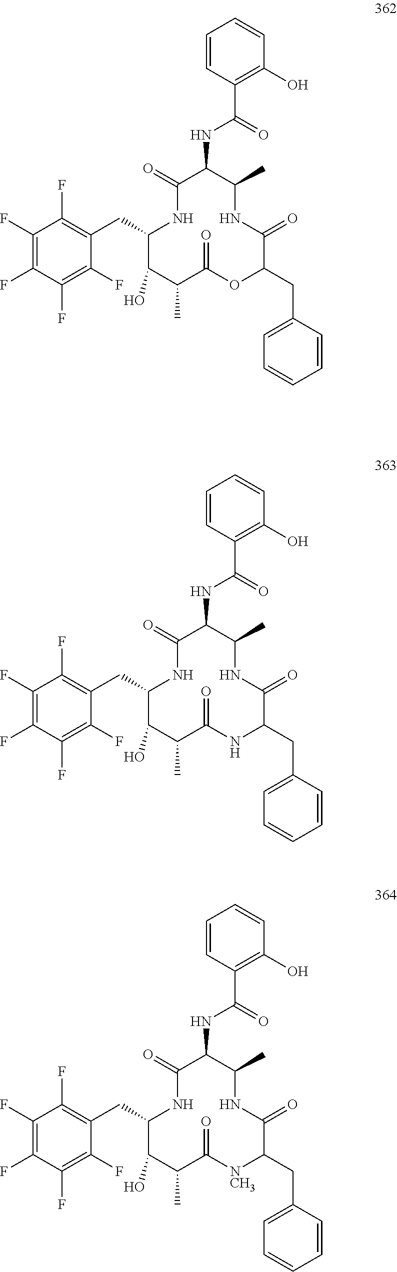 [SMILES: C[C@H]1NC(=O)C(Cc2ccccc2)OC(=O)[C@H](C)[C@H](O)[C@H](Cc2c(F)c(F)c(F)c(F)c2F)NC(=O)[C@H]1NC(=O)c1ccccc1O ; C[C@H]1NC(=O)C(Cc2ccccc2)NC(=O)[C@H](C)[C@H](O)[C@H](Cc2c(F)c(F)c(F)c(F)c2F)NC(=O)[C@H]1NC(=O)c1ccccc1O ; C[C@H]1NC(=O)C(Cc2ccccc2)N(C)C(=O)[C@H](C)[C@H](O)[C@H](Cc2c(F)c(F)c(F)c(F)c2F)NC(=O)[C@H]1NC(=O)c1ccccc1O]

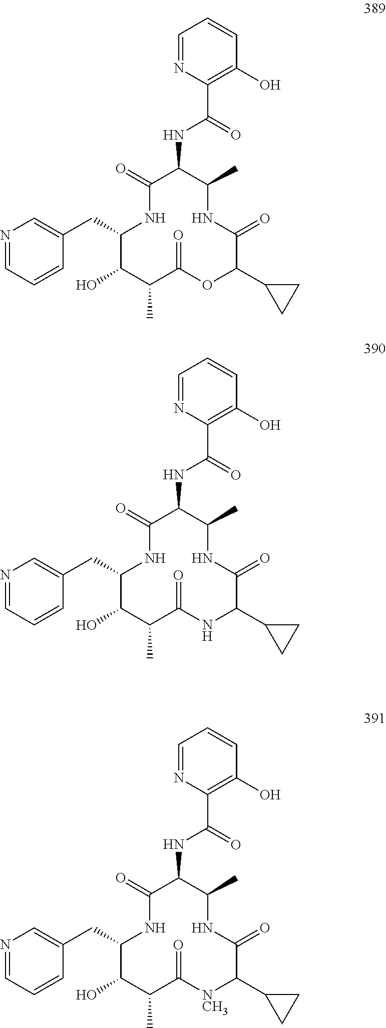 [SMILES: C[C@H]1NC(=O)C(C2CC2)OC(=O)[C@H](C)[C@H](O)[C@H](Cc2cccnc2)NC(=O)[C@H]1NC(=O)c1ncccc1O ; C[C@H]1NC(=O)C(C2CC2)NC(=O)[C@H](C)[C@H](O)[C@H](Cc2cccnc2)NC(=O)[C@H]1NC(=O)c1ncccc1O ; C[C@H]1NC(=O)C(C2CC2)N(C)C(=O)[C@H](C)[C@H](O)[C@H](Cc2cccnc2)NC(=O)[C@H]1NC(=O)c1ncccc1O]

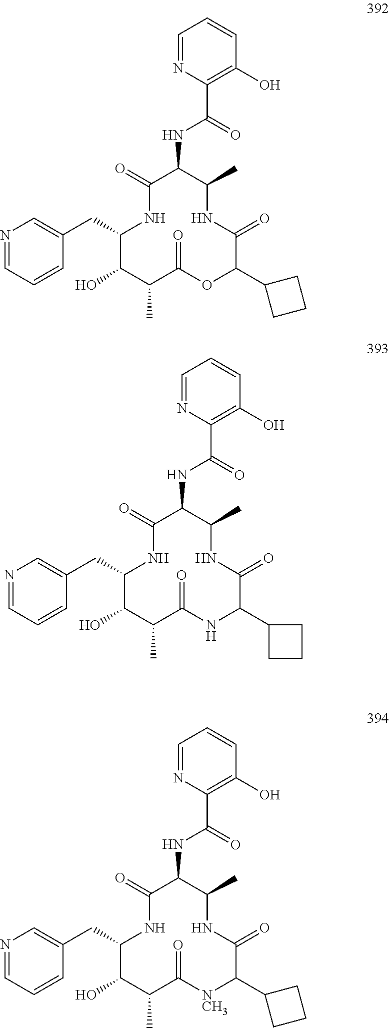 [SMILES: C[C@H]1NC(=O)C(C2CCC2)OC(=O)[C@H](C)[C@H](O)[C@H](Cc2cccnc2)NC(=O)[C@H]1NC(=O)c1ncccc1O ; C[C@H]1NC(=O)C(C2CCC2)NC(=O)[C@H](C)[C@H](O)[C@H](Cc2cccnc2)NC(=O)[C@H]1NC(=O)c1ncccc1O ; C[C@H]1NC(=O)C(C2CCC2)N(C)C(=O)[C@H](C)[C@H](O)[C@H](Cc2cccnc2)NC(=O)[C@H]1NC(=O)c1ncccc1O]

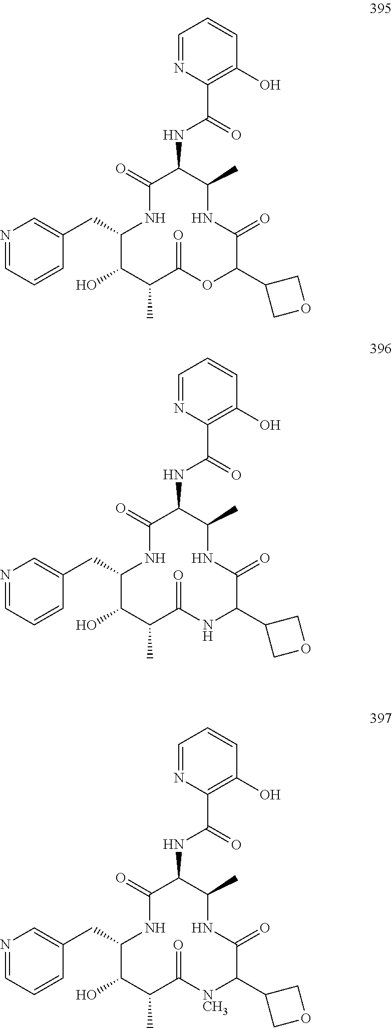 [SMILES: C[C@H]1NC(=O)C(C2COC2)OC(=O)[C@H](C)[C@H](O)[C@H](Cc2cccnc2)NC(=O)[C@H]1NC(=O)c1ncccc1O ; C[C@H]1NC(=O)C(C2COC2)NC(=O)[C@H](C)[C@H](O)[C@H](Cc2cccnc2)NC(=O)[C@H]1NC(=O)c1ncccc1O ; C[C@H]1NC(=O)C(C2COC2)N(C)C(=O)[C@H](C)[C@H](O)[C@H](Cc2cccnc2)NC(=O)[C@H]1NC(=O)c1ncccc1O]

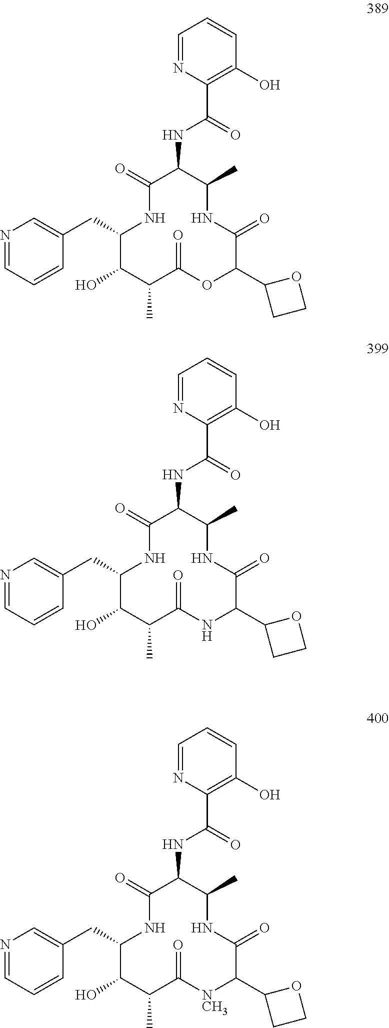 [SMILES: C[C@H]1NC(=O)C(C2CCO2)OC(=O)[C@H](C)[C@H](O)[C@H](Cc2cccnc2)NC(=O)[C@H]1NC(=O)c1ncccc1O ; C[C@H]1NC(=O)C(C2CCO2)NC(=O)[C@H](C)[C@H](O)[C@H](Cc2cccnc2)NC(=O)[C@H]1NC(=O)c1ncccc1O ; C[C@H]1NC(=O)C(C2CCO2)N(C)C(=O)[C@H](C)[C@H](O)[C@H](Cc2cccnc2)NC(=O)[C@H]1NC(=O)c1ncccc1O]

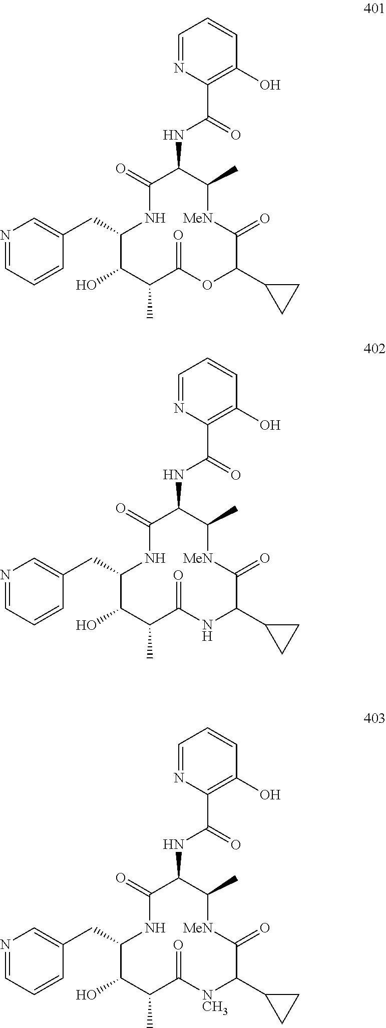 [SMILES: C[C@@H]1[C@H](NC(=O)c2ncccc2O)C(=O)N[C@@H](Cc2cccnc2)[C@@H](O)[C@@H](C)C(=O)OC(C2CC2)C(=O)N1C ; C[C@@H]1[C@H](NC(=O)c2ncccc2O)C(=O)N[C@@H](Cc2cccnc2)[C@@H](O)[C@@H](C)C(=O)NC(C2CC2)C(=O)N1C ; C[C@@H]1[C@H](NC(=O)c2ncccc2O)C(=O)N[C@@H](Cc2cccnc2)[C@@H](O)[C@@H](C)C(=O)N(C)C(C2CC2)C(=O)N1C]

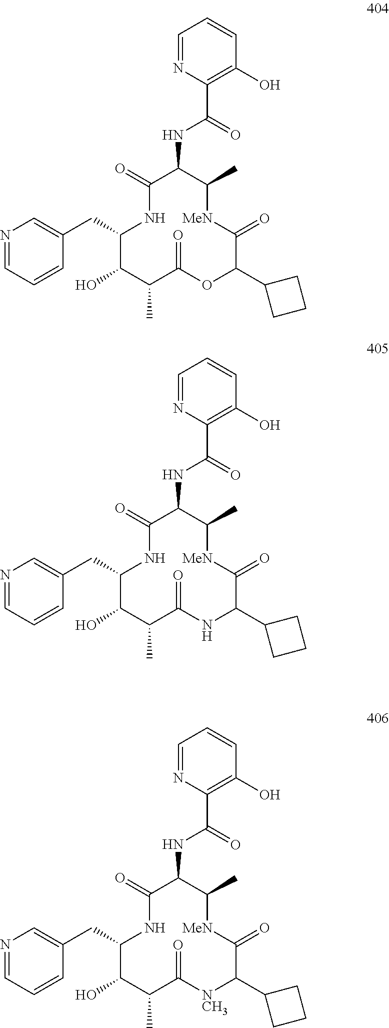 [SMILES: C[C@@H]1[C@H](NC(=O)c2ncccc2O)C(=O)N[C@@H](Cc2cccnc2)[C@@H](O)[C@@H](C)C(=O)OC(C2CCC2)C(=O)N1C ; C[C@@H]1[C@H](NC(=O)c2ncccc2O)C(=O)N[C@@H](Cc2cccnc2)[C@@H](O)[C@@H](C)C(=O)NC(C2CCC2)C(=O)N1C ; C[C@@H]1[C@H](NC(=O)c2ncccc2O)C(=O)N[C@@H](Cc2cccnc2)[C@@H](O)[C@@H](C)C(=O)N(C)C(C2CCC2)C(=O)N1C]

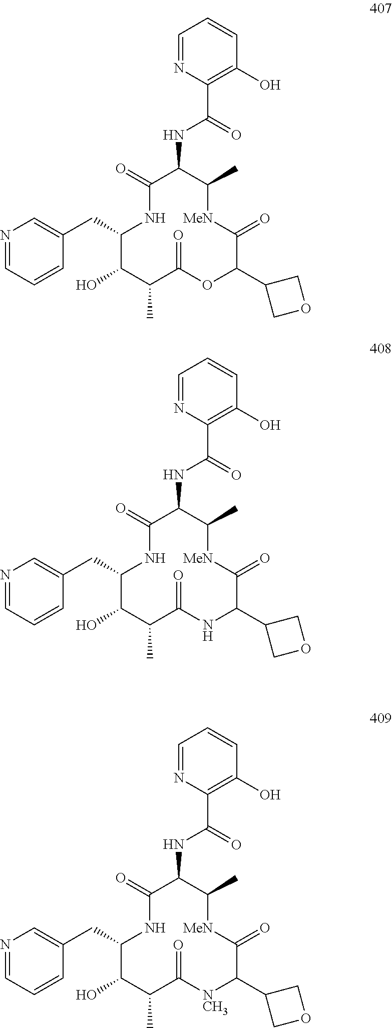 [SMILES: C[C@@H]1[C@H](NC(=O)c2ncccc2O)C(=O)N[C@@H](Cc2cccnc2)[C@@H](O)[C@@H](C)C(=O)OC(C2COC2)C(=O)N1C ; C[C@@H]1[C@H](NC(=O)c2ncccc2O)C(=O)N[C@@H](Cc2cccnc2)[C@@H](O)[C@@H](C)C(=O)NC(C2COC2)C(=O)N1C ; C[C@@H]1[C@H](NC(=O)c2ncccc2O)C(=O)N[C@@H](Cc2cccnc2)[C@@H](O)[C@@H](C)C(=O)N(C)C(C2COC2)C(=O)N1C]

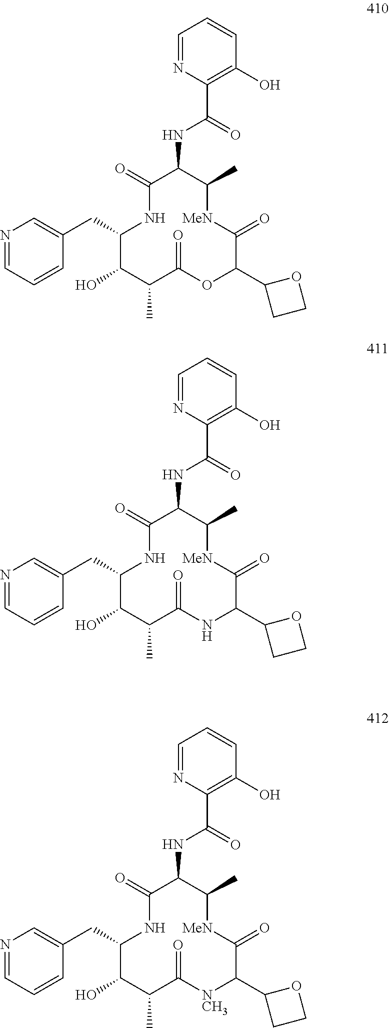 [SMILES: C[C@@H]1[C@H](NC(=O)c2ncccc2O)C(=O)N[C@@H](Cc2cccnc2)[C@@H](O)[C@@H](C)C(=O)OC(C2CCO2)C(=O)N1C ; C[C@@H]1[C@H](NC(=O)c2ncccc2O)C(=O)N[C@@H](Cc2cccnc2)[C@@H](O)[C@@H](C)C(=O)NC(C2CCO2)C(=O)N1C ; C[C@@H]1[C@H](NC(=O)c2ncccc2O)C(=O)N[C@@H](Cc2cccnc2)[C@@H](O)[C@@H](C)C(=O)N(C)C(C2CCO2)C(=O)N1C]

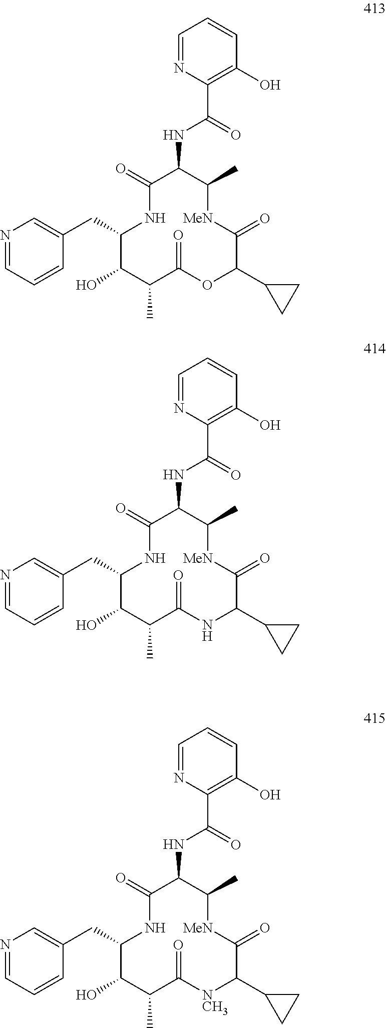 [SMILES: C[C@@H]1[C@H](NC(=O)c2ncccc2O)C(=O)N[C@@H](Cc2cccnc2)[C@@H](O)[C@@H](C)C(=O)OC(C2CC2)C(=O)N1C ; C[C@@H]1[C@H](NC(=O)c2ncccc2O)C(=O)N[C@@H](Cc2cccnc2)[C@@H](O)[C@@H](C)C(=O)NC(C2CC2)C(=O)N1C ; C[C@@H]1[C@H](NC(=O)c2ncccc2O)C(=O)N[C@@H](Cc2cccnc2)[C@@H](O)[C@@H](C)C(=O)N(C)C(C2CC2)C(=O)N1C]

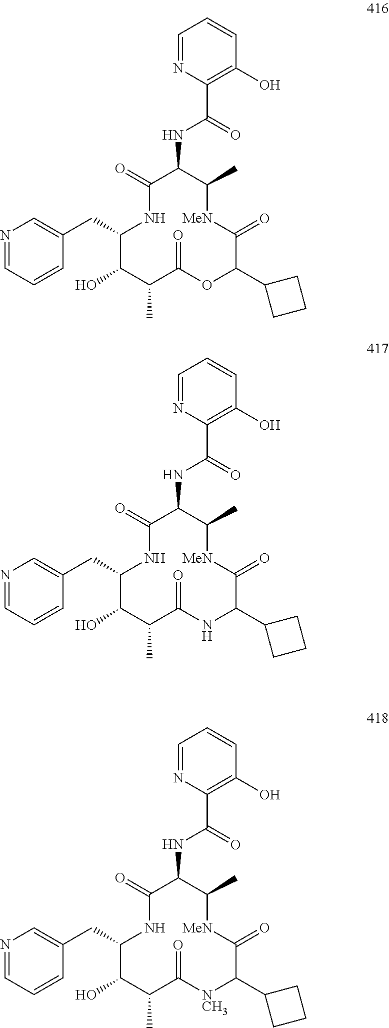 [SMILES: C[C@@H]1[C@H](NC(=O)c2ncccc2O)C(=O)N[C@@H](Cc2cccnc2)[C@@H](O)[C@@H](C)C(=O)OC(C2CCC2)C(=O)N1C ; C[C@@H]1[C@H](NC(=O)c2ncccc2O)C(=O)N[C@@H](Cc2cccnc2)[C@@H](O)[C@@H](C)C(=O)NC(C2CCC2)C(=O)N1C ; C[C@@H]1[C@H](NC(=O)c2ncccc2O)C(=O)N[C@@H](Cc2cccnc2)[C@@H](O)[C@@H](C)C(=O)N(C)C(C2CCC2)C(=O)N1C]

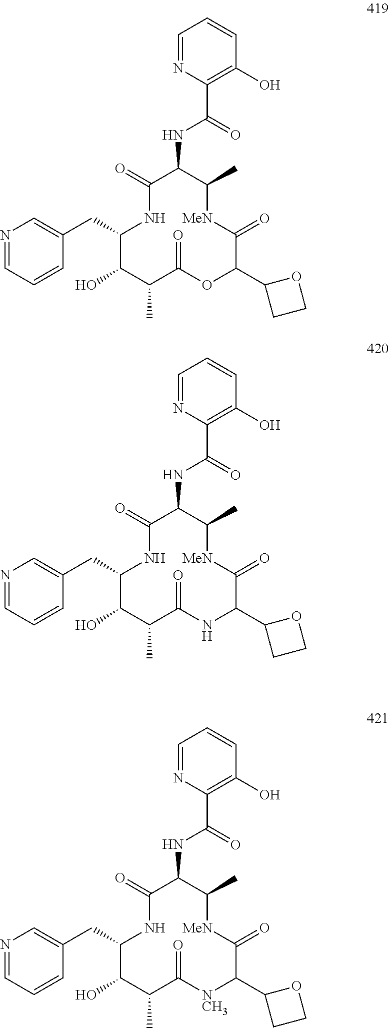 [SMILES: C[C@@H]1[C@H](NC(=O)c2ncccc2O)C(=O)N[C@@H](Cc2cccnc2)[C@@H](O)[C@@H](C)C(=O)OC(C2CCO2)C(=O)N1C ; C[C@@H]1[C@H](NC(=O)c2ncccc2O)C(=O)N[C@@H](Cc2cccnc2)[C@@H](O)[C@@H](C)C(=O)NC(C2CCO2)C(=O)N1C ; C[C@@H]1[C@H](NC(=O)c2ncccc2O)C(=O)N[C@@H](Cc2cccnc2)[C@@H](O)[C@@H](C)C(=O)N(C)C(C2CCO2)C(=O)N1C]

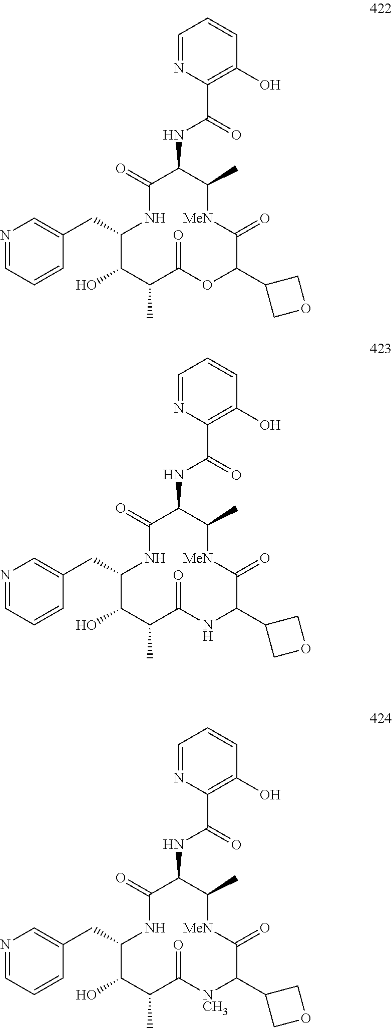 [SMILES: C[C@@H]1[C@H](NC(=O)c2ncccc2O)C(=O)N[C@@H](Cc2cccnc2)[C@@H](O)[C@@H](C)C(=O)OC(C2COC2)C(=O)N1C ; C[C@@H]1[C@H](NC(=O)c2ncccc2O)C(=O)N[C@@H](Cc2cccnc2)[C@@H](O)[C@@H](C)C(=O)NC(C2COC2)C(=O)N1C ; C[C@@H]1[C@H](NC(=O)c2ncccc2O)C(=O)N[C@@H](Cc2cccnc2)[C@@H](O)[C@@H](C)C(=O)N(C)C(C2COC2)C(=O)N1C]

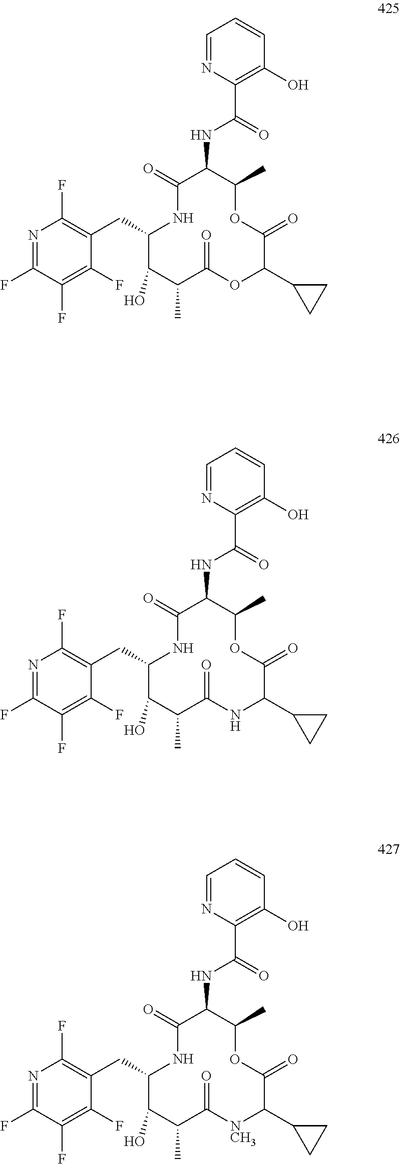 [SMILES: C[C@H]1OC(=O)C(C2CC2)OC(=O)[C@H](C)[C@H](O)[C@H](Cc2c(F)nc(F)c(F)c2F)NC(=O)[C@H]1NC(=O)c1ncccc1O ; C[C@H]1OC(=O)C(C2CC2)NC(=O)[C@H](C)[C@H](O)[C@H](Cc2c(F)nc(F)c(F)c2F)NC(=O)[C@H]1NC(=O)c1ncccc1O ; C[C@H]1OC(=O)C(C2CC2)N(C)C(=O)[C@H](C)[C@H](O)[C@H](Cc2c(F)nc(F)c(F)c2F)NC(=O)[C@H]1NC(=O)c1ncccc1O]

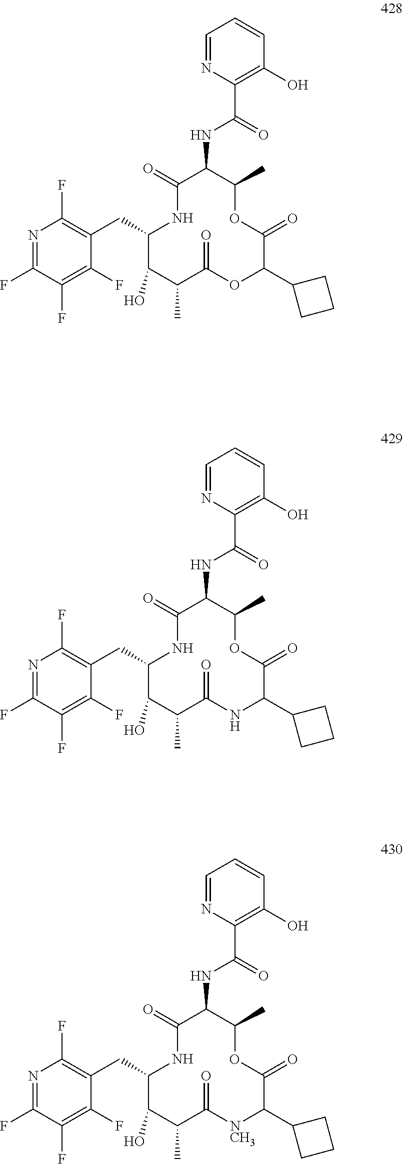 [SMILES: C[C@H]1OC(=O)C(C2CCC2)OC(=O)[C@H](C)[C@H](O)[C@H](Cc2c(F)nc(F)c(F)c2F)NC(=O)[C@H]1NC(=O)c1ncccc1O ; C[C@H]1OC(=O)C(C2CCC2)NC(=O)[C@H](C)[C@H](O)[C@H](Cc2c(F)nc(F)c(F)c2F)NC(=O)[C@H]1NC(=O)c1ncccc1O ; C[C@H]1OC(=O)C(C2CCC2)N(C)C(=O)[C@H](C)[C@H](O)[C@H](Cc2c(F)nc(F)c(F)c2F)NC(=O)[C@H]1NC(=O)c1ncccc1O]

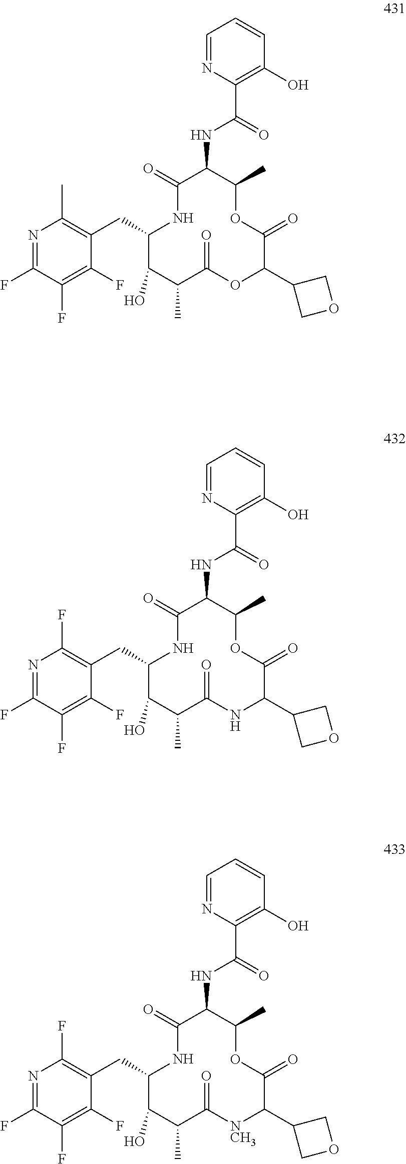 [SMILES: Cc1nc(F)c(F)c(F)c1C[C@@H]1NC(=O)[C@@H](NC(=O)c2ncccc2O)[C@@H](C)OC(=O)C(C2COC2)OC(=O)[C@H](C)[C@@H]1O ; C[C@H]1OC(=O)C(C2COC2)NC(=O)[C@H](C)[C@H](O)[C@H](Cc2c(F)nc(F)c(F)c2F)NC(=O)[C@H]1NC(=O)c1ncccc1O ; C[C@H]1OC(=O)C(C2COC2)N(C)C(=O)[C@H](C)[C@H](O)[C@H](Cc2c(F)nc(F)c(F)c2F)NC(=O)[C@H]1NC(=O)c1ncccc1O]

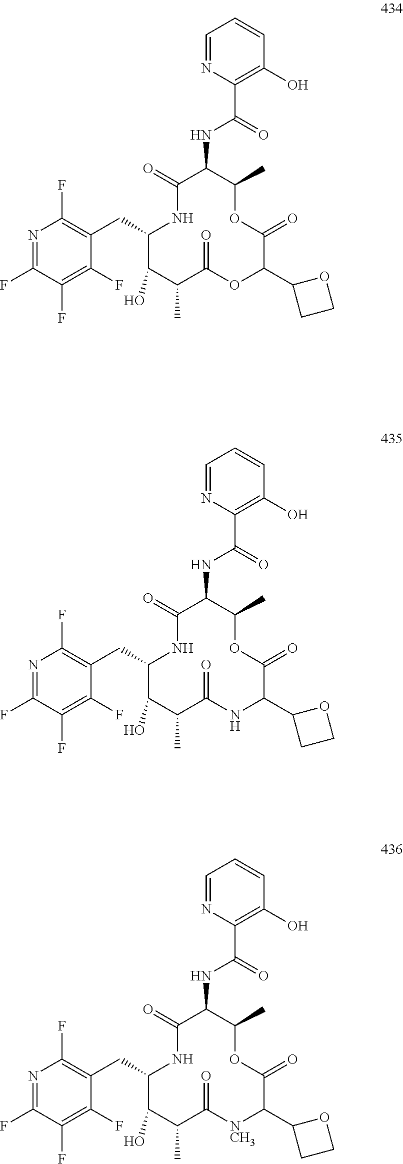 [SMILES: C[C@H]1OC(=O)C(C2CCO2)OC(=O)[C@H](C)[C@H](O)[C@H](Cc2c(F)nc(F)c(F)c2F)NC(=O)[C@H]1NC(=O)c1ncccc1O ; C[C@H]1OC(=O)C(C2CCO2)NC(=O)[C@H](C)[C@H](O)[C@H](Cc2c(F)nc(F)c(F)c2F)NC(=O)[C@H]1NC(=O)c1ncccc1O ; C[C@H]1OC(=O)C(C2CCO2)N(C)C(=O)[C@H](C)[C@H](O)[C@H](Cc2c(F)nc(F)c(F)c2F)NC(=O)[C@H]1NC(=O)c1ncccc1O]

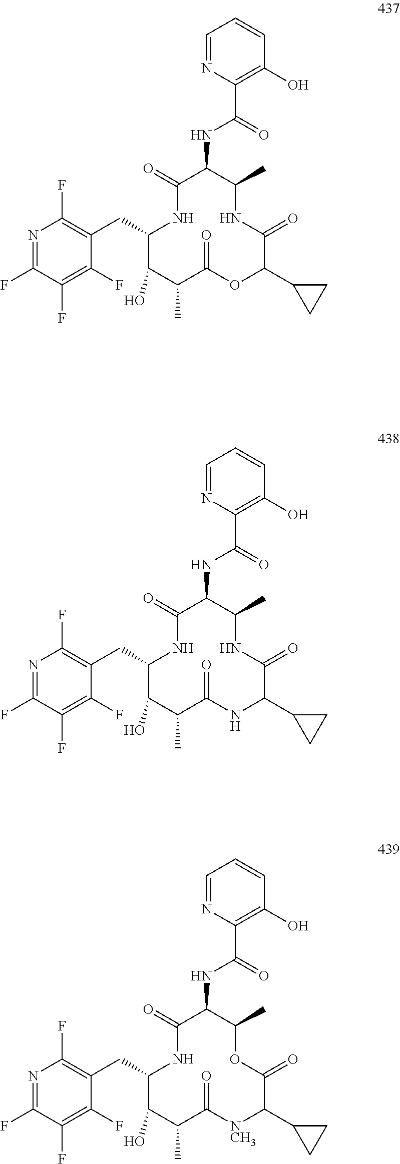 [SMILES: C[C@H]1NC(=O)C(C2CC2)OC(=O)[C@H](C)[C@H](O)[C@H](Cc2c(F)nc(F)c(F)c2F)NC(=O)[C@H]1NC(=O)c1ncccc1O ; C[C@H]1NC(=O)C(C2CC2)NC(=O)[C@H](C)[C@H](O)[C@H](Cc2c(F)nc(F)c(F)c2F)NC(=O)[C@H]1NC(=O)c1ncccc1O ; C[C@H]1OC(=O)C(C2CC2)N(C)C(=O)[C@H](C)[C@H](O)[C@H](Cc2c(F)nc(F)c(F)c2F)NC(=O)[C@H]1NC(=O)c1ncccc1O]

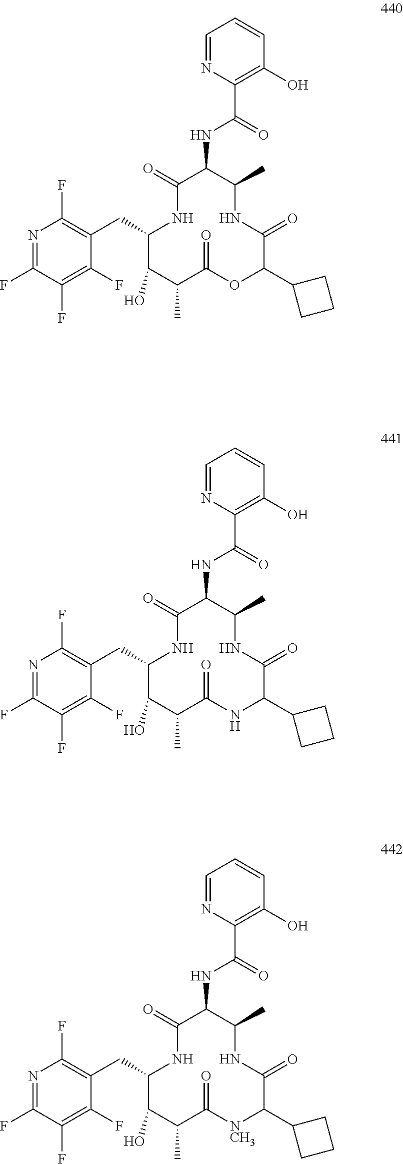 [SMILES: C[C@H]1NC(=O)C(C2CCC2)OC(=O)[C@H](C)[C@H](O)[C@H](Cc2c(F)nc(F)c(F)c2F)NC(=O)[C@H]1NC(=O)c1ncccc1O ; C[C@H]1NC(=O)C(C2CCC2)NC(=O)[C@H](C)[C@H](O)[C@H](Cc2c(F)nc(F)c(F)c2F)NC(=O)[C@H]1NC(=O)c1ncccc1O ; C[C@H]1NC(=O)C(C2CCC2)N(C)C(=O)[C@H](C)[C@H](O)[C@H](Cc2c(F)nc(F)c(F)c2F)NC(=O)[C@H]1NC(=O)c1ncccc1O]

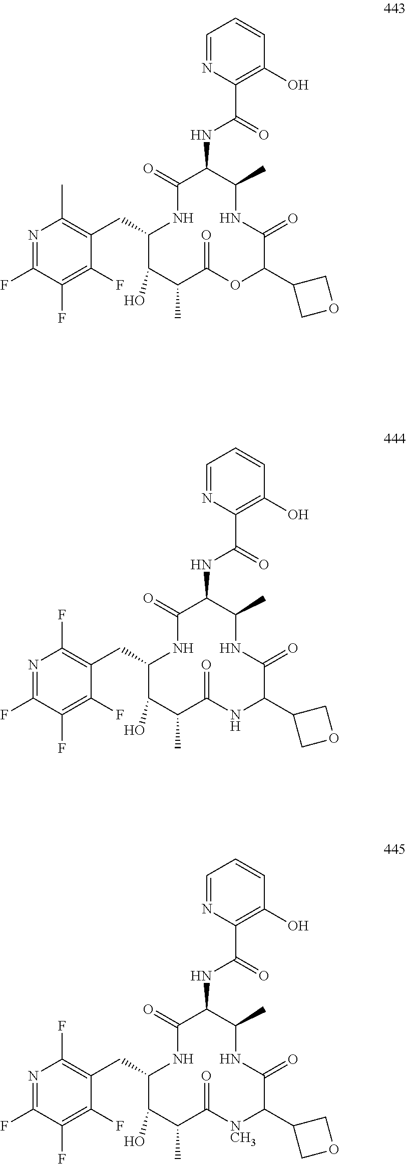 [SMILES: Cc1nc(F)c(F)c(F)c1C[C@@H]1NC(=O)[C@@H](NC(=O)c2ncccc2O)[C@@H](C)NC(=O)C(C2COC2)OC(=O)[C@H](C)[C@@H]1O ; C[C@H]1NC(=O)C(C2COC2)NC(=O)[C@H](C)[C@H](O)[C@H](Cc2c(F)nc(F)c(F)c2F)NC(=O)[C@H]1NC(=O)c1ncccc1O ; C[C@H]1NC(=O)C(C2COC2)N(C)C(=O)[C@H](C)[C@H](O)[C@H](Cc2c(F)nc(F)c(F)c2F)NC(=O)[C@H]1NC(=O)c1ncccc1O]

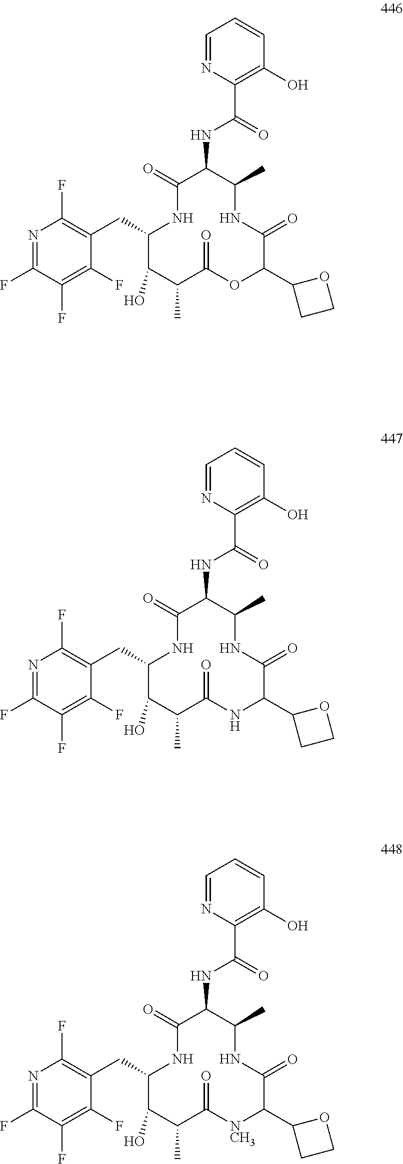 [SMILES: C[C@H]1NC(=O)C(C2CCO2)OC(=O)[C@H](C)[C@H](O)[C@H](Cc2c(F)nc(F)c(F)c2F)NC(=O)[C@H]1NC(=O)c1ncccc1O ; C[C@H]1NC(=O)C(C2CCO2)NC(=O)[C@H](C)[C@H](O)[C@H](Cc2c(F)nc(F)c(F)c2F)NC(=O)[C@H]1NC(=O)c1ncccc1O ; C[C@H]1NC(=O)C(C2CCO2)N(C)C(=O)[C@H](C)[C@H](O)[C@H](Cc2c(F)nc(F)c(F)c2F)NC(=O)[C@H]1NC(=O)c1ncccc1O]

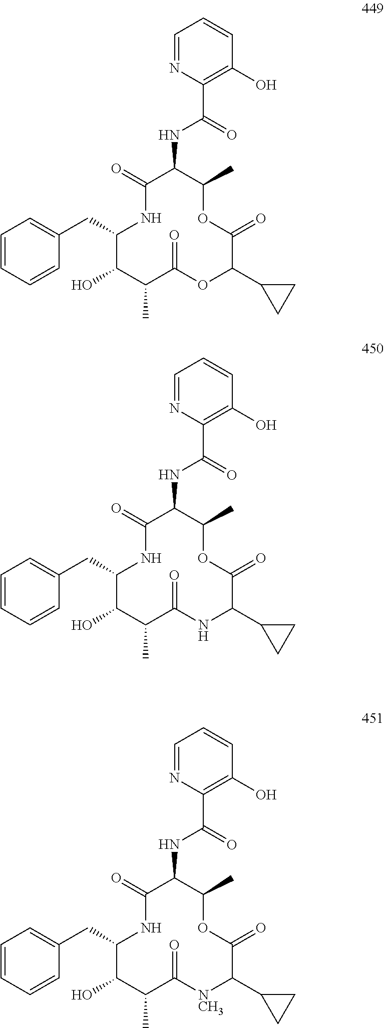 [SMILES: C[C@H]1OC(=O)C(C2CC2)OC(=O)[C@H](C)[C@H](O)[C@H](Cc2ccccc2)NC(=O)[C@H]1NC(=O)c1ncccc1O ; C[C@H]1OC(=O)C(C2CC2)NC(=O)[C@H](C)[C@H](O)[C@H](Cc2ccccc2)NC(=O)[C@H]1NC(=O)c1ncccc1O ; C[C@H]1OC(=O)C(C2CC2)N(C)C(=O)[C@H](C)[C@H](O)[C@H](Cc2ccccc2)NC(=O)[C@H]1NC(=O)c1ncccc1O]

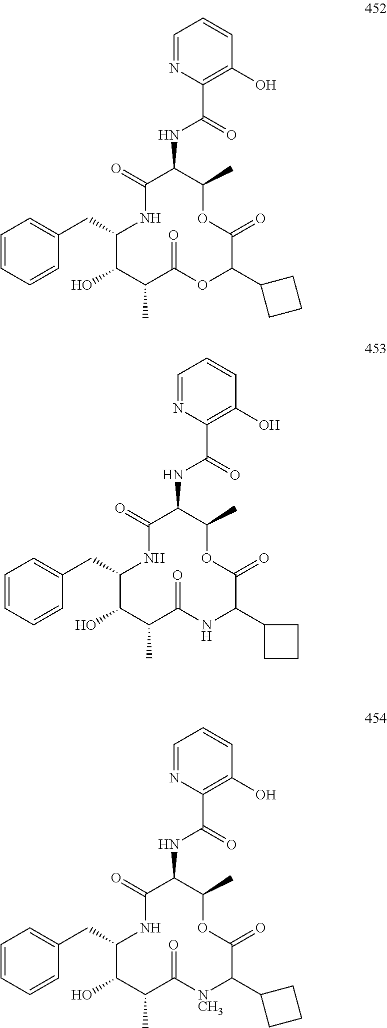 [SMILES: C[C@H]1OC(=O)C(C2CCC2)OC(=O)[C@H](C)[C@H](O)[C@H](Cc2ccccc2)NC(=O)[C@H]1NC(=O)c1ncccc1O ; C[C@H]1OC(=O)C(C2CCC2)NC(=O)[C@H](C)[C@H](O)[C@H](Cc2ccccc2)NC(=O)[C@H]1NC(=O)c1ncccc1O ; C[C@H]1OC(=O)C(C2CCC2)N(C)C(=O)[C@H](C)[C@H](O)[C@H](Cc2ccccc2)NC(=O)[C@H]1NC(=O)c1ncccc1O]

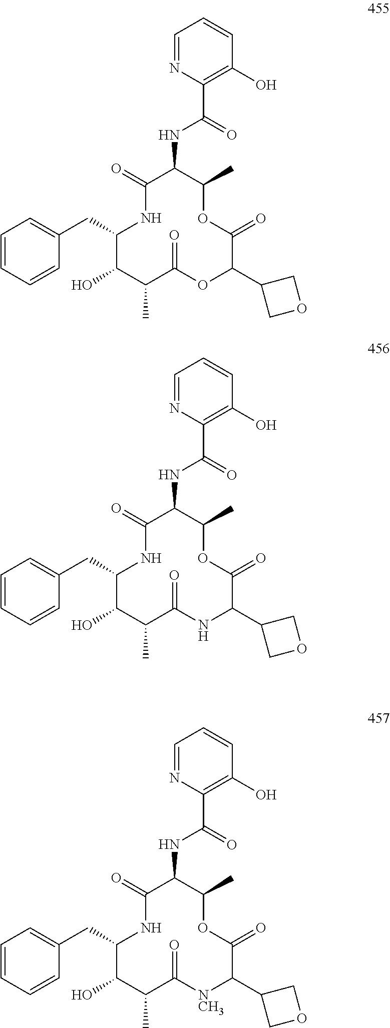 [SMILES: C[C@H]1OC(=O)C(C2COC2)OC(=O)[C@H](C)[C@H](O)[C@H](Cc2ccccc2)NC(=O)[C@H]1NC(=O)c1ncccc1O ; C[C@H]1OC(=O)C(C2COC2)NC(=O)[C@H](C)[C@H](O)[C@H](Cc2ccccc2)NC(=O)[C@H]1NC(=O)c1ncccc1O ; C[C@H]1OC(=O)C(C2COC2)N(C)C(=O)[C@H](C)[C@H](O)[C@H](Cc2ccccc2)NC(=O)[C@H]1NC(=O)c1ncccc1O]

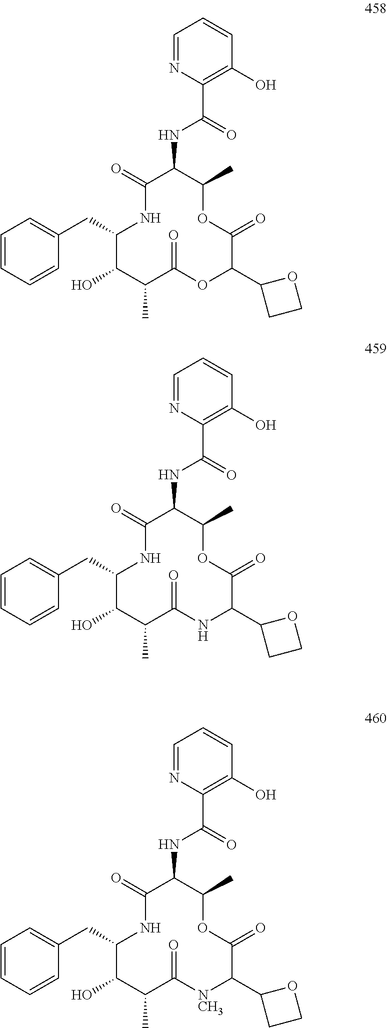 [SMILES: C[C@H]1OC(=O)C(C2CCO2)OC(=O)[C@H](C)[C@H](O)[C@H](Cc2ccccc2)NC(=O)[C@H]1NC(=O)c1ncccc1O ; C[C@H]1OC(=O)C(C2CCO2)NC(=O)[C@H](C)[C@H](O)[C@H](Cc2ccccc2)NC(=O)[C@H]1NC(=O)c1ncccc1O ; C[C@H]1OC(=O)C(C2CCO2)N(C)C(=O)[C@H](C)[C@H](O)[C@H](Cc2ccccc2)NC(=O)[C@H]1NC(=O)c1ncccc1O]

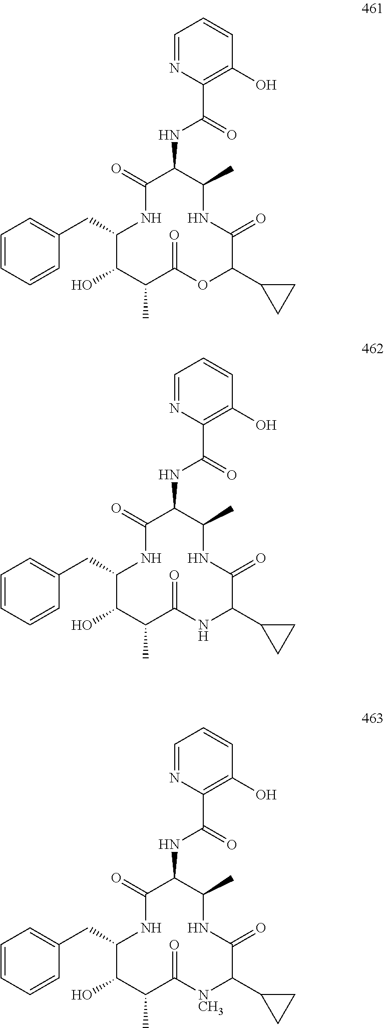 [SMILES: C[C@H]1NC(=O)C(C2CC2)OC(=O)[C@H](C)[C@H](O)[C@H](Cc2ccccc2)NC(=O)[C@H]1NC(=O)c1ncccc1O ; C[C@H]1NC(=O)C(C2CC2)NC(=O)[C@H](C)[C@H](O)[C@H](Cc2ccccc2)NC(=O)[C@H]1NC(=O)c1ncccc1O ; C[C@H]1NC(=O)C(C2CC2)N(C)C(=O)[C@H](C)[C@H](O)[C@H](Cc2ccccc2)NC(=O)[C@H]1NC(=O)c1ncccc1O]

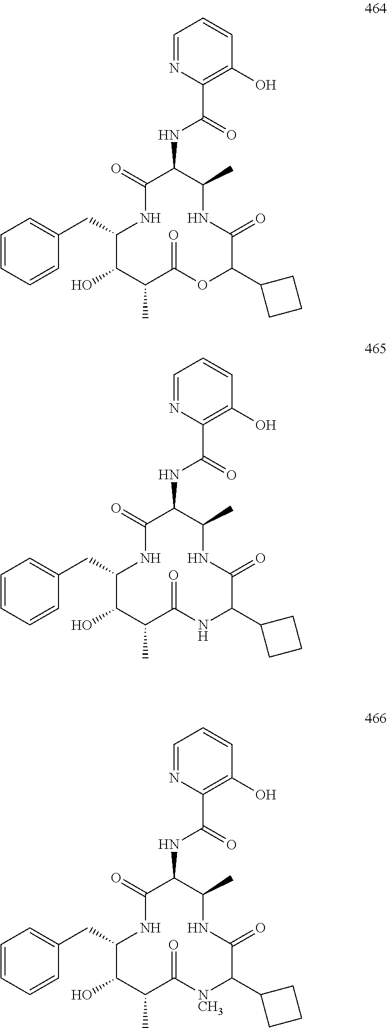 [SMILES: C[C@H]1NC(=O)C(C2CCC2)OC(=O)[C@H](C)[C@H](O)[C@H](Cc2ccccc2)NC(=O)[C@H]1NC(=O)c1ncccc1O ; C[C@H]1NC(=O)C(C2CCC2)NC(=O)[C@H](C)[C@H](O)[C@H](Cc2ccccc2)NC(=O)[C@H]1NC(=O)c1ncccc1O ; C[C@H]1NC(=O)C(C2CCC2)N(C)C(=O)[C@H](C)[C@H](O)[C@H](Cc2ccccc2)NC(=O)[C@H]1NC(=O)c1ncccc1O]

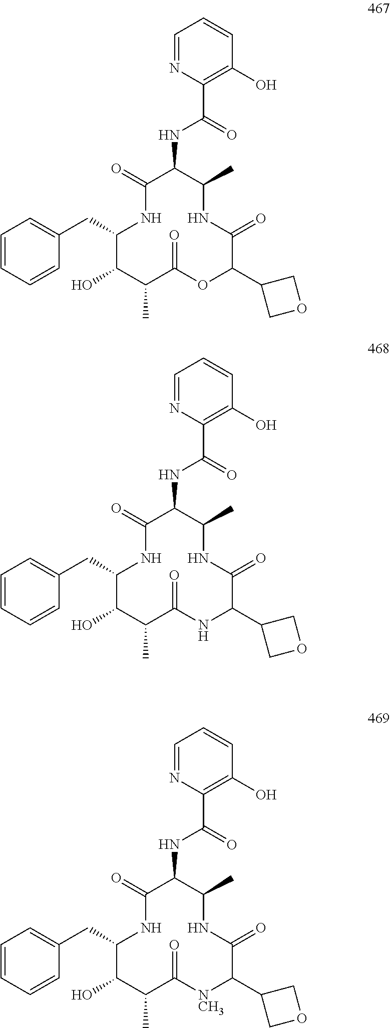 [SMILES: C[C@H]1NC(=O)C(C2COC2)OC(=O)[C@H](C)[C@H](O)[C@H](Cc2ccccc2)NC(=O)[C@H]1NC(=O)c1ncccc1O ; C[C@H]1NC(=O)C(C2COC2)NC(=O)[C@H](C)[C@H](O)[C@H](Cc2ccccc2)NC(=O)[C@H]1NC(=O)c1ncccc1O ; C[C@H]1NC(=O)C(C2COC2)N(C)C(=O)[C@H](C)[C@H](O)[C@H](Cc2ccccc2)NC(=O)[C@H]1NC(=O)c1ncccc1O]

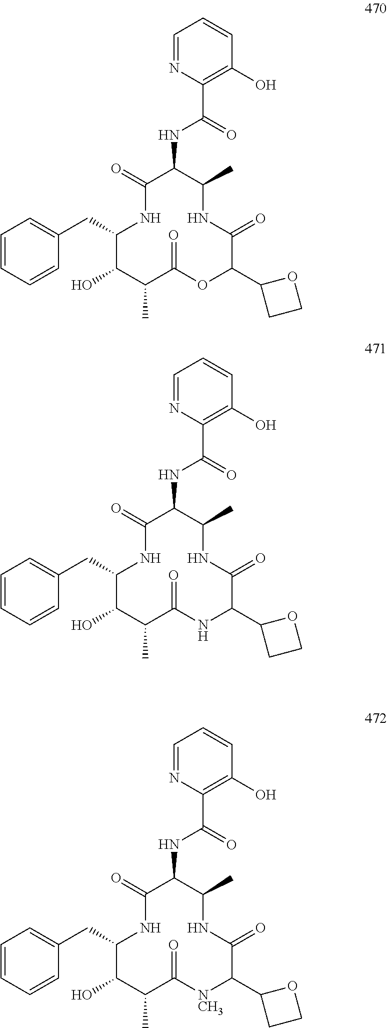 [SMILES: C[C@H]1NC(=O)C(C2CCO2)OC(=O)[C@H](C)[C@H](O)[C@H](Cc2ccccc2)NC(=O)[C@H]1NC(=O)c1ncccc1O ; C[C@H]1NC(=O)C(C2CCO2)NC(=O)[C@H](C)[C@H](O)[C@H](Cc2ccccc2)NC(=O)[C@H]1NC(=O)c1ncccc1O ; C[C@H]1NC(=O)C(C2CCO2)N(C)C(=O)[C@H](C)[C@H](O)[C@H](Cc2ccccc2)NC(=O)[C@H]1NC(=O)c1ncccc1O]

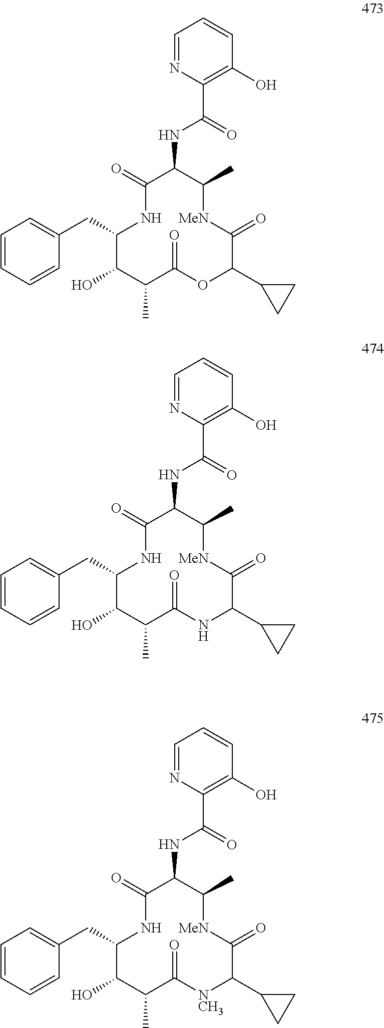 [SMILES: C[C@@H]1[C@H](NC(=O)c2ncccc2O)C(=O)N[C@@H](Cc2ccccc2)[C@@H](O)[C@@H](C)C(=O)OC(C2CC2)C(=O)N1C ; C[C@@H]1[C@H](NC(=O)c2ncccc2O)C(=O)N[C@@H](Cc2ccccc2)[C@@H](O)[C@@H](C)C(=O)NC(C2CC2)C(=O)N1C ; C[C@@H]1[C@H](NC(=O)c2ncccc2O)C(=O)N[C@@H](Cc2ccccc2)[C@@H](O)[C@@H](C)C(=O)N(C)C(C2CC2)C(=O)N1C]

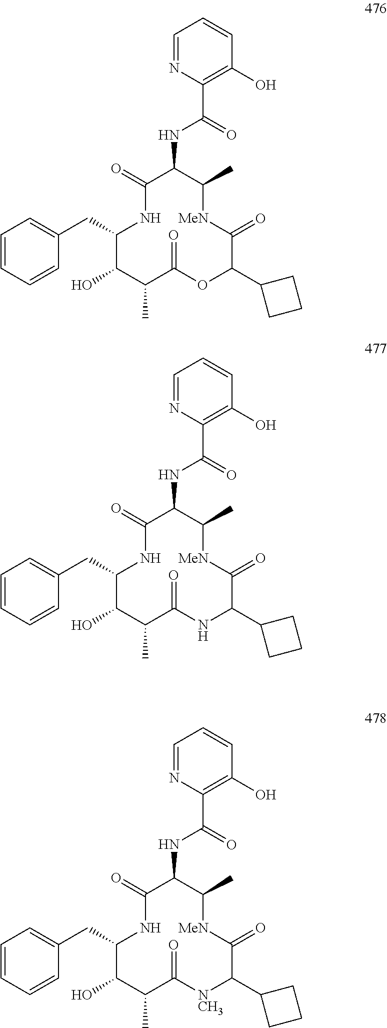 [SMILES: C[C@@H]1[C@H](NC(=O)c2ncccc2O)C(=O)N[C@@H](Cc2ccccc2)[C@@H](O)[C@@H](C)C(=O)OC(C2CCC2)C(=O)N1C ; C[C@@H]1[C@H](NC(=O)c2ncccc2O)C(=O)N[C@@H](Cc2ccccc2)[C@@H](O)[C@@H](C)C(=O)NC(C2CCC2)C(=O)N1C ; C[C@@H]1[C@H](NC(=O)c2ncccc2O)C(=O)N[C@@H](Cc2ccccc2)[C@@H](O)[C@@H](C)C(=O)N(C)C(C2CCC2)C(=O)N1C]

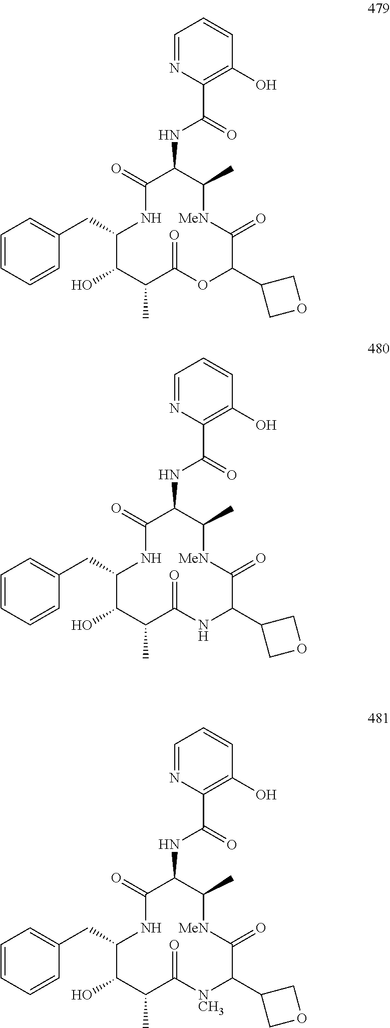 [SMILES: C[C@@H]1[C@H](NC(=O)c2ncccc2O)C(=O)N[C@@H](Cc2ccccc2)[C@@H](O)[C@@H](C)C(=O)OC(C2COC2)C(=O)N1C ; C[C@@H]1[C@H](NC(=O)c2ncccc2O)C(=O)N[C@@H](Cc2ccccc2)[C@@H](O)[C@@H](C)C(=O)NC(C2COC2)C(=O)N1C ; C[C@@H]1[C@H](NC(=O)c2ncccc2O)C(=O)N[C@@H](Cc2ccccc2)[C@@H](O)[C@@H](C)C(=O)N(C)C(C2COC2)C(=O)N1C]

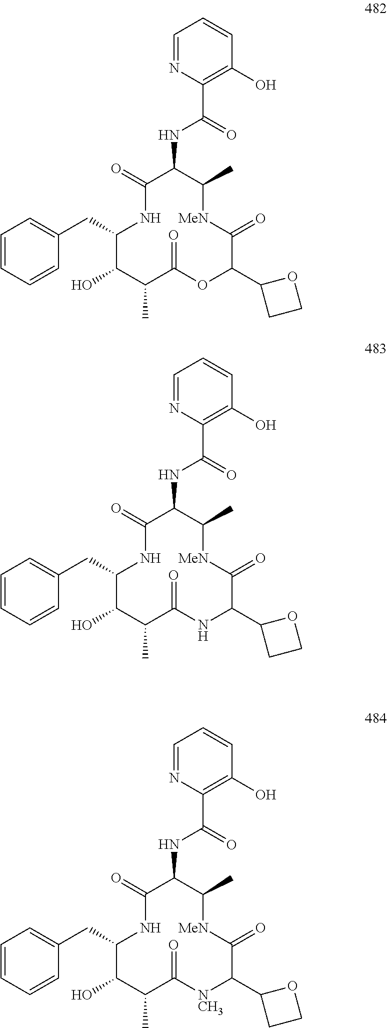 [SMILES: C[C@@H]1[C@H](NC(=O)c2ncccc2O)C(=O)N[C@@H](Cc2ccccc2)[C@@H](O)[C@@H](C)C(=O)OC(C2CCO2)C(=O)N1C ; C[C@@H]1[C@H](NC(=O)c2ncccc2O)C(=O)N[C@@H](Cc2ccccc2)[C@@H](O)[C@@H](C)C(=O)NC(C2CCO2)C(=O)N1C ; C[C@@H]1[C@H](NC(=O)c2ncccc2O)C(=O)N[C@@H](Cc2ccccc2)[C@@H](O)[C@@H](C)C(=O)N(C)C(C2CCO2)C(=O)N1C]

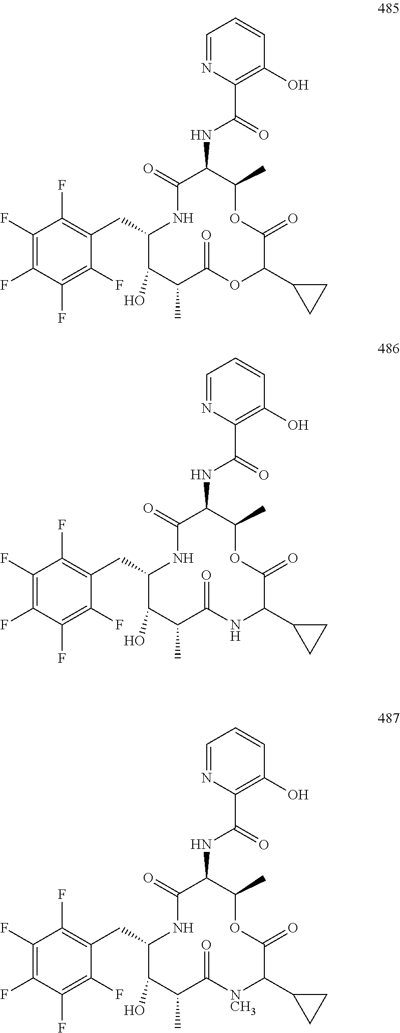 [SMILES: C[C@H]1OC(=O)C(C2CC2)OC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(C2CC2)NC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(C2CC2)N(C)C(=O)[C@H](C)[C@H](O)[C@H](Cc2c(F)c(F)c(F)c(F)c2F)NC(=O)[C@H]1NC(=O)c1ncccc1O]

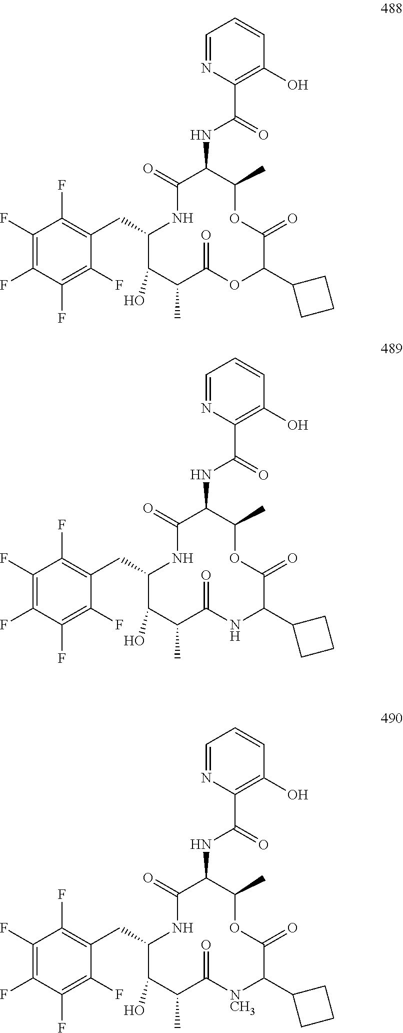 [SMILES: C[C@H]1OC(=O)C(C2CCC2)OC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(C2CCC2)NC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(C2CCC2)N(C)C(=O)[C@H](C)[C@H](O)[C@H](Cc2c(F)c(F)c(F)c(F)c2F)NC(=O)[C@H]1NC(=O)c1ncccc1O]

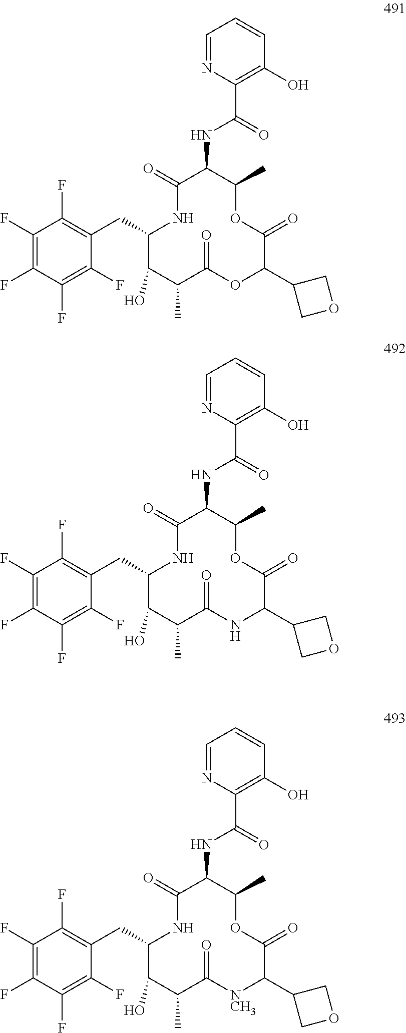 [SMILES: C[C@H]1OC(=O)C(C2COC2)OC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(C2COC2)NC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(C2COC2)N(C)C(=O)[C@H](C)[C@H](O)[C@H](Cc2c(F)c(F)c(F)c(F)c2F)NC(=O)[C@H]1NC(=O)c1ncccc1O]

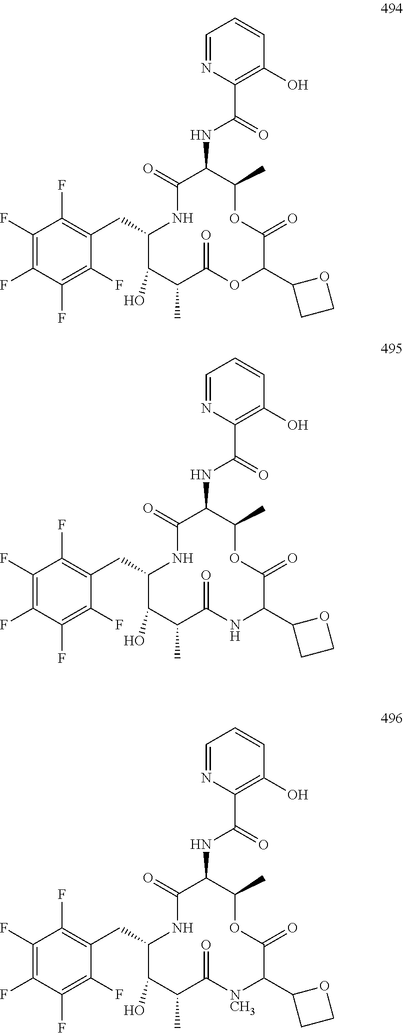 [SMILES: C[C@H]1OC(=O)C(C2CCO2)OC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(C2CCO2)NC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(C2CCO2)N(C)C(=O)[C@H](C)[C@H](O)[C@H](Cc2c(F)c(F)c(F)c(F)c2F)NC(=O)[C@H]1NC(=O)c1ncccc1O]

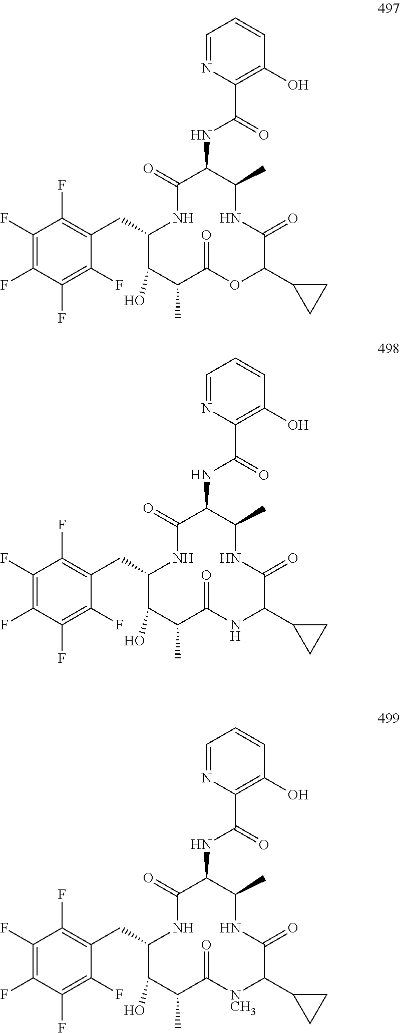 [SMILES: C[C@H]1NC(=O)C(C2CC2)OC(=O)[C@H](C)[C@H](O)[C@H](Cc2c(F)c(F)c(F)c(F)c2F)NC(=O)[C@H]1NC(=O)c1ncccc1O ; C[C@H]1NC(=O)C(C2CC2)NC(=O)[C@H](C)[C@H](O)[C@H](Cc2c(F)c(F)c(F)c(F)c2F)NC(=O)[C@H]1NC(=O)c1ncccc1O ; C[C@H]1NC(=O)C(C2CC2)N(C)C(=O)[C@H](C)[C@H](O)[C@H](Cc2c(F)c(F)c(F)c(F)c2F)NC(=O)[C@H]1NC(=O)c1ncccc1O]

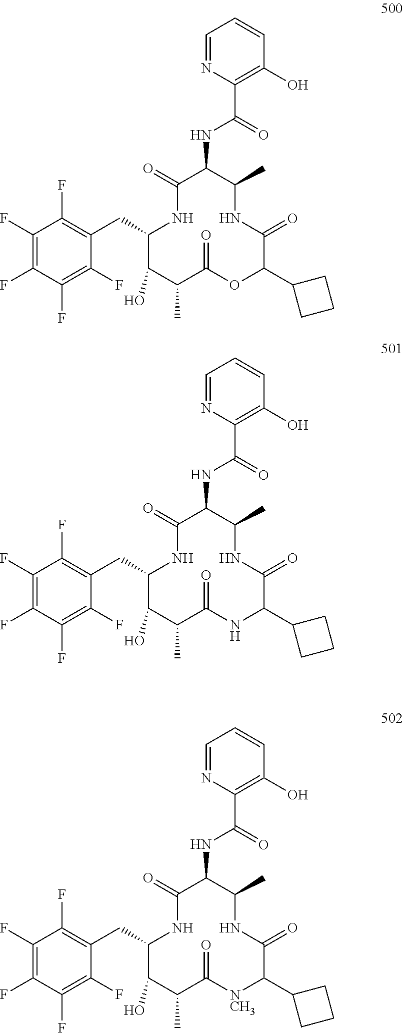 [SMILES: C[C@H]1NC(=O)C(C2CCC2)OC(=O)[C@H](C)[C@H](O)[C@H](Cc2c(F)c(F)c(F)c(F)c2F)NC(=O)[C@H]1NC(=O)c1ncccc1O ; C[C@H]1NC(=O)C(C2CCC2)NC(=O)[C@H](C)[C@H](O)[C@H](Cc2c(F)c(F)c(F)c(F)c2F)NC(=O)[C@H]1NC(=O)c1ncccc1O ; C[C@H]1NC(=O)C(C2CCC2)N(C)C(=O)[C@H](C)[C@H](O)[C@H](Cc2c(F)c(F)c(F)c(F)c2F)NC(=O)[C@H]1NC(=O)c1ncccc1O]

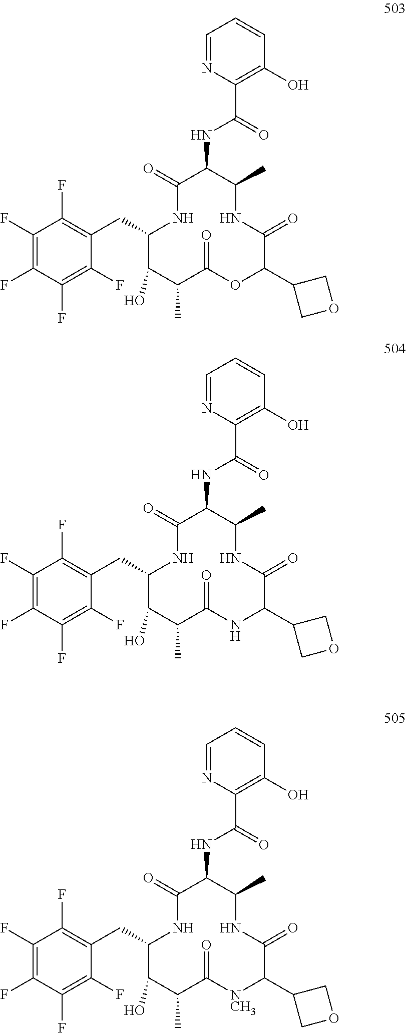 [SMILES: C[C@H]1NC(=O)C(C2COC2)OC(=O)[C@H](C)[C@H](O)[C@H](Cc2c(F)c(F)c(F)c(F)c2F)NC(=O)[C@H]1NC(=O)c1ncccc1O ; C[C@H]1NC(=O)C(C2COC2)NC(=O)[C@H](C)[C@H](O)[C@H](Cc2c(F)c(F)c(F)c(F)c2F)NC(=O)[C@H]1NC(=O)c1ncccc1O ; C[C@H]1NC(=O)C(C2COC2)N(C)C(=O)[C@H](C)[C@H](O)[C@H](Cc2c(F)c(F)c(F)c(F)c2F)NC(=O)[C@H]1NC(=O)c1ncccc1O]

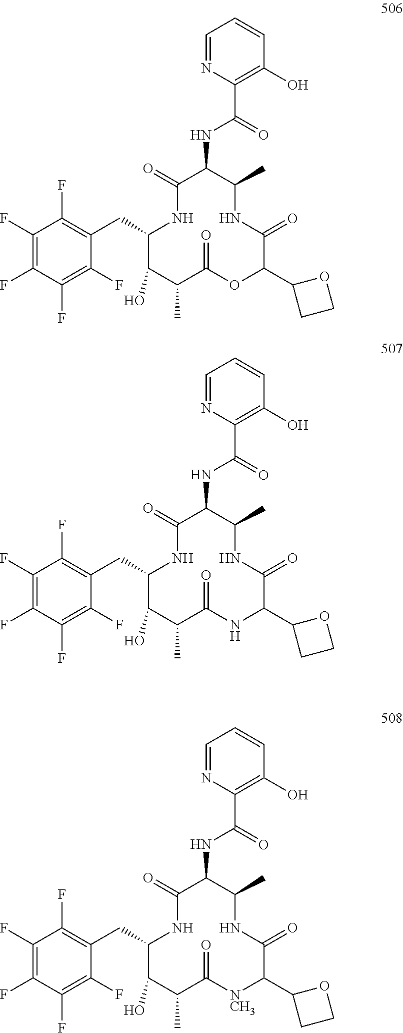 [SMILES: C[C@H]1NC(=O)C(C2CCO2)OC(=O)[C@H](C)[C@H](O)[C@H](Cc2c(F)c(F)c(F)c(F)c2F)NC(=O)[C@H]1NC(=O)c1ncccc1O ; C[C@H]1NC(=O)C(C2CCO2)NC(=O)[C@H](C)[C@H](O)[C@H](Cc2c(F)c(F)c(F)c(F)c2F)NC(=O)[C@H]1NC(=O)c1ncccc1O ; C[C@H]1NC(=O)C(C2CCO2)N(C)C(=O)[C@H](C)[C@H](O)[C@H](Cc2c(F)c(F)c(F)c(F)c2F)NC(=O)[C@H]1NC(=O)c1ncccc1O]

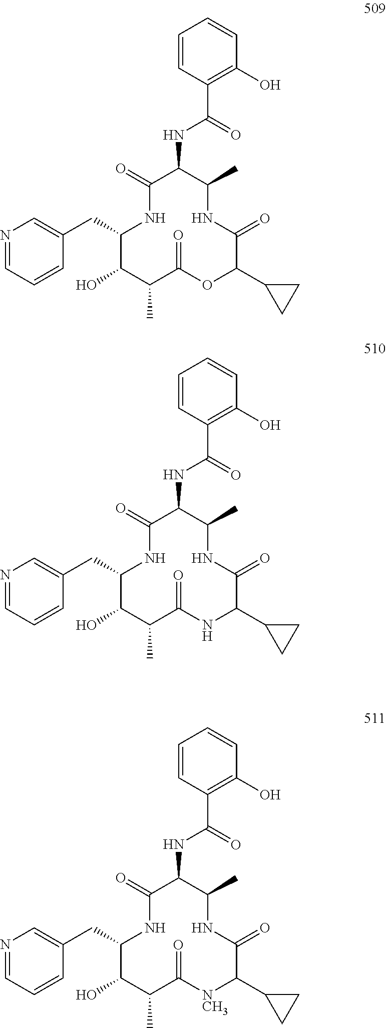 [SMILES: C[C@H]1NC(=O)C(C2CC2)OC(=O)[C@H](C)[C@H](O)[C@H](Cc2cccnc2)NC(=O)[C@H]1NC(=O)c1ccccc1O ; C[C@H]1NC(=O)C(C2CC2)NC(=O)[C@H](C)[C@H](O)[C@H](Cc2cccnc2)NC(=O)[C@H]1NC(=O)c1ccccc1O ; C[C@H]1NC(=O)C(C2CC2)N(C)C(=O)[C@H](C)[C@H](O)[C@H](Cc2cccnc2)NC(=O)[C@H]1NC(=O)c1ccccc1O]

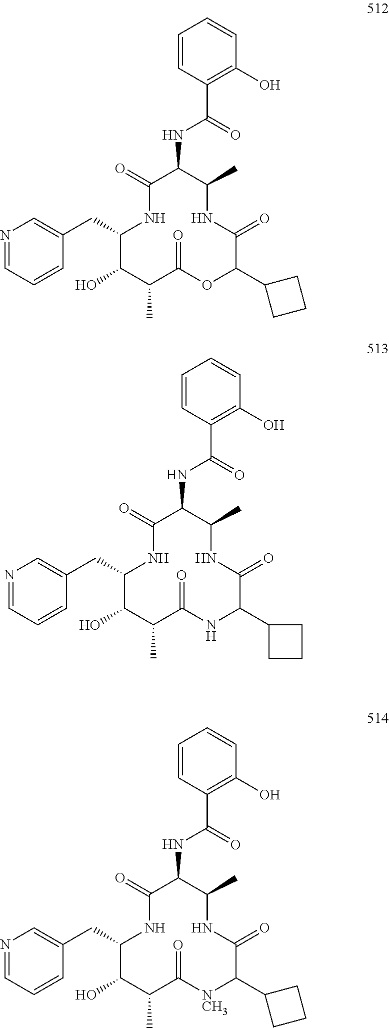 [SMILES: C[C@H]1NC(=O)C(C2CCC2)OC(=O)[C@H](C)[C@H](O)[C@H](Cc2cccnc2)NC(=O)[C@H]1NC(=O)c1ccccc1O ; C[C@H]1NC(=O)C(C2CCC2)NC(=O)[C@H](C)[C@H](O)[C@H](Cc2cccnc2)NC(=O)[C@H]1NC(=O)c1ccccc1O ; C[C@H]1NC(=O)C(C2CCC2)N(C)C(=O)[C@H](C)[C@H](O)[C@H](Cc2cccnc2)NC(=O)[C@H]1NC(=O)c1ccccc1O]

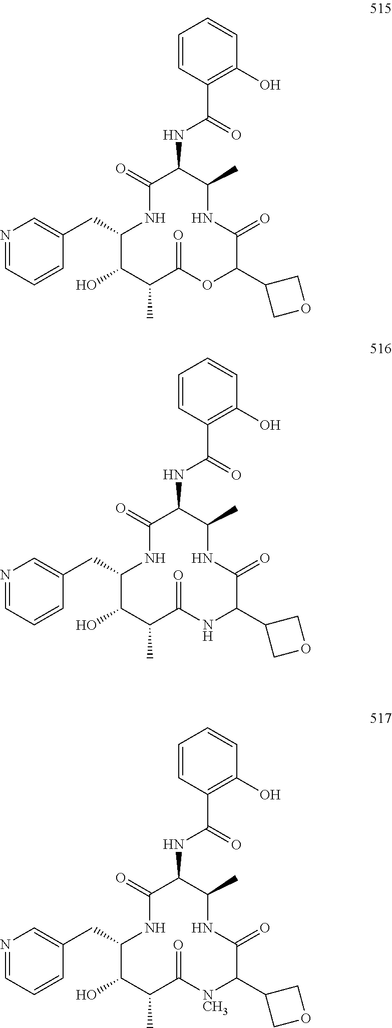 [SMILES: C[C@H]1NC(=O)C(C2COC2)OC(=O)[C@H](C)[C@H](O)[C@H](Cc2cccnc2)NC(=O)[C@H]1NC(=O)c1ccccc1O ; C[C@H]1NC(=O)C(C2COC2)NC(=O)[C@H](C)[C@H](O)[C@H](Cc2cccnc2)NC(=O)[C@H]1NC(=O)c1ccccc1O ; C[C@H]1NC(=O)C(C2COC2)N(C)C(=O)[C@H](C)[C@H](O)[C@H](Cc2cccnc2)NC(=O)[C@H]1NC(=O)c1ccccc1O]

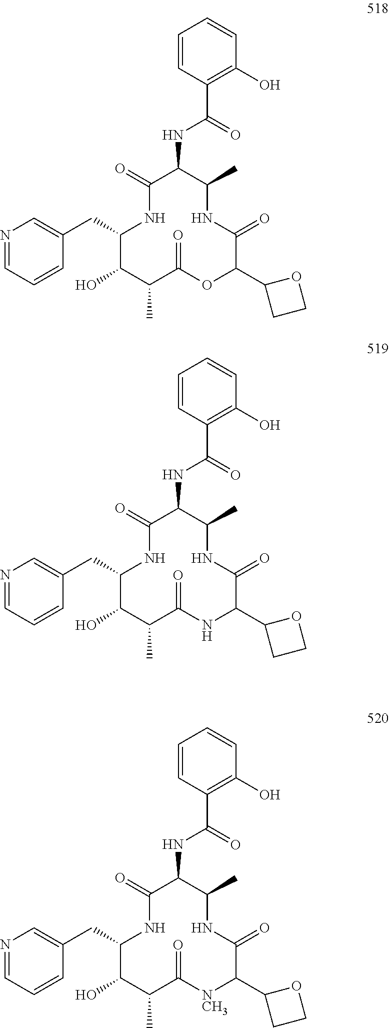 [SMILES: C[C@H]1NC(=O)C(C2CCO2)OC(=O)[C@H](C)[C@H](O)[C@H](Cc2cccnc2)NC(=O)[C@H]1NC(=O)c1ccccc1O ; C[C@H]1NC(=O)C(C2CCO2)NC(=O)[C@H](C)[C@H](O)[C@H](Cc2cccnc2)NC(=O)[C@H]1NC(=O)c1ccccc1O ; C[C@H]1NC(=O)C(C2CCO2)N(C)C(=O)[C@H](C)[C@H](O)[C@H](Cc2cccnc2)NC(=O)[C@H]1NC(=O)c1ccccc1O]

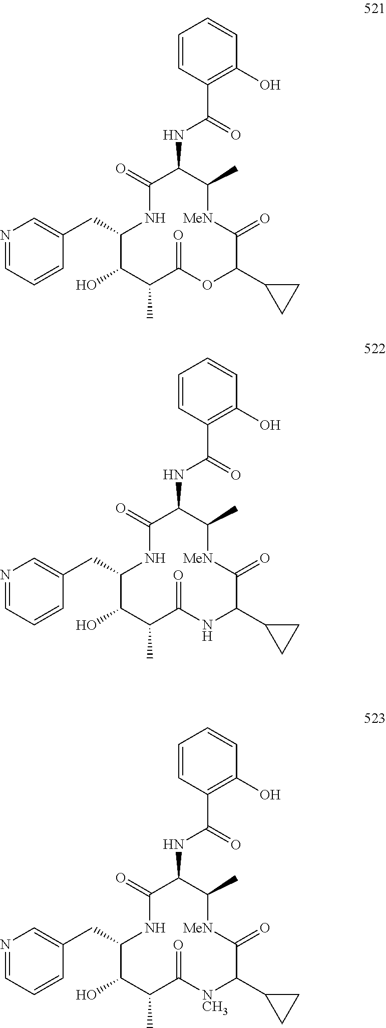 [SMILES: C[C@@H]1[C@H](NC(=O)c2ccccc2O)C(=O)N[C@@H](Cc2cccnc2)[C@@H](O)[C@@H](C)C(=O)OC(C2CC2)C(=O)N1C ; C[C@@H]1[C@H](NC(=O)c2ccccc2O)C(=O)N[C@@H](Cc2cccnc2)[C@@H](O)[C@@H](C)C(=O)NC(C2CC2)C(=O)N1C ; C[C@@H]1[C@H](NC(=O)c2ccccc2O)C(=O)N[C@@H](Cc2cccnc2)[C@@H](O)[C@@H](C)C(=O)N(C)C(C2CC2)C(=O)N1C]

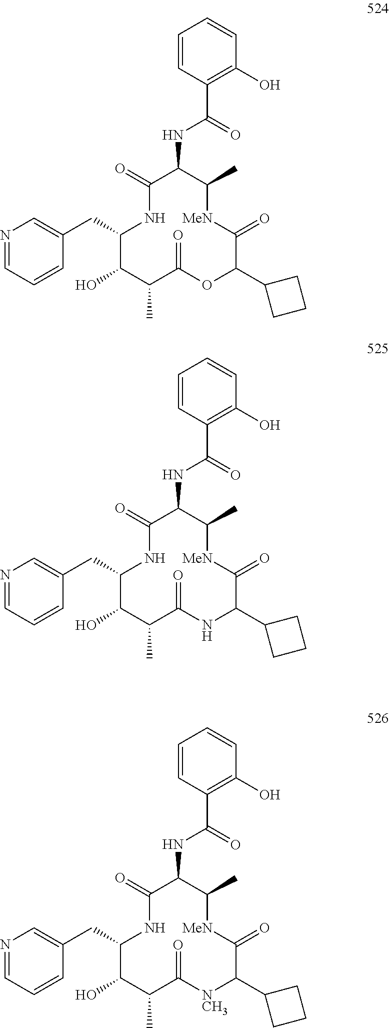 [SMILES: C[C@@H]1[C@H](NC(=O)c2ccccc2O)C(=O)N[C@@H](Cc2cccnc2)[C@@H](O)[C@@H](C)C(=O)OC(C2CCC2)C(=O)N1C ; C[C@@H]1[C@H](NC(=O)c2ccccc2O)C(=O)N[C@@H](Cc2cccnc2)[C@@H](O)[C@@H](C)C(=O)NC(C2CCC2)C(=O)N1C ; C[C@@H]1[C@H](NC(=O)c2ccccc2O)C(=O)N[C@@H](Cc2cccnc2)[C@@H](O)[C@@H](C)C(=O)N(C)C(C2CCC2)C(=O)N1C]

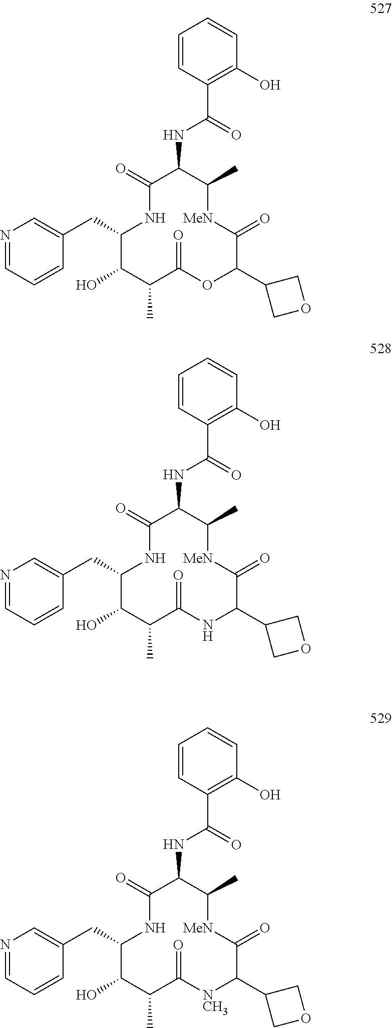 [SMILES: C[C@@H]1[C@H](NC(=O)c2ccccc2O)C(=O)N[C@@H](Cc2cccnc2)[C@@H](O)[C@@H](C)C(=O)OC(C2COC2)C(=O)N1C ; C[C@@H]1[C@H](NC(=O)c2ccccc2O)C(=O)N[C@@H](Cc2cccnc2)[C@@H](O)[C@@H](C)C(=O)NC(C2COC2)C(=O)N1C ; C[C@@H]1[C@H](NC(=O)c2ccccc2O)C(=O)N[C@@H](Cc2cccnc2)[C@@H](O)[C@@H](C)C(=O)N(C)C(C2COC2)C(=O)N1C]

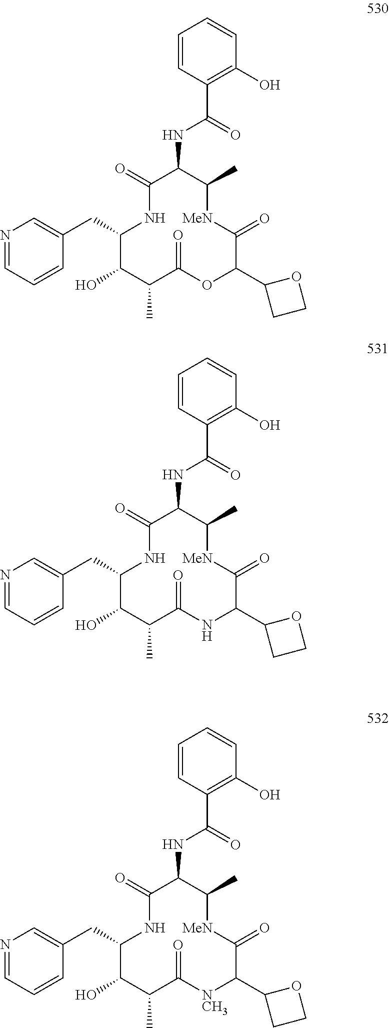 [SMILES: C[C@@H]1[C@H](NC(=O)c2ccccc2O)C(=O)N[C@@H](Cc2cccnc2)[C@@H](O)[C@@H](C)C(=O)OC(C2CCO2)C(=O)N1C ; C[C@@H]1[C@H](NC(=O)c2ccccc2O)C(=O)N[C@@H](Cc2cccnc2)[C@@H](O)[C@@H](C)C(=O)NC(C2CCO2)C(=O)N1C ; C[C@@H]1[C@H](NC(=O)c2ccccc2O)C(=O)N[C@@H](Cc2cccnc2)[C@@H](O)[C@@H](C)C(=O)N(C)C(C2CCO2)C(=O)N1C]

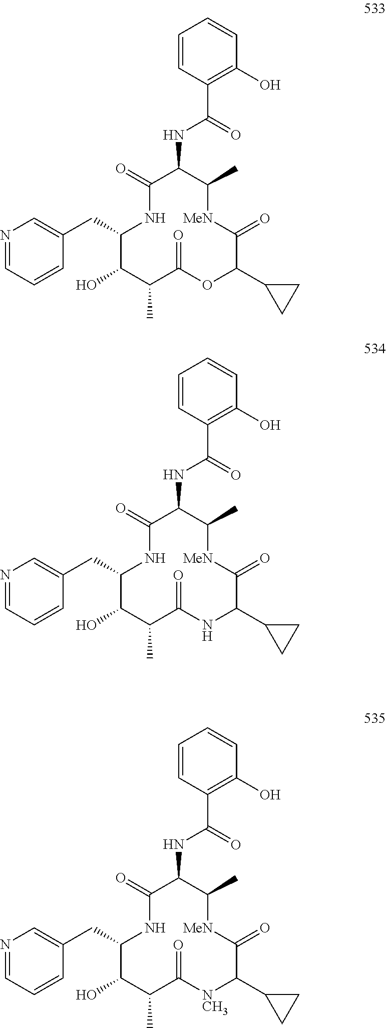 [SMILES: C[C@@H]1[C@H](NC(=O)c2ccccc2O)C(=O)N[C@@H](Cc2cccnc2)[C@@H](O)[C@@H](C)C(=O)OC(C2CC2)C(=O)N1C ; C[C@@H]1[C@H](NC(=O)c2ccccc2O)C(=O)N[C@@H](Cc2cccnc2)[C@@H](O)[C@@H](C)C(=O)NC(C2CC2)C(=O)N1C ; C[C@@H]1[C@H](NC(=O)c2ccccc2O)C(=O)N[C@@H](Cc2cccnc2)[C@@H](O)[C@@H](C)C(=O)N(C)C(C2CC2)C(=O)N1C]

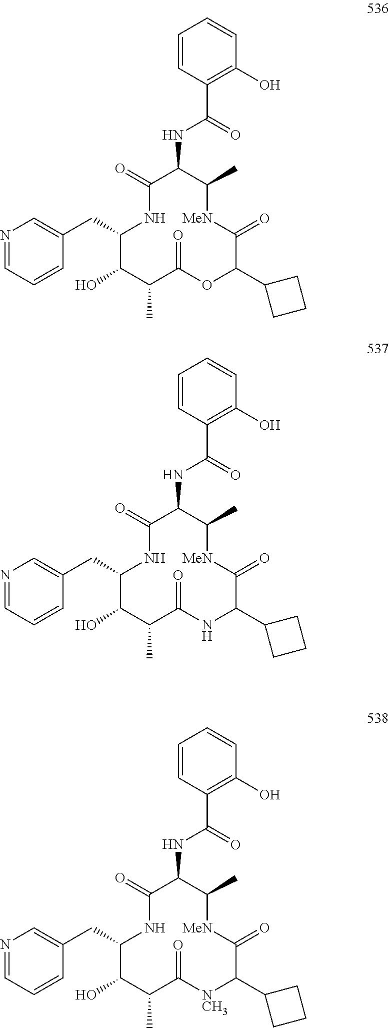 [SMILES: C[C@@H]1[C@H](NC(=O)c2ccccc2O)C(=O)N[C@@H](Cc2cccnc2)[C@@H](O)[C@@H](C)C(=O)OC(C2CCC2)C(=O)N1C ; C[C@@H]1[C@H](NC(=O)c2ccccc2O)C(=O)N[C@@H](Cc2cccnc2)[C@@H](O)[C@@H](C)C(=O)NC(C2CCC2)C(=O)N1C ; C[C@@H]1[C@H](NC(=O)c2ccccc2O)C(=O)N[C@@H](Cc2cccnc2)[C@@H](O)[C@@H](C)C(=O)N(C)C(C2CCC2)C(=O)N1C]

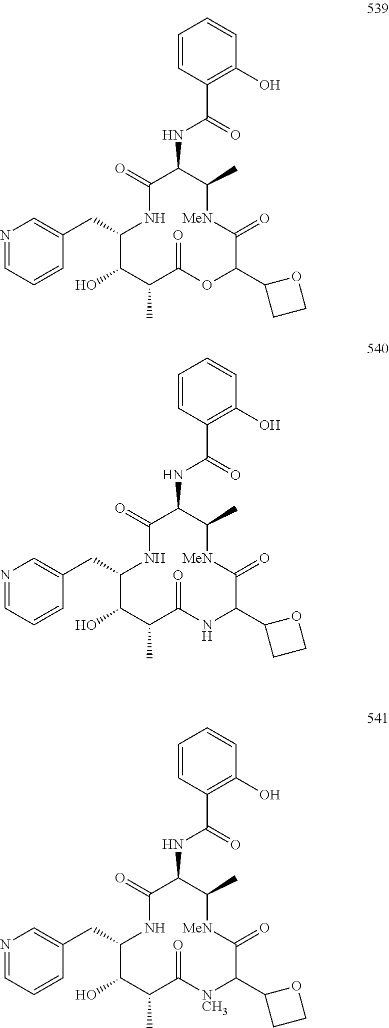 [SMILES: C[C@@H]1[C@H](NC(=O)c2ccccc2O)C(=O)N[C@@H](Cc2cccnc2)[C@@H](O)[C@@H](C)C(=O)OC(C2CCO2)C(=O)N1C ; C[C@@H]1[C@H](NC(=O)c2ccccc2O)C(=O)N[C@@H](Cc2cccnc2)[C@@H](O)[C@@H](C)C(=O)NC(C2CCO2)C(=O)N1C ; C[C@@H]1[C@H](NC(=O)c2ccccc2O)C(=O)N[C@@H](Cc2cccnc2)[C@@H](O)[C@@H](C)C(=O)N(C)C(C2CCO2)C(=O)N1C]

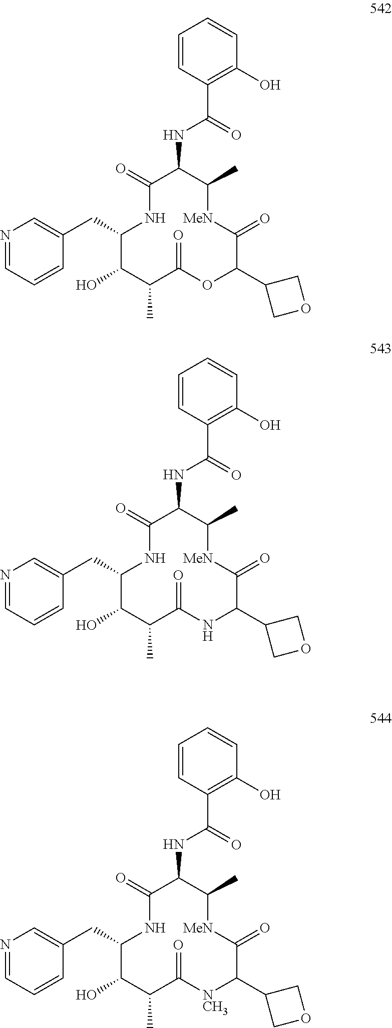 [SMILES: C[C@@H]1[C@H](NC(=O)c2ccccc2O)C(=O)N[C@@H](Cc2cccnc2)[C@@H](O)[C@@H](C)C(=O)OC(C2COC2)C(=O)N1C ; C[C@@H]1[C@H](NC(=O)c2ccccc2O)C(=O)N[C@@H](Cc2cccnc2)[C@@H](O)[C@@H](C)C(=O)NC(C2COC2)C(=O)N1C ; C[C@@H]1[C@H](NC(=O)c2ccccc2O)C(=O)N[C@@H](Cc2cccnc2)[C@@H](O)[C@@H](C)C(=O)N(C)C(C2COC2)C(=O)N1C]

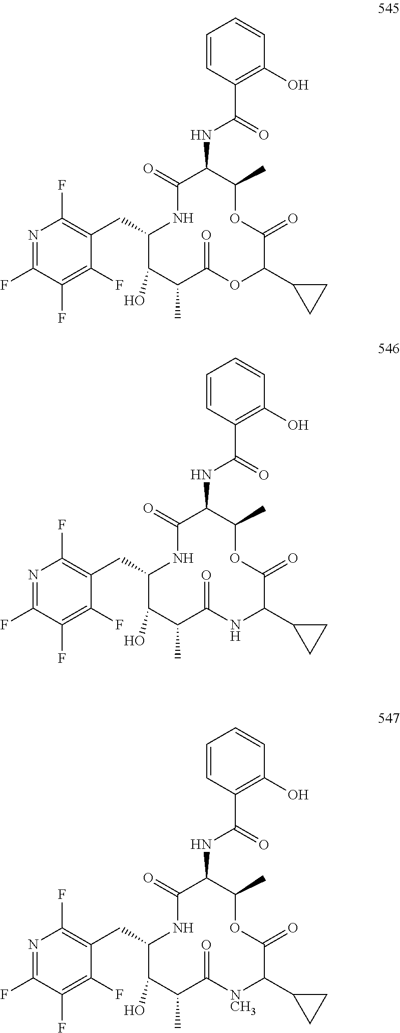 [SMILES: C[C@H]1OC(=O)C(C2CC2)OC(=O)[C@H](C)[C@H](O)[C@H](Cc2c(F)nc(F)c(F)c2F)NC(=O)[C@H]1NC(=O)c1ccccc1O ; C[C@H]1OC(=O)C(C2CC2)NC(=O)[C@H](C)[C@H](O)[C@H](Cc2c(F)nc(F)c(F)c2F)NC(=O)[C@H]1NC(=O)c1ccccc1O ; C[C@H]1OC(=O)C(C2CC2)N(C)C(=O)[C@H](C)[C@H](O)[C@H](Cc2c(F)nc(F)c(F)c2F)NC(=O)[C@H]1NC(=O)c1ccccc1O]

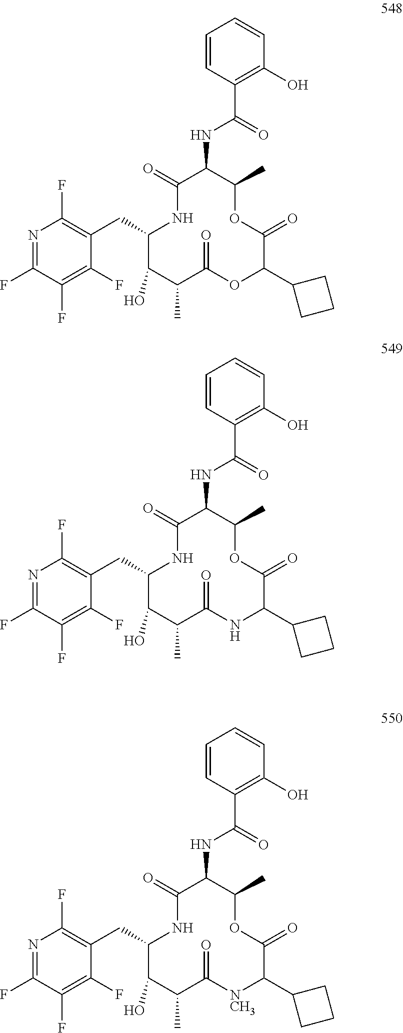 [SMILES: C[C@H]1OC(=O)C(C2CCC2)OC(=O)[C@H](C)[C@H](O)[C@H](Cc2c(F)nc(F)c(F)c2F)NC(=O)[C@H]1NC(=O)c1ccccc1O ; C[C@H]1OC(=O)C(C2CCC2)NC(=O)[C@H](C)[C@H](O)[C@H](Cc2c(F)nc(F)c(F)c2F)NC(=O)[C@H]1NC(=O)c1ccccc1O ; C[C@H]1OC(=O)C(C2CCC2)N(C)C(=O)[C@H](C)[C@H](O)[C@H](Cc2c(F)nc(F)c(F)c2F)NC(=O)[C@H]1NC(=O)c1ccccc1O]

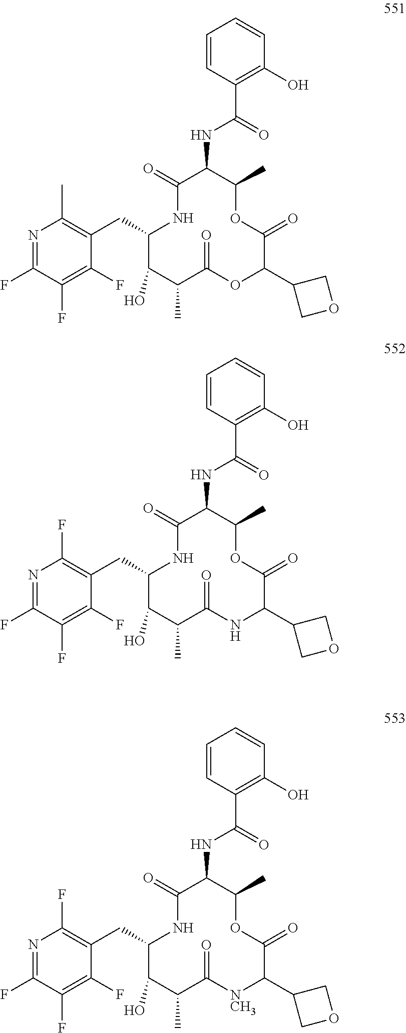 [SMILES: Cc1nc(F)c(F)c(F)c1C[C@@H]1NC(=O)[C@@H](NC(=O)c2ccccc2O)[C@@H](C)OC(=O)C(C2COC2)OC(=O)[C@H](C)[C@@H]1O ; C[C@H]1OC(=O)C(C2COC2)NC(=O)[C@H](C)[C@H](O)[C@H](Cc2c(F)nc(F)c(F)c2F)NC(=O)[C@H]1NC(=O)c1ccccc1O ; C[C@H]1OC(=O)C(C2COC2)N(C)C(=O)[C@H](C)[C@H](O)[C@H](Cc2c(F)nc(F)c(F)c2F)NC(=O)[C@H]1NC(=O)c1ccccc1O]

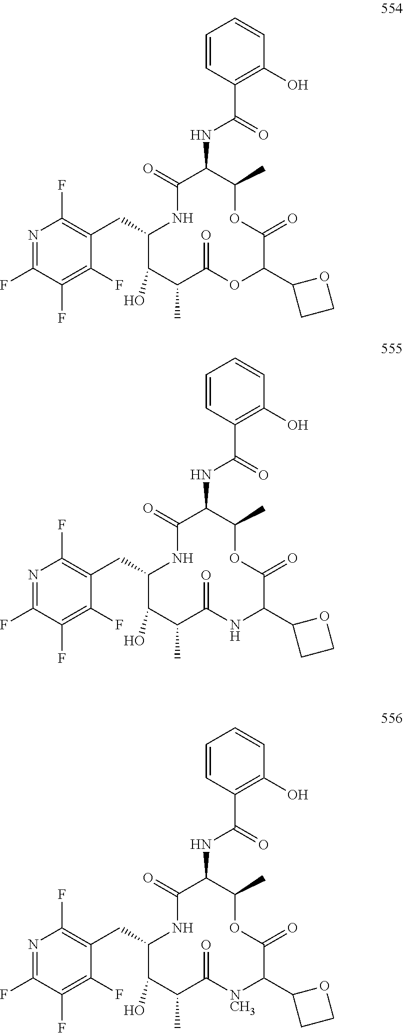 [SMILES: C[C@H]1OC(=O)C(C2CCO2)OC(=O)[C@H](C)[C@H](O)[C@H](Cc2c(F)nc(F)c(F)c2F)NC(=O)[C@H]1NC(=O)c1ccccc1O ; C[C@H]1OC(=O)C(C2CCO2)NC(=O)[C@H](C)[C@H](O)[C@H](Cc2c(F)nc(F)c(F)c2F)NC(=O)[C@H]1NC(=O)c1ccccc1O ; C[C@H]1OC(=O)C(C2CCO2)N(C)C(=O)[C@H](C)[C@H](O)[C@H](Cc2c(F)nc(F)c(F)c2F)NC(=O)[C@H]1NC(=O)c1ccccc1O]

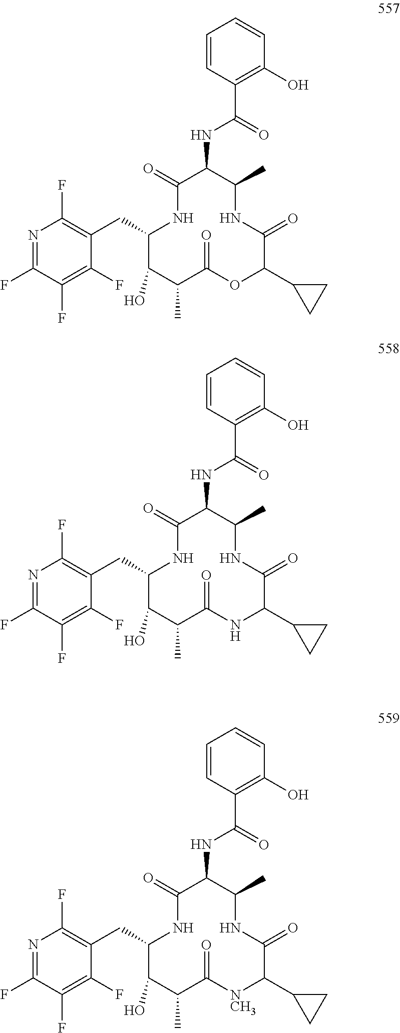 [SMILES: C[C@H]1NC(=O)C(C2CC2)OC(=O)[C@H](C)[C@H](O)[C@H](Cc2c(F)nc(F)c(F)c2F)NC(=O)[C@H]1NC(=O)c1ccccc1O ; C[C@H]1NC(=O)C(C2CC2)NC(=O)[C@H](C)[C@H](O)[C@H](Cc2c(F)nc(F)c(F)c2F)NC(=O)[C@H]1NC(=O)c1ccccc1O ; C[C@H]1NC(=O)C(C2CC2)N(C)C(=O)[C@H](C)[C@H](O)[C@H](Cc2c(F)nc(F)c(F)c2F)NC(=O)[C@H]1NC(=O)c1ccccc1O]

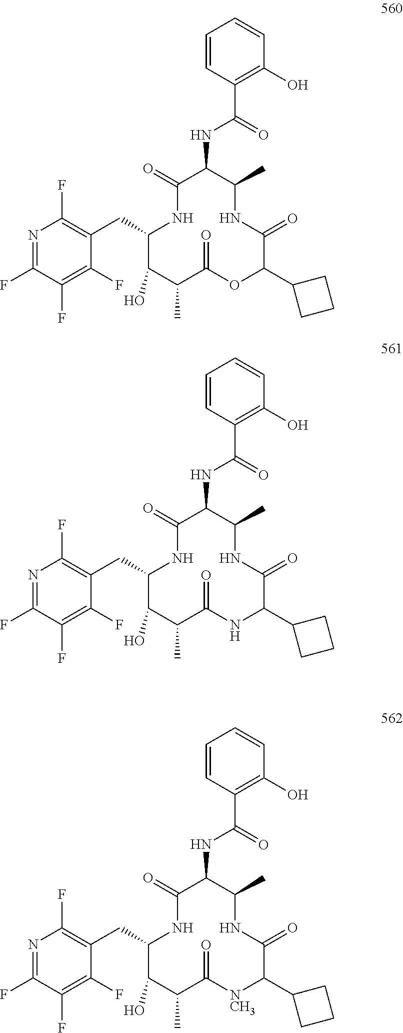 [SMILES: C[C@H]1NC(=O)C(C2CCC2)OC(=O)[C@H](C)[C@H](O)[C@H](Cc2c(F)nc(F)c(F)c2F)NC(=O)[C@H]1NC(=O)c1ccccc1O ; C[C@H]1NC(=O)C(C2CCC2)NC(=O)[C@H](C)[C@H](O)[C@H](Cc2c(F)nc(F)c(F)c2F)NC(=O)[C@H]1NC(=O)c1ccccc1O ; C[C@H]1NC(=O)C(C2CCC2)N(C)C(=O)[C@H](C)[C@H](O)[C@H](Cc2c(F)nc(F)c(F)c2F)NC(=O)[C@H]1NC(=O)c1ccccc1O]

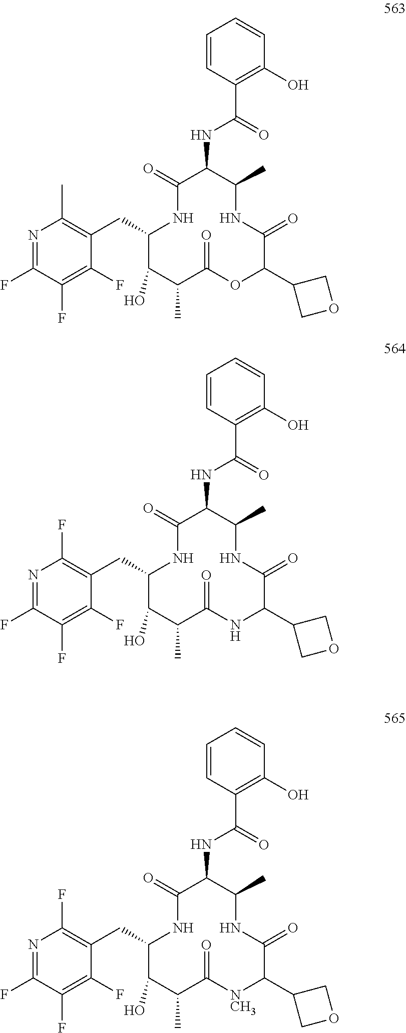 [SMILES: Cc1nc(F)c(F)c(F)c1C[C@@H]1NC(=O)[C@@H](NC(=O)c2ccccc2O)[C@@H](C)NC(=O)C(C2COC2)OC(=O)[C@H](C)[C@@H]1O ; C[C@H]1NC(=O)C(C2COC2)NC(=O)[C@H](C)[C@H](O)[C@H](Cc2c(F)nc(F)c(F)c2F)NC(=O)[C@H]1NC(=O)c1ccccc1O ; C[C@H]1NC(=O)C(C2COC2)N(C)C(=O)[C@H](C)[C@H](O)[C@H](Cc2c(F)nc(F)c(F)c2F)NC(=O)[C@H]1NC(=O)c1ccccc1O]

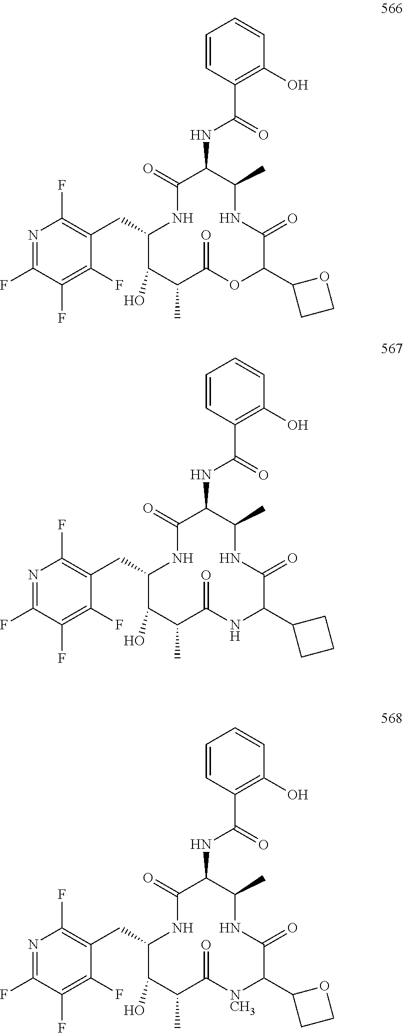 [SMILES: C[C@H]1NC(=O)C(C2CCO2)OC(=O)[C@H](C)[C@H](O)[C@H](Cc2c(F)nc(F)c(F)c2F)NC(=O)[C@H]1NC(=O)c1ccccc1O ; C[C@H]1NC(=O)C(C2CCC2)NC(=O)[C@H](C)[C@H](O)[C@H](Cc2c(F)nc(F)c(F)c2F)NC(=O)[C@H]1NC(=O)c1ccccc1O ; C[C@H]1NC(=O)C(C2CCO2)N(C)C(=O)[C@H](C)[C@H](O)[C@H](Cc2c(F)nc(F)c(F)c2F)NC(=O)[C@H]1NC(=O)c1ccccc1O]

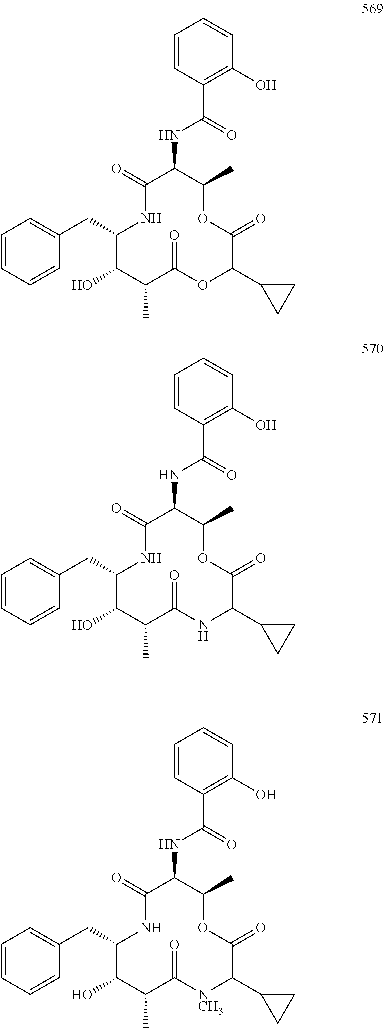 [SMILES: C[C@H]1OC(=O)C(C2CC2)OC(=O)[C@H](C)[C@H](O)[C@H](Cc2ccccc2)NC(=O)[C@H]1NC(=O)c1ccccc1O ; C[C@H]1OC(=O)C(C2CC2)NC(=O)[C@H](C)[C@H](O)[C@H](Cc2ccccc2)NC(=O)[C@H]1NC(=O)c1ccccc1O ; C[C@H]1OC(=O)C(C2CC2)N(C)C(=O)[C@H](C)[C@H](O)[C@H](Cc2ccccc2)NC(=O)[C@H]1NC(=O)c1ccccc1O]

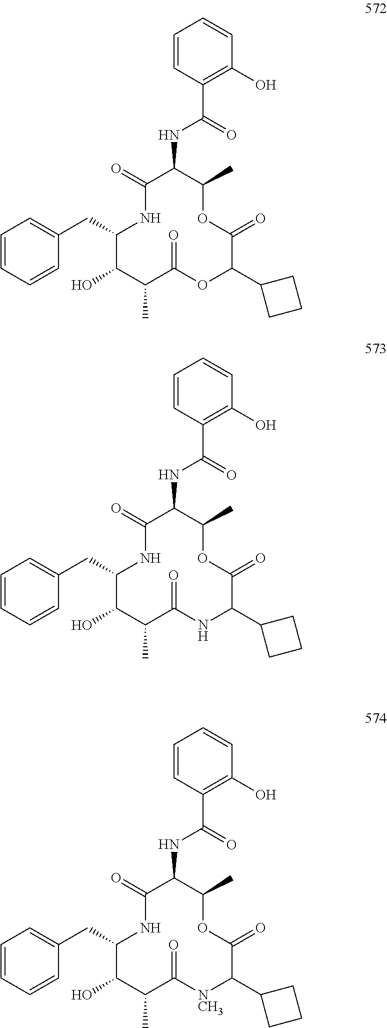 [SMILES: C[C@H]1OC(=O)C(C2CCC2)OC(=O)[C@H](C)[C@H](O)[C@H](Cc2ccccc2)NC(=O)[C@H]1NC(=O)c1ccccc1O ; C[C@H]1OC(=O)C(C2CCC2)NC(=O)[C@H](C)[C@H](O)[C@H](Cc2ccccc2)NC(=O)[C@H]1NC(=O)c1ccccc1O ; C[C@H]1OC(=O)C(C2CCC2)N(C)C(=O)[C@H](C)[C@H](O)[C@H](Cc2ccccc2)NC(=O)[C@H]1NC(=O)c1ccccc1O]

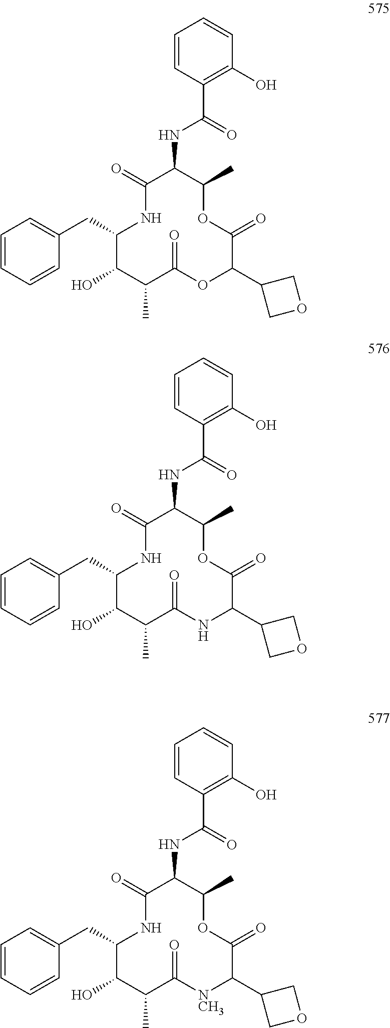 [SMILES: C[C@H]1OC(=O)C(C2COC2)OC(=O)[C@H](C)[C@H](O)[C@H](Cc2ccccc2)NC(=O)[C@H]1NC(=O)c1ccccc1O ; C[C@H]1OC(=O)C(C2COC2)NC(=O)[C@H](C)[C@H](O)[C@H](Cc2ccccc2)NC(=O)[C@H]1NC(=O)c1ccccc1O ; C[C@H]1OC(=O)C(C2COC2)N(C)C(=O)[C@H](C)[C@H](O)[C@H](Cc2ccccc2)NC(=O)[C@H]1NC(=O)c1ccccc1O]

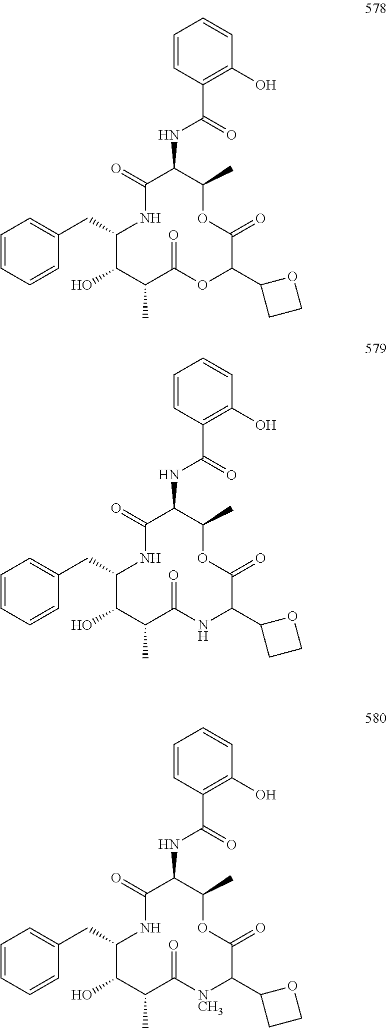 [SMILES: C[C@H]1OC(=O)C(C2CCO2)OC(=O)[C@H](C)[C@H](O)[C@H](Cc2ccccc2)NC(=O)[C@H]1NC(=O)c1ccccc1O ; C[C@H]1OC(=O)C(C2CCO2)NC(=O)[C@H](C)[C@H](O)[C@H](Cc2ccccc2)NC(=O)[C@H]1NC(=O)c1ccccc1O ; C[C@H]1OC(=O)C(C2CCO2)N(C)C(=O)[C@H](C)[C@H](O)[C@H](Cc2ccccc2)NC(=O)[C@H]1NC(=O)c1ccccc1O]

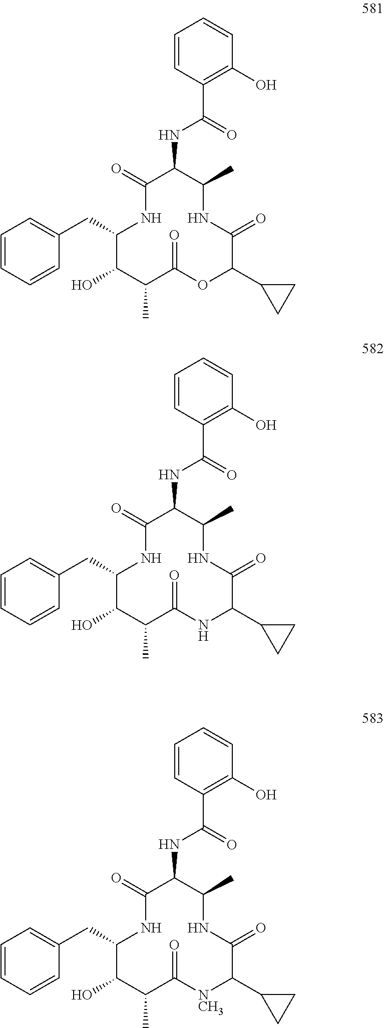 [SMILES: C[C@H]1NC(=O)C(C2CC2)OC(=O)[C@H](C)[C@H](O)[C@H](Cc2ccccc2)NC(=O)[C@H]1NC(=O)c1ccccc1O ; C[C@H]1NC(=O)C(C2CC2)NC(=O)[C@H](C)[C@H](O)[C@H](Cc2ccccc2)NC(=O)[C@H]1NC(=O)c1ccccc1O ; C[C@H]1NC(=O)C(C2CC2)N(C)C(=O)[C@H](C)[C@H](O)[C@H](Cc2ccccc2)NC(=O)[C@H]1NC(=O)c1ccccc1O]

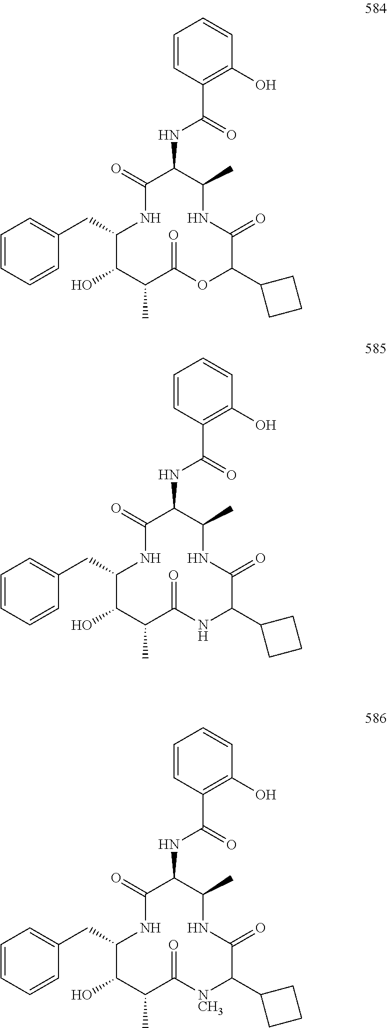 [SMILES: C[C@H]1NC(=O)C(C2CCC2)OC(=O)[C@H](C)[C@H](O)[C@H](Cc2ccccc2)NC(=O)[C@H]1NC(=O)c1ccccc1O ; C[C@H]1NC(=O)C(C2CCC2)NC(=O)[C@H](C)[C@H](O)[C@H](Cc2ccccc2)NC(=O)[C@H]1NC(=O)c1ccccc1O ; C[C@H]1NC(=O)C(C2CCC2)N(C)C(=O)[C@H](C)[C@H](O)[C@H](Cc2ccccc2)NC(=O)[C@H]1NC(=O)c1ccccc1O]

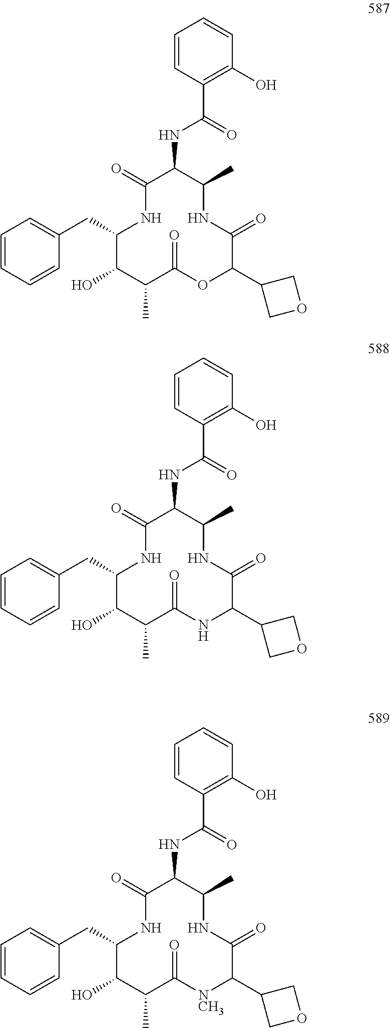 [SMILES: C[C@H]1NC(=O)C(C2COC2)OC(=O)[C@H](C)[C@H](O)[C@H](Cc2ccccc2)NC(=O)[C@H]1NC(=O)c1ccccc1O ; C[C@H]1NC(=O)C(C2COC2)NC(=O)[C@H](C)[C@H](O)[C@H](Cc2ccccc2)NC(=O)[C@H]1NC(=O)c1ccccc1O ; C[C@H]1NC(=O)C(C2COC2)N(C)C(=O)[C@H](C)[C@H](O)[C@H](Cc2ccccc2)NC(=O)[C@H]1NC(=O)c1ccccc1O]

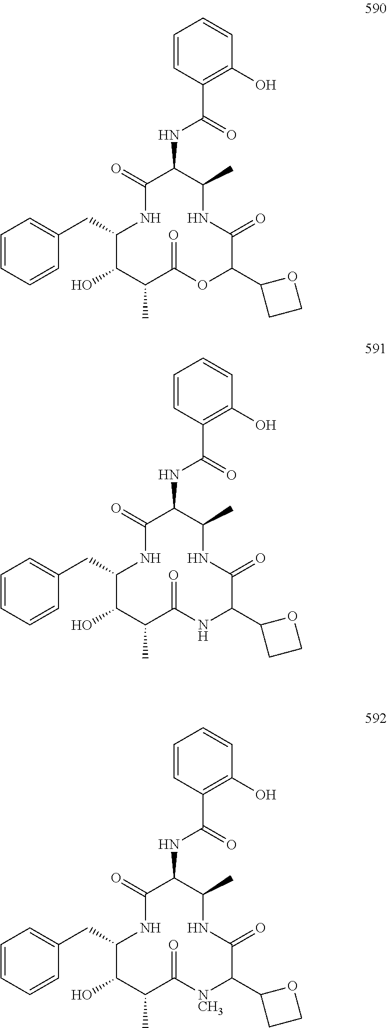 [SMILES: C[C@H]1NC(=O)C(C2CCO2)OC(=O)[C@H](C)[C@H](O)[C@H](Cc2ccccc2)NC(=O)[C@H]1NC(=O)c1ccccc1O ; C[C@H]1NC(=O)C(C2CCO2)NC(=O)[C@H](C)[C@H](O)[C@H](Cc2ccccc2)NC(=O)[C@H]1NC(=O)c1ccccc1O ; C[C@H]1NC(=O)C(C2CCO2)N(C)C(=O)[C@H](C)[C@H](O)[C@H](Cc2ccccc2)NC(=O)[C@H]1NC(=O)c1ccccc1O]

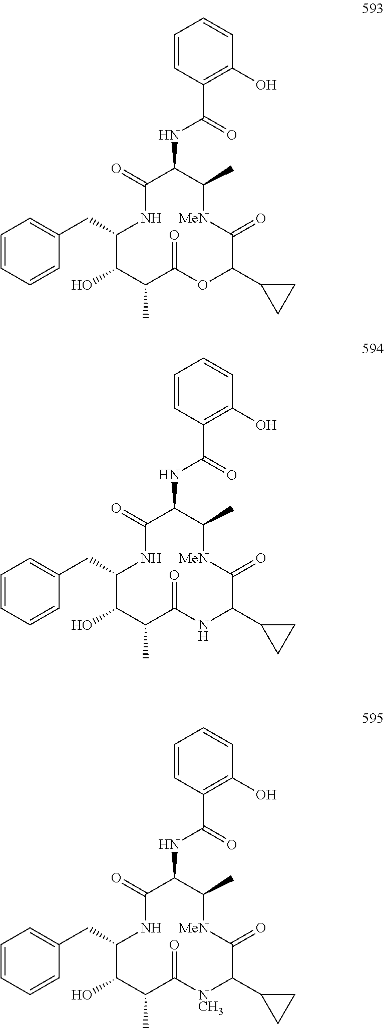 [SMILES: C[C@@H]1[C@H](NC(=O)c2ccccc2O)C(=O)N[C@@H](Cc2ccccc2)[C@@H](O)[C@@H](C)C(=O)OC(C2CC2)C(=O)N1C ; C[C@@H]1[C@H](NC(=O)c2ccccc2O)C(=O)N[C@@H](Cc2ccccc2)[C@@H](O)[C@@H](C)C(=O)NC(C2CC2)C(=O)N1C ; C[C@@H]1[C@H](NC(=O)c2ccccc2O)C(=O)N[C@@H](Cc2ccccc2)[C@@H](O)[C@@H](C)C(=O)N(C)C(C2CC2)C(=O)N1C]

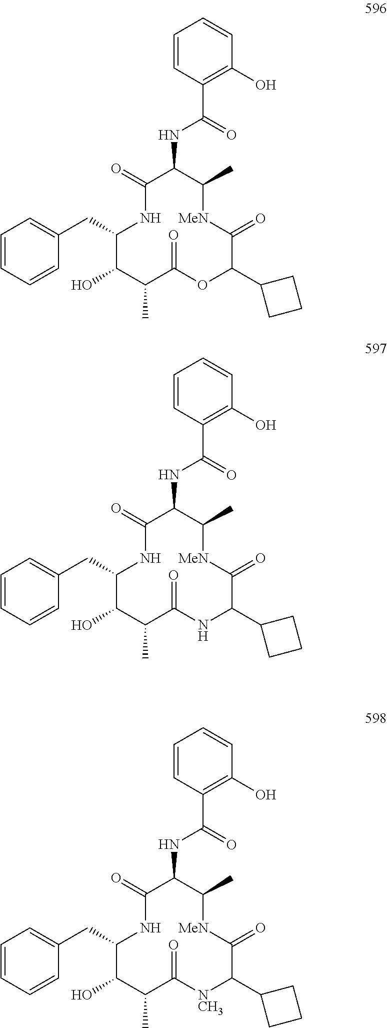 [SMILES: C[C@@H]1[C@H](NC(=O)c2ccccc2O)C(=O)N[C@@H](Cc2ccccc2)[C@@H](O)[C@@H](C)C(=O)OC(C2CCC2)C(=O)N1C ; C[C@@H]1[C@H](NC(=O)c2ccccc2O)C(=O)N[C@@H](Cc2ccccc2)[C@@H](O)[C@@H](C)C(=O)NC(C2CCC2)C(=O)N1C ; C[C@@H]1[C@H](NC(=O)c2ccccc2O)C(=O)N[C@@H](Cc2ccccc2)[C@@H](O)[C@@H](C)C(=O)N(C)C(C2CCC2)C(=O)N1C]

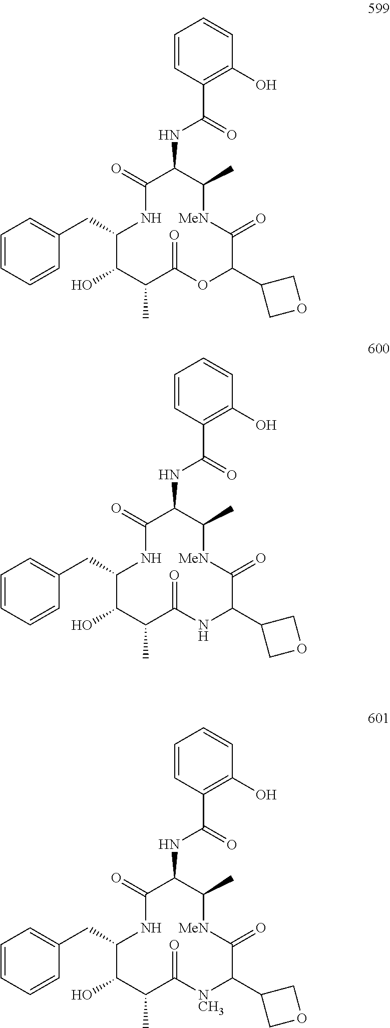 [SMILES: C[C@@H]1[C@H](NC(=O)c2ccccc2O)C(=O)N[C@@H](Cc2ccccc2)[C@@H](O)[C@@H](C)C(=O)OC(C2COC2)C(=O)N1C ; C[C@@H]1[C@H](NC(=O)c2ccccc2O)C(=O)N[C@@H](Cc2ccccc2)[C@@H](O)[C@@H](C)C(=O)NC(C2COC2)C(=O)N1C ; C[C@@H]1[C@H](NC(=O)c2ccccc2O)C(=O)N[C@@H](Cc2ccccc2)[C@@H](O)[C@@H](C)C(=O)N(C)C(C2COC2)C(=O)N1C]

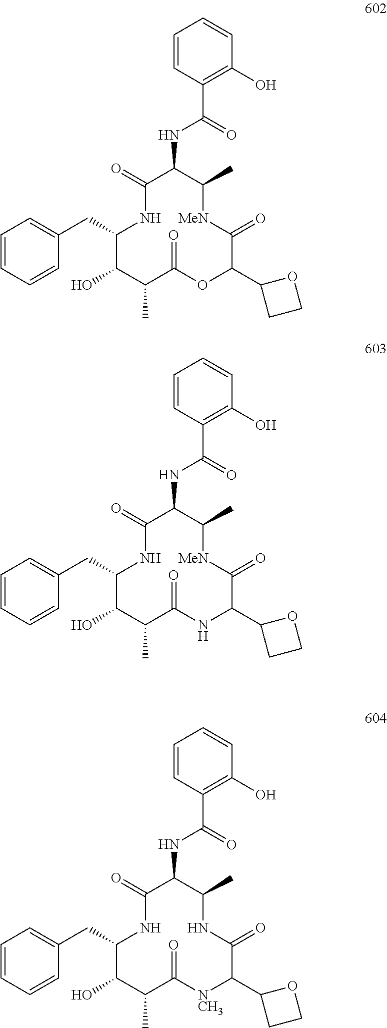 [SMILES: C[C@@H]1[C@H](NC(=O)c2ccccc2O)C(=O)N[C@@H](Cc2ccccc2)[C@@H](O)[C@@H](C)C(=O)OC(C2CCO2)C(=O)N1C ; C[C@@H]1[C@H](NC(=O)c2ccccc2O)C(=O)N[C@@H](Cc2ccccc2)[C@@H](O)[C@@H](C)C(=O)NC(C2CCO2)C(=O)N1C ; C[C@H]1NC(=O)C(C2CCO2)N(C)C(=O)[C@H](C)[C@H](O)[C@H](Cc2ccccc2)NC(=O)[C@H]1NC(=O)c1ccccc1O]

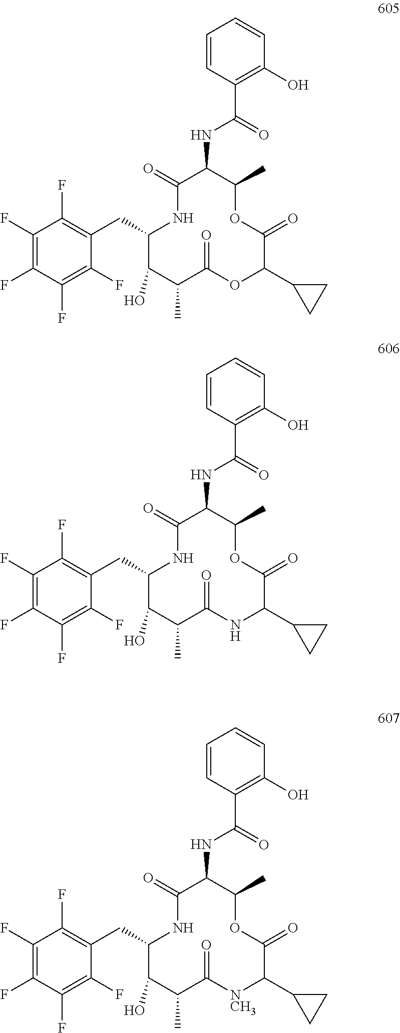 [SMILES: C[C@H]1OC(=O)C(C2CC2)OC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(C2CC2)NC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(C2CC2)N(C)C(=O)[C@H](C)[C@H](O)[C@H](Cc2c(F)c(F)c(F)c(F)c2F)NC(=O)[C@H]1NC(=O)c1ccccc1O]

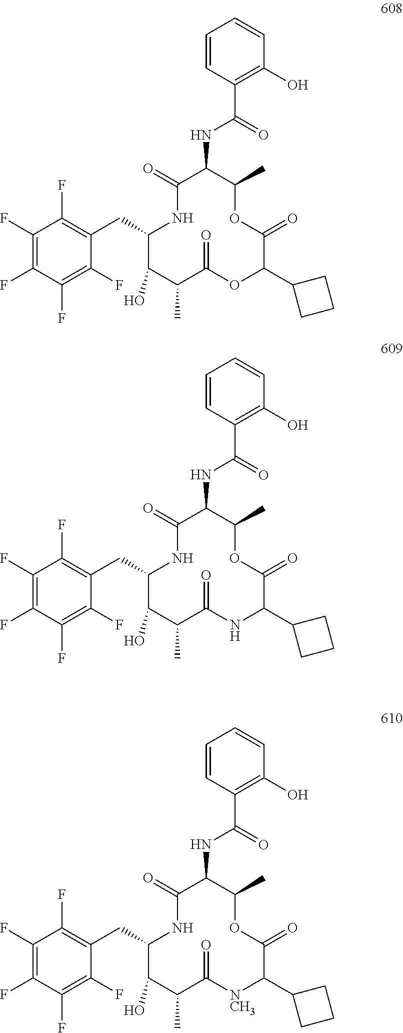 [SMILES: C[C@H]1OC(=O)C(C2CCC2)OC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(C2CCC2)NC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(C2CCC2)N(C)C(=O)[C@H](C)[C@H](O)[C@H](Cc2c(F)c(F)c(F)c(F)c2F)NC(=O)[C@H]1NC(=O)c1ccccc1O]

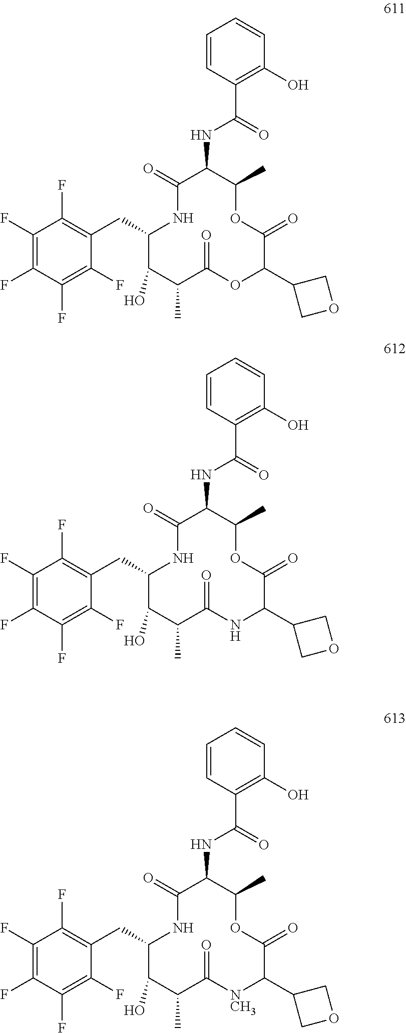 [SMILES: C[C@H]1OC(=O)C(C2COC2)OC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(C2COC2)NC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(C2COC2)N(C)C(=O)[C@H](C)[C@H](O)[C@H](Cc2c(F)c(F)c(F)c(F)c2F)NC(=O)[C@H]1NC(=O)c1ccccc1O]

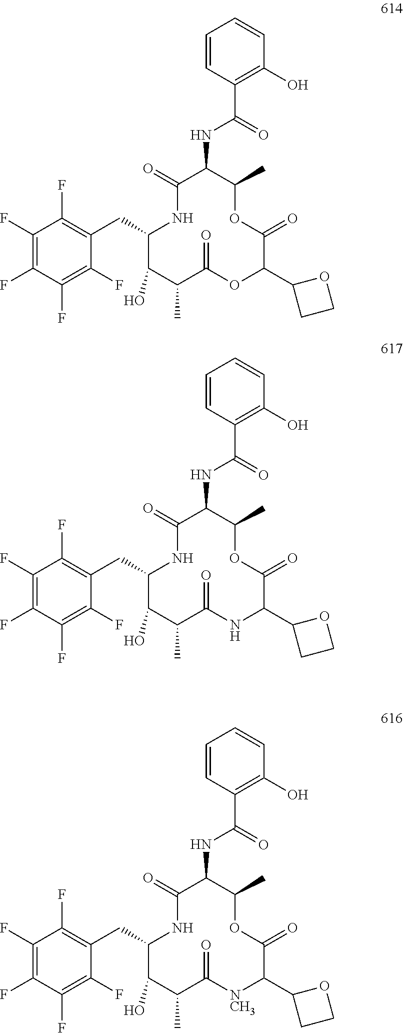 [SMILES: C[C@H]1OC(=O)C(C2CCO2)OC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(C2CCO2)NC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(C2CCO2)N(C)C(=O)[C@H](C)[C@H](O)[C@H](Cc2c(F)c(F)c(F)c(F)c2F)NC(=O)[C@H]1NC(=O)c1ccccc1O]

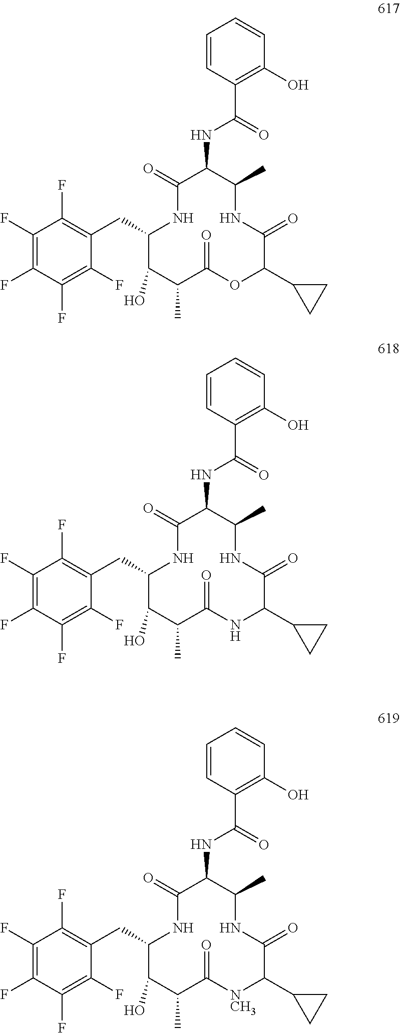 [SMILES: C[C@H]1NC(=O)C(C2CC2)OC(=O)[C@H](C)[C@H](O)[C@H](Cc2c(F)c(F)c(F)c(F)c2F)NC(=O)[C@H]1NC(=O)c1ccccc1O ; C[C@H]1NC(=O)C(C2CC2)NC(=O)[C@H](C)[C@H](O)[C@H](Cc2c(F)c(F)c(F)c(F)c2F)NC(=O)[C@H]1NC(=O)c1ccccc1O ; C[C@H]1NC(=O)C(C2CC2)N(C)C(=O)[C@H](C)[C@H](O)[C@H](Cc2c(F)c(F)c(F)c(F)c2F)NC(=O)[C@H]1NC(=O)c1ccccc1O]

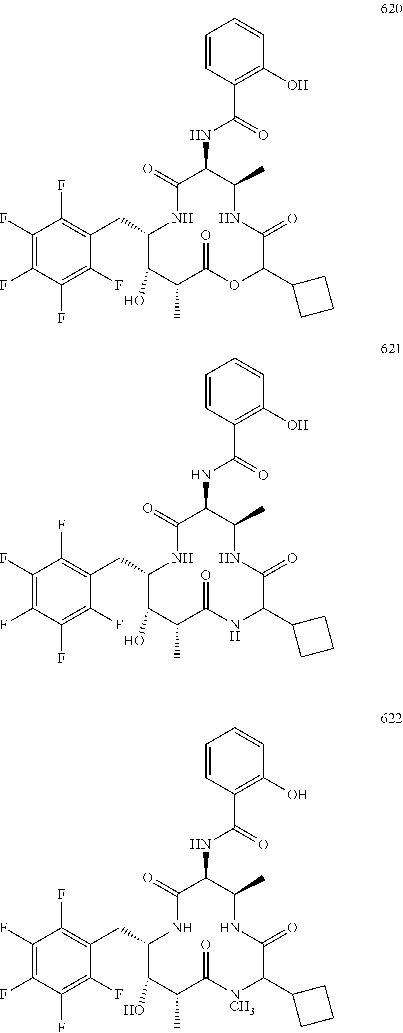 [SMILES: C[C@H]1NC(=O)C(C2CCC2)OC(=O)[C@H](C)[C@H](O)[C@H](Cc2c(F)c(F)c(F)c(F)c2F)NC(=O)[C@H]1NC(=O)c1ccccc1O ; C[C@H]1NC(=O)C(C2CCC2)NC(=O)[C@H](C)[C@H](O)[C@H](Cc2c(F)c(F)c(F)c(F)c2F)NC(=O)[C@H]1NC(=O)c1ccccc1O ; C[C@H]1NC(=O)C(C2CCC2)N(C)C(=O)[C@H](C)[C@H](O)[C@H](Cc2c(F)c(F)c(F)c(F)c2F)NC(=O)[C@H]1NC(=O)c1ccccc1O]

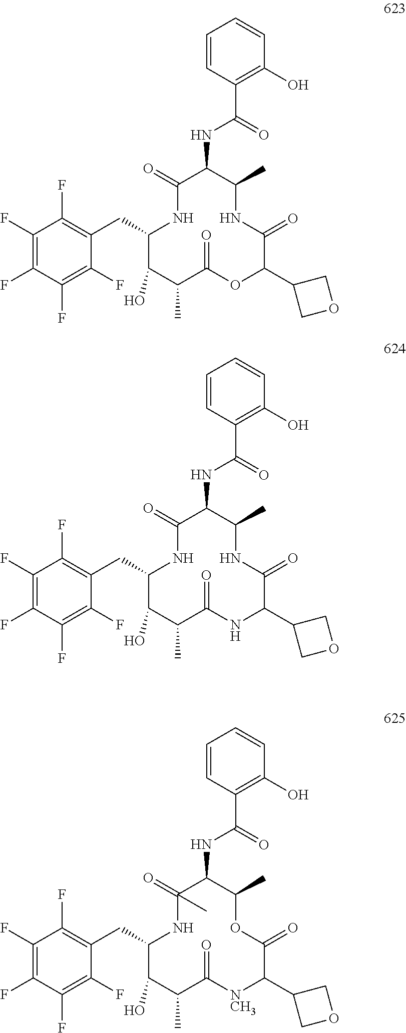

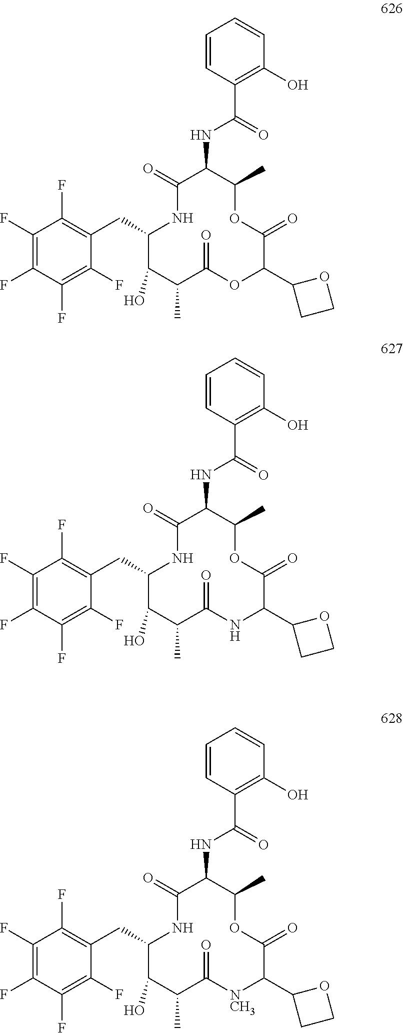 [SMILES: C[C@H]1OC(=O)C(C2CCO2)OC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(C2CCO2)NC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(C2CCO2)N(C)C(=O)[C@H](C)[C@H](O)[C@H](Cc2c(F)c(F)c(F)c(F)c2F)NC(=O)[C@H]1NC(=O)c1ccccc1O]

Further aspects of the invention include pharmaceutical compositions comprising a compound of formula (1) or a pharmaceutically acceptable salt, a hydrate or solvate thereof and a pharmaceutically acceptable carrier. The compounds according to the present invention are suitable as medicaments, preferably as medicaments for the treatment of mycobacterial infections.

In general, compounds of formula (1) are administered either individually, or optionally also in combination with another therapeutic agent, using the known and acceptable methods. Combination with another therapeutic agent includes one or more other anti-microbial and/or anti-fungal active ingredients. In one preferred embodiment the pharmaceutical compositions comprises a compound according to the present invention and compounds selected from the group of rifampicin, pyrazinamide, ethambutol, streptomycin, isonicotinyl, hydrazine, cycloserine, aminoglycosides (e.g., amikacin, kanamycin) or polypeptide antibiotic (e.g., capreomycin), pyrazinamide, ethambutol, fluoroquinolones such as moxifloxacin, rifabutin, cycloserine, thioamides such as prothionamide or, 4-aminosalicylic acid, a macrolide: e.g., clarithromycin, linezolid; interferon-γ, thioridazine, ampicillin, PA-824 (a new compound that is in advanced clinical development) and Bedaquiline (TMC207), a new compound that is in advanced clinical development as well) or mixtures thereof.

Such pharmaceutical compositions may be administered, for example, by one of the following routes: orally, for example in the form of dragees, coated tablets, pills, semi-solid substances, soft or hard capsules, solutions, emulsions or suspensions, parenterally, for example in the form of an injectable solution; rectally in the form of suppositories; by inhalation, for example in the form of a powder formulation or a spray; transdermally or intranasally.

For the preparation of such tablets, pills, semi-solid substances, coated tablet, dragees and hard gelatin capsules, the pharmaceutical composition may be mixed with pharmacologically inert, inorganic or organic pharmaceutical carrier substances, for example with lactose, sucrose, glucose, gelatin, malt, silica gel, starch or derivatives thereof, talcum, stearic acid or salts thereof, skimmed milk powder, and the like. For the preparation of soft capsules, pharmaceutical carrier substances such as, for example, vegetable oils, petroleum, animal or synthetic oils, wax, fat and polyols may be used.

For the preparation of liquid solutions and syrups, pharmaceutical carrier substances such as for example, water, alcohols, aqueous saline solutions, aqueous dextrose solutions, polyols, glycerol, vegetable oils, petroleum and animal or synthetic oils may be used.

For suppositories, pharmaceutical carrier substances such as, for example, vegetable oils, petroleum, animals or synthetic oils, wax, fat and polyols may be used.

For aerosol formulations, compressed gases that are suitable for this purpose, such as for example, oxygen, nitrogen and carbon dioxide may be used. The pharmaceutical composition may also comprise additives for preserving and stabilizing, emulsifiers, sweeteners, flavourings, salts for altering the osmotic pressure, buffers, encapsulation additives and antioxidants.

For the prevention and/or treatment of bacterial infections, especially the treatment of tuberculosis, the dose of the biologically active compound according to the invention may vary within wide limits and may be adjusted to individual requirements. Generally, a dose of 0.1 mg to 1000 mg per day is suitable, a preferred dose being from 20 to 100 mg per day. In suitable cases, the dose may also be below or above the stated values. The daily dose may be administered as a single dose or in multiple doses, for example in two or three doses. A typical individual dose contains approximately 0.1 mg, 10 mg, 50 mg, 100 mg and 250 mg of the active ingredient.

### EXAMPLES

### General Methods

All manipulations were conducted under an argon atmosphere using flame-dried glassware and standard syringe/septa and Schlenk techniques. Absolute solvents were purchased from Fluka (absolute over molecular sieves). Commercial chemicals were used without further purification. Solvents for extractions, flash column chromatography (FC) and thin layer chromatography (TLC) were purchased as commercial grade and distilled prior to use. TLC was performed on Merck TLC aluminum sheets (silica gel 60 F254). Spots were visualized with UV light (λ=254 nm) or through staining with Ce₂(SO₄)₃/phosphomolybdic acid/H₂SO₄ (CPS), vanillin/H₂SO₄ or KMnO₄/K₂CO₃. Chromatographic purification of products (FC) was performed using Fluka silica gel 60 for preparative column chromatography (particle size 40-63 μm).

NMR spectra were recorded on a Bruker Avance 400 MHz NMR spectrometer at 300 K. Chemical shifts (δ) are reported in ppm and are either referenced to the solvent signal as an internal standard (chloroform δ 7.26 ppm for ¹H and δ 77.00 ppm for ¹³C spectra; DMSO-d₆ δ 2.50 ppm for ¹H and δ 39.43 ppm for ¹³C spectra). Data are reported as follows: s=singlet, d=doublet, t=triplet, q=quartet, quint=quintet, sext=sextet, m=multiplet, br=broad signal, J=coupling constant in Hz. All ¹³C-NMR spectra were measured with complete proton decoupling. ¹H- and ¹³C-signals were assigned using two-dimensional correlation experiments (COSY, HMQC, HMBC). IR spectra were recorded on a Jasco FT/IR-6200 instrument as thin film. Optical rotations were measured on a Jasco P-1020 polarimeter operating at the sodium D line (λ=589 nm) and are reported as follows: [α]_{D}^{T}, concentration (c in g/100 mL) and solvent. Melting points were obtained in open capillary tubes using a Büchi melting point apparatus B-540 and are uncorrected. Mass spectra were recorded by the ETH Zürich MS service; HRMS (ESI) spectra were measured on a Bruker Daltonics maxis (UHR-TOF) and HRMS (EI) on a Waters Micromass AutoSpec Ultima instrument.

### 1. Synthesis of Building Block X

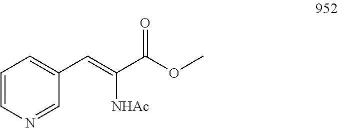 [SMILES: COC(=O)/C(=C/c1cccnc1)NC(C)=O]

Ester 952:

Pyridine-3-carbaldehyde (950) (4.38 mL, 46.7 mmol, 1.00 eq.) followed by Ac₂O (24.8 mL, 243 mmol, 5.20 eq.) were added to a mixture of N-acetylglycine (5.47 g, 46.7 mmol, 1.00 eq.) and NaOAc (4.21 g, 51.4 mmol, 1.10 eq.). The dark brown mixture was stirred at 115° C. for 18 h. 10 mL MeOH were added (strongly exothermic!) in order to dilute the mixture which was then poured into 40 mL MeOH containing 1.5 g NaOAc. The dark brown mixture was stirred at RT for 72 h. The mixture was partitioned between sat. aq. Na₂CO₃ and CHCl₃ and the aq. phase was extracted with CHCl₃. The combined org. phase was concentrated and the crude product was purified by FC (CH₂Cl₂/MeOH 3%→10%) to yield OH-52 (8.06 g, 78%) as yellow crystals which were recrystallized from hexane/EtOAc.

mp: 108-111° C. (Lit.: 110° C., Journal of Crystallographic and Spectroscopic Research, 1988, 18, 75-85)

¹H-NMR: (400 MHz, CDCl₃): δ=9.82 (s, 1H), 8.84 (d, J=2.00 Hz, 1H), 8.61 (dd, J=4.76, 1.56 Hz, 1H), 8.10 (dt, J=8.04, 1.72 Hz, 1H), 7.53 (dd, J=8.00, 4.80 Hz, 1H), 3.81 (s, 1H), 2.10 (s, 1H).

¹³C-NMR: (100 MHz, CDCl₃): δ=168.4, 165.4, 150.6, 149.7, 136.3 (2x), 130.3, 127.9, 123.4, 52.9, 23.6.

IR: (neat, cm⁻¹): 3237, 2995, 2953, 1721, 1670, 1587, 1567, 1510, 1435, 1371, 1338, 1264, 1219, 1192, 1125, 1025, 985, 808, 764, 733, 705, 634, 609, 521.

HR-MS: (ESI): m/z calc. for C₁₁H₁₃N₂O₃ [M+H]⁺ 221.0921. found 221.0920.

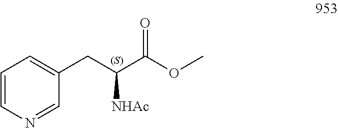

Ester 953: Ester 952 (1.42 g, 6.45 mmol, 1.00 eq.) was dissolved in 75 mL freshly degassed MeOH and HBF₄ (50% in H₂O, 2.22 mL, 9.67 mmol, 1.50 eq.) was added. The solution was transferred into an autoclave and [Rh(COD)(R,R-DIPAMP)]BF₄ (4.88 mg, 6.45 μmol, 0.001 eq.) was added. The autoclave was pressurized with H₂ and subsequently vented 5 times before application of the final pressure of 5 bar. The mixture was heated to 50° C. and stirred for 18 h. The mixture was concentrated, quenched with sat. aq. Na₂CO₃ and extracted with CHCl₃. The crude product was purified by FC (CH₂Cl₂/MeOH 5%) to deliver 953 (1.22 g, 85%) as a yellow solid. The ee was determined by chiral HPLC (AD-H column, isocratic hexane/iPrOH 9:1, 1.0 mL/min, t_{R} major: 10.67 min, t_{R} minor: 15.96 min).

R_{f}: 0.25 (CH₂Cl₂/MeOH 5%)

ee: 87% (determined by HPLC analysis)

mp: 98-101° C. (Lit.: 101-103° C., Tetrahedron Asymmetry, 1996, 7, 117-125)

[α]²⁰_{D}: +100.3 (c 1.19, CHCl₃) (Lit.: +105.1, c=1.08, CHCl₃, Tetrahedron Asymmetry, 1996, 7, 117-125)

¹H-NMR: (400 MHz, CDCl₃): δ 8.48 (dd, J=4.82, 1.66 Hz, 1H), 8.34 (d, J=1.96 Hz, 1H), 7.44 (dt, J=7.82, 1.95 Hz, 1H), 7.22 (ddd, J=7.81, 4.83, 0.63 Hz, 1H), 6.16 (d, J=6.88 Hz, 1H), 4.90 (ddd, J=7.54, 5.75, 1H), 3.74 (s, 3H), 3.18 (dd, J=14.05, 5.84, 2H), 3.08 (dd, J=14.05, 5.64, 2H), 1.99 (s, 3H).

¹³C-NMR: (100 MHz, CDCl₃): δ 171.7, 169.7, 150.4, 148.6, 136.7, 131.6, 123.4, 52.9, 52.5, 35.2, 23.1.

IR: (neat, cm⁻¹): 3267, 3038, 2954, 1742, 1657, 1541, 1481, 1427, 1373, 1282, 1213, 1176, 1132, 1029, 802, 753, 714, 633, 597.

HR-MS: (ESI): m/z calc. for C₁₁H₁₅N₂O₃ [M+H]⁺ 223.1077. found 223.1075.

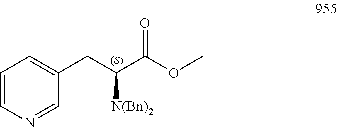

Ester 955: Via Free Amine 954:

To ester 953 (350 mg, 1.58 mmol, 1.00 eq.) dissolved in 8.0 mL MeOH was added SOCl₂ (741 μl, 9.45 mmol, 5.00 eq.) at 0° C. The solution was refluxed at 80° C. for 18 h. The mixture was concentrated and dissolved in toluene. The solvent was removed in vacuo and the yellow solid was portioned between CHCl₃ and sat. aq. Na₂CO₃. The aq. phase was extracted with CHCl₃ and the combined org. phase was concentrated to yield 954 (crude, 243 mg, 86%) as an orange oil. A ¹H- and ¹³C-NMR spectrum confirmed the complete transformation to the free amine 954:

¹H-NMR: (400 MHz, CD₃OD): δ=8.97 (s, 1H), 8.86 (d, J=5.64 Hz, 1H), 8.68 (d, J=8.08 Hz, 1H), 8.14 (dd, J=8.02, 5.86 Hz, 1H), 4.59 (t, J=7.04 Hz, 1H), 3.83 (s, 3H), 3.60 (dd, J=14.69, 7.48 Hz, 2H), 3.51 (dd, J=14.71, 6.58 Hz, 2H).

¹³C-NMR: (100 MHz, CD₃OD): δ=168.0, 148.0, 142.1, 140.5, 136.0, 127.3, 52.6, 52.5, 32.3.

To a solution of free amine 954 (234 mg, 1.30 mmol, 1.00 eq.) in 5 mL MeOH and 0.5 mL AcOH, benzaldehyde (791 μl, 7.79 mmol, 6.00 eq.), NaCNBH₃ (163 mg, 2.60 mmol, 2.00 eq.) and molecular sieves (4 Å) were added at RT and the suspension was stirred for 24 h. The mixture was quenched with sat. aq. NaHCO₃ and extracted with Et₂O. The combined org. phase was dried over MgSO₄ and concentrated. The residue was purified by FC (hexane/EtOAc 4:1→7:3→0:1) to yield 955 (411 mg, 88%) as a colorless oil.

R_{f}: 0.21 (hexane/EtOAc 4:1)

[α]²⁰_{D}: −88.16 (c 1.20, CHCl₃)

¹H-NMR: (400 MHz, CDCl₃): δ 8.49 (dd, J=4.76, 1.64 Hz, 1H), 8.33 (d, J=1.76 Hz, 1H), 7.30-7.19 (m, 11H), 7.14 (ddd, J=7.78, 4.80, 0.74 Hz, 1H), 3.97 (d, J=13.85 Hz, 2H), 3.79 (s, 3H), 3.66 (dd, J=8.58, 6.78 Hz, 1H), 3.57 (d, J=13.85 Hz, 2H), 3.10 (dd, J=14.27, 6.78 Hz, 1H), 3.00 (dd, J=14.29, 8.60 Hz, 1H).

¹³C-NMR: (100 MHz, CDCl₃): δ 172.3, 150.7, 147.7, 138.8, 136.7, 133.8, 128.7, 128.3, 127.1, 123.1, 61.9, 54.5, 51.3, 33.0.

IR: (neat, cm⁻¹): 3028, 2950, 2843, 1730, 1576, 1494, 1479, 1453, 1425, 1374, 1361, 1291, 1214, 1195, 1162, 1128, 1075, 1028, 990, 787, 747, 715, 699.

HR-MS: (ESI): m/z calc. for C₂₃H₂₅N₂O₂ [M+H]+361.1911. found 361.1914.

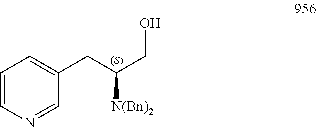

Alcohol 956

955 (943 mg, 2.62 mmol, 1.00 eq.) was dissolved in 17 mL Et₂O and cooled to 0° C. LAH (199 mg, 5.23 mmol, 2.00 eq.) was added and the suspension was stirred at 0° C. for 30 min and quenched with 2 mL H₂O, 2 mL 10% NaOH, 6 mL H₂O. The mixture was filtered, concentrated and purified by FC (hexane/EtOAc 2:3) to yield alcohol 956 (865 mg, 99%) as a colorless oil.

R_{f}: 0.26 (hexane/EtOAc 2:3)

[α]²⁰_{D}: +26.33 (c 1.00, CHCl₃)

¹H-NMR: (400 MHz, CDCl₃): δ 8.38 (dd, J=4.8, 1.6 Hz, 1H), 8.32 (d, J=1.8 Hz, 1H), 7.32-7.28 (m, 1H), 7.28-7.15 (m, 10H), 7.12 (ddd, J=7.8, 4.8, 0.7 Hz, 1H), 3.86 (d, J=13.3 Hz, 2H), 3.55-3.39 (m, 3H), 3.28 (dd, J=10.8, 4.4 Hz, 1H), 3.04-2.94 (m, 2H), 2.85 (s, 1H), 2.40 (dd, J=14.6, 10.6 Hz, 1H).

¹³C-NMR: (100 MHz, CDCl₃): δ 150.4, 147.8, 138.8, 136.3, 134.8, 128.9, 128.6, 127.5, 123.4, 60.7, 60.3, 53.4, 29.2.

IR: (neat, cm⁻¹): 3304 (br.), 3061, 3028, 2930, 2834, 2804, 1578, 1494, 1480, 1453, 1425, 1363, 1129, 1044, 1028, 779, 746, 732, 714, 699.

HR-MS: (ESI): m/z calc. for C₂₂H₂₅N₂O [M+H]⁺ 333.1961. found 333.1952.

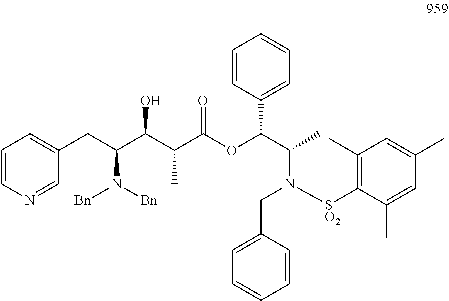 [SMILES: Cc1cc(C)c(S(=O)(=O)N(Cc2ccccc2)[C@@H](C)[C@H](OC(=O)[C@H](C)[C@H](O)[C@H](Cc2cccnc2)N(Cc2ccccc2)Cc2ccccc2)c2ccccc2)c(C)c1]

Ester 959:

Alcohol 956 (40.0 mg, 332 μmol, 1.00 eq.) was dissolved in 1 mL CH₂Cl₂ and DMP (76.6 mg, 424 μmol, 1.50 eq.) was added at 0° C. The suspension was stirred at 0° C. for 30 min and was diluted with Et₂O. The reaction was quenched with 1 mL DMP workup solution (14 g sodium thiosulfate in 1 l 80% sat. aq. NaHCO₃) and stirred for 30 min at 0° C. The aq. phase was extracted 3× with Et₂O. The combined org. phase was washed with H₂O and brine, dried over MgSO₄ and concentrated at 20° C. The obtained aldehyde 957 was dried at 10⁻³ mbar (RT) for 4 h. Propanoic acid (1R,2S)-2-(N-benzyl-1-(3,5-dimethylphenyl)methylsulfonamido)-1-phenylpropyl ester 958 (75.1 mg, 157 μmol, 1.50 eq.) was dissolved in 1 mL CH₂C1, and NEt₃ (54.4 μl, 392 μmol, 3.75 eq.) was added. The solution was cooled to −78° C. and dicyclohexylboron trifluoromethanesulfonate (112 mg, 345 μmol, 3.30 eq.) in 350 μl hexane was added dropwise during 15 min. The resulting solution was stirred at −78° C. for 3 h. Aldehyde 957 (34.5 mg, 104 μmol, 1.00 eq.) dissolved in 0.5 mL CH₂Cl₂ was added dropwise during 20 min and the solution was stirred at −78° C. for 3 h. The mixture was warmed very slowly to 0° C. and stirred at that temperature for 1 h. The reaction was quenched with 1 mL pH 7 buffer, diluted with 4.5 mL MeOH and stirred with 0.45 mL H₂O₂ (50%) for 16 h at RT. The org. solvents were removed in vacuo and the residue was taken up in CH₂Cl₂ and H₂O. The aq. phase was extracted with CH₂Cl₂ and the combined org. phase was dried over MgSO₄. The solvents were removed in vacuo and the residue was purified by FC (hexane/EtOAc 3:2) to yield 959 (all isomers 71.1 mg, 73% over 2 steps, 5:1 ratio of isomers which can be separated by FC with hexane/EtOAc 3:2 as eluent). The analytical data correspond to the desired isomer ester 959 in pure form.

R_{f}: 0.24 (hexane/EtOAc 3:2)

[α]²⁰_{D}: +20.22 (c 1.03, CHCl₃)

¹H-NMR: (400 MHz, CDCl₃): δ 8.40 (d, J=1.7 Hz, 1H), 8.36 (dd, J=4.8, 1.6 Hz, 1H), 7.42 (dt, J=7.8, 1.9 Hz, 1H), 7.30-6.97 (m, 19H), 6.81-6.73 (m, 4H), 5.72 (d, J=4.0 Hz, 1H), 4.62 (d, J=16.5 Hz, 1H), 4.43 (d, J=16.5 Hz, 1H), 4.11 (d, J=13.1 Hz, 1H), 4.00-4.08 (m, 3H), 3.36 (d, J=13.5 Hz, 2H), 3.31-3.23 (m, 1H), 3.20 (d, J=3.3 Hz, 1H), 3.05-2.91 (m, 3H), 2.85-2.76 (m, 1H), 2.40 (s, 6H), 2.19 (s, 3H), 1.05 (d, J=7.0 Hz, 3H), 0.37 (d, J=7.1 Hz, 3H).

¹³C-NMR: (100 MHz, CDCl₃): δ 174.8, 150.6, 147.6, 142.5, 140.3, 139.7, 138.3, 138.2, 136.8, 135.7, 133.5, 132.1, 129.1, 128.4, 128.3, 127.9, 127.5, 127.2, 127.0, 125.9, 123.4, 78.4, 72.9, 59.1, 56.8, 55.6, 48.2, 42.6, 26.9, 22.9, 20.9, 13.1, 12.9.

IR: (neat, cm⁻¹): 3062, 3028, 2979, 2939, 1738, 1604, 1495, 1454, 1378, 1323, 1261, 1205, 1151, 1029, 1013, 931, 857, 751, 730, 699, 661, 568, 538.

HR-MS: (ESI): m/z calc. for C₅₀H₅₆N₃O₅S [M+H]⁺ 810.3935. found 810.3934.

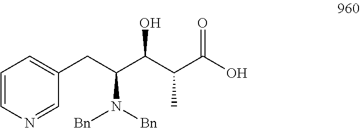 [SMILES: C[C@@H](C(=O)O)[C@H](O)[C@H](Cc1cccnc1)N(Cc1ccccc1)Cc1ccccc1]

Acid 960:

To ester 959 (176 mg, 217 μmol, 1.00 eq.) dissolved in 5.2 mL MeOH/THF/H₂O 3:2:2 was added LiOH.H₂O (45.6 mg, 1.09 mmol, 5.00 eq.) at RT. The clear solution was stirred for 24 h at RT and diluted with Et₂O. The aq. phase was acidified to pH 2 (aq. HCl 1 M) and the cleaved auxiliary was extracted, leaving the product in the aq. phase. The latter was set to pH 7 (sat. aq. NaHCO₃) and the product was extracted with CHCl₃. The org. phase was dried over MgSO₄ and concentrated to yield acid 960 (84.1 mg, 96%) as a yellow, viscous residue.

R_{f}: 0.08 (hexane/EtOAc 3:7)

[α]²⁰_{D}: +35.6 (c 0.540, CHCl₃)

¹H-NMR: (400 MHz, CDCl₃): δ 8.48 (s, 1H), 8.40 (d, J=3.3 Hz, 1H), 7.53 (d, J=7.9 Hz, 1H), 7.29-7.04 (m, 11H), 4.05 (d, J=12.8 Hz, 2H), 3.46 (t, J=5.6 Hz, 1H), 3.39 (d, J=13.4 Hz, 2H), 3.04-2.89 (m, 3H), 2.80-2.64 (m, 1H), 0.84 (d, J=7.1 Hz, 3H).

¹³C-NMR: (100 MHz, CDCl₃): δ 177.7, 149.5, 146.3, 138.8, 137.8, 136.3, 129.2, 128.5, 127.4, 123.8, 73.0, 61.0, 55.1, 42.2, 28.6, 14.6.

IR: (neat, cm⁻¹): 3411, 3062, 3027, 2973, 2936, 2804, 1713, 1494, 1454, 1423, 1376, 1302, 1266, 1196, 1129, 1090, 1075, 1049, 1027, 1007, 983, 751, 700.

HR-MS: (ESI): m/z calc. for C₂₅H₂₉N₂O₃ [M+H]⁺ 405.2173. found 405.2173.

### 3. Synthesis of the Second Building Block

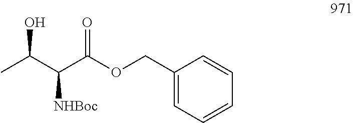 [SMILES: C[C@@H](O)[C@H](NC(=O)OC(C)(C)C)C(=O)OCc1ccccc1]

Protected L-Thr (971):

L-Thr (2.55 g, 21.4 mmol, 1.00 eq.) was dissolved in 75 mL 50% THF/H₂O. Na₂CO₃ (4.78 g, 45.1 mmol, 2.10 eq.) in 20 mL H₂O was added and the mixture was stirred for 10 min at RT. Boc₂O (5.92 mL, 25.8 mmol, 1.20 eq.) was added and the turbid mixture was stirred for 14 h at RT. The mixture was diluted with 15 mL H₂O and the pH was set to 4 (aq. HCl 1 M). The aq. phase was extracted with EtOAc, the pH was lowered to 3 (aq. HCl 1 M), NaCl was added to saturation and the aq. phase was again extracted with EtOAc. The combined organic phase was dried over MgSO₄ and concentrated to yield L-Boc-Thr (3.79 g, 81% crude) as a colorless foam. The protected amino acid (3.79 g, 17.3 mmol, 1.00 eq.) and benzyl bromide (1.63 mL, 18.9 mmol, 1.05 eq.) were dissolved in 100 mL DMF at 0° C. Cs₂CO₃ (2.93 g, 8.99 mmol, 0.52 eq.) was added and the suspension was stirred for 20 h at RT. H₂O was added and the mixture was extracted with EtOAc. The combined org. phase was washed with H₂O, brine, dried over MgSO₄ and concentrated. The resulting oil was purified by FC (hexane/EtOAc 9:1→3:2) to deliver protected L-Thr 971 (4.59 g, 86%) as a colorless oil.

R_{f}: 0.31 (hexane/EtOAc 9:1)

[α]²⁰_{D}: −14.45 (c 1.05, CHCl₃)

¹H-NMR: (400 MHz, CDCl₃): δ 7.37-7.34 (m, 5H), 5.37-5.29 (m, 1H, NH), 5.20 (q, J=11.3 Hz, 2H), 4.35-4.27 (m, 2H), 2.10-2.04 (m, 1H, OH), 1.44 (s, 9H), 1.23 (d, J=6.32 Hz, 3H).

¹³C-NMR: (100 MHz, CDCl₃): δ 171.3, 156.1, 128.6, 128.6, 128.4, 128.2, 80.1, 68.2, 67.2, 58.8, 28.3, 19.9.

IR: (neat, cm⁻¹): 3437, 2978, 2934, 1743, 1715, 1692, 1500, 1456, 1367, 1253, 1160, 112, 1066, 1000, 880, 752, 736, 698.

HR-MS: (ESI): m/z calc. for C₁₆H₂₃NNaO₅ [M+Na]⁺ 332.1468. found 332.1471.

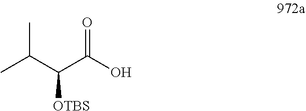

(S)-TBS-Protected Alcohol 972a:

The compound was prepared according to J. Chem. Soc., Perkin Trans. 1, 1996, 1427-1433. (S)-2-Hydroxy-3-methylbutyric acid (not shown) (1.04 g, 8.80 mmol, 1.00 eq.), TBS-Cl (3.19 g, 21.1 mmol, 2.40 eq.) and imidazole (3.64 g, 42.3 mmol, 4.80 eq) were dissolved in 11 mL DMF at RT. The mixture was stirred 24 h. The mixture was diluted with 200 mL EtOAc, washed 3× with 40 mL each sat. aq. citric acid, sat. aq. NaHCO₃ and brine, dried over MgSO₄ and concentrated. The resulting oil was dissolved in 70 mL MeOH and cooled to 0° C. 2 g K₂CO₃ in 24 mL H₂O were added and the mixture was stirred for 2.5 h at RT. The pH of the solution was adjusted to 4 (aq. HCl 1 M) and the aq. phase was extracted with EtOAc (3×80 mL). The combined org. phase was dried over MgSO₄ and concentrated. The colorless oil was purified by FC (hexane/EtOAc 6:1→4:1) to deliver (S)-TBS-protected alcohol 972a (1.54 g, 75%) as a colorless oil.

R_{f}: 0.28 (10% MeOH in CH₂Cl₂)

[α]²⁰_{D}: −16.45 (c 0.811, CH₂Cl₂)

¹H-NMR: (400 MHz, CDCl₃): δ 10.09 (br. S, 1H), 4.06 (d, J=4.0 Hz, 1H), 2.14-2.04 (m, 1H), 0.98 (d, J=6.2 Hz, 3H), 0.94-0.93 (m, 12H, overlapping signals), 0.09 (s, 6H).

¹³C-NMR: (100 MHz, CDCl₃): δ 176.8, 76.7, 32.8, 25.7, 18.7, 18.2, 16.7, −5.2.

HR-MS: (ESI): m/z calc. for C₁₁H₂₃O₃Si [M−H]⁻ 231.1422. found 231.1426.

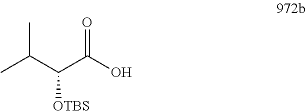

(R)-TBS-Protected Alcohol 972b:

The compound was prepared according to J. Chem. Soc., Perkin Trans. 1, 1996, 1427-1433 while the analytics are compared to Analytics compared to: J. Org. Chem. 1989, 54, 2085-2091. (R)-2-Hydroxy-3-methylbutyric acid (not shown) (305 mg, 2.58 mmol, 1.00 eq.), TBS-Cl (934 mg, 6.20 mmol, 2.40 eq.) and imidazole (1.07 g, 12.4 mmol, 4.80 eq) were dissolved in 3.5 mL DMF at RT. The mixture was stirred 24 h. The mixture was diluted with 65 mL EtOAc, washed 3× with 10 mL sat. aq. citric acid, sat. aq. NaHCO₃ and brine, dried over MgSO₄ and concentrated. The resulting oil was dissolved in 22 mL MeOH and cooled to 0° C. 650 mg K₂CO₃ in 8 mL H₂O were added and the mixture was stirred for 2.5 h at RT. The solution was adjusted to pH 4 (aq. HC 1 M) and the aq. phase was extracted with EtOAc (3×40 mL). The combined org. phase was dried over MgSO₄ and concentrated. The colorless oil was purified by FC (hexane/EtOAc 6:1→4:1) to deliver (R)-TBS-protected alcohol 972b (339 mg, 57% over 2 steps) as a colorless oil.

R_{f}: 0.25 (10% MeOH in CH₂Cl₂)

[α]²_{D}: +18.31 (c 0.942, CH₂Cl₂)

¹H-NMR: (400 MHz, CDCl₃) δ 4.05 (d, J=4.0 Hz, 1H), 2.16-2.01 (m, 1H), 0.97 (d, J=6.9 Hz, 3H), 0.95-0.91 (m, 12H), 0.09 (s, 6H).

¹³C-NMR: (101 MHz, CDCl₃) δ 176.9, 76.7, 32.8, 25.7, 18.8, 16.7, −5.2.

HR-MS: (ESI): m/z calc. for C₁₁H₂₃O₃ Si [M−H]⁻ 231.1422. found 231.1424.

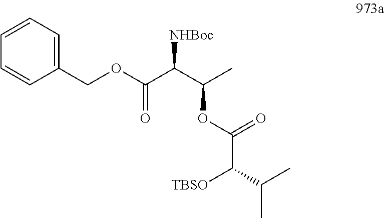 [SMILES: CC(C)[C@H](O[Si](C)(C)C(C)(C)C)C(=O)O[C@H](C)[C@H](NC(=O)OC(C)(C)C)C(=O)OCc1ccccc1]

(S)-TBS-Ether 973a:

To a stirred solution of (S)-TBS-protected alcohol 972a (70.5 mg, 303 μmol, 1.00 eq.), Et₃N (169 μl, 1.21 mmol, 4.00 eq.), and DMAP (74.1 mg, 607 μmol, 2.00 eq.) in 2 mL toluene was added 2,4,6-trichlorobenzoyl chloride (71.2 μl, 455 μmol, 1.50 eq.). As the mixture became turbid (white precipitate) a solution of 971 (98.7 mg, 319 μmol, 1.05 eq.) dissolved in 2 mL toluene was added at RT. The yellow slurry was stirred at RT for 18 h. NaHCO₃ was added and the aq. phase was extracted with EtOAc. The combined org. phase was dried over MgSO₄ and concentrated. The yellow residue was purified by FC (hexane/EtOAc 9.5:1) to deliver (S)-TBS-ether 973a (106 mg, 67%) as a colorless oil.

R_{f}: 0.41 (hexane/EtOAc 9.5:1)

[α]²⁰_{D}: +1.43 (c 1.36, CHCl₃)

¹H-NMR: (400 MHz, CDCl₃): δ 7.42-7.28 (m, 5H), 5.54-5.44 (m, 1H), 5.20 (d, J=9.8 Hz, 1H), 5.16 (d, J=12.2 Hz, 1H), 5.05 (d, J=12.2 Hz, 1H), 4.47 (dd, J=9.8, 1.7 Hz, 1H), 3.94 (d, J=4.2 Hz, 1H), 2.03-1.92 (m, 1H), 1.45 (s, 9H), 1.30 (d, 3H), 0.92-0.90 (m, 12H, overlapping signals), 0.83 (d, J=6.8 Hz, 3H), 0.03 (d, J=6.8 Hz, 6H).

¹³C-NMR: (100 MHz, CDCl₃): δ 172.2, 169.9, 155.9, 135.0, 128.6, 128.5, 128.4, 80.2, 76.6, 70.9, 67.6, 57.3, 32.8, 28.3, 25.7, 19.1, 18.2, 17.0, 16.5, −4.9, −5.4.

IR: (neat, cm⁻¹): 2959, 2931, 2858, 1751, 1722, 1500, 1457, 1386, 1367, 1314, 1251, 1163, 1143, 1112, 1083, 1066, 980, 861, 835, 778, 751, 678.

HR-MS: (ESI): m/z calc. for C₂₇H₄₆NO₇Si [M+H]⁺ 524.3038. found 524.3043.

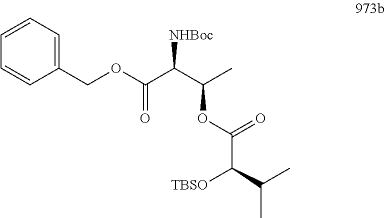 [SMILES: CC(C)[C@@H](O[Si](C)(C)C(C)(C)C)C(=O)O[C@H](C)[C@H](NC(=O)OC(C)(C)C)C(=O)OCc1ccccc1]

(R)-TBS-Ether 973b:

To a stirred solution of (R)-TBS-protected alcohol 972b (250 mg, 1.08 mmol, 1.00 eq.) and Et₃N (449 μl, 3.23 mmol, 3.00 eq.) in 10 mL toluene was added 2,4,6-trichlorobenzoyl chloride (210 μl, 1.35 mmol, 1.25 eq.). As the mixture became turbid (white precipitate) a solution of 971 (350 mg, 1.13 mmol, 1.05 eq.) dissolved in 5 mL toluene and DMAP (263 mg, 2.15 mmol, 2.00 eq.) was added at RT. The yellow slurry was stirred at RT for 18 h. NaHCO₃ was added and the aq. phase was extracted with EtOAc. The combined org. phase was dried over MgSO₄ and concentrated. The yellow residue was purified by FC (hexane/EtOAc 9.5:1) to deliver (R)-TBS-ether 973b (411 mg, 73%) as a colorless oil.

R_{f}: 0.36, (hexane/EtOAc 9.5:1)

[α]²⁰: +44.4 (c 1.41, CHCl₃)

¹H-NMR: (400 MHz, CDCl₃) δ 7.39-7.29 (m, 5H), 5.48 (qd, J=6.3, 2.5 Hz, 1H), 5.23-5.13 (m, 2H), 5.04 (d, J=12.2 Hz, 1H), 4.46 (dd, J=9.7, 2.4 Hz, 1H), 3.93 (d, J=4.1 Hz, 1H), 2.00-1.89 (m, 1H), 1.46 (s, 9H), 1.30 (d, J=6.4 Hz, 3H), 0.92 (s, 12H), 0.82 (d, J=6.8 Hz, 3H), 0.03 (d, J=6.7 Hz, 6H).

¹³C-NMR: (101 MHz, CDCl₃) δ 172.2, 170.0, 155.9, 134.9, 128.6, 128.5, 128.4, 80.3, 76.5, 70.9, 67.6, 57.3, 32.7, 28.3, 25.7, 19.2, 18.3, 17.1, 16.4, −4.9, −5.4.

IR: (neat, cm⁻¹): 3027, 2934, 2805, 1715, 1496, 1455, 1423, 1302, 1266, 1208, 1129, 1075, 1048, 1027, 981, 751, 700, 500, 471, 435.

HR-MS: (ESI): m/z calc. for C₂₇H₄₅NNaO₇Si [M+Na]⁺ 546.2858. found 546.2857.

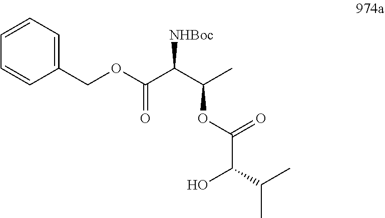 [SMILES: CC(C)[C@H](O)C(=O)O[C@H](C)[C@H](NC(=O)OC(C)(C)C)C(=O)OCc1ccccc1]

(S)-Alcohol 974a:

(S)-TBS-ether 973a (460 mg, 878 μmol, 1.00 eq.) was dissolved in 12 mL anhydrous THF and HF•py (30%, 2 mL in 2 batches) was added at 0° C. The solution was stirred at RT for 1 h. HF•py (30%, 1 mL) was added and the mixture was stirred for 16 h at RT. The mixture was quenched with sat. aq. NaHCO₃ and extracted with EtOAc. The org. phase was dried over MgSO₄ and concentrated. The residue was purified by FC (hexane/EtOAc 4:1) to yield (S)-alcohol 974a (344 mg, 96%) as a colorless oil.

R_{f}: 0.21 (hexane/EtOAc 4:1)

[α]²⁰_{D}: +2.58 (c 1.91, CHCl₃)

¹H-NMR: (400 MHz, CDCl₃): δ 7.41-7.29 (m, 5H), 5.50 (dd, J=6.3, 2.2 Hz, 1H), 5.23-5.05 (m, 3H), 4.52 (dd, J=9.6, 2.0 Hz, 1H), 3.76 (dd, J=5.8, 3.4 Hz, 1H), 2.49 (d, J=5.9 Hz, 1H), 1.98 (qd, J=6.9, 3.4 Hz, 1H), 1.46 (s, 9H), 1.30 (d, J=6.4 Hz, 3H), 0.98 (d, J=6.9 Hz, 3H), 0.80 (d, J=6.8 Hz, 3H).

¹³C-NMR: (101 MHz, CDCl₃): δ 173.8, 169.7, 155.8, 135.0, 128.7, 128.6, 80.5, 74.5, 72.1, 67.7, 57.1, 31.9, 28.3, 18.8, 16.8, 15.7.

IR: (neat, cm⁻¹): 3449, 2971, 2936, 1739, 1716, 1500, 1456, 1384, 1367, 1316, 1248, 1213, 1163, 1083, 1062, 1031, 996, 753, 698.

HR-MS: (ESI): m/z calc. for C₂₁H₃₂NO₇ [M+H]⁺ 524.3038. found 524.3043.

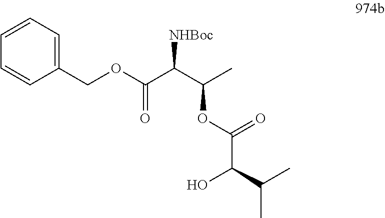 [SMILES: CC(C)[C@@H](O)C(=O)O[C@H](C)[C@H](NC(=O)OC(C)(C)C)C(=O)OCc1ccccc1]

(R)-Alcohol 974b:

(R)-TBS-ether 973b (378 mg, 722 μmol, 1.00 eq.) was dissolved in 10 mL anhydrous THF and HF•py (30%, 3.1 mL in 2 batches) was added at 0° C. The solution was stirred for 16 h at RT. The mixture was quenched with NaHCO₃ and extracted with EtOAc. The org. phase was dried over MgSO₄ and concentrated. The residue was purified by FC (hexane/EtOAc 4:1) to yield (R)-alcohol 974b (254 mg, 86%) as a colorless oil.

R_{f}: 0.23 (hexane/EtOAc 4:1)

[α]²⁰_{D}: +29.2 (c 0.765, CHCl₃)

¹H-NMR: (400 MHz, CDCl₃): δ 7.40-7.30 (m, 5H), 5.49 (qd, J=6.2, 2.5 Hz, 1H), 5.24-5.13 (m, 2H), 5.07 (d, J=12.1 Hz, 1H), 4.52 (dd, J=9.6, 2.4 Hz, 1H), 3.96 (dd, J=5.9, 3.2 Hz, 1H), 2.56 (d, J=6.1 Hz, 1H), 2.01-1.89 (m, 1H), 1.46 (s, 9H), 1.33 (d, J=6.4 Hz, 3H), 0.99 (d, J=6.9 Hz, 3H), 0.76 (d, J=6.8 Hz, 3H).

¹³C-NMR: (101 MHz, CDCl₃): δ 173.8, 169.8, 155.8, 134.8, 128.7, 128.4, 80.5, 75.1, 72.5, 67.8, 57.1, 31.9, 28.3, 18.9, 16.9, 15.5.

IR: (neat, cm⁻¹): 3460, 2974, 2936, 1740, 1717, 1501, 1456, 1384, 1368, 1346, 1315, 1282, 1248, 1213, 1164, 1136, 1085, 1063, 1031, 997, 698.

HR-MS: (ESI): m/z calc. for C₂₁H₃₁NNaO₇ [M+Na]⁺ 432.1993. found 432.1984.

### 4. Synthesis of Analogs 2 and 3

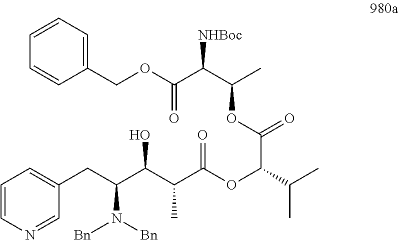 [SMILES: CC(C)[C@H](OC(=O)[C@H](C)[C@H](O)[C@H](Cc1cccnc1)N(Cc1ccccc1)Cc1ccccc1)C(=O)O[C@H](C)[C@H](NC(=O)OC(C)(C)C)C(=O)OCc1ccccc1]

Ester 980a:

To a stirred solution of 960 (34.0 mg, 84.1 μmol, 1.00 eq.) and 2,4,6-trichlorobenzoyl chloride (23.0 μl, 147 μmol, 1.75 eq.) in 0.5 mL THF was added Et₃N (35.1 μl, 252 μmol, 3.00 eq.) at −78° C. The mixture was stirred for 5 min and a solution of 974a (37.9 mg, 92.5 μmol, 1.10 eq.) and DMAP (13.4 mg, 109 μmol, 1.30 eq.) in 0.4 mL toluene was added at −78° C. The clear solution was stirred at −78° C. for 30 min and was slowly warmed to −35° C. The turbid mixture was stirred at that temperature for 45 h and was warmed to 0° C. for the last 25 min. The reaction was quenched at 0° C. with sat. aq. NaHCO₃. The aq. phase was extracted with EtOAc and the combined org. phase was dried over MgSO₄ and concentrated. The yellow oil was purified by FC (hexane/EtOAc 3:2) to yield ester 980a (47.1 mg, 64%) as a colorless film.

R_{f}: 0.21 (hexane/EtOAc 3:2)

[α]²⁰_{D}: +45.0 (c 0.960, CHCl₃)

¹H-NMR: (400 MHz, CDCl₃): δ 8.42 (d, J=1.7 Hz, 1H), 8.32 (dd, J=4.8, 1.6 Hz, 1H), 7.50-7.43 (m, 1H), 7.27-7.03 (m, 16H), 5.43-5.33 (m, 1H), 5.25 (d, J=9.5 Hz, 1H), 5.02 (d, J=12.0 Hz, 1H), 4.93 (d, J=12.0 Hz, 1H), 4.64 (d, J=3.9 Hz, 1H), 4.37 (dd, J=9.6, 2.5 Hz, 1H), 4.13 (d, J=12.1 Hz, 2H), 3.62 (d, J=4.0 Hz, 1H), 3.38-3.27 (m, 3H), 3.16-2.99 (m, 3H), 2.69 (dd, J=8.4, 4.2 Hz, 1H), 2.07-1.92 (m, 1H), 1.30 (s, 9H), 1.13 (d, J=6.4 Hz, 3H), 0.74 (d, J=6.9 Hz, 3H), 0.67 (d, J=6.8 Hz, 3H), 0.22 (d, J=6.9 Hz, 3H).

¹³C-NMR: (101 MHz, CDCl₃) δ 174.5, 169.7, 169.6, 155.9, 150.7, 147.5, 140.0, 136.9, 136.0, 134.8, 129.3, 128.6, 128.5, 128.4, 127.1, 123.4, 80.3, 75.6, 73.7, 72.4, 68.0, 58.8, 57.1, 55.8, 43.7, 29.9, 28.3, 26.9, 18.6, 16.7, 16.6, 13.2.

IR: (neat, cm⁻¹): 2975, 2935, 1736, 1497, 1455, 1423, 1368, 1311, 1251, 1215, 1164, 1129, 1086, 1062, 1028, 985, 937, 752, 700.,

HR-MS: (ESI): m/z calc. for C₄₆H₅₈N₃O₉ [M+H]⁺ 796.4168. found 796.4163.

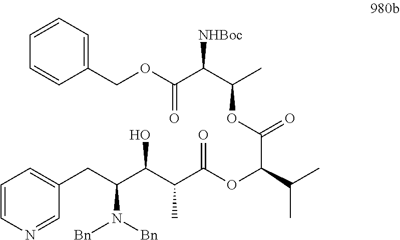 [SMILES: CC(C)[C@@H](OC(=O)[C@H](C)[C@H](O)[C@H](Cc1cccnc1)N(Cc1ccccc1)Cc1ccccc1)C(=O)O[C@H](C)[C@H](NC(=O)OC(C)(C)C)C(=O)OCc1ccccc1]

Ester 980b:

To a stirred solution of 960 (40.0 mg, 98.9 μmol, 1.00 eq.) and 2,4,6-trichlorobenzoyl chloride (27.1 μl, 173 μmol, 1.75 eq.) in 0.6 mL THF was added Et₃N (41.2 μl, 297 μmol, 3.00 eq.) at −78° C. The mixture was stirred for 5 min and a solution of 974b (44.5 mg, 109 μmol, 1.10 eq.) and DMAP (15.7 mg, 129 μmol, 1.30 eq.) in 0.5 mL toluene was added at −78° C. The clear solution was stirred at −78° C. for 30 min and was slowly warmed to −35° C. The turbid mixture was stirred at that temperature for 43 h and was warmed to 0° C. for the last 25 min. The reaction was quenched at 0° C. with sat. aq. NaHCO₃. The aq. phase was extracted with EtOAc and the combined org. phase was dried over MgSO₄ and concentrated. The yellow oil was purified by FC (hexane/EtOAc 3:2) to yield ester 980b (43.0 mg, 50%) as a colorless film.

R_{f}: 0.24 (hexane/EtOAc 3:2)

[α]²): +49.3 (C 1.12, CHCl₃)

¹H-NMR: (400 MHz, CDCl₃): δ 8.45 (d, J=1.7 Hz, 1H), 8.35 (dd, J=4.7, 1.2 Hz, 1H), 7.51 (dt, J=7.7, 1.7 Hz, 1H), 7.28-7.05 (m, 16H), 5.62 (d, J=10.0 Hz, 1H), 5.46 (qd, J=6.2, 2.5 Hz, 1H), 5.01 (d, J=12.1 Hz, 1H), 4.93 (d, J=12.1 Hz, 1H), 4.57 (d, J=3.8 Hz, 1H), 4.40 (dd, J=10.0, 2.4 Hz, 1H), 4.16 (br. s, 2H), 3.82 (d, J=4.0 Hz, 1H), 3.39-3.26 (m, 3H), 3.22-2.97 (m, 3H), 2.77-2.64 (m, 1H), 2.10 (qd, J=10.6, 6.8 Hz, 1H), 1.30 (s, 9H), 1.11 (d, J=6.4 Hz, 3H), 0.87 (d, J=6.9 Hz, 3H), 0.83 (d, J=6.8 Hz, 3H), 0.16 (d, J=6.8 Hz, 3H).

¹³C-NMR: (101 MHz, CDCl₃): δ 175.8, 170.4, 169.0, 156.1, 150.8, 147.6, 140.1, 137.1, 135.8, 134.8, 129.5, 128.6, 128.5, 128.3, 127.1, 123.5, 80.2, 76.6, 73.9, 72.1, 68.0, 59.3, 57.2, 56.0, 42.1, 30.2, 28.3, 27.3, 18.9, 16.8, 16.8, 13.0.

IR: (neat, cm⁻¹): 3489, 2974, 2936, 2359, 1739, 1717, 1497, 1455, 1367, 1316, 1248, 1217, 1162, 1129, 1086, 1061, 987, 943, 753, 700.

HR-MS: (ESI): m/z calc. for C₄₆H₅₈N₃O₉ [M+H]⁺ 796.4168. found 796.4166.

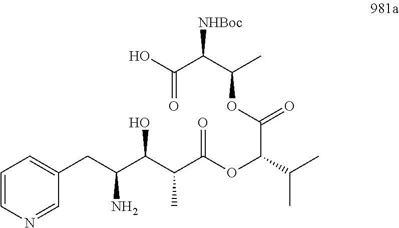 [SMILES: CC(C)[C@H](OC(=O)[C@H](C)[C@H](O)[C@@H](N)Cc1cccnc1)C(=O)O[C@H](C)[C@H](NC(=O)OC(C)(C)C)C(=O)O]

Aminoacid 981a:

980a (24.6 mg, 30.9 μmol, 1.00 eq.) was dissolved in 0.8 mL MeOH and Pd on charcoal (10%, 13.2 mg, 12.4 μmol, 0.400 eq.) was added under Ar. The atmosphere was exchange with H₂ (1 bar) and the mixture was stirred at RT for 5 h. The suspension was filtered over celite, washed with MeOH and concentrated to a white solid (16.4 mg, quant.) which was used crude.

[α]²⁰_{D}: +12.6 (c 1.35, MeOH)

¹H-NMR: (400 MHz, D₂O) δ 8.50 (d, J=1.7 Hz, 2H), 7.88 (d, J=7.9 Hz, 1H), 7.52 (dd, J=7.8, 5.0 Hz, 1H), 5.36 (dt, J=10.0, 5.9 Hz, 1H), 4.82-4.80 (m, 1H), 4.14-4.05 (m, 1H), 3.92-3.74 (m, 2H), 3.23 (dd, J=14.5, 6.9 Hz, 1H), 3.08 (dd, J=14.5, 7.6 Hz, 1H), 2.99 (p, J=7.0 Hz, 1H), 2.28-2.14 (m, 1H), 1.45-1.43 (m, 1H), 1.42 (s, 9H), 1.22 (d, J=7.1 Hz, 6H), 0.94 (t, J=6.4 Hz, 6H).

¹³C-NMR: (101 MHz, D₂O): δ 175.6, 175.1, 170.6, 157.6, 148.7, 147.5, 139.0, 132.0, 124.9, 81.3, 78.1, 73.9, 70.8, 59.3, 53.5, 42.4, 32.8, 29.7 27.6, 17.7, 16.6, 16.3, 13.5.

IR: (neat, cm⁻¹): 3401, 2975, 2937, 1720, 1596, 1501, 1389, 1250, 1171, 1131, 1055, 715.

HR-MS: (ESI): m/z calc. for C₂₅H₄₀N₃O₉ [M+H]⁺ 526.2759. found 526.2752.

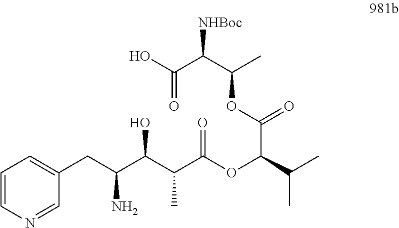 [SMILES: CC(C)[C@@H](OC(=O)[C@H](C)[C@H](O)[C@@H](N)Cc1cccnc1)C(=O)O[C@H](C)[C@H](NC(=O)OC(C)(C)C)C(=O)O]

Aminoacid 981b:

980b (41.2 mg, 51.8 μmol, 1.00 eq.) was dissolved in 1.0 mL MeOH and Pd on charcoal (10%, 22.0 mg, 20.7 μmol, 0.400 eq.) was added under Ar. The atmosphere was exchange with H₂ (1 atm) and the mixture was stirred at RT for 5 h. The suspension was filtered over celite, washed with MeOH and concentrated to a white solid (27.6 mg, quant.) which was used crude.

[α]²⁰_{D}: +15.3 (c 1.32, MeOH)

¹H-NMR: (400 MHz, D₂O): δ 8.82 (d, J=1.4 Hz, 1H), 8.78 (d, J=5.6 Hz, 1H), 8.65-8.58 (m, 1H), 8.10 (dd, J=8.0, 5.9 Hz, 1H), 5.61-5.44 (m, 1H), 4.96 (d, J=4.2 Hz, 1H), 4.34 (d, J=2.9 Hz, 1H), 4.09-3.99 (m, 1H), 3.88 (t, J=5.7 Hz, 1H), 3.53 (dd, J=15.0, 6.0 Hz, 1H), 3.27 (dd, J=15.0, 8.7 Hz, 1H), 3.07 (p, J=6.9 Hz, 1H), 2.28-2.15 (m, 1H), 1.45 (s, 9H), 1.33 (d, J=6.4 Hz, 3H), 1.26 (d, J=7.0 Hz, 3H), 0.95 (d, J=6.9 Hz, 3H), 0.88 (d, J=6.8 Hz, 3H).

¹³C-NMR: (101 MHz, D₂O): δ 174.7, 174.1, 170.7, 158.0, 147.6, 141.8, 140.8, 136.0, 127.7, 81.7, 77.8, 73.1, 71.4, 57.9, 53.3, 42.0, 32.5, 29.8, 27.6, 17.8, 16.3, 16.1, 13.4.

IR: (ATR, film): 3362, 2974, 2935, 2881, 1722, 1505, 1469, 1369, 1311, 1252, 1168, 1129, 1058, 992, 685, 549.

IR: (neat, cm⁻¹): 3362, 2974, 2935, 2881, 1722, 1505, 1469, 1369, 1311, 1252, 1168, 1129, 1058, 992, 685, 549.

HR-MS: (ESI): m/z calc. for C₂₅H₄₀N₃O₉ [M+H]⁺ 526.2759. found 526.2756.

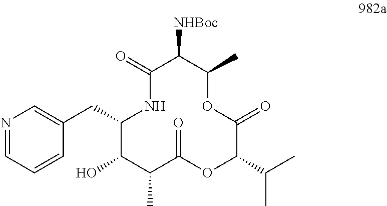 [SMILES: CC(C)[C@@H]1OC(=O)[C@H](C)[C@H](O)[C@H](Cc2cccnc2)NC(=O)[C@@H](NC(=O)OC(C)(C)C)[C@@H](C)OC1=O]

Protected Amine 982a

To a solution of DIPEA (23.1 μl, 87.3 μmol, 2.60 eq.) and HATU (33.2 mg, 87.3 μmol, 1.70 eq.) in 20 mL CH₂Cl₂ and 0.2 mL DMF was added 981a (27.0 mg, 51.4 μmol, 1.00 eq.) in 15 mL CH₂Cl₂ and 0.1 mL DMF over 4 h at RT (pale yellow color develops). The solution was stirred for 18 h at RT. Sat. aq. NaHCO₃ was added and the aq. phase was extracted with CH₂Cl₂. The combined org. phase was dried over MgSO₄, concentrated and purified by FC (hexane/EtOAc 0.5:10) to deliver 982a (20.5 mg, 79%) as an orange film.

R_{f}: 0.23 (hexane/EtOAc 05:10)

[α]²⁰_{D}: −44.9 (c 1.03, MeOH)

¹H-NMR: (400 MHz, MeOD): δ 8.46 (d, J=1.6 Hz, 1H), 8.38 (d, J=3.9 Hz, 1H), 7.78 (d, J=7.8 Hz, 1H), 7.35 (dd, J=7.7, 5.0 Hz, 1H), 4.90-4.84 (m, 1H), 4.54 (d, J=2.7 Hz, 1H), 4.35 (d, J=4.6 Hz, 1H), 4.04 (t, J=7.2 Hz, 1H), 3.65 (s, 1H), 3.05 (dd, J=13.6, 6.4 Hz, 1H), 2.95 (dd, J=13.6, 8.0 Hz, 1H), 2.44 (qd, J=7.0, 2.0 Hz, 1H), 2.30 (dq, J=13.5, 6.8 Hz, 1H), 1.45 (s, 9H), 1.31 (d, J=7.2 Hz, 3H), 1.19 (d, J=6.2 Hz, 3H), 1.07 (dd, J=6.8, 4.4 Hz, 6H).

¹³C-NMR: (101 MHz, MeOD): δ 175.5, 173.0, 169.5, 151.1, 148.1, 139.3, 136.2, 130.8, 125.2, 81.2, 79.8, 73.3, 72.1, 57.6, 56.9, 47.5, 37.0, 31.0, 28.7, 19.4, 17.8, 17.2, 13.1.

IR: (neat, cm⁻¹): 3423, 2973, 2933, 1722, 1671, 1492, 1369, 1252, 1169, 1051, 1020, 847, 771, 716, 608, 561, 535, 510, 446.

HR-MS: (ESI): m/z calc. for C₂₅H₃₈N₃O₈ [M+H]⁺ 508.2653. found 508.2655.

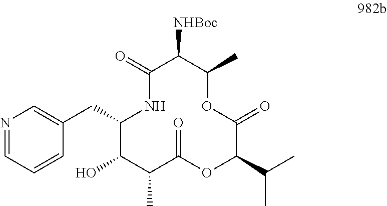 [SMILES: CC(C)[C@H]1OC(=O)[C@H](C)[C@H](O)[C@H](Cc2cccnc2)NC(=O)[C@@H](NC(=O)OC(C)(C)C)[C@@H](C)OC1=O]

Protected Amine 982b

To a solution of DIPEA (33.9 μl, 196 μmol, 2.60 eq.) and HATU (48.7 mg, 128 μmol, 1.70 eq.) in 30 mL CH₂Cl₂ and 0.3 mL DMF was added 981b (39.6 mg, 75.3 μmol, 1.00 eq.) in 20 mL CH₂Cl₂ and 0.2 mL DMF over 4 h at RT (pale yellow color develops). The solution was stirred for 18 h at RT. Sat. aq. NaHCO₃ was added and the aq. phase was extracted with CH₂Cl₂. The combined org. phase was dried over MgSO₄, concentrated purified by FC (hexane/EtOAc 0.5:10) to deliver 25 (24.1 mg, 63%) as an orange film.

R_{f}: 0.19 (hexane/EtOAc 05:10)

[α]²⁰_{D}: −24.2 (c 1.21, MeOH)

¹H-NMR: (400 MHz, MeOD): δ 8.46 (d, J=1.7 Hz, 1H), 8.37 (dd, J=4.9, 1.4 Hz, 1H), 7.79-7.69 (m, 1H), 7.33 (dd, J=7.7, 5.0 Hz, 1H), 5.27-5.12 (m, 1H), 4.68 (d, J=5.1 Hz, 1H), 4.24 (d, J=5.9 Hz, 1H), 4.08 (td, J=7.6, 1.6 Hz, 1H), 3.63 (s, 1H), 3.06-2.91 (m, 2H), 2.59 (qd, J=7.2, 1.0 Hz, 1H), 2.27-2.16 (m, 1H), 1.45 (s, 9H), 1.37 (d, J=7.4 Hz, 3H), 1.29 (d, J=6.5 Hz, 3H), 0.99 (d, J=4.2 Hz, 3H), 0.97 (d, J=4.0 Hz, 3H).

¹³C-NMR: (101 MHz, MeOD): δ 178.4, 170.9, 169.3, 157.1, 151.1, 148.0, 139.3, 136.3, 125.0, 81.1, 79.1, 75.7, 70.9, 57.7, 56.8, 42.4, 36.4, 31.1, 28.7, 18.7, 18.0, 17.8, 15.0.

IR: (neat, cm⁻¹): 3350, 2974, 2935, 1744, 1717, 1673, 1503, 1459, 1388, 1370, 1251, 1169, 1049, 1023, 847, 558.

HR-MS: (ESI): m/z calc. for C₂₅H₃₈N₃O₈ [M+H]+526.2759. found 526.2756.

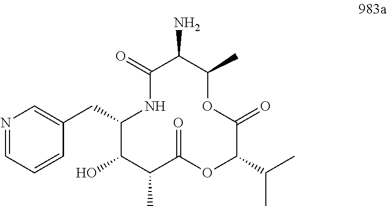 [SMILES: CC(C)[C@@H]1OC(=O)[C@H](C)[C@H](O)[C@H](Cc2cccnc2)NC(=O)[C@@H](N)[C@@H](C)OC1=O]

Amine 983a:

982a (20.5 mg, 40.4 μmol, 1.00 eq.) was dissolved in 2 mL CH₂Cl₂ and TFA (309 μl, 4.04 mmol, 100 eq.) was added at 0° C. The solution was stirred for 2.4 h at RT. The solvents were removed in vacuo (22.0 mg, quant.) to a pale yellow oil which was used crude.

[α]²⁰_{D}: −39.9 (c 0.820, MeOH)

¹H-NMR: (400 MHz, MeOD): δ 8.83 (s, 1H), 8.74 (d, J=5.6 Hz, 1H), 8.57 (d, J=8.1 Hz, 1H), 8.01 (dd, J=8.0, 5.8 Hz, 1H), 5.11-5.01 (m, 1H), 4.55 (d, J=3.0 Hz, 1H), 4.30 (dd, J=12.6, 6.4 Hz, 1H), 4.22 (d, J=4.8 Hz, 1H), 3.79 (d, J=1.9 Hz, 1H), 3.25 (dd, J=13.8, 7.1 Hz, 1H), 3.17 (dd, J=13.9, 7.1 Hz, 1H), 2.57 (qd, J=7.1, 2.3 Hz, 1H), 2.30 (dq, J=13.3, 6.7 Hz, 1H), 1.34 (d, J=7.2 Hz, 3H), 1.28 (d, J=6.0 Hz, 3H), 1.12-0.99 (m, 6H).

¹³C-NMR: (100 MHz, MeOD): δ 175.4, 169.0, 166.4, 148.9, 143.3, 140.8, 140.6, 128.2, 79.7, 73.8, 70.2, 56.0, 55.7, 37.9, 36.5, 31.0, 19.3, 17.8, 16.9, 13.0.

IR: (neat, cm⁻¹): 2973, 2933, 1735, 1673, 1526, 1473, 1282, 1201, 1135, 1069, 837, 798, 757, 722, 483, 470, 458, 444, 409.

HR-MS: (ESI): m/z calc. for C₂₀H₂₉N₃NaO₆ [M+Na]⁺ 430.1949. found 430.1938.

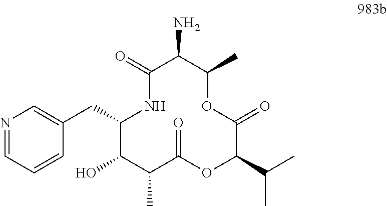 [SMILES: CC(C)[C@H]1OC(=O)[C@H](C)[C@H](O)[C@H](Cc2cccnc2)NC(=O)[C@@H](N)[C@@H](C)OC1=O]

Amine 983b:

982b (5.00 mg, 9.90 μmol, 1.00 eq.) was dissolved in 0.6 mL CH₂Cl₂ and TFA (75.4 μl, 985 mmol, 100 eq.) was added at 0° C. The solution was stirred for 3 h at RT. The solvents were removed in vacuo to deliver a yellow oil (10 mg, quant.).

[α]²⁰_{D}: −4.23 (c 0.965, MeOH)

¹H-NMR: (400 MHz, MeOD): δ 8.78 (s, 1H), 8.71 (d, J=5.2 Hz, 1H), 8.51-8.44 (m, 1H), 7.94 (dd, J=7.9, 5.7 Hz, 1H), 5.37-5.27 (m, 1H), 4.67 (d, J=6.0 Hz, 1H), 4.32 (td, J=7.2, 1.8 Hz, 1H), 4.04 (d, J=5.7 Hz, 1H), 3.67 (t, J=1.6 Hz, 1H), 3.24 (dd, J=13.9, 7.0 Hz, 1H), 3.13 (dd, J=13.9, 7.6 Hz, 1H), 2.65 (qd, J=7.3, 1.2 Hz, 1H), 2.28-2.16 (m, 1H), 1.42 (d, J=6.5 Hz, 3H), 1.38 (d, J=7.4 Hz, 3H), 1.01 (d, J=4.7 Hz, 3H), 0.99 (d, J=4.8 Hz, 3H).

¹³C-NMR: (101 MHz, MeOD): δ 178.6, 169.0, 166.9, 147.1, 144.7, 142.3, 130.8, 127.8, 79.6, 75.0, 69.0, 57.4, 55.3, 42.2, 36.8, 30.9, 18.5, 17.9, 17.7, 15.1.

IR: (neat, cm⁻¹): 3358, 2971, 2935, 1745, 1672, 1537, 1472, 1392, 1263, 1173, 1138, 1056, 837, 798, 723, 706, 600, 549, 508, 469, 458.

HR-MS: (ESI): m/z calc. for C₂₀H₂₉N₃NaO₆ [M+Na]+. found.

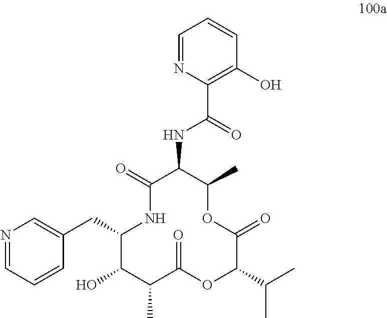 [SMILES: CC(C)[C@@H]1OC(=O)[C@H](C)[C@H](O)[C@H](Cc2cccnc2)NC(=O)[C@@H](NC(=O)c2ncccc2O)[C@@H](C)OC1=O]

Analog 100a:

To a solution of 3-hydroxypyridine-2-carboxylic acid 984 (6.20 mg, 44.5 μmol, 1.10 eq.), HATU (18.5 mg, 48.6 μmol, 1.20 eq.) and DIPEA (21.0 μl, 122 μmol, 3.00 eq.) in 0.4 mL MeCN (dark green) was added 983a (16.5 mg, 40.5 μmol, 1.00 eq.) in 1.7 mL MeCN at RT. The mixture was stirred for 24 h at RT. The mixture was diluted with CH₂Cl₂ and sat. aq. NaHCO₃. The aq. phase was extracted with CH₂Cl₂ and the combined org. phase was dried over MgSO₄ and concentrated. The orange oil was purified by FC (CH₂Cl₂/MeOH 5%) to yield 100a (12.0 mg, 56%) as a colorless film. The samples prepared for biological testing were purified by reversed phase HPLC (column: Symmetry® C18 5 μm, 19×100 mm, gradient: 30%-100% MeCN in H₂O in 14 min, flow: 25 mL/min, room temperature, t_{R}=6.78 min) to a purity>98%.

R_{f}: 0.30 (CH₂Cl₂/MeOH 5%)

[α]²⁰_{D}: −60.5 (c 0.110, MeOH)

¹H-NMR: (500 MHz, DMSO-d₆): δ 11.84 (s, 1H), 8.58 (d, J=6.4 Hz, 1H), 8.44 (d, J=1.8 Hz, 1H), 8.30 (dd, J=4.8, 1.6 Hz, 1H), 8.18 (dd, J=4.3, 1.2 Hz, 1H), 7.65 (dt, J=7.8, 1.8 Hz, 1H), 7.60 (d, J=3.4 Hz, 1H), 7.56 (dd, J=8.5, 4.4 Hz, 1H), 7.45 (dd, J=8.5, 1.2 Hz, 1H), 7.21 (dd, J=7.7, 4.8 Hz, 1H), 5.49 (s, 1H), 5.02 (p, J=5.9 Hz, 1H), 4.88 (dd, J=6.9, 5.4 Hz, 1H), 4.54 (d, J=5.3 Hz, 1H), 4.05 (q, J=7.6 Hz, 1H), 3.63 (d, J=4.1 Hz, 1H), 2.85 (qd, J=13.5, 7.2 Hz, 2H), 2.50-2.44 (m, 1H), 2.28-2.17 (m, 1H), 1.23 (d, J=7.1 Hz, 3H), 1.09 (d, J=6.2 Hz, 3H), 1.01 (dd, J=6.7, 4.6 Hz, 6H).

¹³C-NMR: (126 MHz, DMSO-d₆): δ 174.0, 168.3, 168.2, 166.7, 157.7, 150.8, 147.8, 140.7, 137.1, 134.6, 130.9, 130.2, 126.8, 123.7, 78.1, 71.7, 69.7, 55.2, 54.0, 45.8, 36.0, 29.8, 19.1, 17.9, 16.6, 13.1.

IR: (neat, cm⁻¹): 3373, 2977, 2942, 1732, 1650, 1510, 1450, 1293, 1257, 1062, 1026, 1013, 811, 783, 717, 662, 589.

HR-MS: (ESI): m/z calc. for C₂₆H₃₃N₄O₈ [M+H]⁺ 526.2759. found 526.2756.

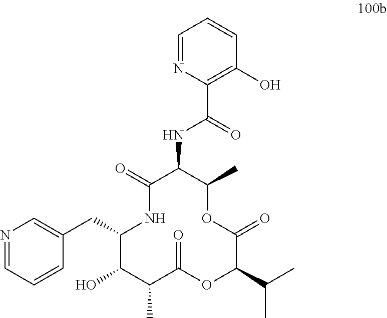 [SMILES: CC(C)[C@H]1OC(=O)[C@H](C)[C@H](O)[C@H](Cc2cccnc2)NC(=O)[C@@H](NC(=O)c2ncccc2O)[C@@H](C)OC1=O]

Compound 100b:

To a solution of 3-hydroxypyridine-2-carboxylic acid 984 (1.50 mg, 10.8 μmol, 1.10 eq.), HATU (4.48 mg, 11.8 μmol, 1.20 eq.) and DIPEA (5.10 μl, 29.5 Mmol, 0.3.00 eq.) in 100 μl MeCN was added 983b (4.00 mg, 9.82 μmol, 1.00 eq.) in 0.4 mL MeCN at RT. The mixture was stirred for 18 h at RT. The mixture was diluted with CH₂Cl₂ and sat. aq. NaHCO₃. The aq. phase was extracted with CH₂Cl₂ and the combined org. phase was dried over MgSO₄ and concentrated. The green oil was purified by FC (CH₂Cl₂/MeOH 5%) to yield 100b (2.7 mg, 52% over 2 steps) as a colorless film. The samples prepared for biological testing were purified by reversed phase HPLC (column: Symmetry® C18 5 μm, 19×100 mm, gradient: 30%-100% MeCN in H₂O in 14 min, flow: 25 mL/min, room temperature, t_{R}=6.88 min) to a purity>98%.

R_{f}: 0.25 (CH₂Cl₂/MeOH 5%)

[α]²⁰_{D}: −29.7 (c 0.110, MeOH)

¹H-NMR: (500 MHz, DMSO-d₆): δ 11.80 (s, 1H), 8.37 (d, J=1.8 Hz, 1H), 8.30 (d, J=4.7 Hz, 1H), 8.21-8.16 (m, 2H), 8.10 (d, J=9.4 Hz, 1H), 7.58 (dd, J=8.5, 4.4 Hz, 1H), 7.55 (dt, J=7.8, 1.9 Hz, 1H), 7.46 (dd, J=8.5, 1.3 Hz, 1H), 7.07 (dd, J=7.7, 4.9 Hz, 1H), 5.29 (p, J=6.4 Hz, 1H), 4.81-4.64 (m, 3H), 4.15-4.03 (m, 1H), 3.62 (d, J=9.1 Hz, 1H), 2.86 (dd, J=13.5, 6.0 Hz, 1H), 2.77 (dd, J=13.4, 8.7 Hz, 1H), 2.66-2.56 (m, 1H), 2.19-2.06 (m, J=6.7 Hz, 1H), 1.28 (d, J=7.3 Hz, 3H), 1.21 (d, J=6.5 Hz, 3H), 0.92 (dd, J=6.8, 2.0 Hz, 6H)

¹³C-NMR: ¹³C NMR (126 MHz, DMSO-d₆): δ 176.2, 168.1, 167.5, 157.7, 150.8, 147.6, 140.6, 137.0, 134.0, 130.8, 130.2, 126.8, 123.4, 77.5, 73.9, 68.8, 56.1, 53.3, 41.4, 35.2, 29.8, 18.5, 17.7, 17.4, 15.0.

IR: (neat, cm⁻¹): 3370, 2971, 2938, 1745, 1650, 1522, 1450, 1386, 1296, 1254, 1168, 1062, 810, 778, 715, 656.

### Determination of the Minimal Inhibitory Concentration (MIC) of Pyridomycin and Analogues.

The drug susceptibility of Mycobacterium tuberculosis strain H37Rv was determined using the resazurin microtitre assay (REMA) (Palomino, Antimicrob. Agents Chemother. 46, 2720-2722 (2002)). Briefly, bacteria were diluted from frozen stocks to an OD600 of 0.0001, and grown in a 96-well plate in the presence of serial compound dilutions. After 10 generations (7 days for M. tuberculosis) bacterial viability was determined using 10 μL of resazurin (0.025% (w/v), and calculated as a percentage of resazurin turnover in the absence of compound. The MIC was determined as the minimal concentration of compound that caused background resazurin reduction.

MIC Pyridomycin: 0.39 μg/ml

MIC (S)-Analog 100a: 12.5 μg/ml

MIC (R)-Analog 100b: 1.56 μg/ml

1. A compound of formula (1) 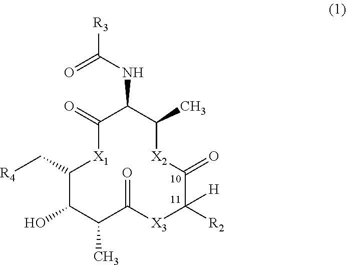
wherein
X₁ represents O or NR₆,
X₂ represents O or NR₆,
X₃ represents O or NR₁,
R₁ represents H or C₁ to C₃ alkyl,
R₂ represents H, or a linear or branched C₁-C₈ alkyl optionally comprising one or more heteroatoms, cyclopropyl, cyclobutyl, cyclohexyl or oxetanyl or an amino acid side chain or a protected amino acid side chain, or
R₁ and R₂ may form together a 5 or 6 membered ring system which may be saturated, partly saturated or unsaturated, said ring system being optionally substituted,
R₃ represents H, cyclopentyl, cyclohexyl, aryl or hydroxyaryl, said aryl or hydroxyaryl being optionally substituted by fluorine, or linear or branched C₁ to C₈ alkyl optionally comprising a hetero atom,
R₄ represents phenyl or 5- or 6-membered heterocycles comprising one or more nitrogen or oxygen atoms optionally substituted with 1 to 4, respectively 5 fluorine atoms, and
R₆ represents H, or a linear or branched alkyl chain having 1 to 3 carbon atoms.

2. Compound according to claim 1, wherein R₂ represents H, or a linear or branched C₁-C₈ alkyl optionally comprising one or more heteroatoms, cyclopropyl, cyclobutyl or oxetanyl or an amino acid side chain or a protected amino acid side chain, or
R₁ and R₂ may form together a 5 or 6 membered ring system which may be saturated, partly saturated or unsaturated, said ring system being optionally substituted.

3. Compound according to claim 1, wherein X₃ represents O.

4. Compound according to claim 1, wherein X₃ represents NR₁ and R₁ is hydrogen, methyl, ethyl, propyl or isopropyl.

5. Compound according to claim 1, wherein R₂ is the side chain of an amino acid selected from the group of alanine, valine, leucine, isoleucine, tryptophan, phenylalanine, methionine, serine or a protected serine, tyrosine or a protected tyrosine, threonine or a protected threonine, cysteine or a protected threonine, asparagine, glutamin, aspartate, glutamate, lysine, arginine and histidine.

6. Compound according to claim 1, wherein R₂ is selected from the group of cyclopropyl, cyclobutyl or oxetanyl.

7. Compound according to claim 1, wherein R₄ is a 2-, 3-, or 4-pyridyl residue having four substituents R₅₍ᵢ₎, R₅₍ᵢᵢ₎, R₅₍ᵢᵢᵢ₎ and R₅₍ᵢᵥ₎ and R₅₍ᵢ₎, R₅₍ᵢᵢ₎, R₅₍ᵢᵢᵢ₎ and R₅₍ᵢᵥ₎ are independently from each other hydrogen or fluorine.

8. Compound according to claim 1, wherein R₄ is phenyl residue having five substituents R₅₍ᵢ₎, R₅₍ᵢᵢ₎, R₅₍ᵢᵢᵢ₎ and R₅₍ᵢᵥ₎ and R₅₍ᵢᵥ₎; and R₅₍ᵢ₎, R₅₍ᵢᵢ₎, R₅₍ᵢᵢᵢ₎, R₅₍ᵢᵥ₎ and R₅₍ᵥ₎ are independently from each other hydrogen or fluorine.

9. Compound according to claim 1, wherein X₂ represents O.

10. Compound according to claim 1, wherein X₂ represents NR₆, and R₆ represents H, or a linear or branched alkyl chain having 1 to 3 carbon atoms.

11. Compound according to claim 1, wherein R₃ is aryl.

12. Compound according to claim 11, wherein the aryl residue is optionally substituted by fluorine atoms.

13. Compound according to claim 1, wherein the compound has in C11 position R-configuration.

14. Compound according to claim 1, wherein the compound has in C11 position S-configuration.

15. Compound of formula

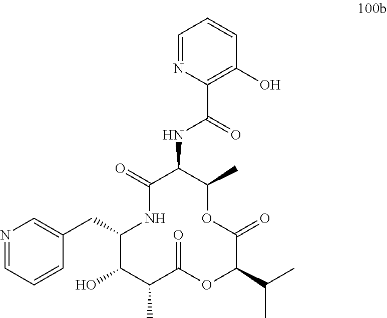 [SMILES: CC(C)[C@H]1OC(=O)[C@H](C)[C@H](O)[C@H](Cc2cccnc2)NC(=O)[C@@H](NC(=O)c2ncccc2O)[C@@H](C)OC1=O]

16. Compound according to claim 1 selected from the group of

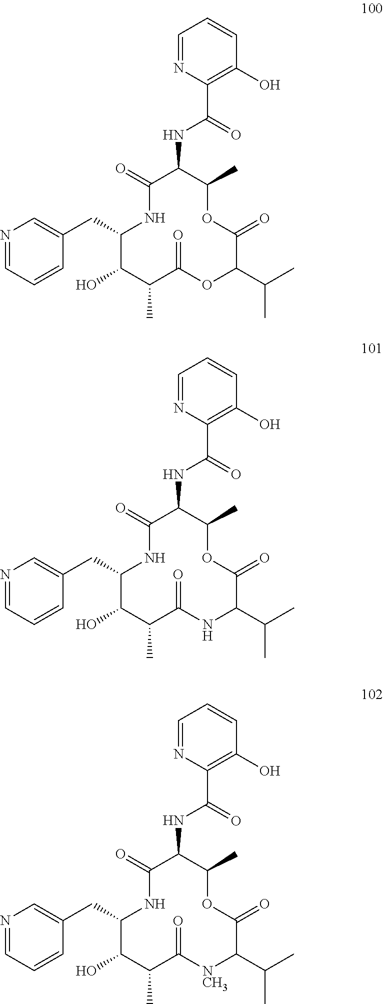 [SMILES: CC(C)C1OC(=O)[C@H](C)[C@H](O)[C@H](Cc2cccnc2)NC(=O)[C@@H](NC(=O)c2ncccc2O)[C@@H](C)OC1=O ; CC(C)C1NC(=O)[C@H](C)[C@H](O)[C@H](Cc2cccnc2)NC(=O)[C@@H](NC(=O)c2ncccc2O)[C@@H](C)OC1=O ; CC(C)C1C(=O)O[C@H](C)[C@H](NC(=O)c2ncccc2O)C(=O)N[C@@H](Cc2cccnc2)[C@@H](O)[C@@H](C)C(=O)N1C]

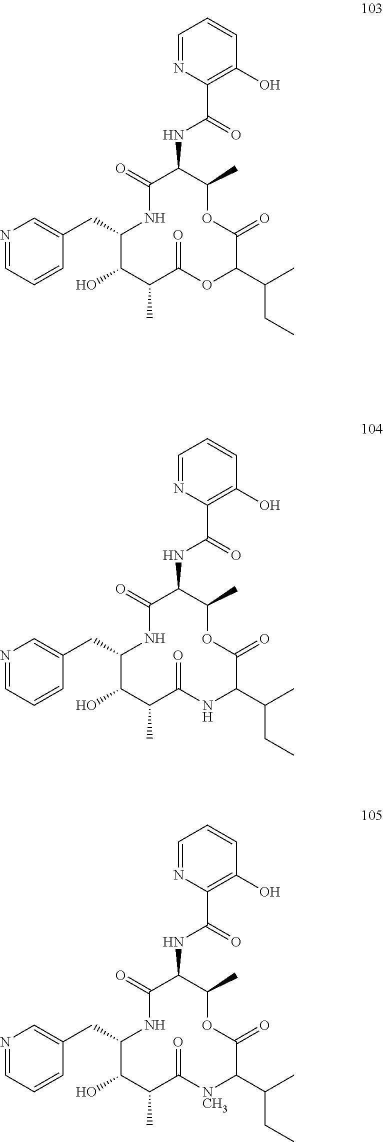 [SMILES: CCC(C)C1OC(=O)[C@H](C)[C@H](O)[C@H](Cc2cccnc2)NC(=O)[C@@H](NC(=O)c2ncccc2O)[C@@H](C)OC1=O ; CCC(C)C1NC(=O)[C@H](C)[C@H](O)[C@H](Cc2cccnc2)NC(=O)[C@@H](NC(=O)c2ncccc2O)[C@@H](C)OC1=O ; CCC(C)C1C(=O)O[C@H](C)[C@H](NC(=O)c2ncccc2O)C(=O)N[C@@H](Cc2cccnc2)[C@@H](O)[C@@H](C)C(=O)N1C]

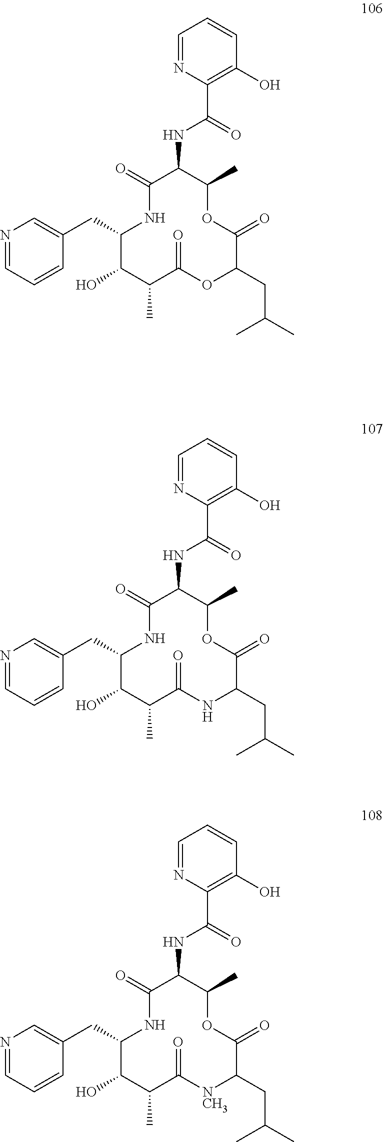 [SMILES: CC(C)CC1OC(=O)[C@H](C)[C@H](O)[C@H](Cc2cccnc2)NC(=O)[C@@H](NC(=O)c2ncccc2O)[C@@H](C)OC1=O ; CC(C)CC1NC(=O)[C@H](C)[C@H](O)[C@H](Cc2cccnc2)NC(=O)[C@@H](NC(=O)c2ncccc2O)[C@@H](C)OC1=O ; CC(C)CC1C(=O)O[C@H](C)[C@H](NC(=O)c2ncccc2O)C(=O)N[C@@H](Cc2cccnc2)[C@@H](O)[C@@H](C)C(=O)N1C]

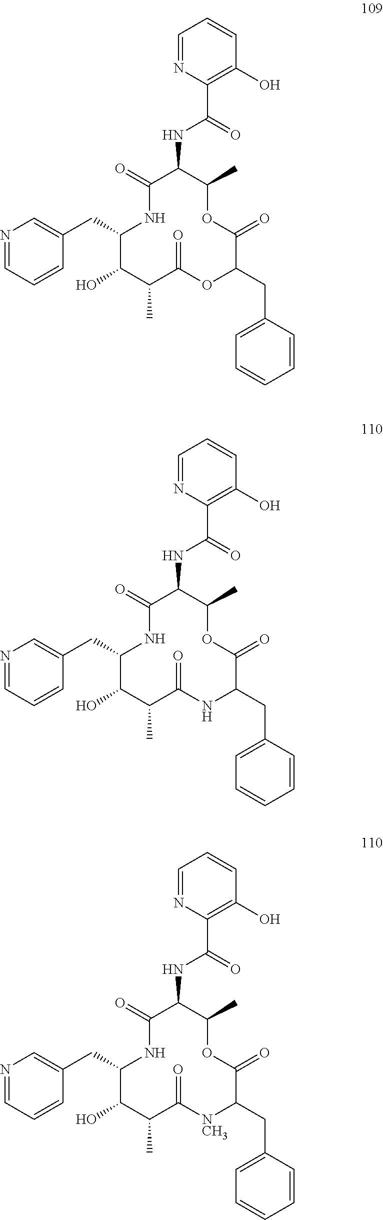 [SMILES: C[C@H]1OC(=O)C(Cc2ccccc2)OC(=O)[C@H](C)[C@H](O)[C@H](Cc2cccnc2)NC(=O)[C@H]1NC(=O)c1ncccc1O ; C[C@H]1OC(=O)C(Cc2ccccc2)NC(=O)[C@H](C)[C@H](O)[C@H](Cc2cccnc2)NC(=O)[C@H]1NC(=O)c1ncccc1O ; C[C@H]1OC(=O)C(Cc2ccccc2)N(C)C(=O)[C@H](C)[C@H](O)[C@H](Cc2cccnc2)NC(=O)[C@H]1NC(=O)c1ncccc1O]

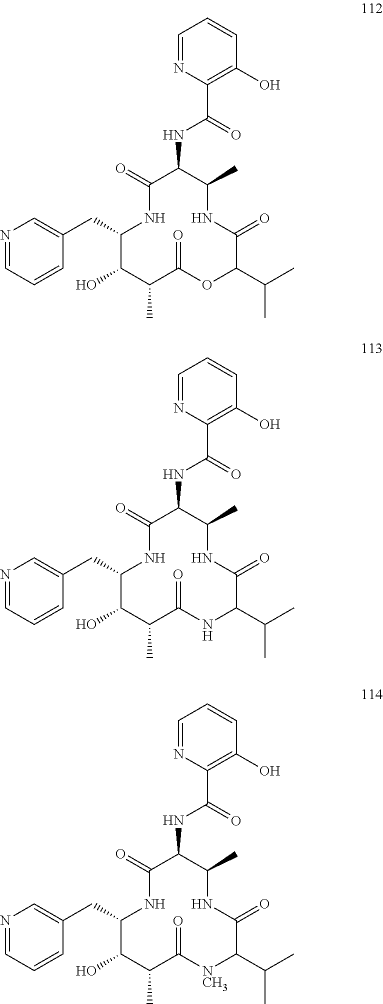 [SMILES: CC(C)C1OC(=O)[C@H](C)[C@H](O)[C@H](Cc2cccnc2)NC(=O)[C@@H](NC(=O)c2ncccc2O)[C@@H](C)NC1=O ; CC(C)C1NC(=O)[C@H](C)[C@H](O)[C@H](Cc2cccnc2)NC(=O)[C@@H](NC(=O)c2ncccc2O)[C@@H](C)NC1=O ; CC(C)C1C(=O)N[C@H](C)[C@H](NC(=O)c2ncccc2O)C(=O)N[C@@H](Cc2cccnc2)[C@@H](O)[C@@H](C)C(=O)N1C]

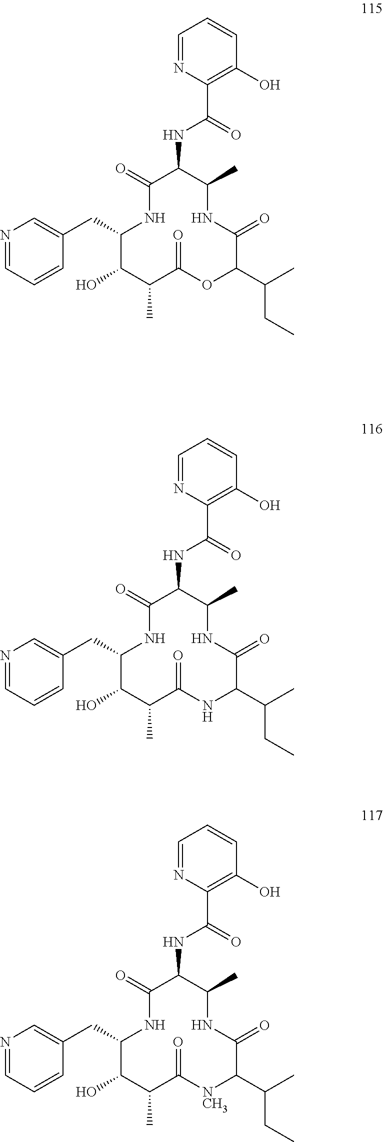 [SMILES: CCC(C)C1OC(=O)[C@H](C)[C@H](O)[C@H](Cc2cccnc2)NC(=O)[C@@H](NC(=O)c2ncccc2O)[C@@H](C)NC1=O ; CCC(C)C1NC(=O)[C@H](C)[C@H](O)[C@H](Cc2cccnc2)NC(=O)[C@@H](NC(=O)c2ncccc2O)[C@@H](C)NC1=O ; CCC(C)C1C(=O)N[C@H](C)[C@H](NC(=O)c2ncccc2O)C(=O)N[C@@H](Cc2cccnc2)[C@@H](O)[C@@H](C)C(=O)N1C]

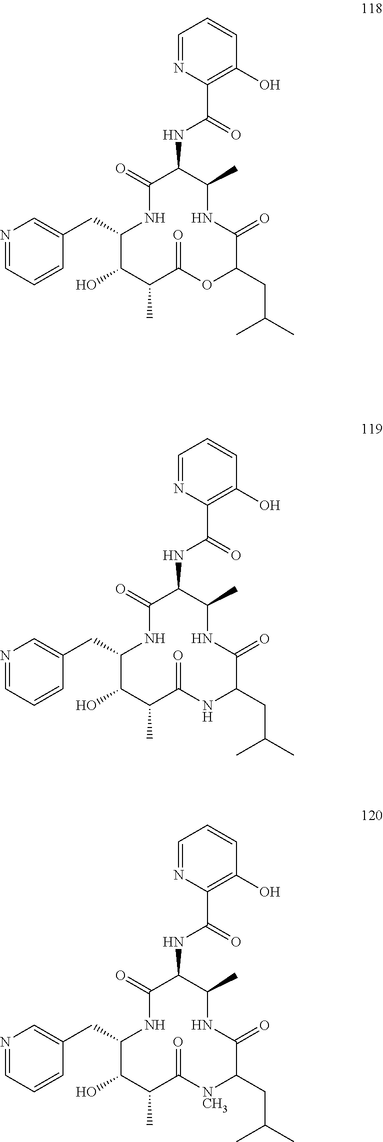 [SMILES: CC(C)CC1OC(=O)[C@H](C)[C@H](O)[C@H](Cc2cccnc2)NC(=O)[C@@H](NC(=O)c2ncccc2O)[C@@H](C)NC1=O ; CC(C)CC1NC(=O)[C@H](C)[C@H](O)[C@H](Cc2cccnc2)NC(=O)[C@@H](NC(=O)c2ncccc2O)[C@@H](C)NC1=O ; CC(C)CC1C(=O)N[C@H](C)[C@H](NC(=O)c2ncccc2O)C(=O)N[C@@H](Cc2cccnc2)[C@@H](O)[C@@H](C)C(=O)N1C]

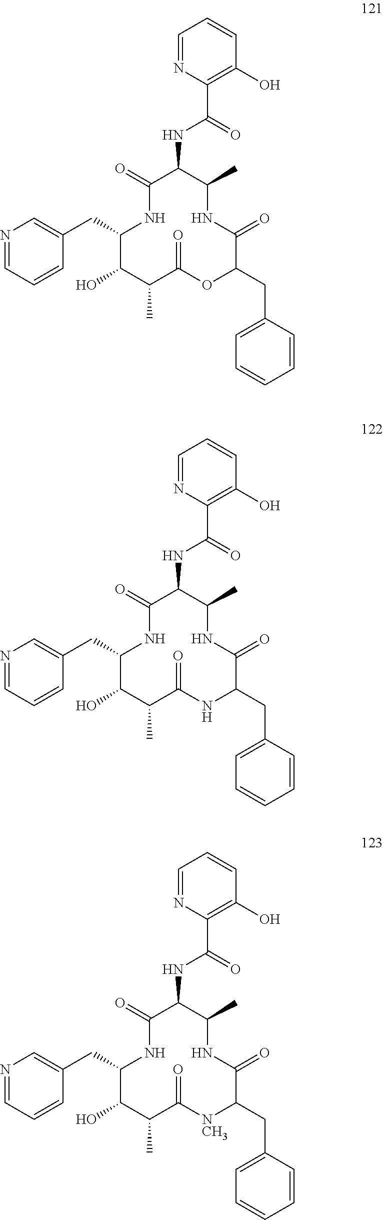 [SMILES: C[C@H]1NC(=O)C(Cc2ccccc2)OC(=O)[C@H](C)[C@H](O)[C@H](Cc2cccnc2)NC(=O)[C@H]1NC(=O)c1ncccc1O ; C[C@H]1NC(=O)C(Cc2ccccc2)NC(=O)[C@H](C)[C@H](O)[C@H](Cc2cccnc2)NC(=O)[C@H]1NC(=O)c1ncccc1O ; C[C@H]1NC(=O)C(Cc2ccccc2)N(C)C(=O)[C@H](C)[C@H](O)[C@H](Cc2cccnc2)NC(=O)[C@H]1NC(=O)c1ncccc1O]

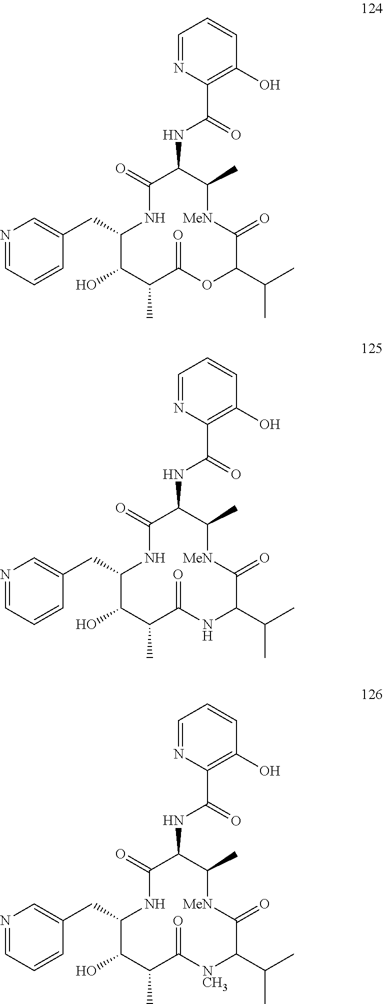 [SMILES: CC(C)C1OC(=O)[C@H](C)[C@H](O)[C@H](Cc2cccnc2)NC(=O)[C@@H](NC(=O)c2ncccc2O)[C@@H](C)N(C)C1=O ; CC(C)C1NC(=O)[C@H](C)[C@H](O)[C@H](Cc2cccnc2)NC(=O)[C@@H](NC(=O)c2ncccc2O)[C@@H](C)N(C)C1=O ; CC(C)C1C(=O)N(C)[C@H](C)[C@H](NC(=O)c2ncccc2O)C(=O)N[C@@H](Cc2cccnc2)[C@@H](O)[C@@H](C)C(=O)N1C]

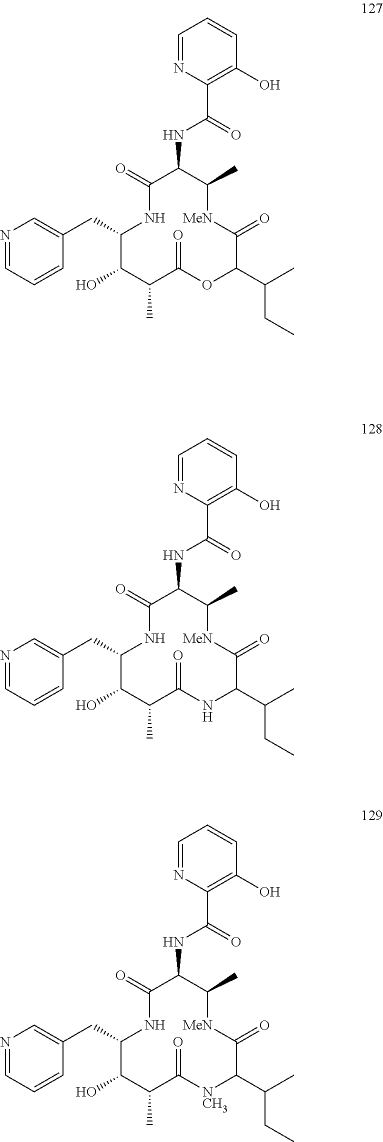 [SMILES: CCC(C)C1OC(=O)[C@H](C)[C@H](O)[C@H](Cc2cccnc2)NC(=O)[C@@H](NC(=O)c2ncccc2O)[C@@H](C)N(C)C1=O ; CCC(C)C1NC(=O)[C@H](C)[C@H](O)[C@H](Cc2cccnc2)NC(=O)[C@@H](NC(=O)c2ncccc2O)[C@@H](C)N(C)C1=O ; CCC(C)C1C(=O)N(C)[C@H](C)[C@H](NC(=O)c2ncccc2O)C(=O)N[C@@H](Cc2cccnc2)[C@@H](O)[C@@H](C)C(=O)N1C]

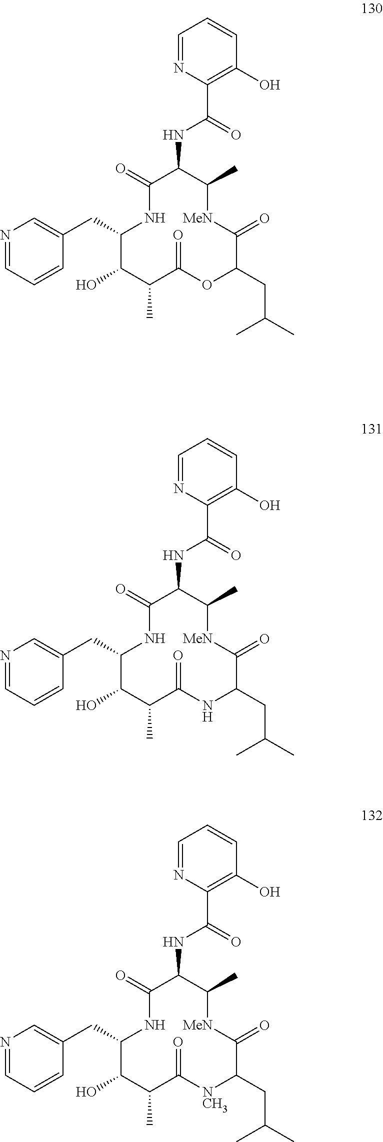 [SMILES: CC(C)CC1OC(=O)[C@H](C)[C@H](O)[C@H](Cc2cccnc2)NC(=O)[C@@H](NC(=O)c2ncccc2O)[C@@H](C)N(C)C1=O ; CC(C)CC1NC(=O)[C@H](C)[C@H](O)[C@H](Cc2cccnc2)NC(=O)[C@@H](NC(=O)c2ncccc2O)[C@@H](C)N(C)C1=O ; CC(C)CC1C(=O)N(C)[C@H](C)[C@H](NC(=O)c2ncccc2O)C(=O)N[C@@H](Cc2cccnc2)[C@@H](O)[C@@H](C)C(=O)N1C]

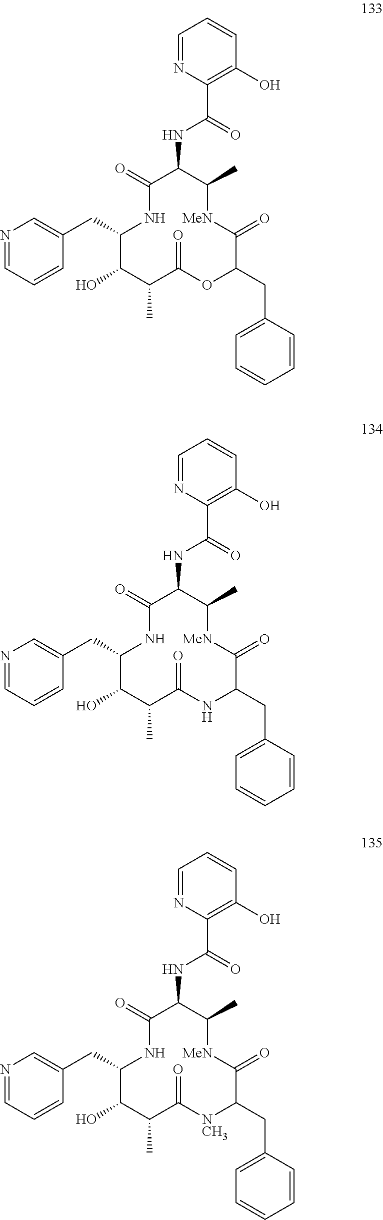 [SMILES: C[C@@H]1[C@H](NC(=O)c2ncccc2O)C(=O)N[C@@H](Cc2cccnc2)[C@@H](O)[C@@H](C)C(=O)OC(Cc2ccccc2)C(=O)N1C ; C[C@@H]1[C@H](NC(=O)c2ncccc2O)C(=O)N[C@@H](Cc2cccnc2)[C@@H](O)[C@@H](C)C(=O)NC(Cc2ccccc2)C(=O)N1C ; C[C@@H]1[C@H](NC(=O)c2ncccc2O)C(=O)N[C@@H](Cc2cccnc2)[C@@H](O)[C@@H](C)C(=O)N(C)C(Cc2ccccc2)C(=O)N1C]

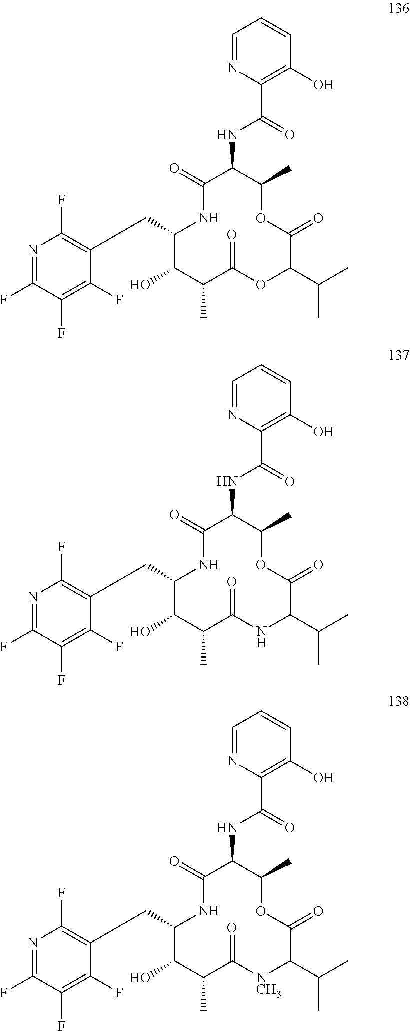 [SMILES: CC(C)C1OC(=O)[C@H](C)[C@H](O)[C@H](Cc2c(F)nc(F)c(F)c2F)NC(=O)[C@@H](NC(=O)c2ncccc2O)[C@@H](C)OC1=O ; CC(C)C1NC(=O)[C@H](C)[C@H](O)[C@H](Cc2c(F)nc(F)c(F)c2F)NC(=O)[C@@H](NC(=O)c2ncccc2O)[C@@H](C)OC1=O ; CC(C)C1C(=O)O[C@H](C)[C@H](NC(=O)c2ncccc2O)C(=O)N[C@@H](Cc2c(F)nc(F)c(F)c2F)[C@@H](O)[C@@H](C)C(=O)N1C]

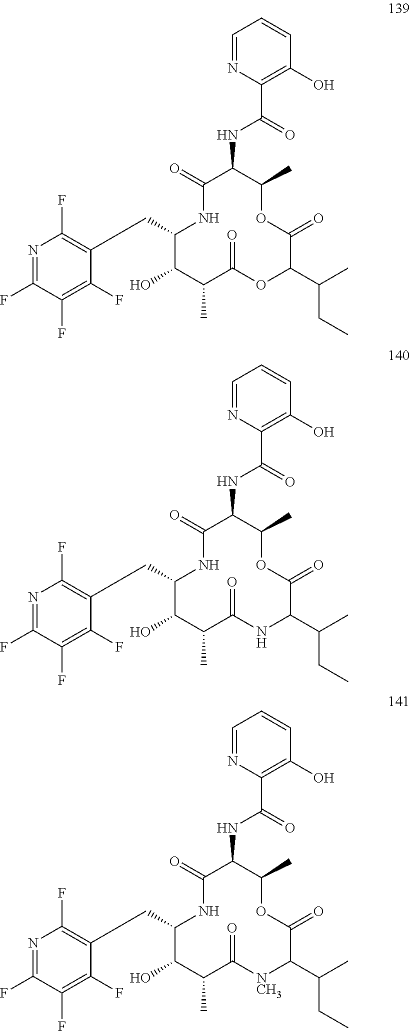 [SMILES: CCC(C)C1OC(=O)[C@H](C)[C@H](O)[C@H](Cc2c(F)nc(F)c(F)c2F)NC(=O)[C@@H](NC(=O)c2ncccc2O)[C@@H](C)OC1=O ; CCC(C)C1NC(=O)[C@H](C)[C@H](O)[C@H](Cc2c(F)nc(F)c(F)c2F)NC(=O)[C@@H](NC(=O)c2ncccc2O)[C@@H](C)OC1=O ; CCC(C)C1C(=O)O[C@H](C)[C@H](NC(=O)c2ncccc2O)C(=O)N[C@@H](Cc2c(F)nc(F)c(F)c2F)[C@@H](O)[C@@H](C)C(=O)N1C]

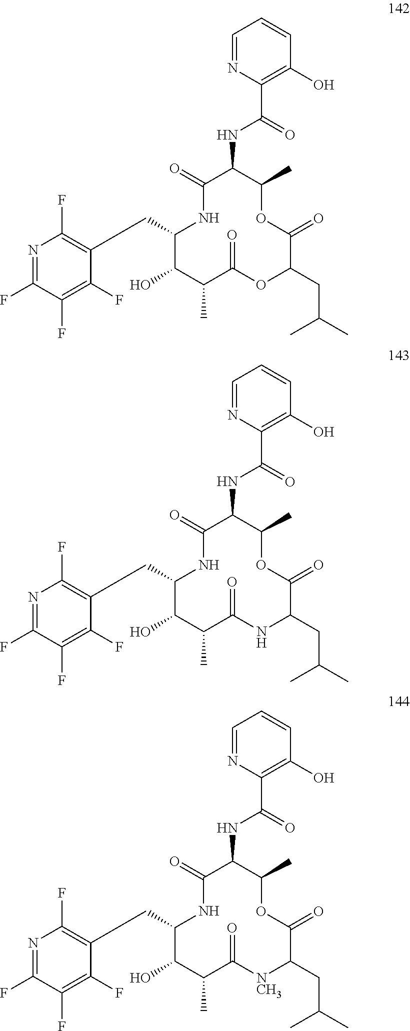

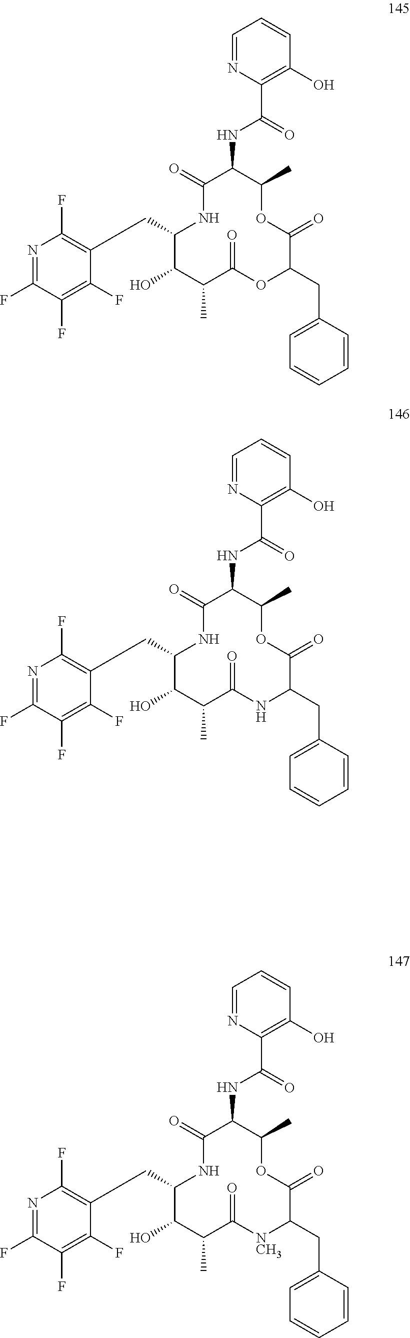 [SMILES: C[C@H]1OC(=O)C(Cc2ccccc2)OC(=O)[C@H](C)[C@H](O)[C@H](Cc2c(F)nc(F)c(F)c2F)NC(=O)[C@H]1NC(=O)c1ncccc1O ; C[C@H]1OC(=O)C(Cc2ccccc2)NC(=O)[C@H](C)[C@H](O)[C@H](Cc2c(F)nc(F)c(F)c2F)NC(=O)[C@H]1NC(=O)c1ncccc1O ; C[C@H]1OC(=O)C(Cc2ccccc2)N(C)C(=O)[C@H](C)[C@H](O)[C@H](Cc2c(F)nc(F)c(F)c2F)NC(=O)[C@H]1NC(=O)c1ncccc1O]

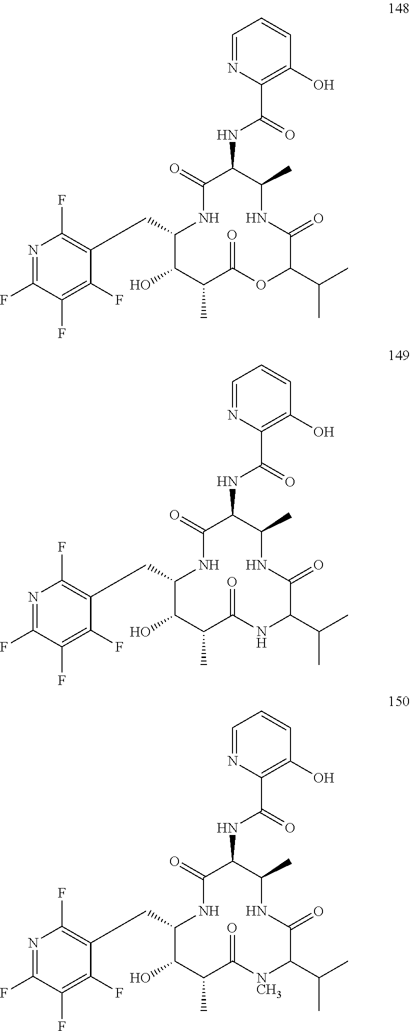 [SMILES: CC(C)C1OC(=O)[C@H](C)[C@H](O)[C@H](Cc2c(F)nc(F)c(F)c2F)NC(=O)[C@@H](NC(=O)c2ncccc2O)[C@@H](C)NC1=O ; CC(C)C1NC(=O)[C@H](C)[C@H](O)[C@H](Cc2c(F)nc(F)c(F)c2F)NC(=O)[C@@H](NC(=O)c2ncccc2O)[C@@H](C)NC1=O ; CC(C)C1C(=O)N[C@H](C)[C@H](NC(=O)c2ncccc2O)C(=O)N[C@@H](Cc2c(F)nc(F)c(F)c2F)[C@@H](O)[C@@H](C)C(=O)N1C]

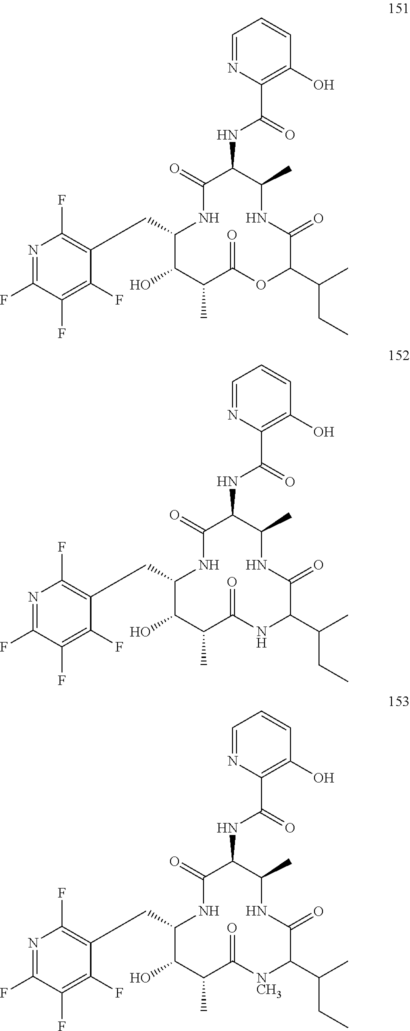 [SMILES: CCC(C)C1OC(=O)[C@H](C)[C@H](O)[C@H](Cc2c(F)nc(F)c(F)c2F)NC(=O)[C@@H](NC(=O)c2ncccc2O)[C@@H](C)NC1=O ; CCC(C)C1NC(=O)[C@H](C)[C@H](O)[C@H](Cc2c(F)nc(F)c(F)c2F)NC(=O)[C@@H](NC(=O)c2ncccc2O)[C@@H](C)NC1=O ; CCC(C)C1C(=O)N[C@H](C)[C@H](NC(=O)c2ncccc2O)C(=O)N[C@@H](Cc2c(F)nc(F)c(F)c2F)[C@@H](O)[C@@H](C)C(=O)N1C]

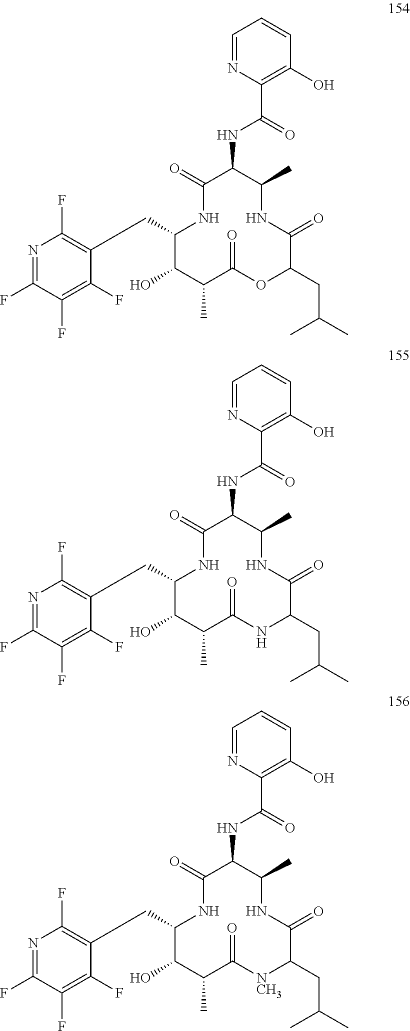

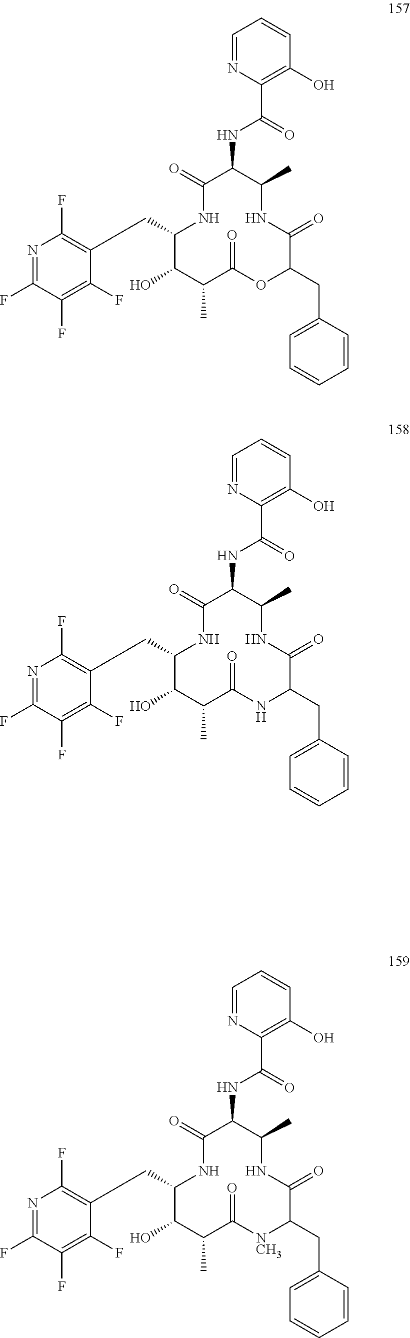 [SMILES: C[C@H]1NC(=O)C(Cc2ccccc2)OC(=O)[C@H](C)[C@H](O)[C@H](Cc2c(F)nc(F)c(F)c2F)NC(=O)[C@H]1NC(=O)c1ncccc1O ; C[C@H]1NC(=O)C(Cc2ccccc2)NC(=O)[C@H](C)[C@H](O)[C@H](Cc2c(F)nc(F)c(F)c2F)NC(=O)[C@H]1NC(=O)c1ncccc1O ; C[C@H]1NC(=O)C(Cc2ccccc2)N(C)C(=O)[C@H](C)[C@H](O)[C@H](Cc2c(F)nc(F)c(F)c2F)NC(=O)[C@H]1NC(=O)c1ncccc1O]

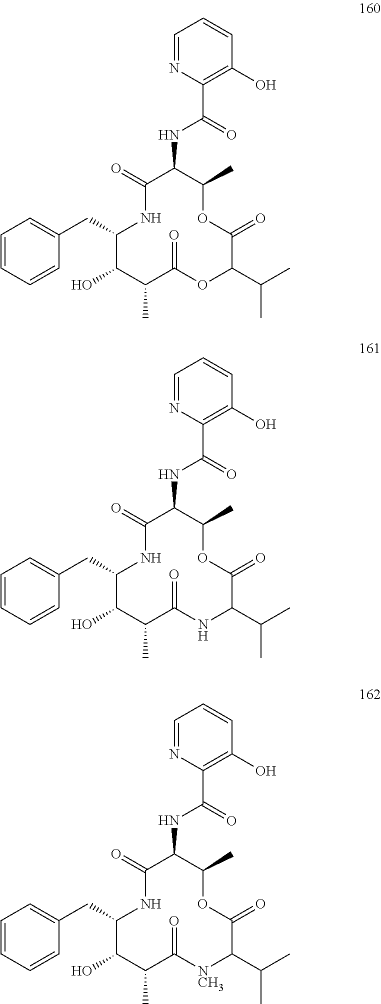 [SMILES: CC(C)C1OC(=O)[C@H](C)[C@H](O)[C@H](Cc2ccccc2)NC(=O)[C@@H](NC(=O)c2ncccc2O)[C@@H](C)OC1=O ; CC(C)C1NC(=O)[C@H](C)[C@H](O)[C@H](Cc2ccccc2)NC(=O)[C@@H](NC(=O)c2ncccc2O)[C@@H](C)OC1=O ; CC(C)C1C(=O)O[C@H](C)[C@H](NC(=O)c2ncccc2O)C(=O)N[C@@H](Cc2ccccc2)[C@@H](O)[C@@H](C)C(=O)N1C]

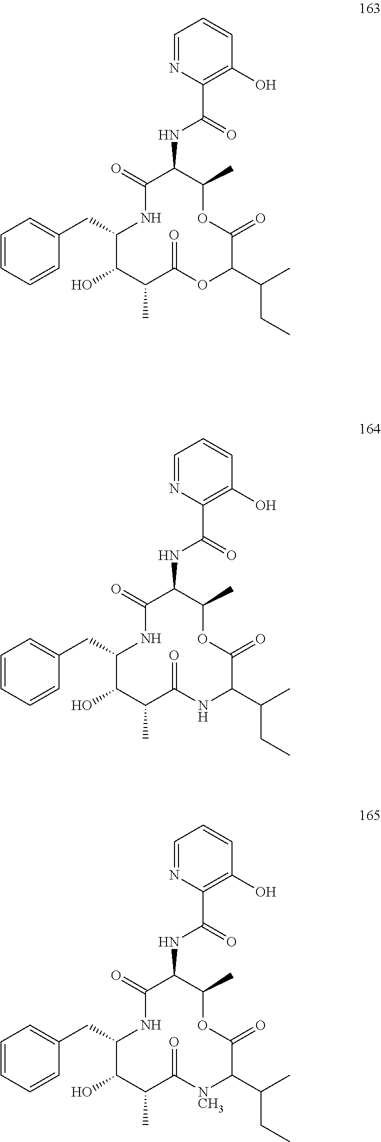 [SMILES: CCC(C)C1OC(=O)[C@H](C)[C@H](O)[C@H](Cc2ccccc2)NC(=O)[C@@H](NC(=O)c2ncccc2O)[C@@H](C)OC1=O ; CCC(C)C1NC(=O)[C@H](C)[C@H](O)[C@H](Cc2ccccc2)NC(=O)[C@@H](NC(=O)c2ncccc2O)[C@@H](C)OC1=O ; CCC(C)C1C(=O)O[C@H](C)[C@H](NC(=O)c2ncccc2O)C(=O)N[C@@H](Cc2ccccc2)[C@@H](O)[C@@H](C)C(=O)N1C]

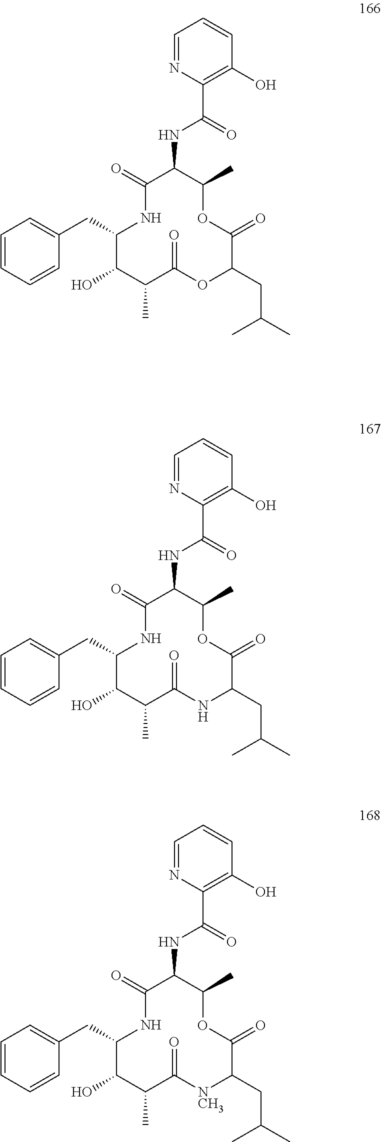 [SMILES: CC(C)CC1OC(=O)[C@H](C)[C@H](O)[C@H](Cc2ccccc2)NC(=O)[C@@H](NC(=O)c2ncccc2O)[C@@H](C)OC1=O ; CC(C)CC1NC(=O)[C@H](C)[C@H](O)[C@H](Cc2ccccc2)NC(=O)[C@@H](NC(=O)c2ncccc2O)[C@@H](C)OC1=O ; CC(C)CC1C(=O)O[C@H](C)[C@H](NC(=O)c2ncccc2O)C(=O)N[C@@H](Cc2ccccc2)[C@@H](O)[C@@H](C)C(=O)N1C]

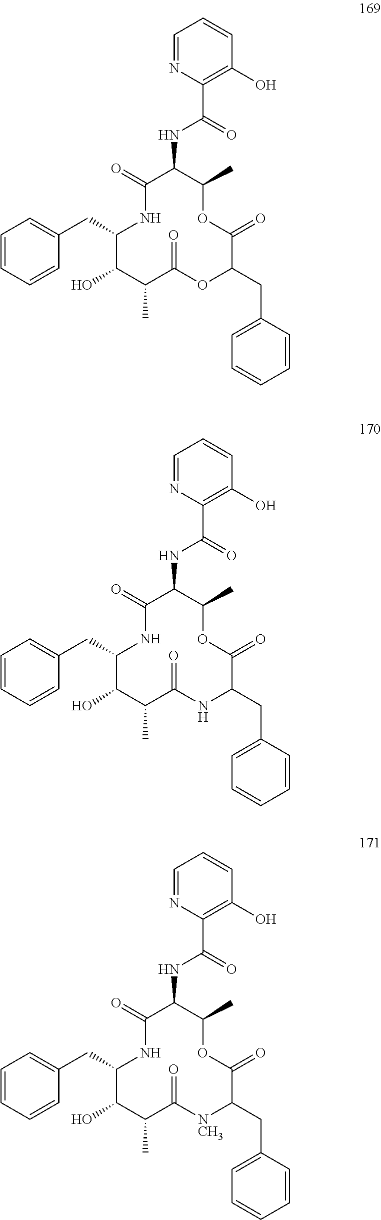 [SMILES: C[C@H]1OC(=O)C(Cc2ccccc2)OC(=O)[C@H](C)[C@H](O)[C@H](Cc2ccccc2)NC(=O)[C@H]1NC(=O)c1ncccc1O ; C[C@H]1OC(=O)C(Cc2ccccc2)NC(=O)[C@H](C)[C@H](O)[C@H](Cc2ccccc2)NC(=O)[C@H]1NC(=O)c1ncccc1O ; C[C@H]1OC(=O)C(Cc2ccccc2)N(C)C(=O)[C@H](C)[C@H](O)[C@H](Cc2ccccc2)NC(=O)[C@H]1NC(=O)c1ncccc1O]

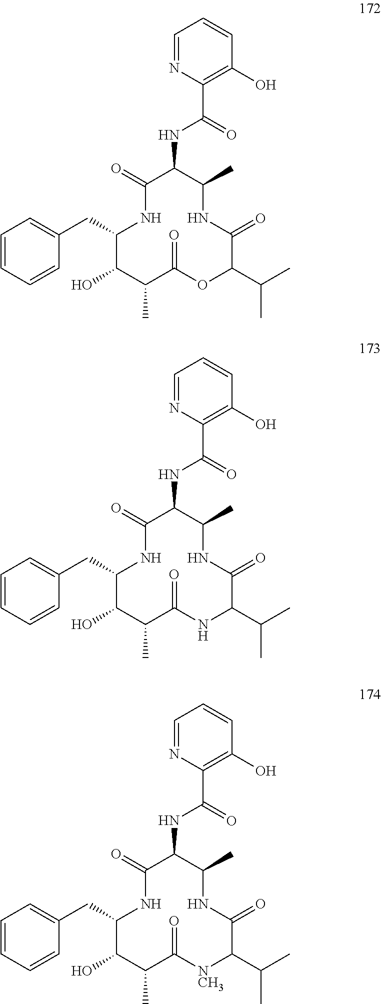 [SMILES: CC(C)C1OC(=O)[C@H](C)[C@H](O)[C@H](Cc2ccccc2)NC(=O)[C@@H](NC(=O)c2ncccc2O)[C@@H](C)NC1=O ; CC(C)C1NC(=O)[C@H](C)[C@H](O)[C@H](Cc2ccccc2)NC(=O)[C@@H](NC(=O)c2ncccc2O)[C@@H](C)NC1=O ; CC(C)C1C(=O)N[C@H](C)[C@H](NC(=O)c2ncccc2O)C(=O)N[C@@H](Cc2ccccc2)[C@@H](O)[C@@H](C)C(=O)N1C]

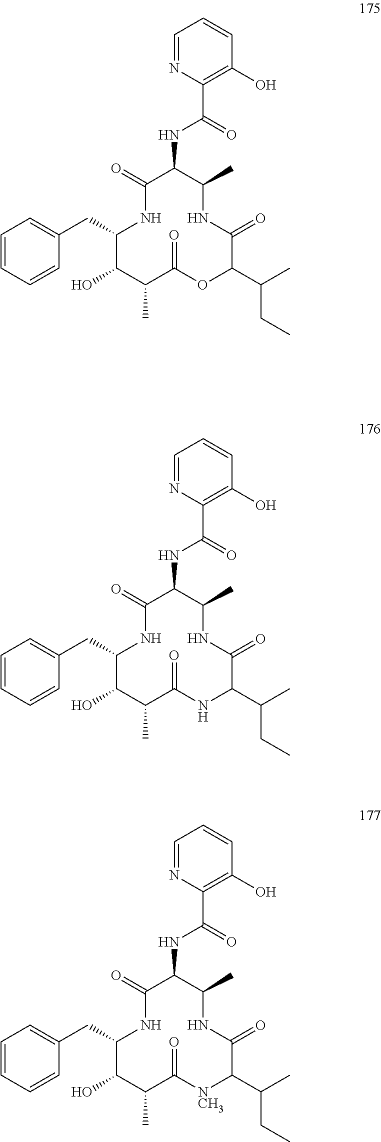 [SMILES: CCC(C)C1OC(=O)[C@H](C)[C@H](O)[C@H](Cc2ccccc2)NC(=O)[C@@H](NC(=O)c2ncccc2O)[C@@H](C)NC1=O ; CCC(C)C1NC(=O)[C@H](C)[C@H](O)[C@H](Cc2ccccc2)NC(=O)[C@@H](NC(=O)c2ncccc2O)[C@@H](C)NC1=O ; CCC(C)C1C(=O)N[C@H](C)[C@H](NC(=O)c2ncccc2O)C(=O)N[C@@H](Cc2ccccc2)[C@@H](O)[C@@H](C)C(=O)N1C]

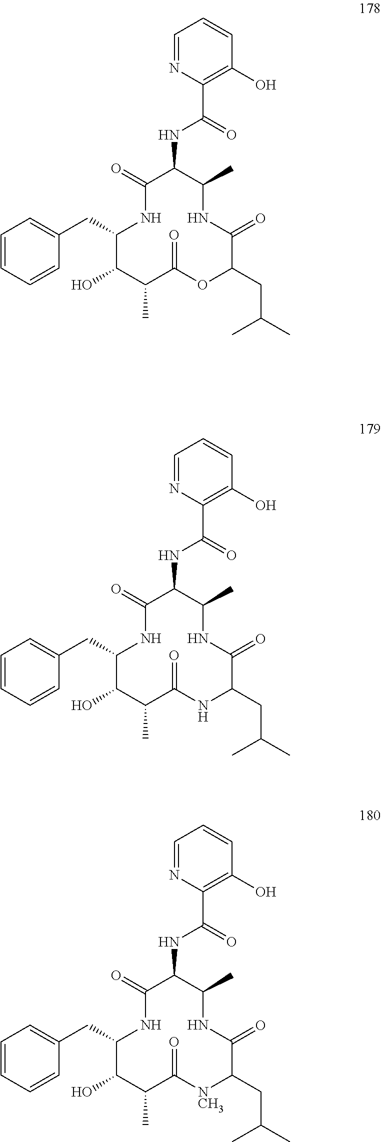 [SMILES: CC(C)CC1OC(=O)[C@H](C)[C@H](O)[C@H](Cc2ccccc2)NC(=O)[C@@H](NC(=O)c2ncccc2O)[C@@H](C)NC1=O ; CC(C)CC1NC(=O)[C@H](C)[C@H](O)[C@H](Cc2ccccc2)NC(=O)[C@@H](NC(=O)c2ncccc2O)[C@@H](C)NC1=O ; CC(C)CC1C(=O)N[C@H](C)[C@H](NC(=O)c2ncccc2O)C(=O)N[C@@H](Cc2ccccc2)[C@@H](O)[C@@H](C)C(=O)N1C]

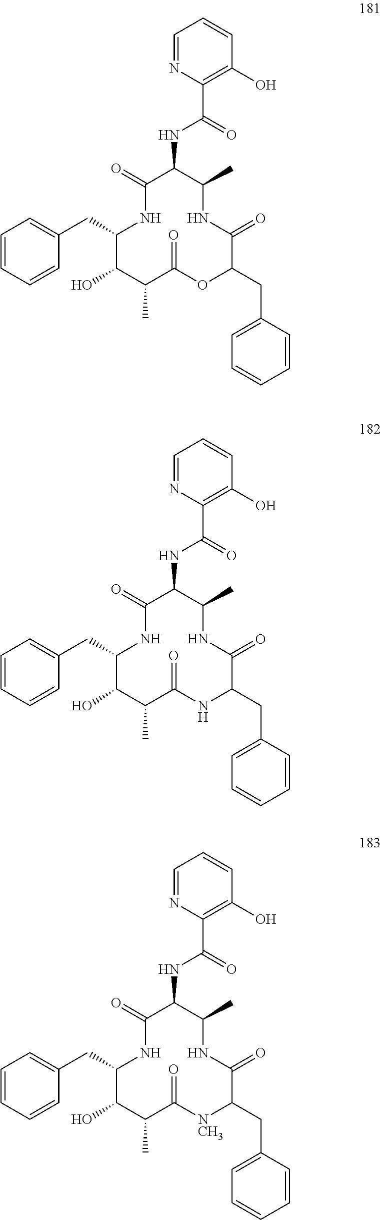 [SMILES: C[C@H]1NC(=O)C(Cc2ccccc2)OC(=O)[C@H](C)[C@H](O)[C@H](Cc2ccccc2)NC(=O)[C@H]1NC(=O)c1ncccc1O ; C[C@H]1NC(=O)C(Cc2ccccc2)NC(=O)[C@H](C)[C@H](O)[C@H](Cc2ccccc2)NC(=O)[C@H]1NC(=O)c1ncccc1O ; C[C@H]1NC(=O)C(Cc2ccccc2)N(C)C(=O)[C@H](C)[C@H](O)[C@H](Cc2ccccc2)NC(=O)[C@H]1NC(=O)c1ncccc1O]

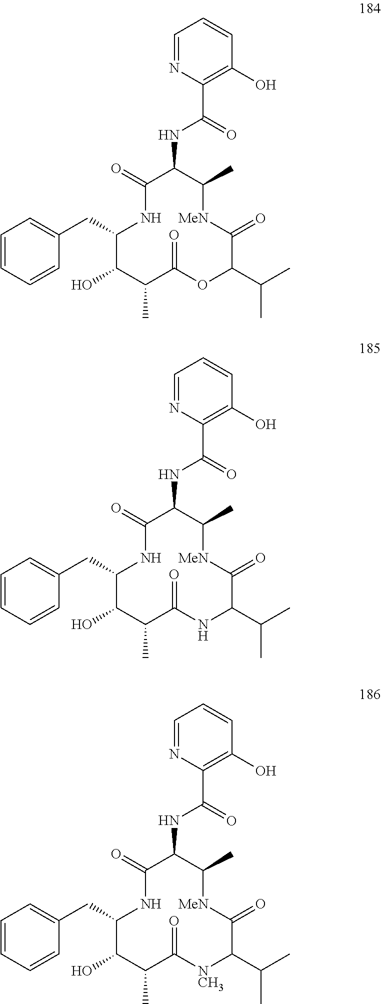 [SMILES: CC(C)C1OC(=O)[C@H](C)[C@H](O)[C@H](Cc2ccccc2)NC(=O)[C@@H](NC(=O)c2ncccc2O)[C@@H](C)N(C)C1=O ; CC(C)C1NC(=O)[C@H](C)[C@H](O)[C@H](Cc2ccccc2)NC(=O)[C@@H](NC(=O)c2ncccc2O)[C@@H](C)N(C)C1=O ; CC(C)C1C(=O)N(C)[C@H](C)[C@H](NC(=O)c2ncccc2O)C(=O)N[C@@H](Cc2ccccc2)[C@@H](O)[C@@H](C)C(=O)N1C]

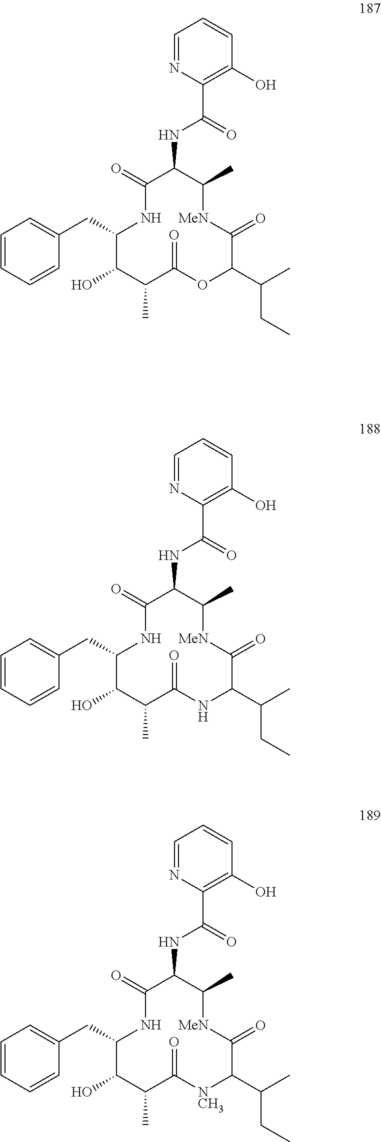 [SMILES: CCC(C)C1OC(=O)[C@H](C)[C@H](O)[C@H](Cc2ccccc2)NC(=O)[C@@H](NC(=O)c2ncccc2O)[C@@H](C)N(C)C1=O ; CCC(C)C1NC(=O)[C@H](C)[C@H](O)[C@H](Cc2ccccc2)NC(=O)[C@@H](NC(=O)c2ncccc2O)[C@@H](C)N(C)C1=O ; CCC(C)C1C(=O)N(C)[C@H](C)[C@H](NC(=O)c2ncccc2O)C(=O)N[C@@H](Cc2ccccc2)[C@@H](O)[C@@H](C)C(=O)N1C]

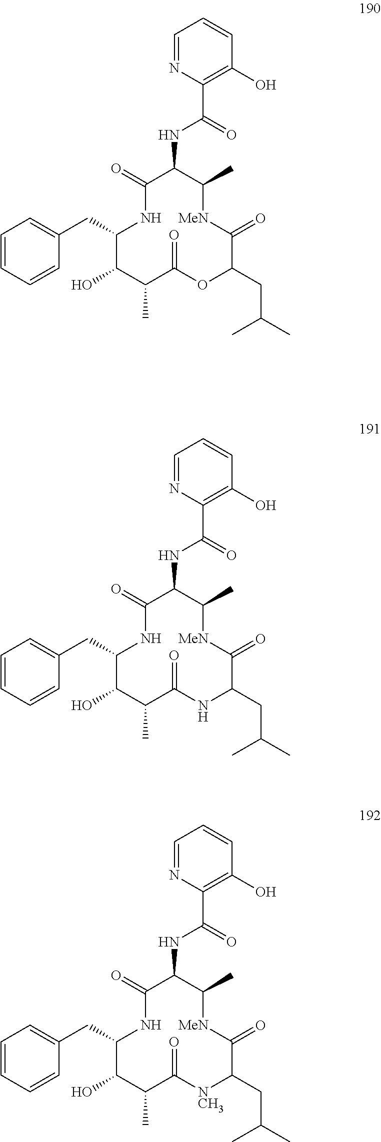 [SMILES: CC(C)CC1OC(=O)[C@H](C)[C@H](O)[C@H](Cc2ccccc2)NC(=O)[C@@H](NC(=O)c2ncccc2O)[C@@H](C)N(C)C1=O ; CC(C)CC1NC(=O)[C@H](C)[C@H](O)[C@H](Cc2ccccc2)NC(=O)[C@@H](NC(=O)c2ncccc2O)[C@@H](C)N(C)C1=O ; CC(C)CC1C(=O)N(C)[C@H](C)[C@H](NC(=O)c2ncccc2O)C(=O)N[C@@H](Cc2ccccc2)[C@@H](O)[C@@H](C)C(=O)N1C]

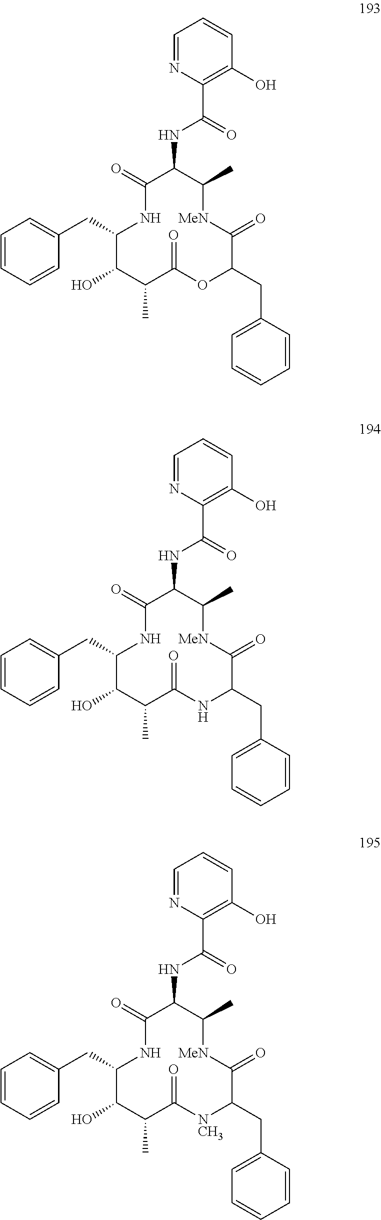 [SMILES: C[C@@H]1[C@H](NC(=O)c2ncccc2O)C(=O)N[C@@H](Cc2ccccc2)[C@@H](O)[C@@H](C)C(=O)OC(Cc2ccccc2)C(=O)N1C ; C[C@@H]1[C@H](NC(=O)c2ncccc2O)C(=O)N[C@@H](Cc2ccccc2)[C@@H](O)[C@@H](C)C(=O)NC(Cc2ccccc2)C(=O)N1C ; C[C@@H]1[C@H](NC(=O)c2ncccc2O)C(=O)N[C@@H](Cc2ccccc2)[C@@H](O)[C@@H](C)C(=O)N(C)C(Cc2ccccc2)C(=O)N1C]

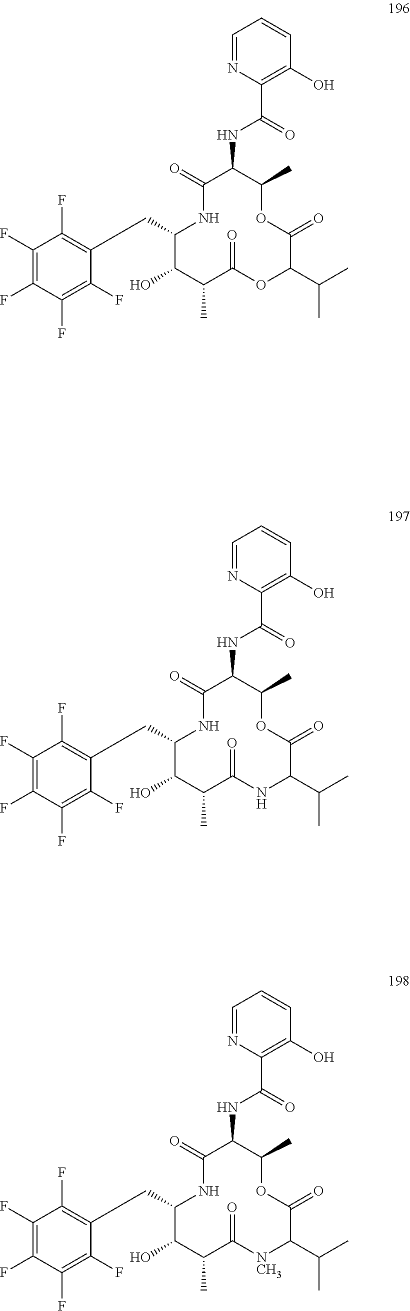 [SMILES: CC(C)C1OC(=O)[C@H](C)[C@H](O)[C@H](Cc2c(F)c(F)c(F)c(F)c2F)NC(=O)[C@@H](NC(=O)c2ncccc2O)[C@@H](C)OC1=O ; CC(C)C1NC(=O)[C@H](C)[C@H](O)[C@H](Cc2c(F)c(F)c(F)c(F)c2F)NC(=O)[C@@H](NC(=O)c2ncccc2O)[C@@H](C)OC1=O ; CC(C)C1C(=O)O[C@H](C)[C@H](NC(=O)c2ncccc2O)C(=O)N[C@@H](Cc2c(F)c(F)c(F)c(F)c2F)[C@@H](O)[C@@H](C)C(=O)N1C]

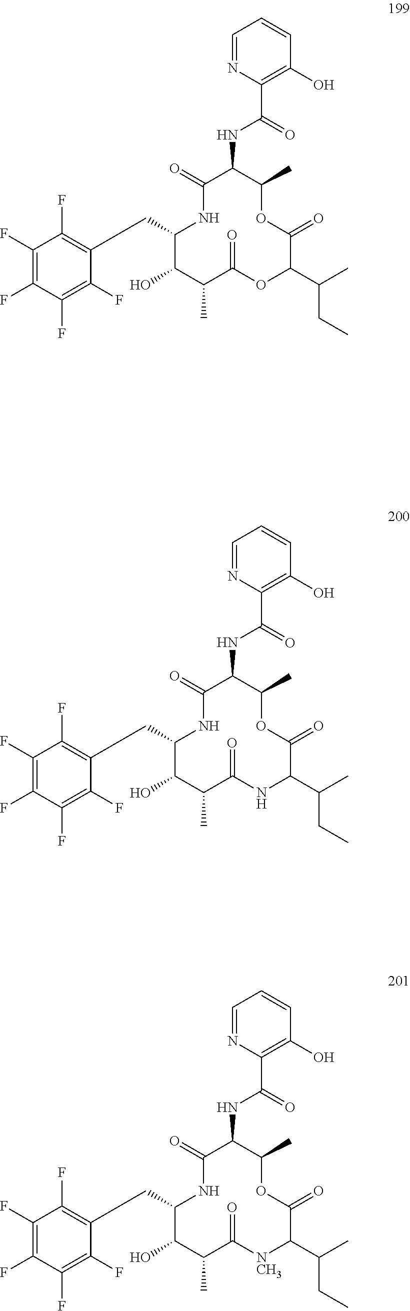 [SMILES: CCC(C)C1OC(=O)[C@H](C)[C@H](O)[C@H](Cc2c(F)c(F)c(F)c(F)c2F)NC(=O)[C@@H](NC(=O)c2ncccc2O)[C@@H](C)OC1=O ; CCC(C)C1NC(=O)[C@H](C)[C@H](O)[C@H](Cc2c(F)c(F)c(F)c(F)c2F)NC(=O)[C@@H](NC(=O)c2ncccc2O)[C@@H](C)OC1=O ; CCC(C)C1C(=O)O[C@H](C)[C@H](NC(=O)c2ncccc2O)C(=O)N[C@@H](Cc2c(F)c(F)c(F)c(F)c2F)[C@@H](O)[C@@H](C)C(=O)N1C]

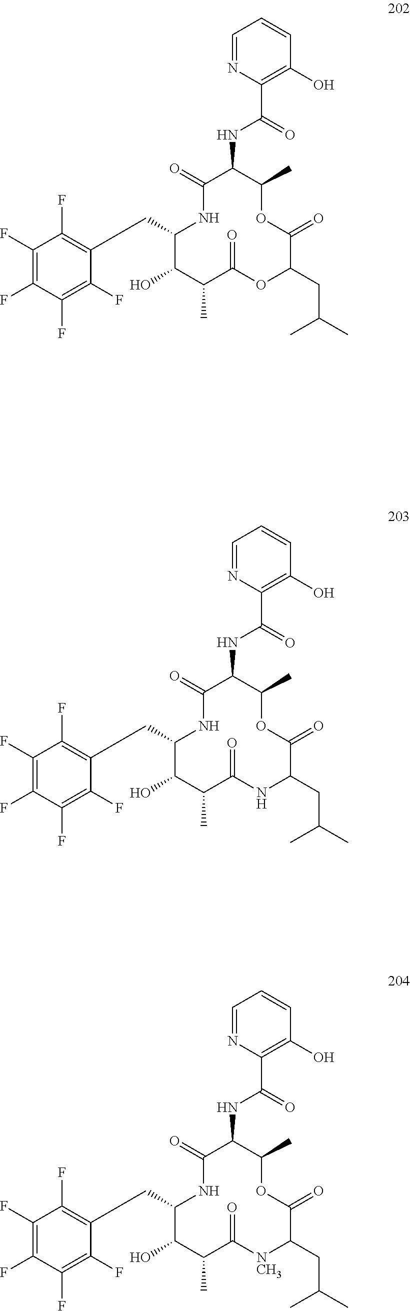 [SMILES: CC(C)CC1OC(=O)[C@H](C)[C@H](O)[C@H](Cc2c(F)c(F)c(F)c(F)c2F)NC(=O)[C@@H](NC(=O)c2ncccc2O)[C@@H](C)OC1=O ; CC(C)CC1NC(=O)[C@H](C)[C@H](O)[C@H](Cc2c(F)c(F)c(F)c(F)c2F)NC(=O)[C@@H](NC(=O)c2ncccc2O)[C@@H](C)OC1=O ; CC(C)CC1C(=O)O[C@H](C)[C@H](NC(=O)c2ncccc2O)C(=O)N[C@@H](Cc2c(F)c(F)c(F)c(F)c2F)[C@@H](O)[C@@H](C)C(=O)N1C]

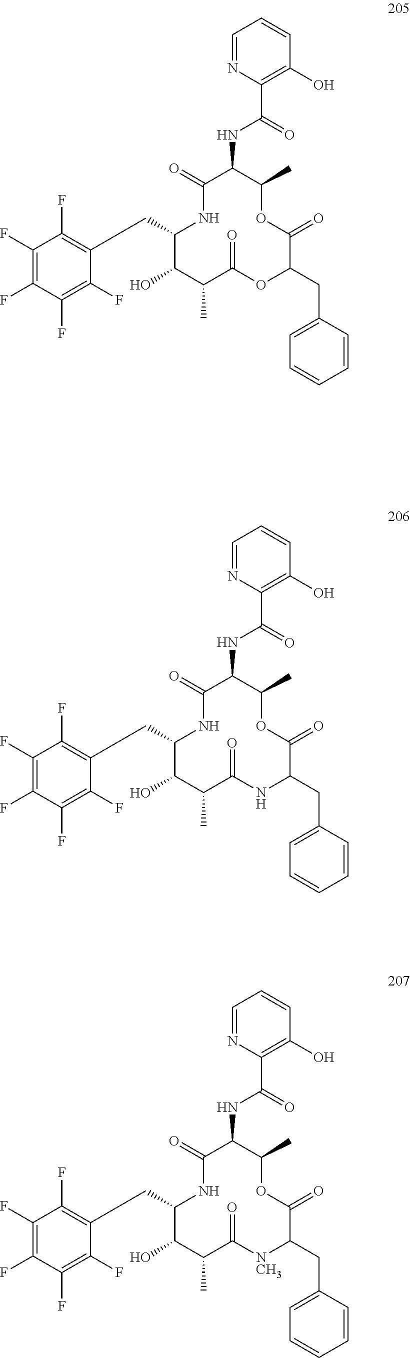 [SMILES: C[C@H]1OC(=O)C(Cc2ccccc2)OC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(Cc2ccccc2)NC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(Cc2ccccc2)N(C)C(=O)[C@H](C)[C@H](O)[C@H](Cc2c(F)c(F)c(F)c(F)c2F)NC(=O)[C@H]1NC(=O)c1ncccc1O]

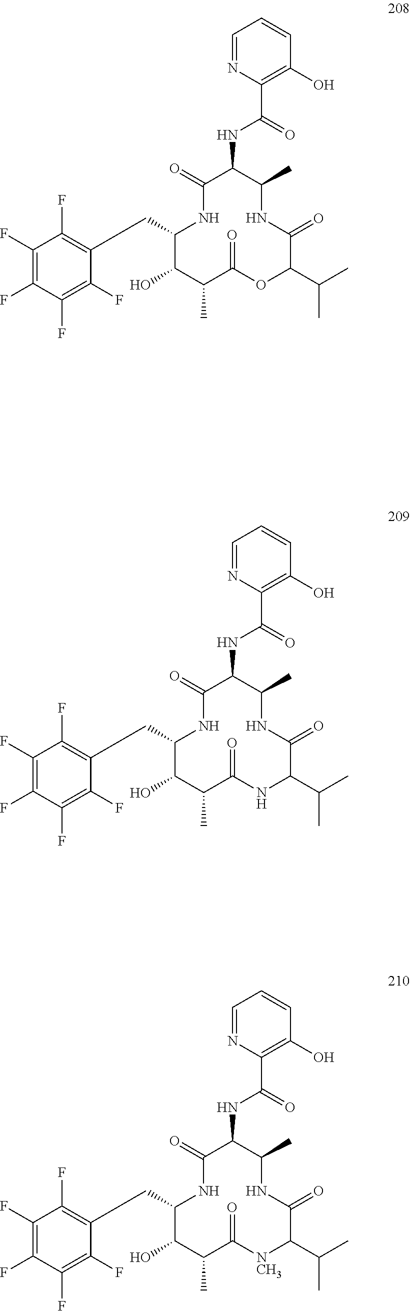 [SMILES: CC(C)C1OC(=O)[C@H](C)[C@H](O)[C@H](Cc2c(F)c(F)c(F)c(F)c2F)NC(=O)[C@@H](NC(=O)c2ncccc2O)[C@@H](C)NC1=O ; CC(C)C1NC(=O)[C@H](C)[C@H](O)[C@H](Cc2c(F)c(F)c(F)c(F)c2F)NC(=O)[C@@H](NC(=O)c2ncccc2O)[C@@H](C)NC1=O ; CC(C)C1C(=O)N[C@H](C)[C@H](NC(=O)c2ncccc2O)C(=O)N[C@@H](Cc2c(F)c(F)c(F)c(F)c2F)[C@@H](O)[C@@H](C)C(=O)N1C]

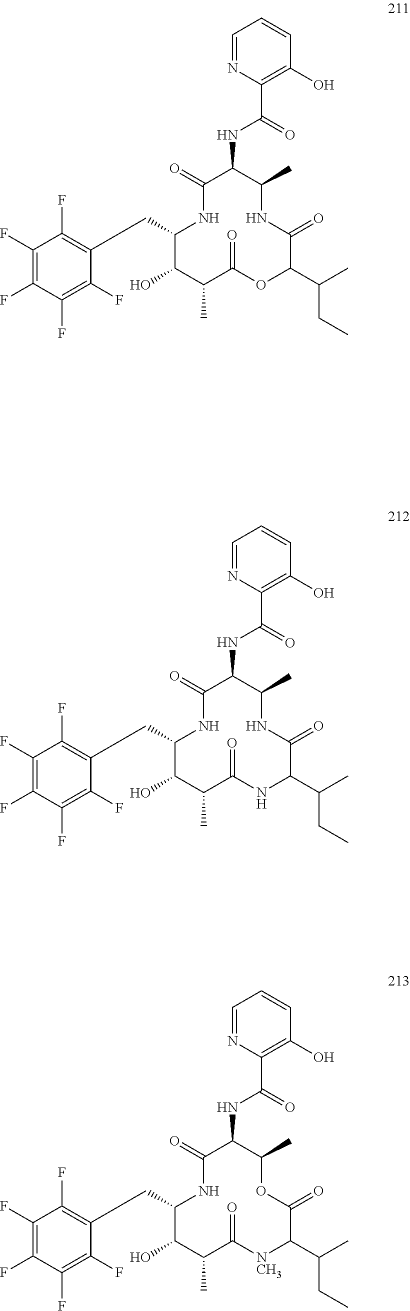 [SMILES: CCC(C)C1OC(=O)[C@H](C)[C@H](O)[C@H](Cc2c(F)c(F)c(F)c(F)c2F)NC(=O)[C@@H](NC(=O)c2ncccc2O)[C@@H](C)NC1=O ; CCC(C)C1NC(=O)[C@H](C)[C@H](O)[C@H](Cc2c(F)c(F)c(F)c(F)c2F)NC(=O)[C@@H](NC(=O)c2ncccc2O)[C@@H](C)NC1=O ; CCC(C)C1C(=O)O[C@H](C)[C@H](NC(=O)c2ncccc2O)C(=O)N[C@@H](Cc2c(F)c(F)c(F)c(F)c2F)[C@@H](O)[C@@H](C)C(=O)N1C]

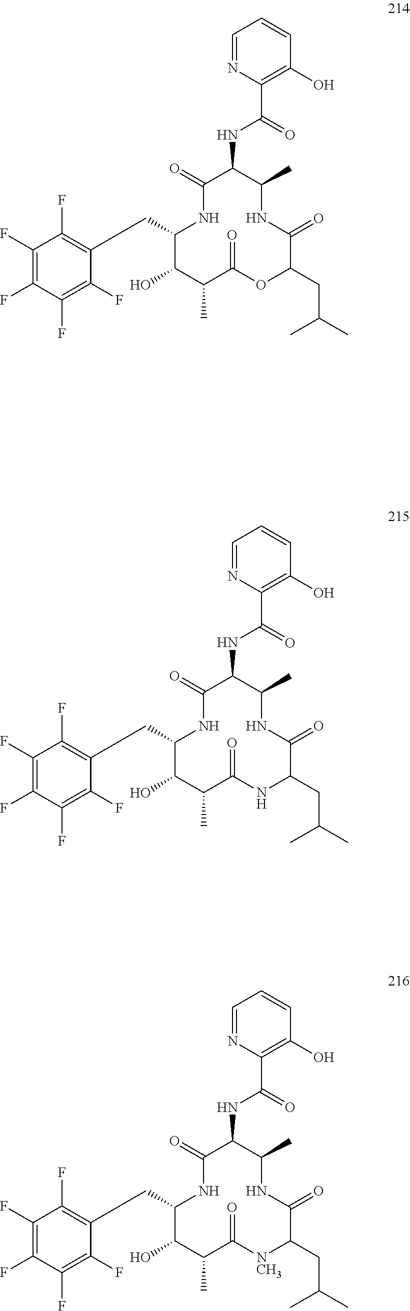 [SMILES: CC(C)CC1OC(=O)[C@H](C)[C@H](O)[C@H](Cc2c(F)c(F)c(F)c(F)c2F)NC(=O)[C@@H](NC(=O)c2ncccc2O)[C@@H](C)NC1=O ; CC(C)CC1NC(=O)[C@H](C)[C@H](O)[C@H](Cc2c(F)c(F)c(F)c(F)c2F)NC(=O)[C@@H](NC(=O)c2ncccc2O)[C@@H](C)NC1=O ; CC(C)CC1C(=O)N[C@H](C)[C@H](NC(=O)c2ncccc2O)C(=O)N[C@@H](Cc2c(F)c(F)c(F)c(F)c2F)[C@@H](O)[C@@H](C)C(=O)N1C]

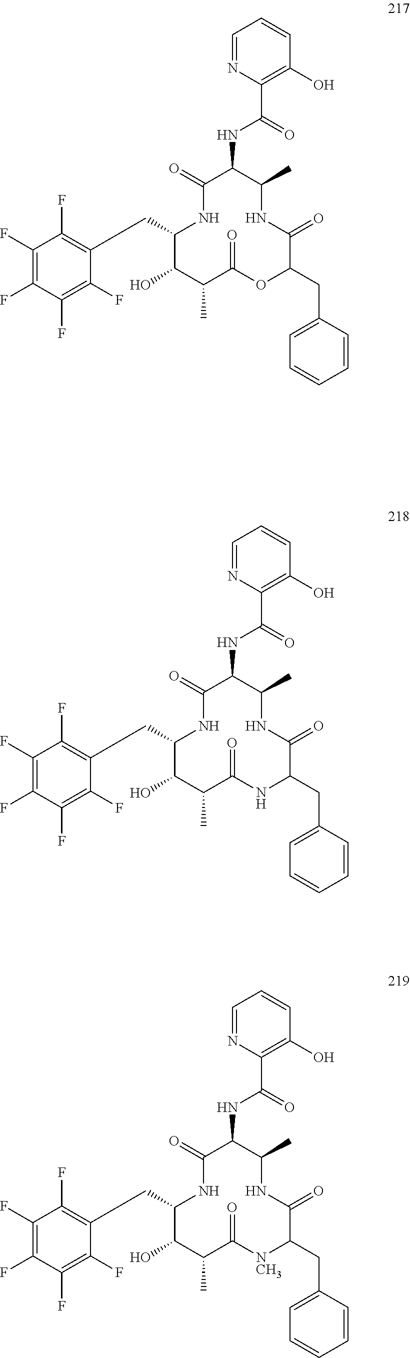 [SMILES: C[C@H]1NC(=O)C(Cc2ccccc2)OC(=O)[C@H](C)[C@H](O)[C@H](Cc2c(F)c(F)c(F)c(F)c2F)NC(=O)[C@H]1NC(=O)c1ncccc1O ; C[C@H]1NC(=O)C(Cc2ccccc2)NC(=O)[C@H](C)[C@H](O)[C@H](Cc2c(F)c(F)c(F)c(F)c2F)NC(=O)[C@H]1NC(=O)c1ncccc1O ; C[C@H]1NC(=O)C(Cc2ccccc2)N(C)C(=O)[C@H](C)[C@H](O)[C@H](Cc2c(F)c(F)c(F)c(F)c2F)NC(=O)[C@H]1NC(=O)c1ncccc1O]

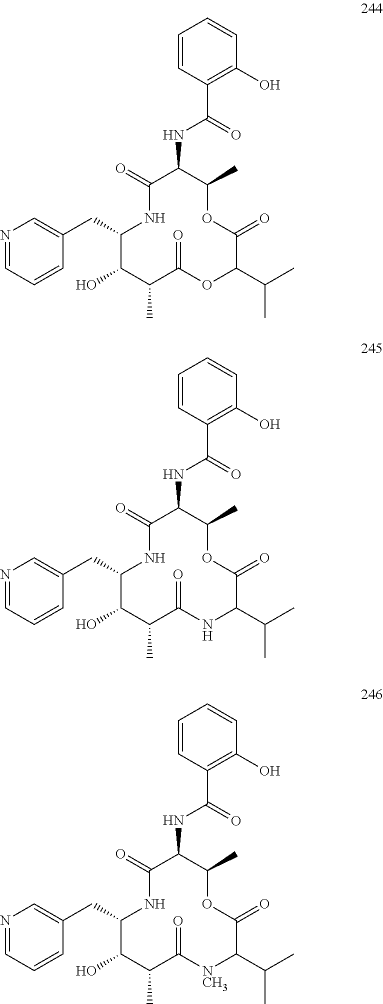 [SMILES: CC(C)C1OC(=O)[C@H](C)[C@H](O)[C@H](Cc2cccnc2)NC(=O)[C@@H](NC(=O)c2ccccc2O)[C@@H](C)OC1=O ; CC(C)C1NC(=O)[C@H](C)[C@H](O)[C@H](Cc2cccnc2)NC(=O)[C@@H](NC(=O)c2ccccc2O)[C@@H](C)OC1=O ; CC(C)C1C(=O)O[C@H](C)[C@H](NC(=O)c2ccccc2O)C(=O)N[C@@H](Cc2cccnc2)[C@@H](O)[C@@H](C)C(=O)N1C]

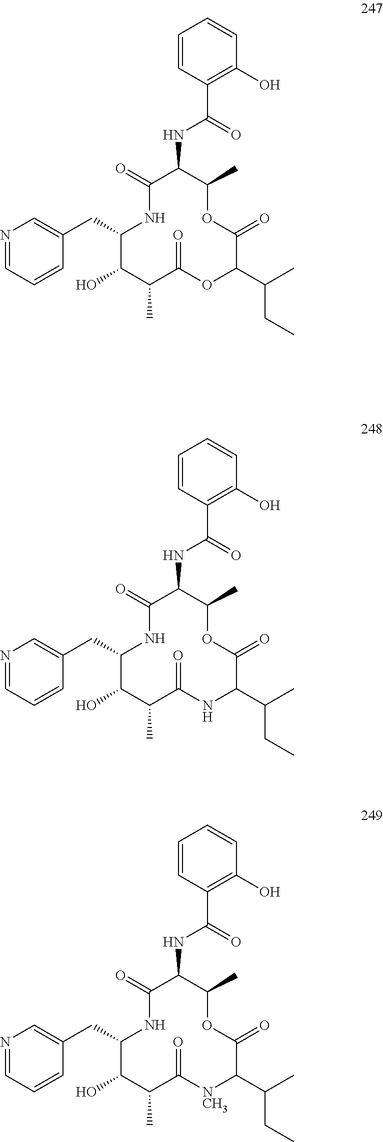 [SMILES: CCC(C)C1OC(=O)[C@H](C)[C@H](O)[C@H](Cc2cccnc2)NC(=O)[C@@H](NC(=O)c2ccccc2O)[C@@H](C)OC1=O ; CCC(C)C1NC(=O)[C@H](C)[C@H](O)[C@H](Cc2cccnc2)NC(=O)[C@@H](NC(=O)c2ccccc2O)[C@@H](C)OC1=O ; CCC(C)C1C(=O)O[C@H](C)[C@H](NC(=O)c2ccccc2O)C(=O)N[C@@H](Cc2cccnc2)[C@@H](O)[C@@H](C)C(=O)N1C]

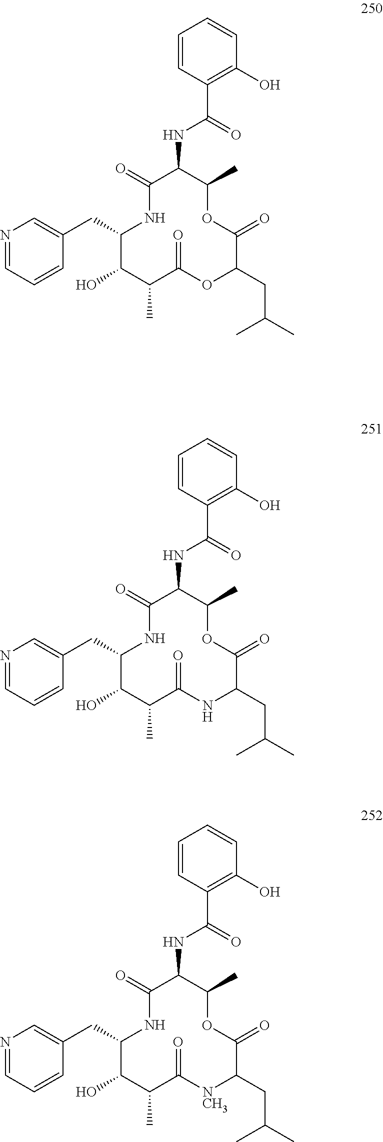 [SMILES: CC(C)CC1OC(=O)[C@H](C)[C@H](O)[C@H](Cc2cccnc2)NC(=O)[C@@H](NC(=O)c2ccccc2O)[C@@H](C)OC1=O ; CC(C)CC1NC(=O)[C@H](C)[C@H](O)[C@H](Cc2cccnc2)NC(=O)[C@@H](NC(=O)c2ccccc2O)[C@@H](C)OC1=O ; CC(C)CC1C(=O)O[C@H](C)[C@H](NC(=O)c2ccccc2O)C(=O)N[C@@H](Cc2cccnc2)[C@@H](O)[C@@H](C)C(=O)N1C]

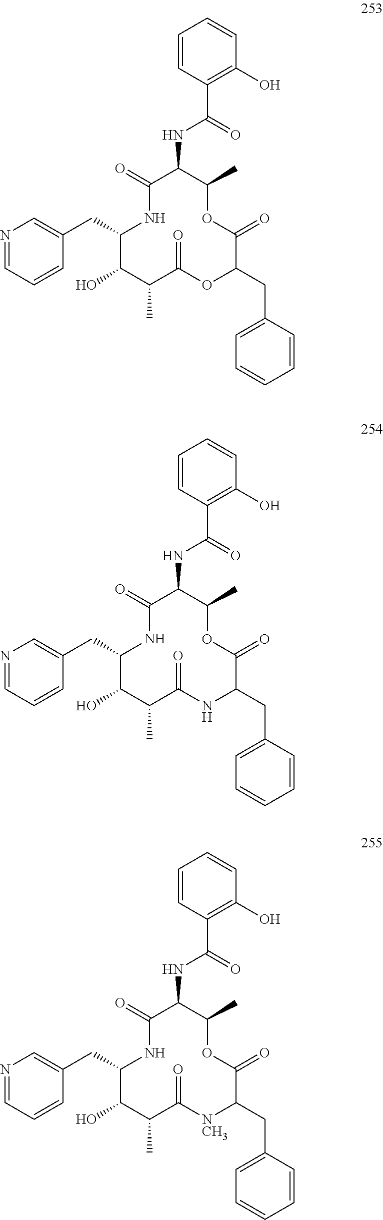 [SMILES: C[C@H]1OC(=O)C(Cc2ccccc2)OC(=O)[C@H](C)[C@H](O)[C@H](Cc2cccnc2)NC(=O)[C@H]1NC(=O)c1ccccc1O ; C[C@H]1OC(=O)C(Cc2ccccc2)NC(=O)[C@H](C)[C@H](O)[C@H](Cc2cccnc2)NC(=O)[C@H]1NC(=O)c1ccccc1O ; C[C@H]1OC(=O)C(Cc2ccccc2)N(C)C(=O)[C@H](C)[C@H](O)[C@H](Cc2cccnc2)NC(=O)[C@H]1NC(=O)c1ccccc1O]

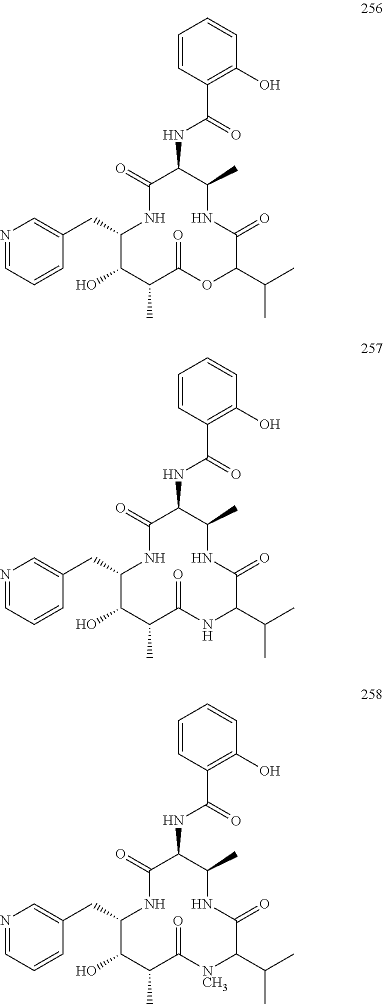 [SMILES: CC(C)C1OC(=O)[C@H](C)[C@H](O)[C@H](Cc2cccnc2)NC(=O)[C@@H](NC(=O)c2ccccc2O)[C@@H](C)NC1=O ; CC(C)C1NC(=O)[C@H](C)[C@H](O)[C@H](Cc2cccnc2)NC(=O)[C@@H](NC(=O)c2ccccc2O)[C@@H](C)NC1=O ; CC(C)C1C(=O)N[C@H](C)[C@H](NC(=O)c2ccccc2O)C(=O)N[C@@H](Cc2cccnc2)[C@@H](O)[C@@H](C)C(=O)N1C]

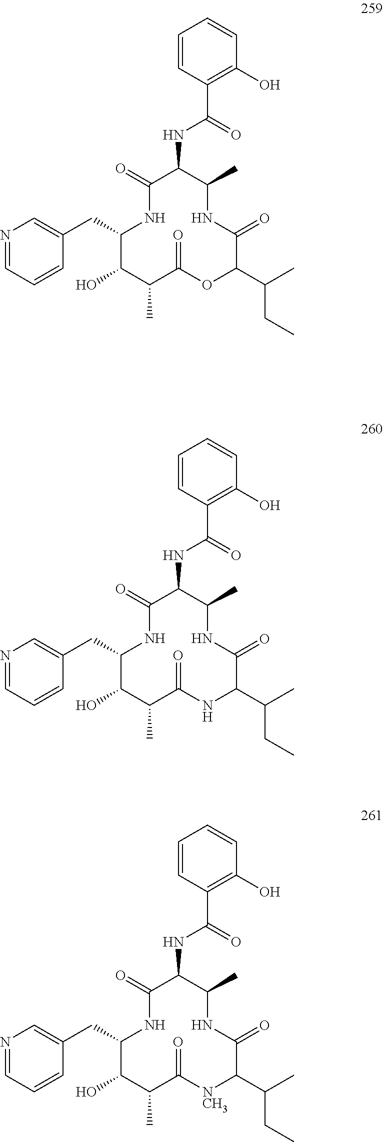 [SMILES: CCC(C)C1OC(=O)[C@H](C)[C@H](O)[C@H](Cc2cccnc2)NC(=O)[C@@H](NC(=O)c2ccccc2O)[C@@H](C)NC1=O ; CCC(C)C1NC(=O)[C@H](C)[C@H](O)[C@H](Cc2cccnc2)NC(=O)[C@@H](NC(=O)c2ccccc2O)[C@@H](C)NC1=O ; CCC(C)C1C(=O)N[C@H](C)[C@H](NC(=O)c2ccccc2O)C(=O)N[C@@H](Cc2cccnc2)[C@@H](O)[C@@H](C)C(=O)N1C]

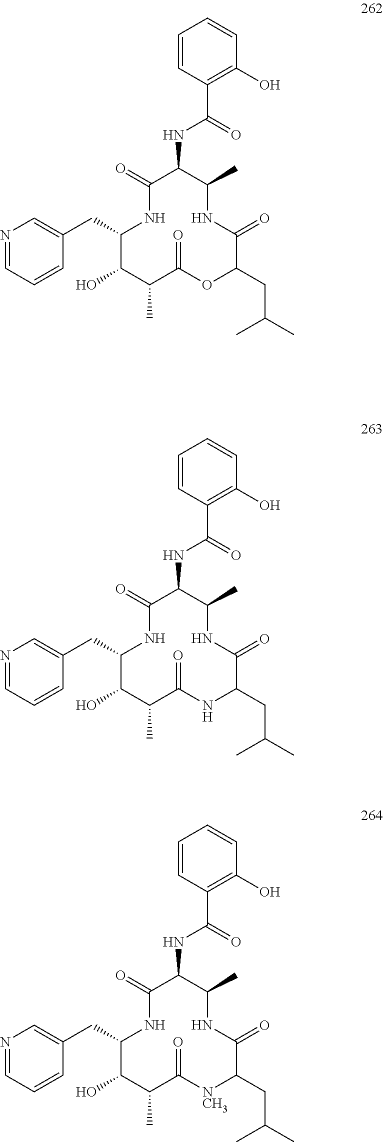 [SMILES: CC(C)CC1OC(=O)[C@H](C)[C@H](O)[C@H](Cc2cccnc2)NC(=O)[C@@H](NC(=O)c2ccccc2O)[C@@H](C)NC1=O ; CC(C)CC1NC(=O)[C@H](C)[C@H](O)[C@H](Cc2cccnc2)NC(=O)[C@@H](NC(=O)c2ccccc2O)[C@@H](C)NC1=O ; CC(C)CC1C(=O)N[C@H](C)[C@H](NC(=O)c2ccccc2O)C(=O)N[C@@H](Cc2cccnc2)[C@@H](O)[C@@H](C)C(=O)N1C]

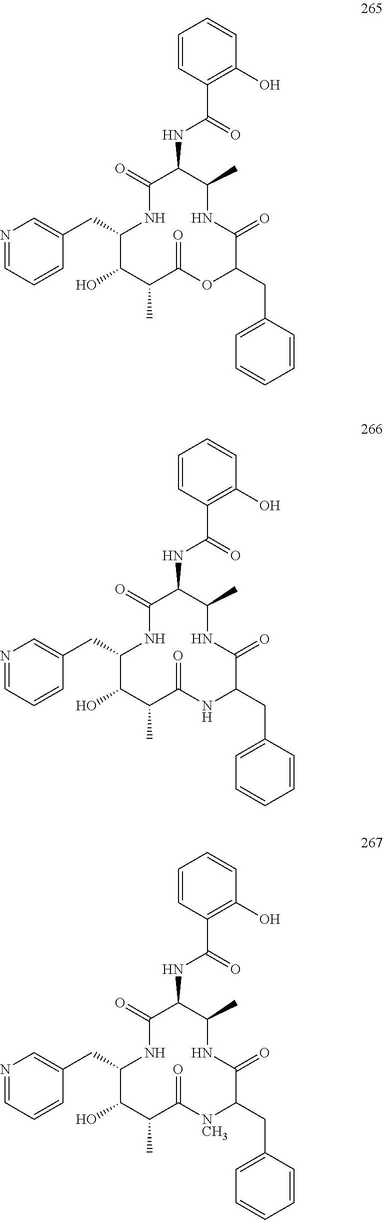 [SMILES: C[C@H]1NC(=O)C(Cc2ccccc2)OC(=O)[C@H](C)[C@H](O)[C@H](Cc2cccnc2)NC(=O)[C@H]1NC(=O)c1ccccc1O ; C[C@H]1NC(=O)C(Cc2ccccc2)NC(=O)[C@H](C)[C@H](O)[C@H](Cc2cccnc2)NC(=O)[C@H]1NC(=O)c1ccccc1O ; C[C@H]1NC(=O)C(Cc2ccccc2)N(C)C(=O)[C@H](C)[C@H](O)[C@H](Cc2cccnc2)NC(=O)[C@H]1NC(=O)c1ccccc1O]

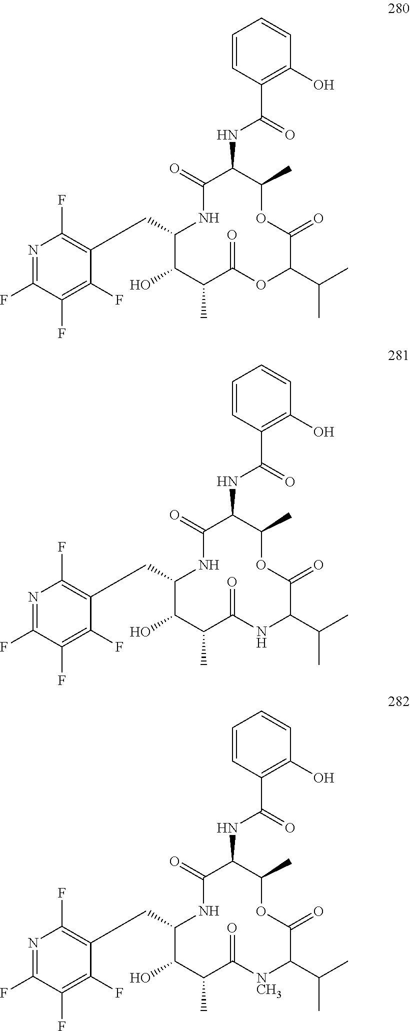 [SMILES: CC(C)C1OC(=O)[C@H](C)[C@H](O)[C@H](Cc2c(F)nc(F)c(F)c2F)NC(=O)[C@@H](NC(=O)c2ccccc2O)[C@@H](C)OC1=O ; CC(C)C1NC(=O)[C@H](C)[C@H](O)[C@H](Cc2c(F)nc(F)c(F)c2F)NC(=O)[C@@H](NC(=O)c2ccccc2O)[C@@H](C)OC1=O ; CC(C)C1C(=O)O[C@H](C)[C@H](NC(=O)c2ccccc2O)C(=O)N[C@@H](Cc2c(F)nc(F)c(F)c2F)[C@@H](O)[C@@H](C)C(=O)N1C]

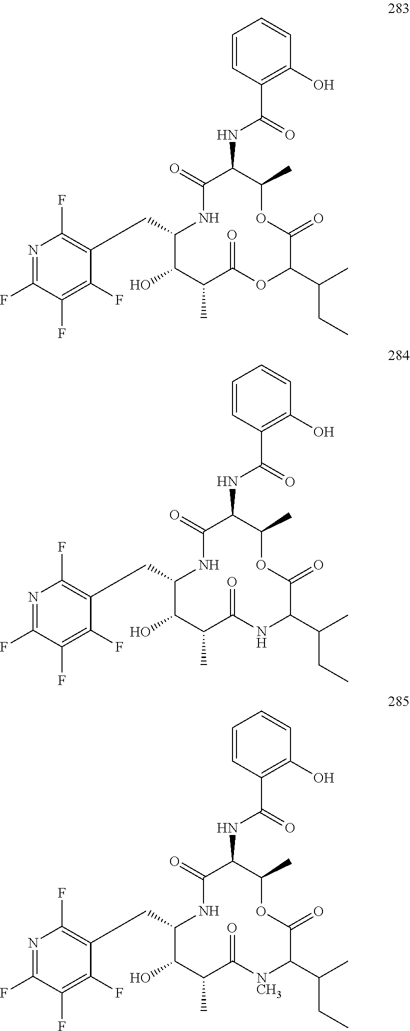 [SMILES: CCC(C)C1OC(=O)[C@H](C)[C@H](O)[C@H](Cc2c(F)nc(F)c(F)c2F)NC(=O)[C@@H](NC(=O)c2ccccc2O)[C@@H](C)OC1=O ; CCC(C)C1NC(=O)[C@H](C)[C@H](O)[C@H](Cc2c(F)nc(F)c(F)c2F)NC(=O)[C@@H](NC(=O)c2ccccc2O)[C@@H](C)OC1=O ; CCC(C)C1C(=O)O[C@H](C)[C@H](NC(=O)c2ccccc2O)C(=O)N[C@@H](Cc2c(F)nc(F)c(F)c2F)[C@@H](O)[C@@H](C)C(=O)N1C]

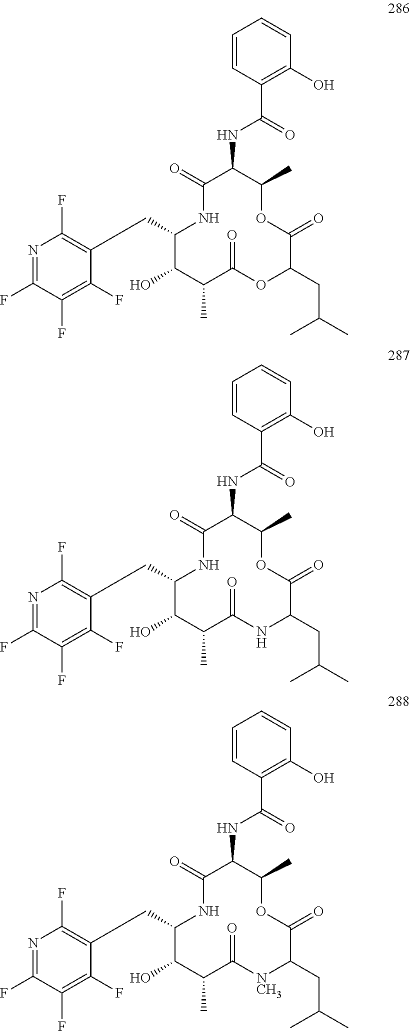

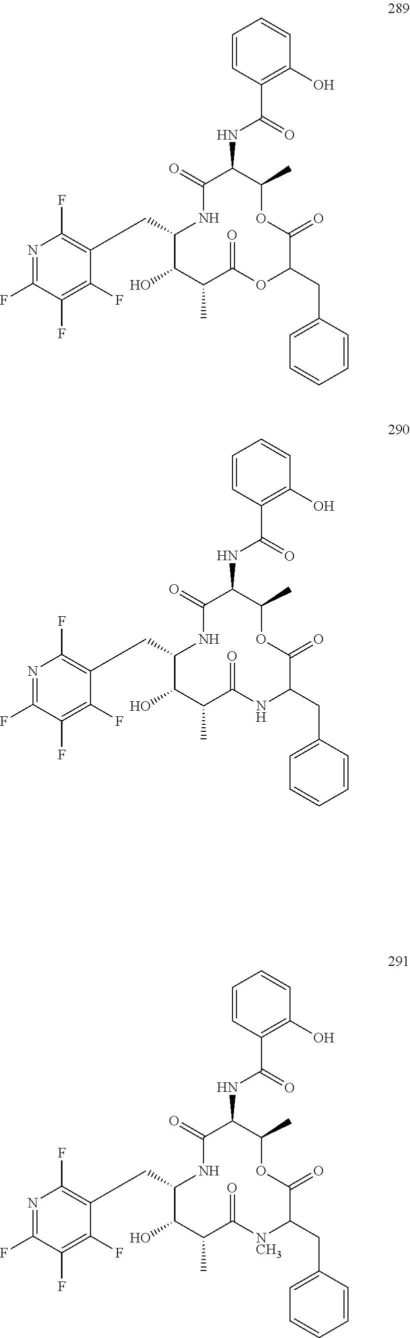 [SMILES: C[C@H]1OC(=O)C(Cc2ccccc2)OC(=O)[C@H](C)[C@H](O)[C@H](Cc2c(F)nc(F)c(F)c2F)NC(=O)[C@H]1NC(=O)c1ccccc1O ; C[C@H]1OC(=O)C(Cc2ccccc2)NC(=O)[C@H](C)[C@H](O)[C@H](Cc2c(F)nc(F)c(F)c2F)NC(=O)[C@H]1NC(=O)c1ccccc1O ; C[C@H]1OC(=O)C(Cc2ccccc2)N(C)C(=O)[C@H](C)[C@H](O)[C@H](Cc2c(F)nc(F)c(F)c2F)NC(=O)[C@H]1NC(=O)c1ccccc1O]

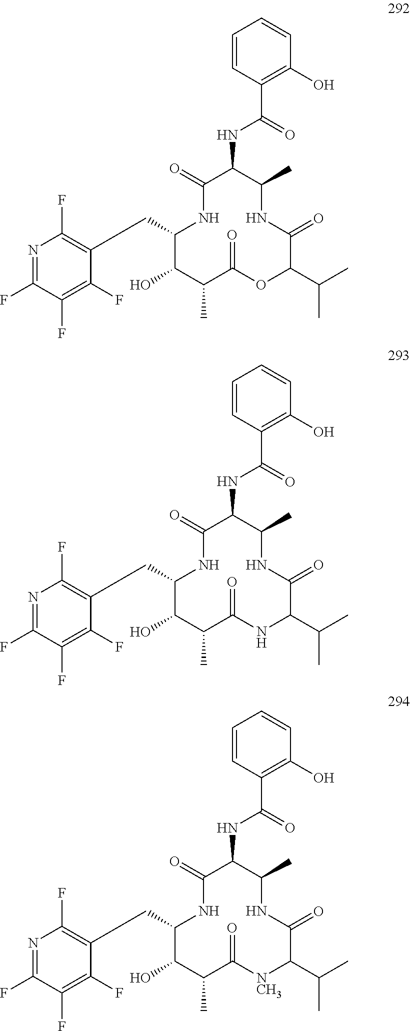 [SMILES: CC(C)C1OC(=O)[C@H](C)[C@H](O)[C@H](Cc2c(F)nc(F)c(F)c2F)NC(=O)[C@@H](NC(=O)c2ccccc2O)[C@@H](C)NC1=O ; CC(C)C1NC(=O)[C@H](C)[C@H](O)[C@H](Cc2c(F)nc(F)c(F)c2F)NC(=O)[C@@H](NC(=O)c2ccccc2O)[C@@H](C)NC1=O ; CC(C)C1C(=O)N[C@H](C)[C@H](NC(=O)c2ccccc2O)C(=O)N[C@@H](Cc2c(F)nc(F)c(F)c2F)[C@@H](O)[C@@H](C)C(=O)N1C]

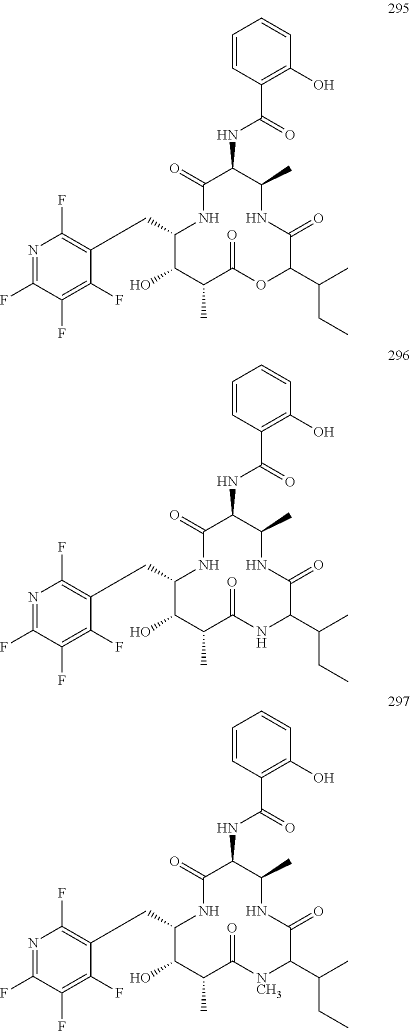 [SMILES: CCC(C)C1OC(=O)[C@H](C)[C@H](O)[C@H](Cc2c(F)nc(F)c(F)c2F)NC(=O)[C@@H](NC(=O)c2ccccc2O)[C@@H](C)NC1=O ; CCC(C)C1NC(=O)[C@H](C)[C@H](O)[C@H](Cc2c(F)nc(F)c(F)c2F)NC(=O)[C@@H](NC(=O)c2ccccc2O)[C@@H](C)NC1=O ; CCC(C)C1C(=O)N[C@H](C)[C@H](NC(=O)c2ccccc2O)C(=O)N[C@@H](Cc2c(F)nc(F)c(F)c2F)[C@@H](O)[C@@H](C)C(=O)N1C]

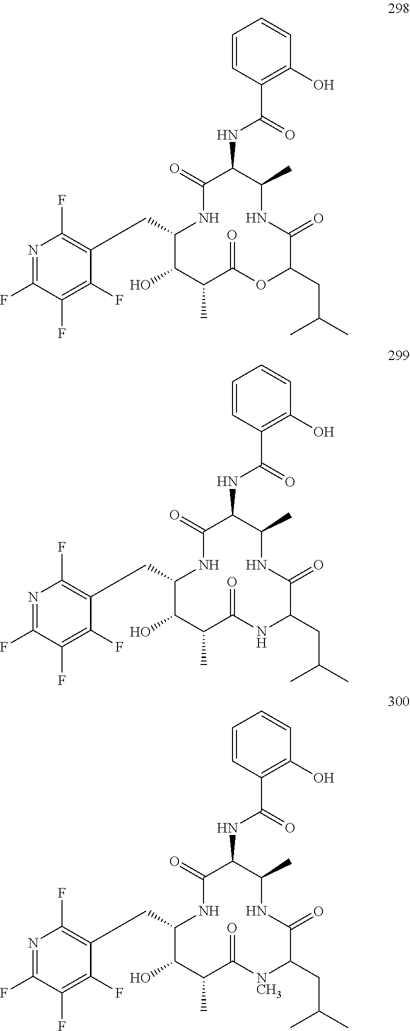

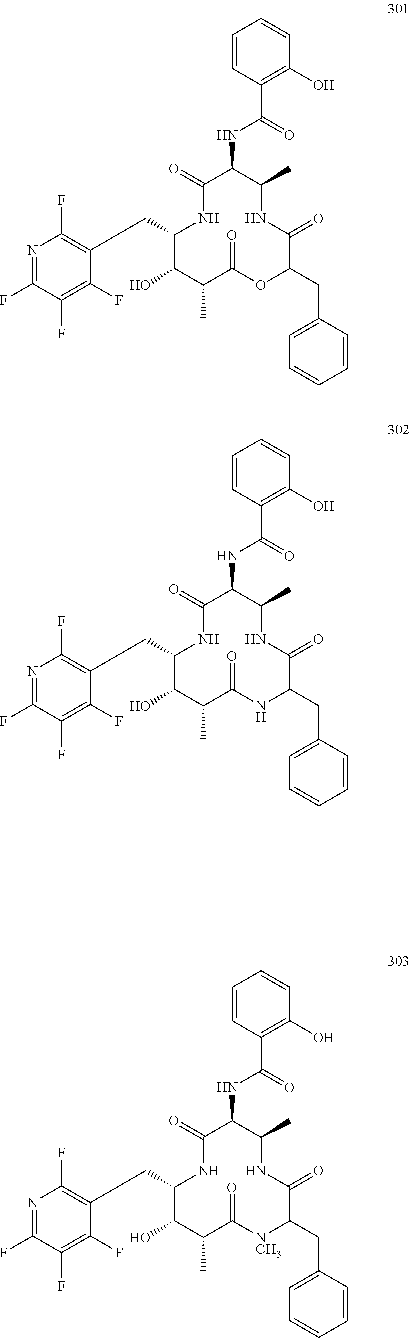 [SMILES: C[C@H]1NC(=O)C(Cc2ccccc2)OC(=O)[C@H](C)[C@H](O)[C@H](Cc2c(F)nc(F)c(F)c2F)NC(=O)[C@H]1NC(=O)c1ccccc1O ; C[C@H]1NC(=O)C(Cc2ccccc2)NC(=O)[C@H](C)[C@H](O)[C@H](Cc2c(F)nc(F)c(F)c2F)NC(=O)[C@H]1NC(=O)c1ccccc1O ; C[C@H]1NC(=O)C(Cc2ccccc2)N(C)C(=O)[C@H](C)[C@H](O)[C@H](Cc2c(F)nc(F)c(F)c2F)NC(=O)[C@H]1NC(=O)c1ccccc1O]

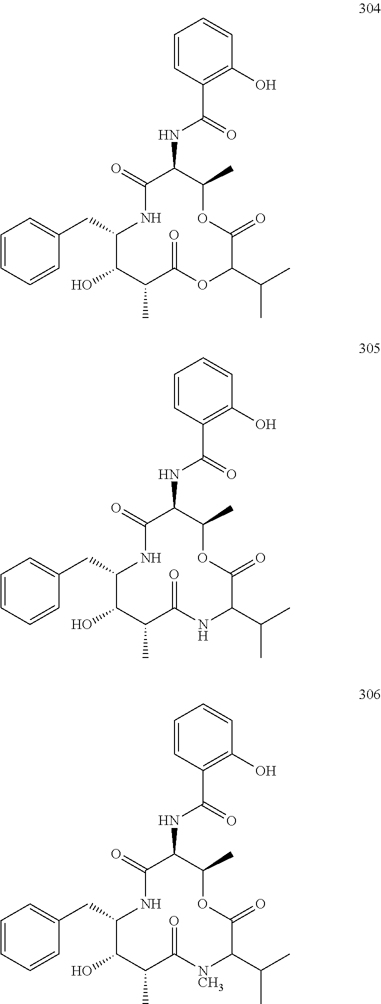 [SMILES: CC(C)C1OC(=O)[C@H](C)[C@H](O)[C@H](Cc2ccccc2)NC(=O)[C@@H](NC(=O)c2ccccc2O)[C@@H](C)OC1=O ; CC(C)C1NC(=O)[C@H](C)[C@H](O)[C@H](Cc2ccccc2)NC(=O)[C@@H](NC(=O)c2ccccc2O)[C@@H](C)OC1=O ; CC(C)C1C(=O)O[C@H](C)[C@H](NC(=O)c2ccccc2O)C(=O)N[C@@H](Cc2ccccc2)[C@@H](O)[C@@H](C)C(=O)N1C]

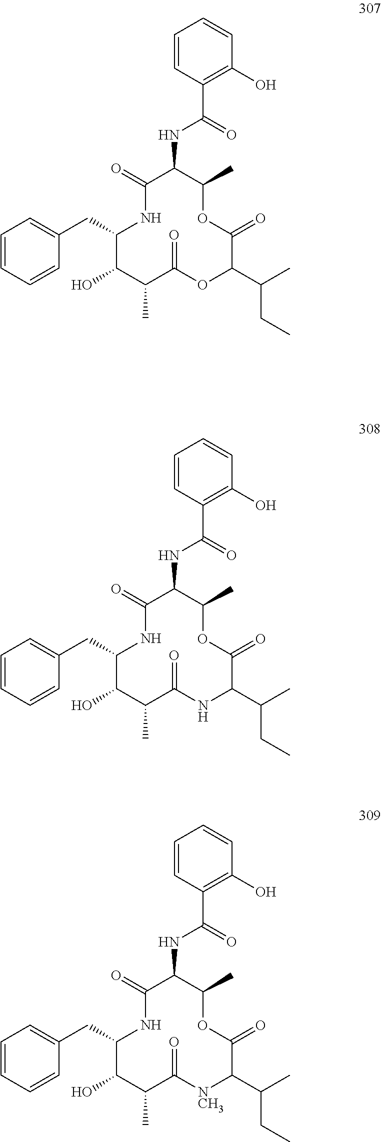 [SMILES: CCC(C)C1OC(=O)[C@H](C)[C@H](O)[C@H](Cc2ccccc2)NC(=O)[C@@H](NC(=O)c2ccccc2O)[C@@H](C)OC1=O ; CCC(C)C1NC(=O)[C@H](C)[C@H](O)[C@H](Cc2ccccc2)NC(=O)[C@@H](NC(=O)c2ccccc2O)[C@@H](C)OC1=O ; CCC(C)C1C(=O)O[C@H](C)[C@H](NC(=O)c2ccccc2O)C(=O)N[C@@H](Cc2ccccc2)[C@@H](O)[C@@H](C)C(=O)N1C]

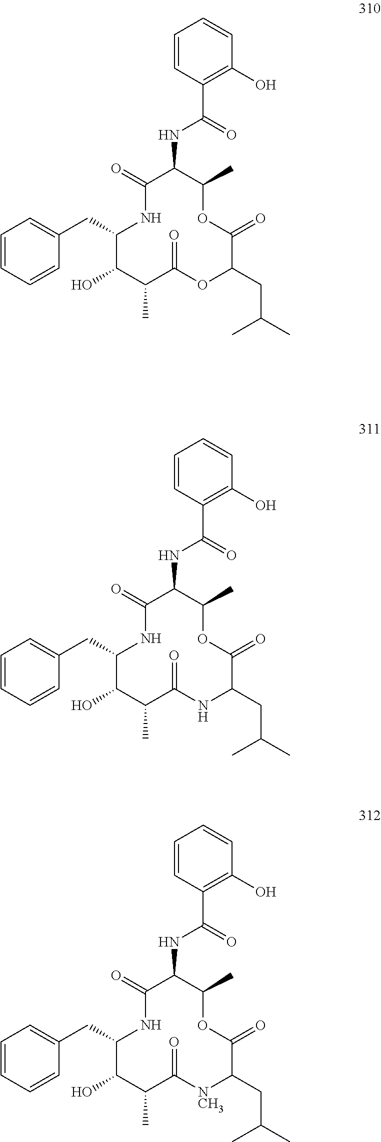 [SMILES: CC(C)CC1OC(=O)[C@H](C)[C@H](O)[C@H](Cc2ccccc2)NC(=O)[C@@H](NC(=O)c2ccccc2O)[C@@H](C)OC1=O ; CC(C)CC1NC(=O)[C@H](C)[C@H](O)[C@H](Cc2ccccc2)NC(=O)[C@@H](NC(=O)c2ccccc2O)[C@@H](C)OC1=O ; CC(C)CC1C(=O)O[C@H](C)[C@H](NC(=O)c2ccccc2O)C(=O)N[C@@H](Cc2ccccc2)[C@@H](O)[C@@H](C)C(=O)N1C]

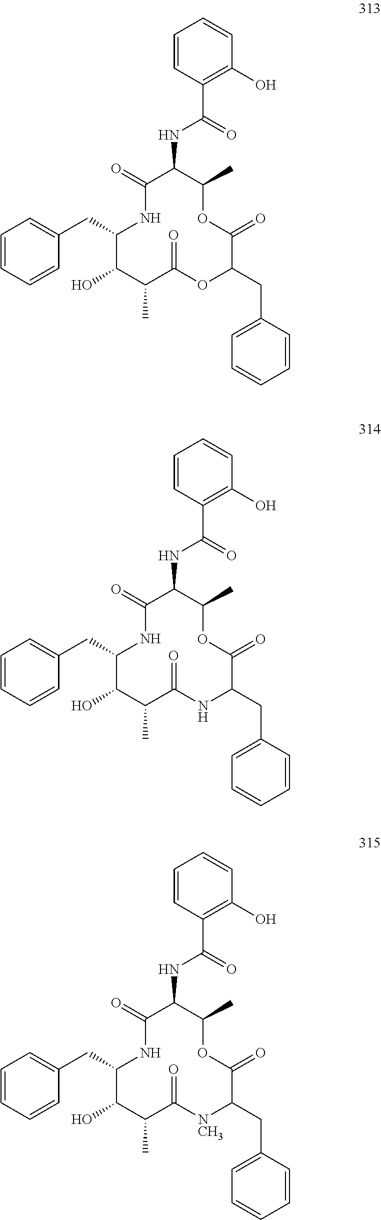 [SMILES: C[C@H]1OC(=O)C(Cc2ccccc2)OC(=O)[C@H](C)[C@H](O)[C@H](Cc2ccccc2)NC(=O)[C@H]1NC(=O)c1ccccc1O ; C[C@H]1OC(=O)C(Cc2ccccc2)NC(=O)[C@H](C)[C@H](O)[C@H](Cc2ccccc2)NC(=O)[C@H]1NC(=O)c1ccccc1O ; C[C@H]1OC(=O)C(Cc2ccccc2)N(C)C(=O)[C@H](C)[C@H](O)[C@H](Cc2ccccc2)NC(=O)[C@H]1NC(=O)c1ccccc1O]

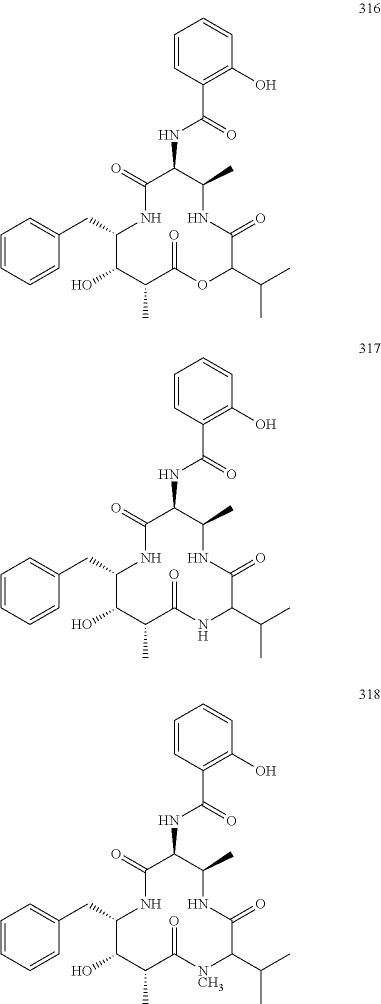 [SMILES: CC(C)C1OC(=O)[C@H](C)[C@H](O)[C@H](Cc2ccccc2)NC(=O)[C@@H](NC(=O)c2ccccc2O)[C@@H](C)NC1=O ; CC(C)C1NC(=O)[C@H](C)[C@H](O)[C@H](Cc2ccccc2)NC(=O)[C@@H](NC(=O)c2ccccc2O)[C@@H](C)NC1=O ; CC(C)C1C(=O)N[C@H](C)[C@H](NC(=O)c2ccccc2O)C(=O)N[C@@H](Cc2ccccc2)[C@@H](O)[C@@H](C)C(=O)N1C]

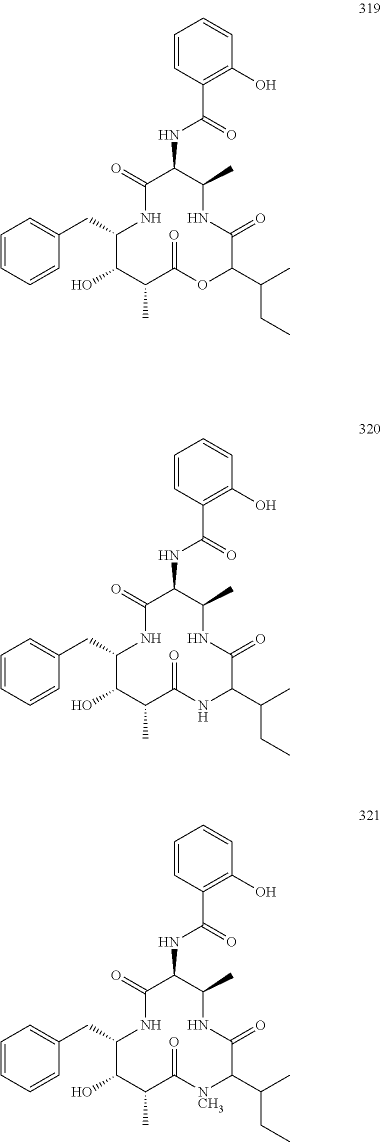 [SMILES: CCC(C)C1OC(=O)[C@H](C)[C@H](O)[C@H](Cc2ccccc2)NC(=O)[C@@H](NC(=O)c2ccccc2O)[C@@H](C)NC1=O ; CCC(C)C1NC(=O)[C@H](C)[C@H](O)[C@H](Cc2ccccc2)NC(=O)[C@@H](NC(=O)c2ccccc2O)[C@@H](C)NC1=O ; CCC(C)C1C(=O)N[C@H](C)[C@H](NC(=O)c2ccccc2O)C(=O)N[C@@H](Cc2ccccc2)[C@@H](O)[C@@H](C)C(=O)N1C]

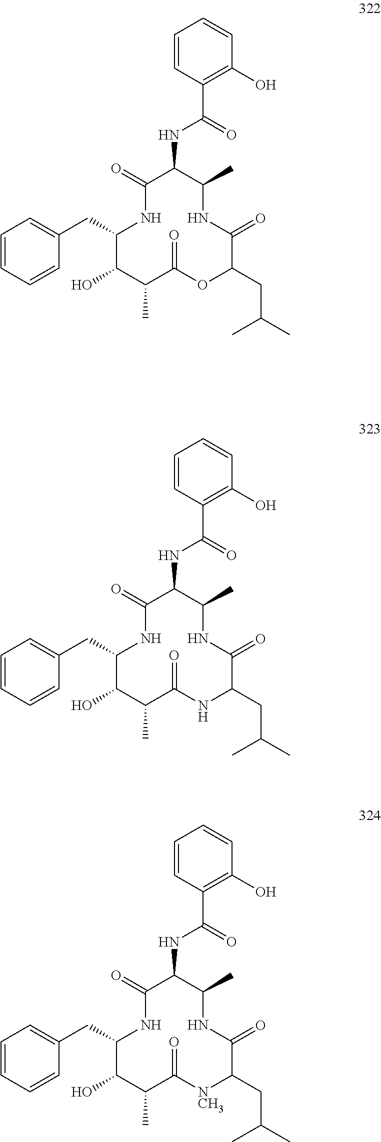 [SMILES: CC(C)CC1OC(=O)[C@H](C)[C@H](O)[C@H](Cc2ccccc2)NC(=O)[C@@H](NC(=O)c2ccccc2O)[C@@H](C)NC1=O ; CC(C)CC1NC(=O)[C@H](C)[C@H](O)[C@H](Cc2ccccc2)NC(=O)[C@@H](NC(=O)c2ccccc2O)[C@@H](C)NC1=O ; CC(C)CC1C(=O)N[C@H](C)[C@H](NC(=O)c2ccccc2O)C(=O)N[C@@H](Cc2ccccc2)[C@@H](O)[C@@H](C)C(=O)N1C]

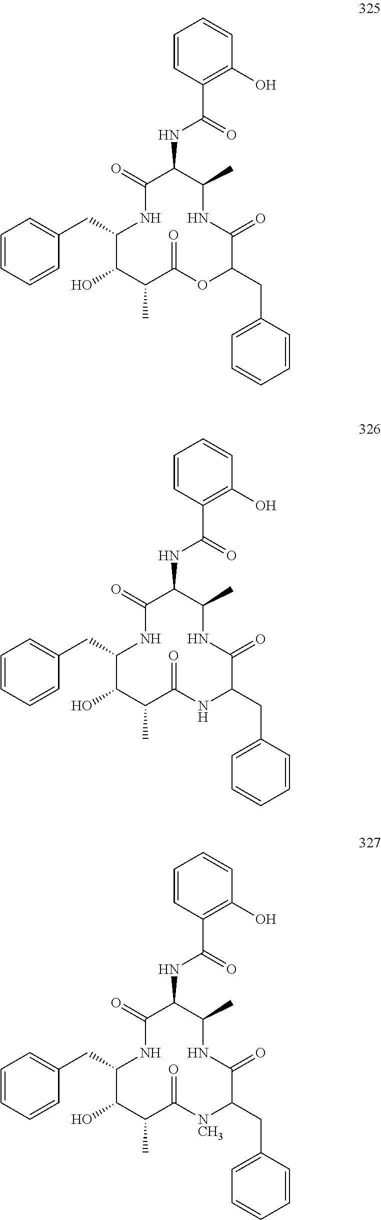 [SMILES: C[C@H]1NC(=O)C(Cc2ccccc2)OC(=O)[C@H](C)[C@H](O)[C@H](Cc2ccccc2)NC(=O)[C@H]1NC(=O)c1ccccc1O ; C[C@H]1NC(=O)C(Cc2ccccc2)NC(=O)[C@H](C)[C@H](O)[C@H](Cc2ccccc2)NC(=O)[C@H]1NC(=O)c1ccccc1O ; C[C@H]1NC(=O)C(Cc2ccccc2)N(C)C(=O)[C@H](C)[C@H](O)[C@H](Cc2ccccc2)NC(=O)[C@H]1NC(=O)c1ccccc1O]

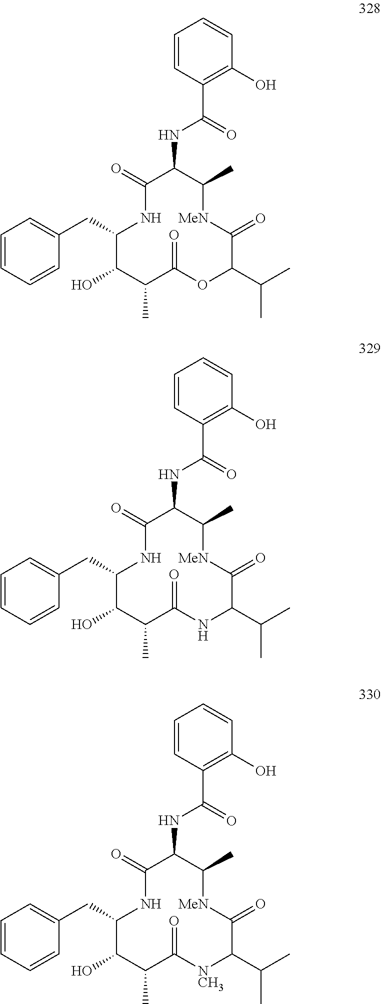 [SMILES: CC(C)C1OC(=O)[C@H](C)[C@H](O)[C@H](Cc2ccccc2)NC(=O)[C@@H](NC(=O)c2ccccc2O)[C@@H](C)N(C)C1=O ; CC(C)C1NC(=O)[C@H](C)[C@H](O)[C@H](Cc2ccccc2)NC(=O)[C@@H](NC(=O)c2ccccc2O)[C@@H](C)N(C)C1=O ; CC(C)C1C(=O)N(C)[C@H](C)[C@H](NC(=O)c2ccccc2O)C(=O)N[C@@H](Cc2ccccc2)[C@@H](O)[C@@H](C)C(=O)N1C]

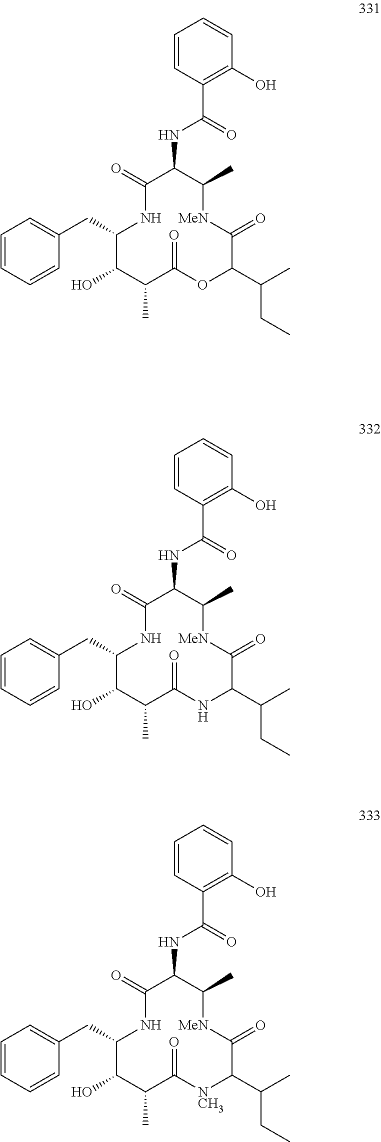 [SMILES: CCC(C)C1OC(=O)[C@H](C)[C@H](O)[C@H](Cc2ccccc2)NC(=O)[C@@H](NC(=O)c2ccccc2O)[C@@H](C)N(C)C1=O ; CCC(C)C1NC(=O)[C@H](C)[C@H](O)[C@H](Cc2ccccc2)NC(=O)[C@@H](NC(=O)c2ccccc2O)[C@@H](C)N(C)C1=O ; CCC(C)C1C(=O)N(C)[C@H](C)[C@H](NC(=O)c2ccccc2O)C(=O)N[C@@H](Cc2ccccc2)[C@@H](O)[C@@H](C)C(=O)N1C]

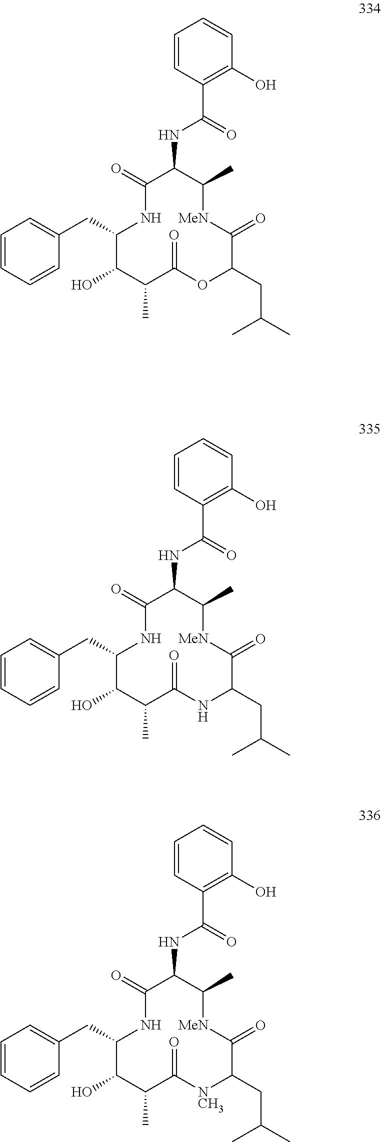 [SMILES: CC(C)CC1OC(=O)[C@H](C)[C@H](O)[C@H](Cc2ccccc2)NC(=O)[C@@H](NC(=O)c2ccccc2O)[C@@H](C)N(C)C1=O ; CC(C)CC1NC(=O)[C@H](C)[C@H](O)[C@H](Cc2ccccc2)NC(=O)[C@@H](NC(=O)c2ccccc2O)[C@@H](C)N(C)C1=O ; CC(C)CC1C(=O)N(C)[C@H](C)[C@H](NC(=O)c2ccccc2O)C(=O)N[C@@H](Cc2ccccc2)[C@@H](O)[C@@H](C)C(=O)N1C]

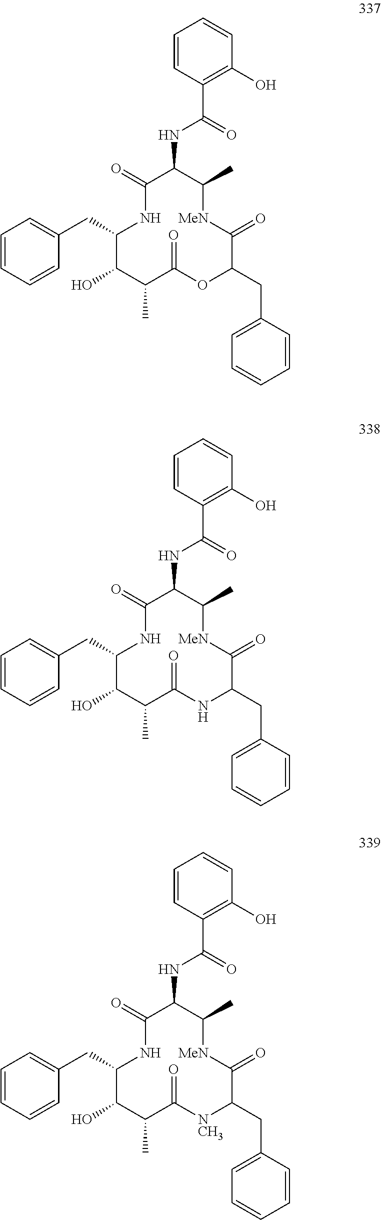 [SMILES: C[C@@H]1[C@H](NC(=O)c2ccccc2O)C(=O)N[C@@H](Cc2ccccc2)[C@@H](O)[C@@H](C)C(=O)OC(Cc2ccccc2)C(=O)N1C ; C[C@@H]1[C@H](NC(=O)c2ccccc2O)C(=O)N[C@@H](Cc2ccccc2)[C@@H](O)[C@@H](C)C(=O)NC(Cc2ccccc2)C(=O)N1C ; C[C@@H]1[C@H](NC(=O)c2ccccc2O)C(=O)N[C@@H](Cc2ccccc2)[C@@H](O)[C@@H](C)C(=O)N(C)C(Cc2ccccc2)C(=O)N1C]

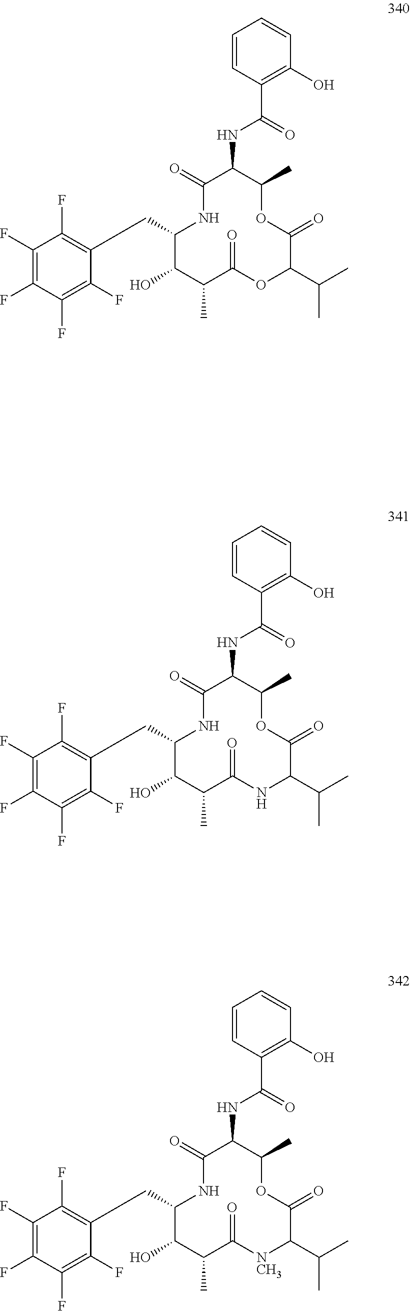 [SMILES: CC(C)C1OC(=O)[C@H](C)[C@H](O)[C@H](Cc2c(F)c(F)c(F)c(F)c2F)NC(=O)[C@@H](NC(=O)c2ccccc2O)[C@@H](C)OC1=O ; CC(C)C1NC(=O)[C@H](C)[C@H](O)[C@H](Cc2c(F)c(F)c(F)c(F)c2F)NC(=O)[C@@H](NC(=O)c2ccccc2O)[C@@H](C)OC1=O ; CC(C)C1C(=O)O[C@H](C)[C@H](NC(=O)c2ccccc2O)C(=O)N[C@@H](Cc2c(F)c(F)c(F)c(F)c2F)[C@@H](O)[C@@H](C)C(=O)N1C]

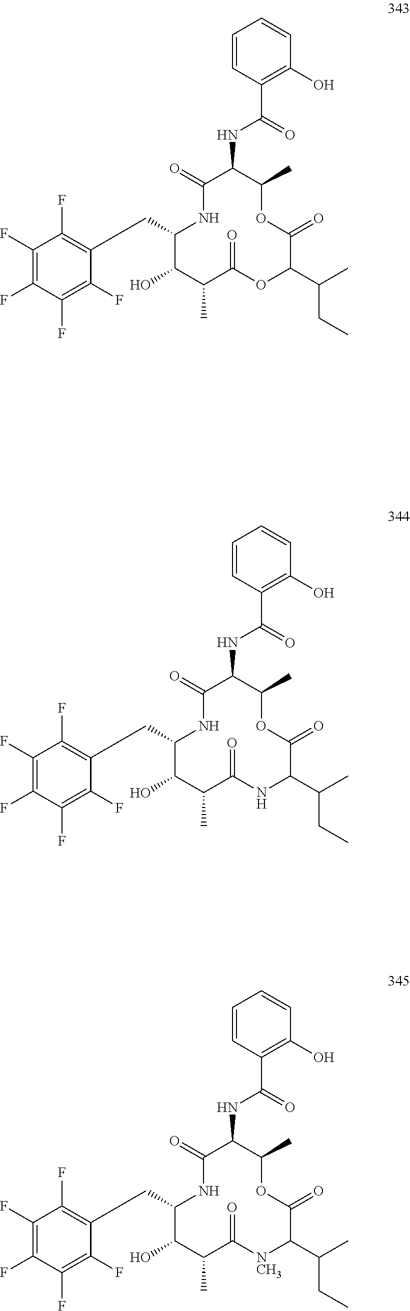 [SMILES: CCC(C)C1OC(=O)[C@H](C)[C@H](O)[C@H](Cc2c(F)c(F)c(F)c(F)c2F)NC(=O)[C@@H](NC(=O)c2ccccc2O)[C@@H](C)OC1=O ; CCC(C)C1NC(=O)[C@H](C)[C@H](O)[C@H](Cc2c(F)c(F)c(F)c(F)c2F)NC(=O)[C@@H](NC(=O)c2ccccc2O)[C@@H](C)OC1=O ; CCC(C)C1C(=O)O[C@H](C)[C@H](NC(=O)c2ccccc2O)C(=O)N[C@@H](Cc2c(F)c(F)c(F)c(F)c2F)[C@@H](O)[C@@H](C)C(=O)N1C]

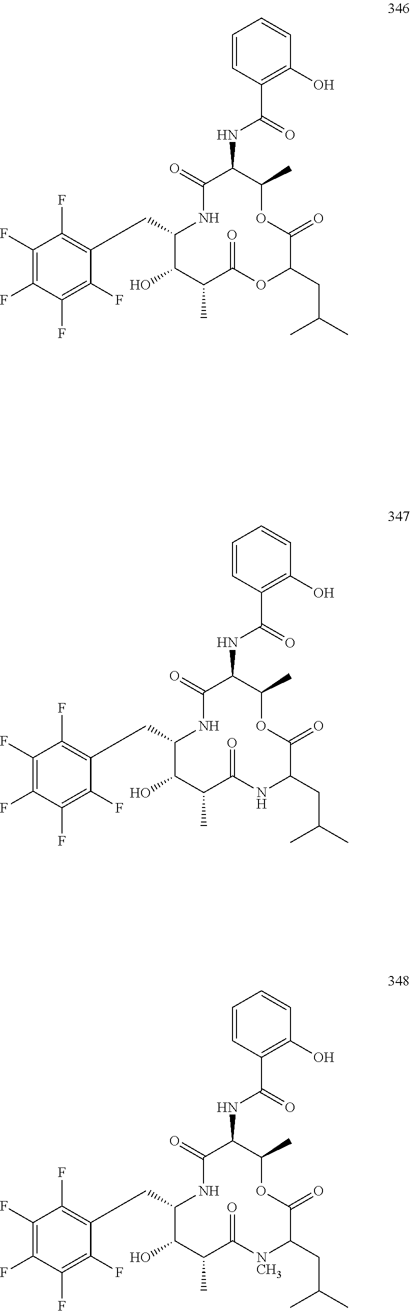 [SMILES: CC(C)CC1OC(=O)[C@H](C)[C@H](O)[C@H](Cc2c(F)c(F)c(F)c(F)c2F)NC(=O)[C@@H](NC(=O)c2ccccc2O)[C@@H](C)OC1=O ; CC(C)CC1NC(=O)[C@H](C)[C@H](O)[C@H](Cc2c(F)c(F)c(F)c(F)c2F)NC(=O)[C@@H](NC(=O)c2ccccc2O)[C@@H](C)OC1=O ; CC(C)CC1C(=O)O[C@H](C)[C@H](NC(=O)c2ccccc2O)C(=O)N[C@@H](Cc2c(F)c(F)c(F)c(F)c2F)[C@@H](O)[C@@H](C)C(=O)N1C]

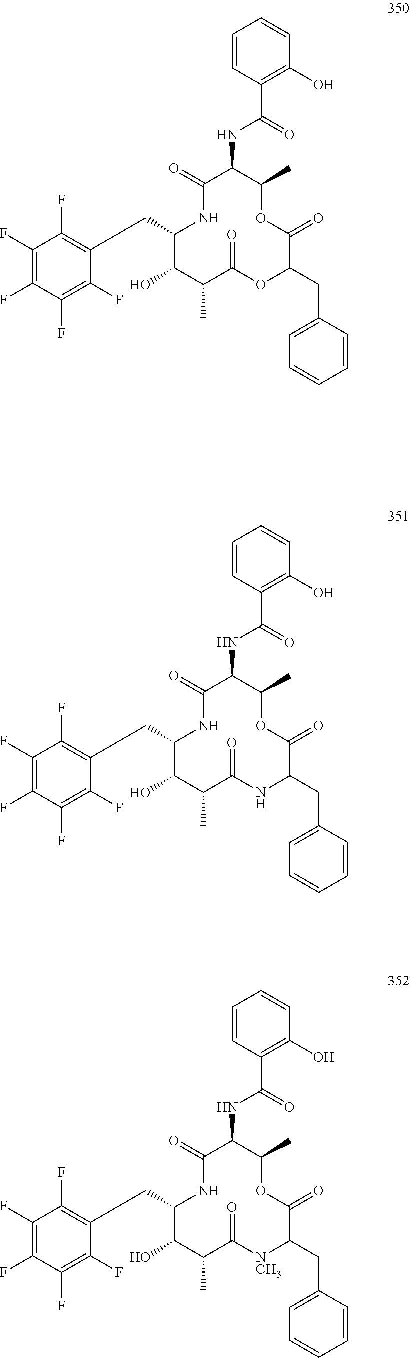 [SMILES: C[C@H]1OC(=O)C(Cc2ccccc2)OC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(Cc2ccccc2)NC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(Cc2ccccc2)N(C)C(=O)[C@H](C)[C@H](O)[C@H](Cc2c(F)c(F)c(F)c(F)c2F)NC(=O)[C@H]1NC(=O)c1ccccc1O]

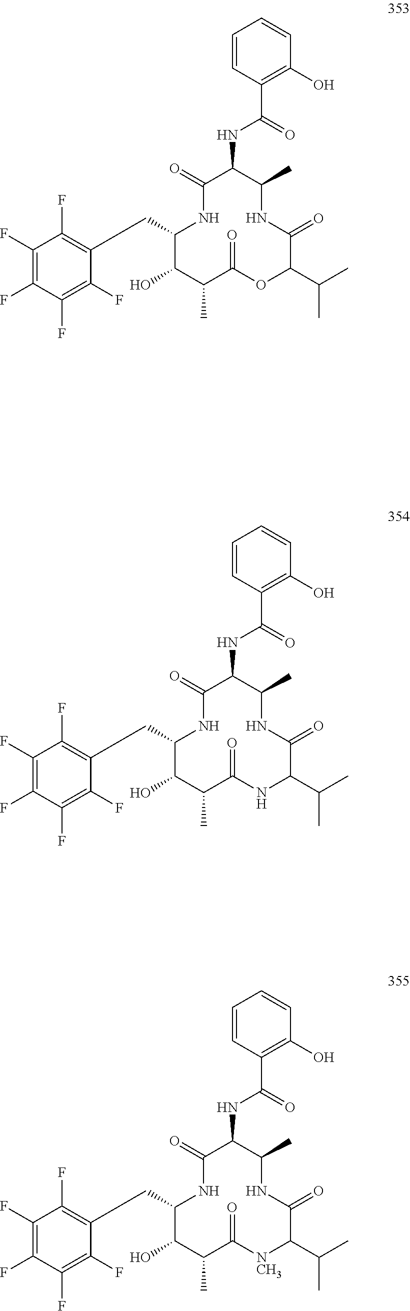 [SMILES: CC(C)C1OC(=O)[C@H](C)[C@H](O)[C@H](Cc2c(F)c(F)c(F)c(F)c2F)NC(=O)[C@@H](NC(=O)c2ccccc2O)[C@@H](C)NC1=O ; CC(C)C1NC(=O)[C@H](C)[C@H](O)[C@H](Cc2c(F)c(F)c(F)c(F)c2F)NC(=O)[C@@H](NC(=O)c2ccccc2O)[C@@H](C)NC1=O ; CC(C)C1C(=O)N[C@H](C)[C@H](NC(=O)c2ccccc2O)C(=O)N[C@@H](Cc2c(F)c(F)c(F)c(F)c2F)[C@@H](O)[C@@H](C)C(=O)N1C]

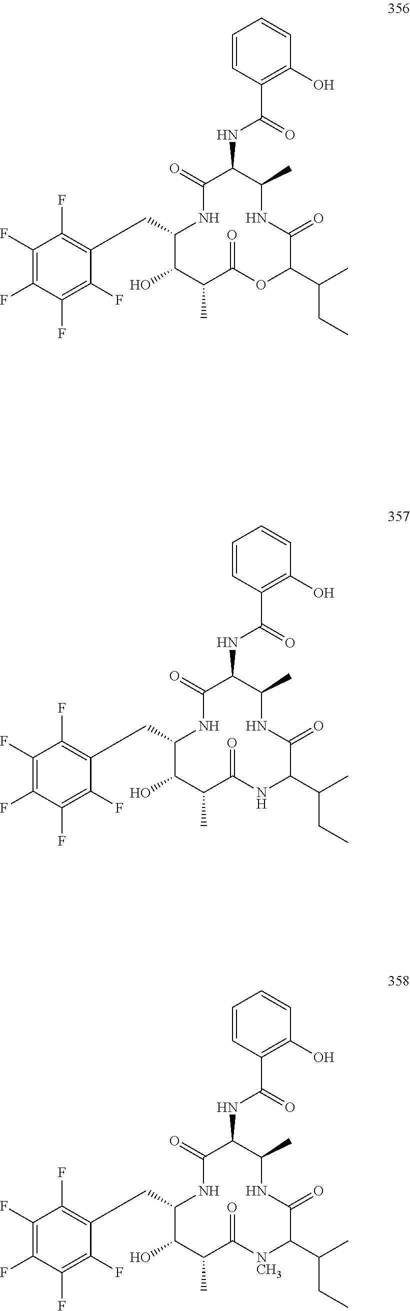 [SMILES: CCC(C)C1OC(=O)[C@H](C)[C@H](O)[C@H](Cc2c(F)c(F)c(F)c(F)c2F)NC(=O)[C@@H](NC(=O)c2ccccc2O)[C@@H](C)NC1=O ; CCC(C)C1NC(=O)[C@H](C)[C@H](O)[C@H](Cc2c(F)c(F)c(F)c(F)c2F)NC(=O)[C@@H](NC(=O)c2ccccc2O)[C@@H](C)NC1=O ; CCC(C)C1C(=O)N[C@H](C)[C@H](NC(=O)c2ccccc2O)C(=O)N[C@@H](Cc2c(F)c(F)c(F)c(F)c2F)[C@@H](O)[C@@H](C)C(=O)N1C]

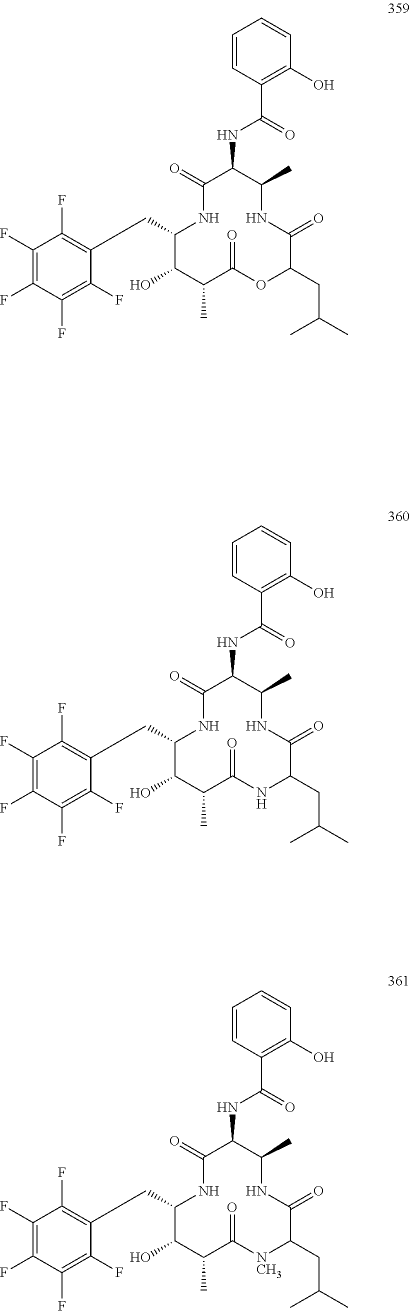 [SMILES: CC(C)CC1OC(=O)[C@H](C)[C@H](O)[C@H](Cc2c(F)c(F)c(F)c(F)c2F)NC(=O)[C@@H](NC(=O)c2ccccc2O)[C@@H](C)NC1=O ; CC(C)CC1NC(=O)[C@H](C)[C@H](O)[C@H](Cc2c(F)c(F)c(F)c(F)c2F)NC(=O)[C@@H](NC(=O)c2ccccc2O)[C@@H](C)NC1=O ; CC(C)CC1C(=O)N[C@H](C)[C@H](NC(=O)c2ccccc2O)C(=O)N[C@@H](Cc2c(F)c(F)c(F)c(F)c2F)[C@@H](O)[C@@H](C)C(=O)N1C]

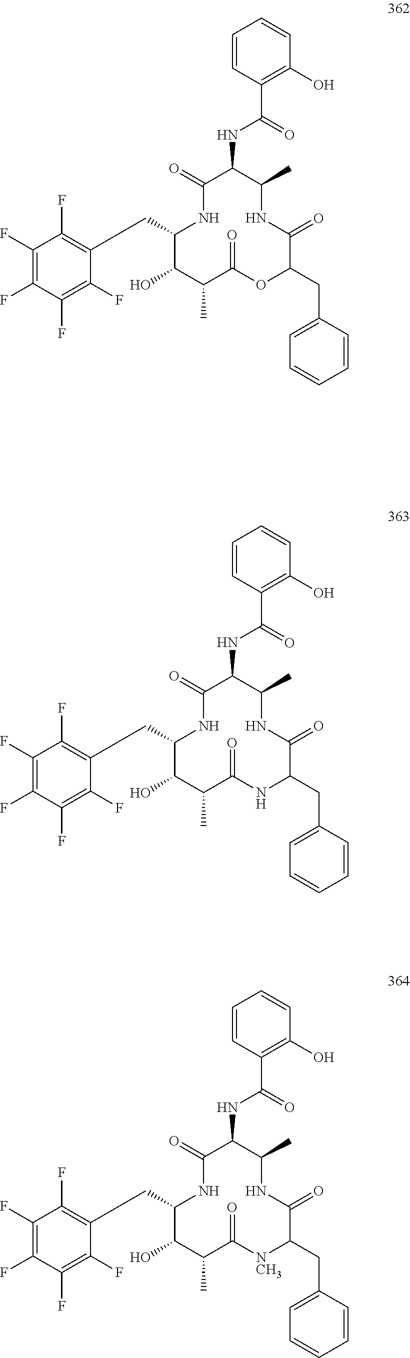 [SMILES: C[C@H]1NC(=O)C(Cc2ccccc2)OC(=O)[C@H](C)[C@H](O)[C@H](Cc2c(F)c(F)c(F)c(F)c2F)NC(=O)[C@H]1NC(=O)c1ccccc1O ; C[C@H]1NC(=O)C(Cc2ccccc2)NC(=O)[C@H](C)[C@H](O)[C@H](Cc2c(F)c(F)c(F)c(F)c2F)NC(=O)[C@H]1NC(=O)c1ccccc1O ; C[C@H]1NC(=O)C(Cc2ccccc2)N(C)C(=O)[C@H](C)[C@H](O)[C@H](Cc2c(F)c(F)c(F)c(F)c2F)NC(=O)[C@H]1NC(=O)c1ccccc1O]

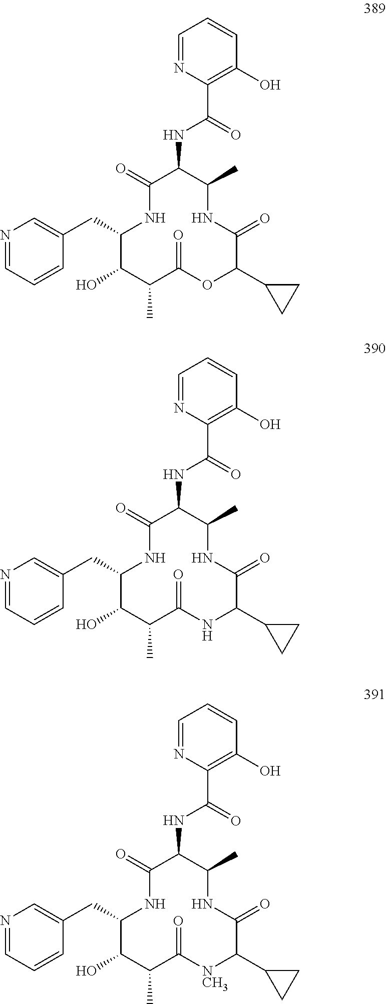 [SMILES: C[C@H]1NC(=O)C(C2CC2)OC(=O)[C@H](C)[C@H](O)[C@H](Cc2cccnc2)NC(=O)[C@H]1NC(=O)c1ncccc1O ; C[C@H]1NC(=O)C(C2CC2)NC(=O)[C@H](C)[C@H](O)[C@H](Cc2cccnc2)NC(=O)[C@H]1NC(=O)c1ncccc1O ; C[C@H]1NC(=O)C(C2CC2)N(C)C(=O)[C@H](C)[C@H](O)[C@H](Cc2cccnc2)NC(=O)[C@H]1NC(=O)c1ncccc1O]

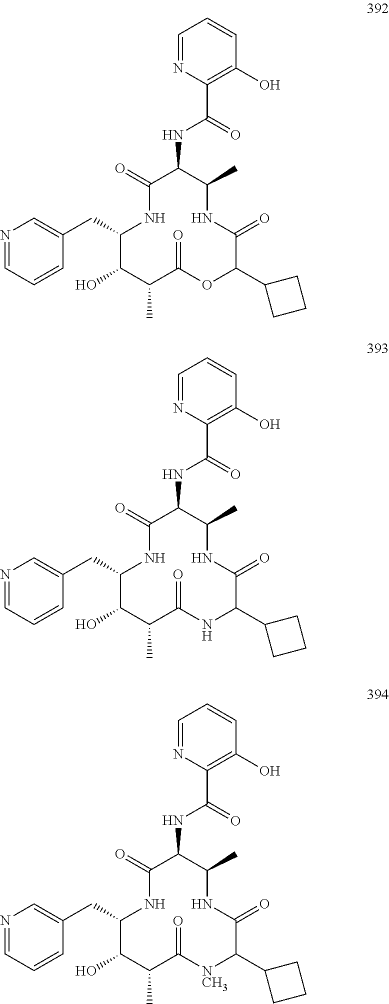 [SMILES: C[C@H]1NC(=O)C(C2CCC2)OC(=O)[C@H](C)[C@H](O)[C@H](Cc2cccnc2)NC(=O)[C@H]1NC(=O)c1ncccc1O ; C[C@H]1NC(=O)C(C2CCC2)NC(=O)[C@H](C)[C@H](O)[C@H](Cc2cccnc2)NC(=O)[C@H]1NC(=O)c1ncccc1O ; C[C@H]1NC(=O)C(C2CCC2)N(C)C(=O)[C@H](C)[C@H](O)[C@H](Cc2cccnc2)NC(=O)[C@H]1NC(=O)c1ncccc1O]

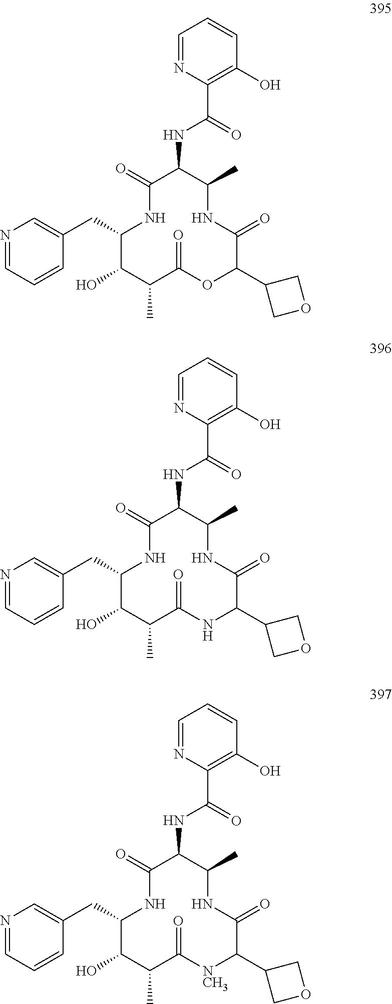 [SMILES: C[C@H]1NC(=O)C(C2COC2)OC(=O)[C@H](C)[C@H](O)[C@H](Cc2cccnc2)NC(=O)[C@H]1NC(=O)c1ncccc1O ; C[C@H]1NC(=O)C(C2COC2)NC(=O)[C@H](C)[C@H](O)[C@H](Cc2cccnc2)NC(=O)[C@H]1NC(=O)c1ncccc1O ; C[C@H]1NC(=O)C(C2COC2)N(C)C(=O)[C@H](C)[C@H](O)[C@H](Cc2cccnc2)NC(=O)[C@H]1NC(=O)c1ncccc1O]

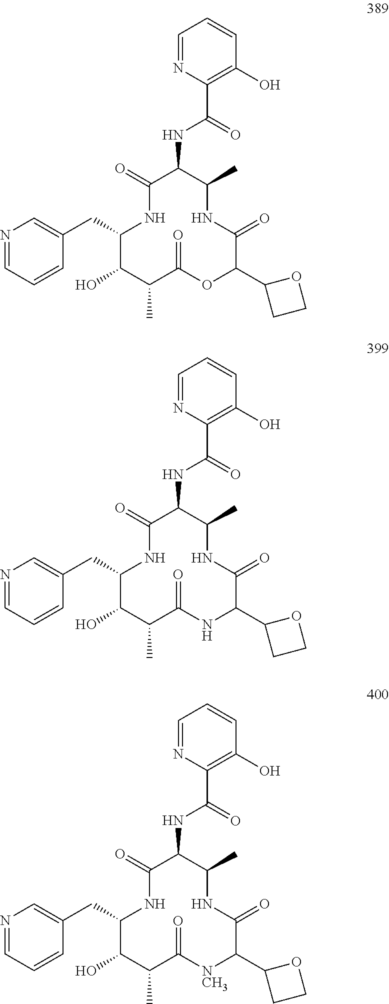 [SMILES: C[C@H]1NC(=O)C(C2CCO2)OC(=O)[C@H](C)[C@H](O)[C@H](Cc2cccnc2)NC(=O)[C@H]1NC(=O)c1ncccc1O ; C[C@H]1NC(=O)C(C2CCO2)NC(=O)[C@H](C)[C@H](O)[C@H](Cc2cccnc2)NC(=O)[C@H]1NC(=O)c1ncccc1O ; C[C@H]1NC(=O)C(C2CCO2)N(C)C(=O)[C@H](C)[C@H](O)[C@H](Cc2cccnc2)NC(=O)[C@H]1NC(=O)c1ncccc1O]

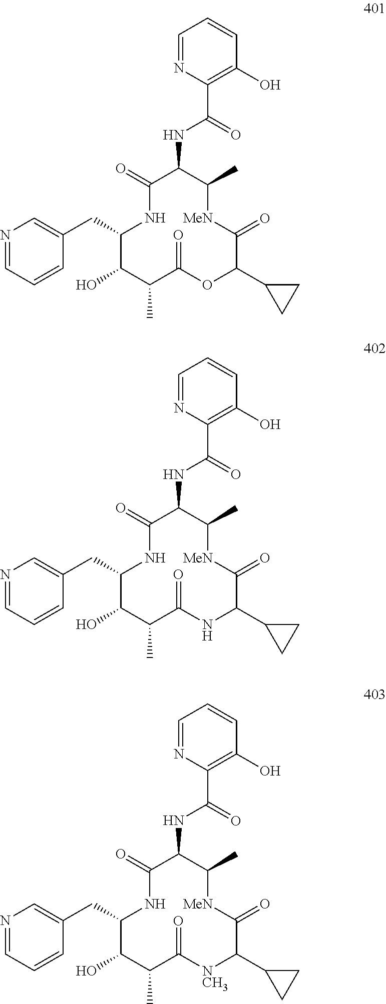 [SMILES: C[C@@H]1[C@H](NC(=O)c2ncccc2O)C(=O)N[C@@H](Cc2cccnc2)[C@@H](O)[C@@H](C)C(=O)OC(C2CC2)C(=O)N1C ; C[C@@H]1[C@H](NC(=O)c2ncccc2O)C(=O)N[C@@H](Cc2cccnc2)[C@@H](O)[C@@H](C)C(=O)NC(C2CC2)C(=O)N1C ; C[C@@H]1[C@H](NC(=O)c2ncccc2O)C(=O)N[C@@H](Cc2cccnc2)[C@@H](O)[C@@H](C)C(=O)N(C)C(C2CC2)C(=O)N1C]

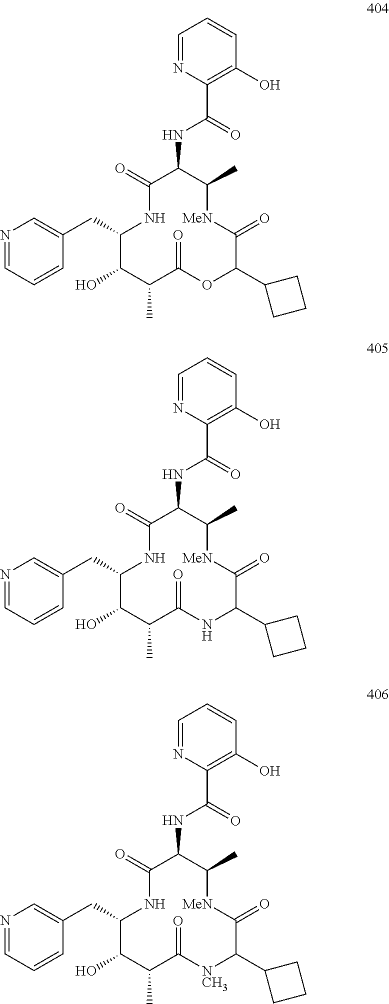 [SMILES: C[C@@H]1[C@H](NC(=O)c2ncccc2O)C(=O)N[C@@H](Cc2cccnc2)[C@@H](O)[C@@H](C)C(=O)OC(C2CCC2)C(=O)N1C ; C[C@@H]1[C@H](NC(=O)c2ncccc2O)C(=O)N[C@@H](Cc2cccnc2)[C@@H](O)[C@@H](C)C(=O)NC(C2CCC2)C(=O)N1C ; C[C@@H]1[C@H](NC(=O)c2ncccc2O)C(=O)N[C@@H](Cc2cccnc2)[C@@H](O)[C@@H](C)C(=O)N(C)C(C2CCC2)C(=O)N1C]

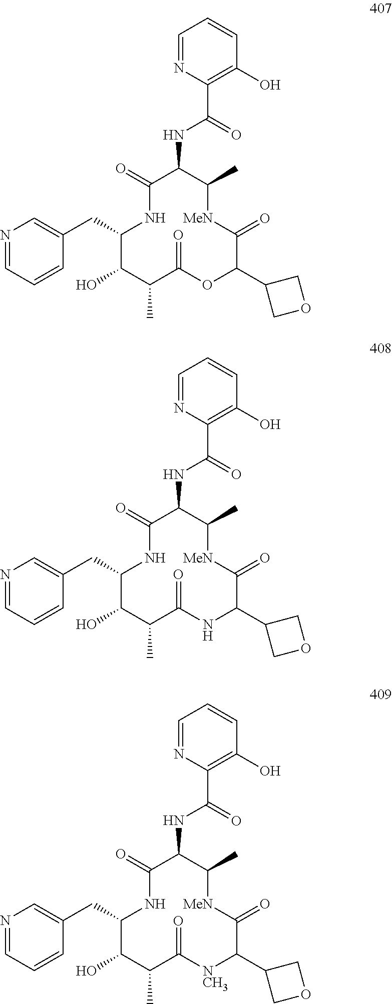 [SMILES: C[C@@H]1[C@H](NC(=O)c2ncccc2O)C(=O)N[C@@H](Cc2cccnc2)[C@@H](O)[C@@H](C)C(=O)OC(C2COC2)C(=O)N1C ; C[C@@H]1[C@H](NC(=O)c2ncccc2O)C(=O)N[C@@H](Cc2cccnc2)[C@@H](O)[C@@H](C)C(=O)NC(C2COC2)C(=O)N1C ; C[C@@H]1[C@H](NC(=O)c2ncccc2O)C(=O)N[C@@H](Cc2cccnc2)[C@@H](O)[C@@H](C)C(=O)N(C)C(C2COC2)C(=O)N1C]

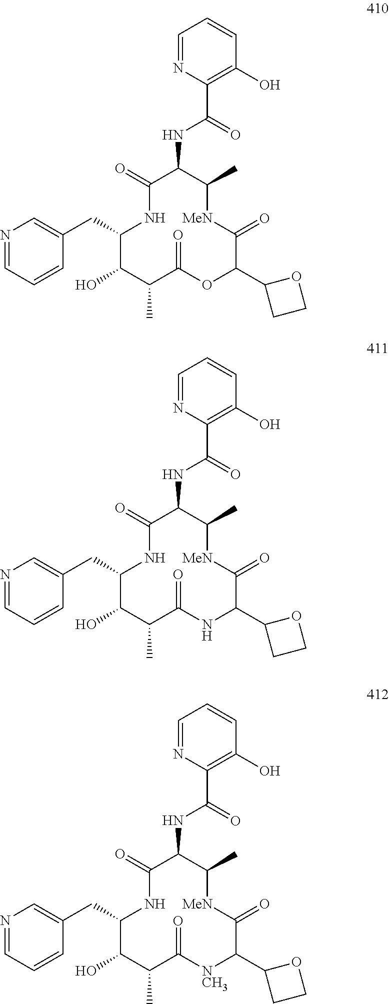 [SMILES: C[C@@H]1[C@H](NC(=O)c2ncccc2O)C(=O)N[C@@H](Cc2cccnc2)[C@@H](O)[C@@H](C)C(=O)OC(C2CCO2)C(=O)N1C ; C[C@@H]1[C@H](NC(=O)c2ncccc2O)C(=O)N[C@@H](Cc2cccnc2)[C@@H](O)[C@@H](C)C(=O)NC(C2CCO2)C(=O)N1C ; C[C@@H]1[C@H](NC(=O)c2ncccc2O)C(=O)N[C@@H](Cc2cccnc2)[C@@H](O)[C@@H](C)C(=O)N(C)C(C2CCO2)C(=O)N1C]

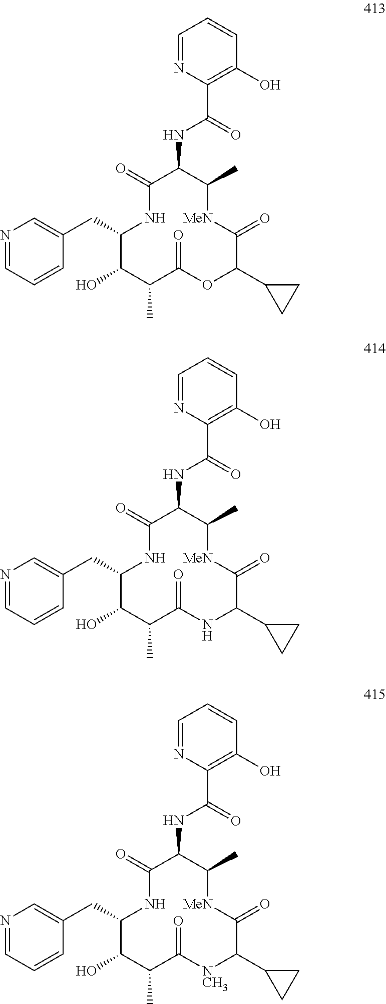 [SMILES: C[C@@H]1[C@H](NC(=O)c2ncccc2O)C(=O)N[C@@H](Cc2cccnc2)[C@@H](O)[C@@H](C)C(=O)OC(C2CC2)C(=O)N1C ; C[C@@H]1[C@H](NC(=O)c2ncccc2O)C(=O)N[C@@H](Cc2cccnc2)[C@@H](O)[C@@H](C)C(=O)NC(C2CC2)C(=O)N1C ; C[C@@H]1[C@H](NC(=O)c2ncccc2O)C(=O)N[C@@H](Cc2cccnc2)[C@@H](O)[C@@H](C)C(=O)N(C)C(C2CC2)C(=O)N1C]

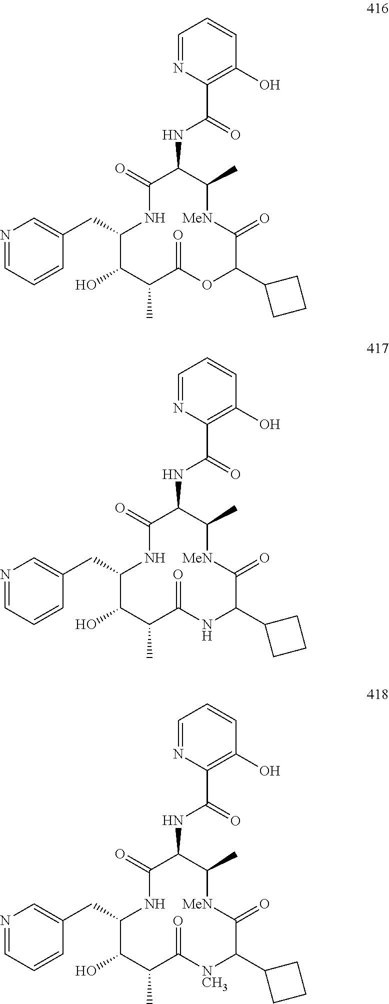 [SMILES: C[C@@H]1[C@H](NC(=O)c2ncccc2O)C(=O)N[C@@H](Cc2cccnc2)[C@@H](O)[C@@H](C)C(=O)OC(C2CCC2)C(=O)N1C ; C[C@@H]1[C@H](NC(=O)c2ncccc2O)C(=O)N[C@@H](Cc2cccnc2)[C@@H](O)[C@@H](C)C(=O)NC(C2CCC2)C(=O)N1C ; C[C@@H]1[C@H](NC(=O)c2ncccc2O)C(=O)N[C@@H](Cc2cccnc2)[C@@H](O)[C@@H](C)C(=O)N(C)C(C2CCC2)C(=O)N1C]

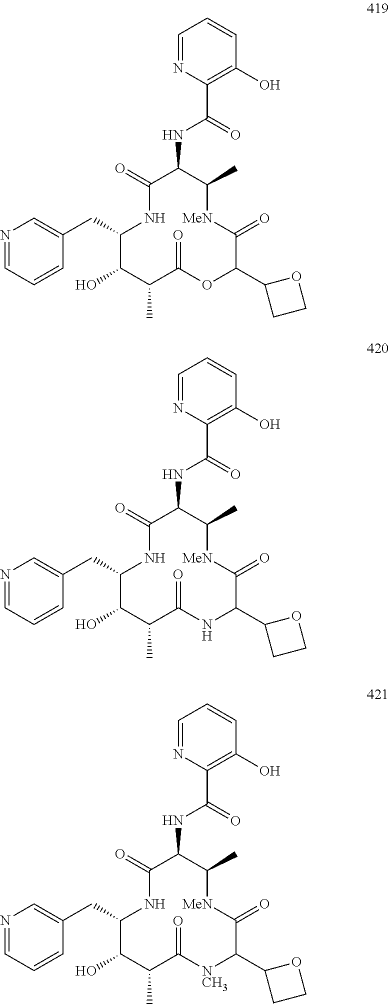 [SMILES: C[C@@H]1[C@H](NC(=O)c2ncccc2O)C(=O)N[C@@H](Cc2cccnc2)[C@@H](O)[C@@H](C)C(=O)OC(C2CCO2)C(=O)N1C ; C[C@@H]1[C@H](NC(=O)c2ncccc2O)C(=O)N[C@@H](Cc2cccnc2)[C@@H](O)[C@@H](C)C(=O)NC(C2CCO2)C(=O)N1C ; C[C@@H]1[C@H](NC(=O)c2ncccc2O)C(=O)N[C@@H](Cc2cccnc2)[C@@H](O)[C@@H](C)C(=O)N(C)C(C2CCO2)C(=O)N1C]

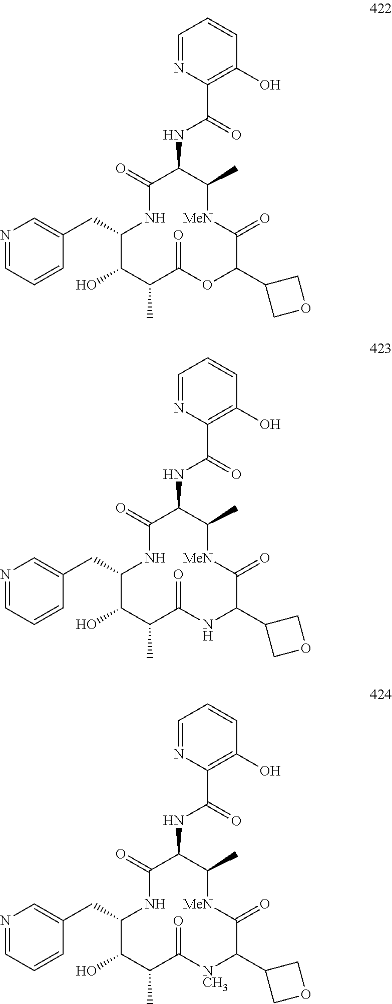 [SMILES: C[C@@H]1[C@H](NC(=O)c2ncccc2O)C(=O)N[C@@H](Cc2cccnc2)[C@@H](O)[C@@H](C)C(=O)OC(C2COC2)C(=O)N1C ; C[C@@H]1[C@H](NC(=O)c2ncccc2O)C(=O)N[C@@H](Cc2cccnc2)[C@@H](O)[C@@H](C)C(=O)NC(C2COC2)C(=O)N1C ; C[C@@H]1[C@H](NC(=O)c2ncccc2O)C(=O)N[C@@H](Cc2cccnc2)[C@@H](O)[C@@H](C)C(=O)N(C)C(C2COC2)C(=O)N1C]

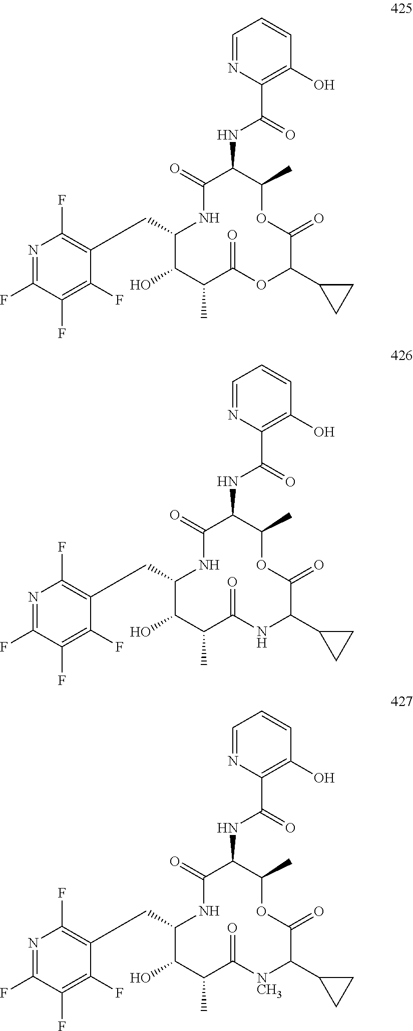 [SMILES: C[C@H]1OC(=O)C(C2CC2)OC(=O)[C@H](C)[C@H](O)[C@H](Cc2c(F)nc(F)c(F)c2F)NC(=O)[C@H]1NC(=O)c1ncccc1O ; C[C@H]1OC(=O)C(C2CC2)NC(=O)[C@H](C)[C@H](O)[C@H](Cc2c(F)nc(F)c(F)c2F)NC(=O)[C@H]1NC(=O)c1ncccc1O ; C[C@H]1OC(=O)C(C2CC2)N(C)C(=O)[C@H](C)[C@H](O)[C@H](Cc2c(F)nc(F)c(F)c2F)NC(=O)[C@H]1NC(=O)c1ncccc1O]

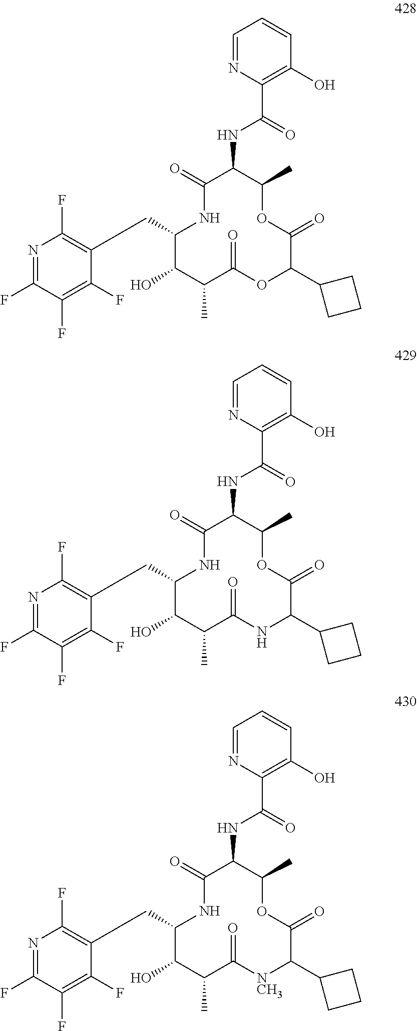 [SMILES: C[C@H]1OC(=O)C(C2CCC2)OC(=O)[C@H](C)[C@H](O)[C@H](Cc2c(F)nc(F)c(F)c2F)NC(=O)[C@H]1NC(=O)c1ncccc1O ; C[C@H]1OC(=O)C(C2CCC2)NC(=O)[C@H](C)[C@H](O)[C@H](Cc2c(F)nc(F)c(F)c2F)NC(=O)[C@H]1NC(=O)c1ncccc1O ; C[C@H]1OC(=O)C(C2CCC2)N(C)C(=O)[C@H](C)[C@H](O)[C@H](Cc2c(F)nc(F)c(F)c2F)NC(=O)[C@H]1NC(=O)c1ncccc1O]

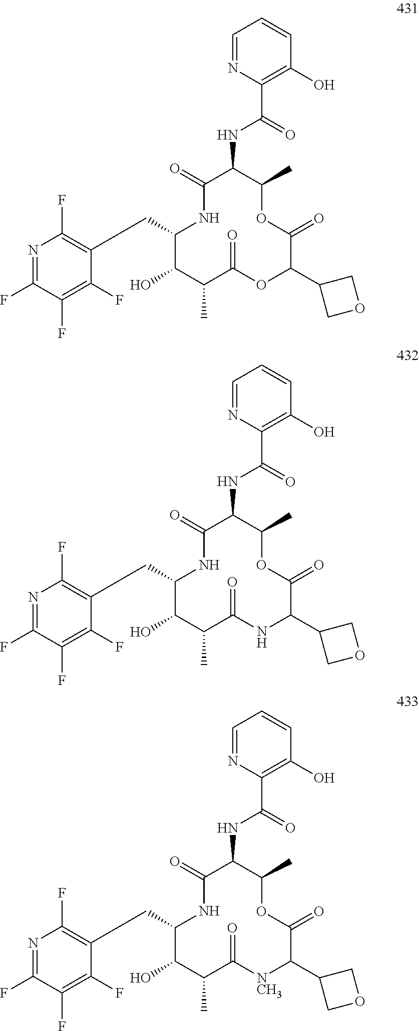 [SMILES: C[C@H]1OC(=O)C(C2COC2)OC(=O)[C@H](C)[C@H](O)[C@H](Cc2c(F)nc(F)c(F)c2F)NC(=O)[C@H]1NC(=O)c1ncccc1O ; C[C@H]1OC(=O)C(C2COC2)NC(=O)[C@H](C)[C@H](O)[C@H](Cc2c(F)nc(F)c(F)c2F)NC(=O)[C@H]1NC(=O)c1ncccc1O ; C[C@H]1OC(=O)C(C2COC2)N(C)C(=O)[C@H](C)[C@H](O)[C@H](Cc2c(F)nc(F)c(F)c2F)NC(=O)[C@H]1NC(=O)c1ncccc1O]

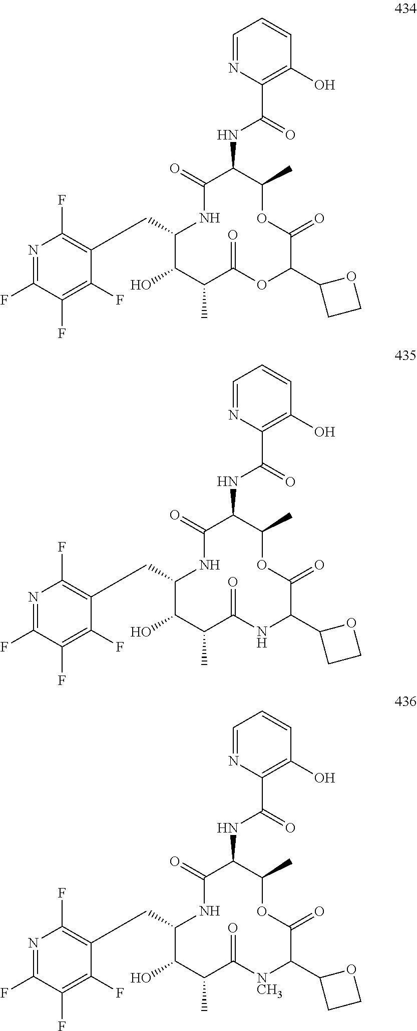 [SMILES: C[C@H]1OC(=O)C(C2CCO2)OC(=O)[C@H](C)[C@H](O)[C@H](Cc2c(F)nc(F)c(F)c2F)NC(=O)[C@H]1NC(=O)c1ncccc1O ; C[C@H]1OC(=O)C(C2CCO2)NC(=O)[C@H](C)[C@H](O)[C@H](Cc2c(F)nc(F)c(F)c2F)NC(=O)[C@H]1NC(=O)c1ncccc1O ; C[C@H]1OC(=O)C(C2CCO2)N(C)C(=O)[C@H](C)[C@H](O)[C@H](Cc2c(F)nc(F)c(F)c2F)NC(=O)[C@H]1NC(=O)c1ncccc1O]

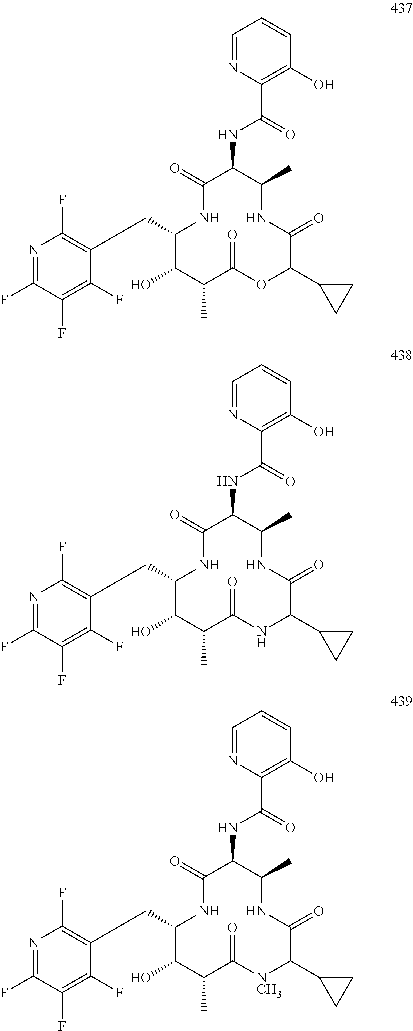 [SMILES: C[C@H]1NC(=O)C(C2CC2)OC(=O)[C@H](C)[C@H](O)[C@H](Cc2c(F)nc(F)c(F)c2F)NC(=O)[C@H]1NC(=O)c1ncccc1O ; C[C@H]1NC(=O)C(C2CC2)NC(=O)[C@H](C)[C@H](O)[C@H](Cc2c(F)nc(F)c(F)c2F)NC(=O)[C@H]1NC(=O)c1ncccc1O ; C[C@H]1NC(=O)C(C2CC2)N(C)C(=O)[C@H](C)[C@H](O)[C@H](Cc2c(F)nc(F)c(F)c2F)NC(=O)[C@H]1NC(=O)c1ncccc1O]

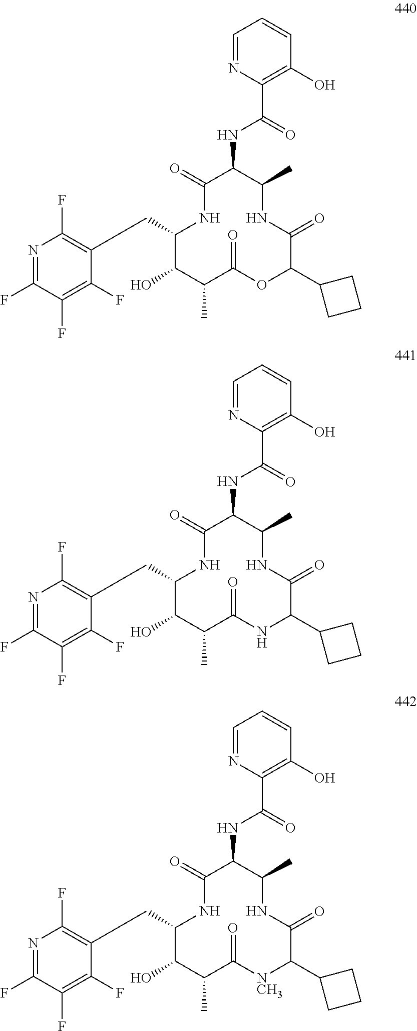 [SMILES: C[C@H]1NC(=O)C(C2CCC2)OC(=O)[C@H](C)[C@H](O)[C@H](Cc2c(F)nc(F)c(F)c2F)NC(=O)[C@H]1NC(=O)c1ncccc1O ; C[C@H]1NC(=O)C(C2CCC2)NC(=O)[C@H](C)[C@H](O)[C@H](Cc2c(F)nc(F)c(F)c2F)NC(=O)[C@H]1NC(=O)c1ncccc1O ; C[C@H]1NC(=O)C(C2CCC2)N(C)C(=O)[C@H](C)[C@H](O)[C@H](Cc2c(F)nc(F)c(F)c2F)NC(=O)[C@H]1NC(=O)c1ncccc1O]

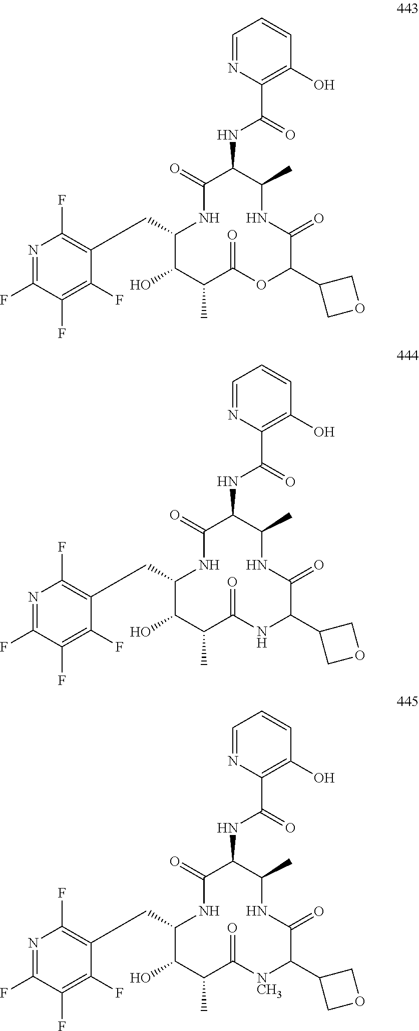 [SMILES: C[C@H]1NC(=O)C(C2COC2)OC(=O)[C@H](C)[C@H](O)[C@H](Cc2c(F)nc(F)c(F)c2F)NC(=O)[C@H]1NC(=O)c1ncccc1O ; C[C@H]1NC(=O)C(C2COC2)NC(=O)[C@H](C)[C@H](O)[C@H](Cc2c(F)nc(F)c(F)c2F)NC(=O)[C@H]1NC(=O)c1ncccc1O ; C[C@H]1NC(=O)C(C2COC2)N(C)C(=O)[C@H](C)[C@H](O)[C@H](Cc2c(F)nc(F)c(F)c2F)NC(=O)[C@H]1NC(=O)c1ncccc1O]

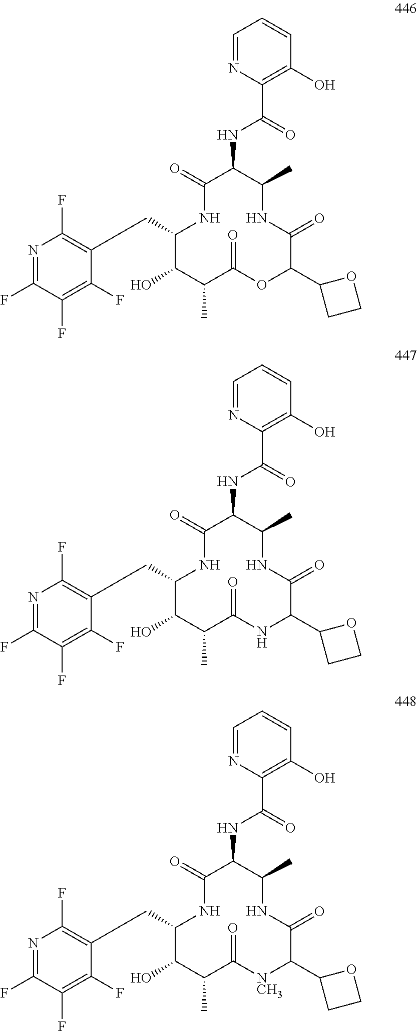 [SMILES: C[C@H]1NC(=O)C(C2CCO2)OC(=O)[C@H](C)[C@H](O)[C@H](Cc2c(F)nc(F)c(F)c2F)NC(=O)[C@H]1NC(=O)c1ncccc1O ; C[C@H]1NC(=O)C(C2CCO2)NC(=O)[C@H](C)[C@H](O)[C@H](Cc2c(F)nc(F)c(F)c2F)NC(=O)[C@H]1NC(=O)c1ncccc1O ; C[C@H]1NC(=O)C(C2CCO2)N(C)C(=O)[C@H](C)[C@H](O)[C@H](Cc2c(F)nc(F)c(F)c2F)NC(=O)[C@H]1NC(=O)c1ncccc1O]

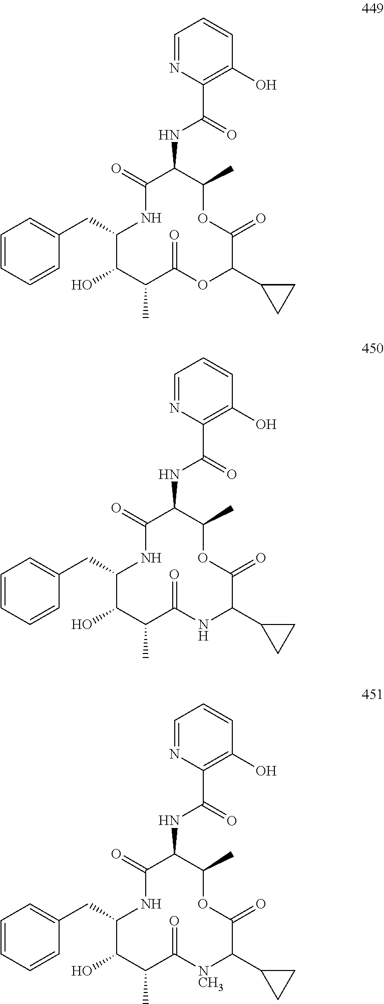 [SMILES: C[C@H]1OC(=O)C(C2CC2)OC(=O)[C@H](C)[C@H](O)[C@H](Cc2ccccc2)NC(=O)[C@H]1NC(=O)c1ncccc1O ; C[C@H]1OC(=O)C(C2CC2)NC(=O)[C@H](C)[C@H](O)[C@H](Cc2ccccc2)NC(=O)[C@H]1NC(=O)c1ncccc1O ; C[C@H]1OC(=O)C(C2CC2)N(C)C(=O)[C@H](C)[C@H](O)[C@H](Cc2ccccc2)NC(=O)[C@H]1NC(=O)c1ncccc1O]

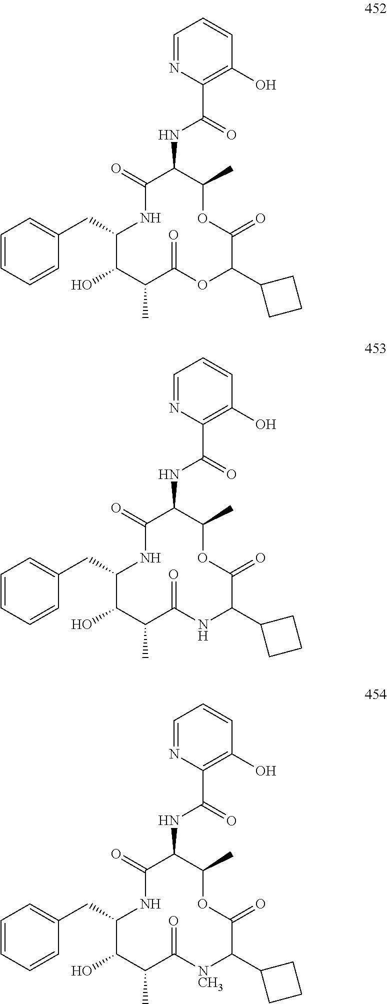 [SMILES: C[C@H]1OC(=O)C(C2CCC2)OC(=O)[C@H](C)[C@H](O)[C@H](Cc2ccccc2)NC(=O)[C@H]1NC(=O)c1ncccc1O ; C[C@H]1OC(=O)C(C2CCC2)NC(=O)[C@H](C)[C@H](O)[C@H](Cc2ccccc2)NC(=O)[C@H]1NC(=O)c1ncccc1O ; C[C@H]1OC(=O)C(C2CCC2)N(C)C(=O)[C@H](C)[C@H](O)[C@H](Cc2ccccc2)NC(=O)[C@H]1NC(=O)c1ncccc1O]

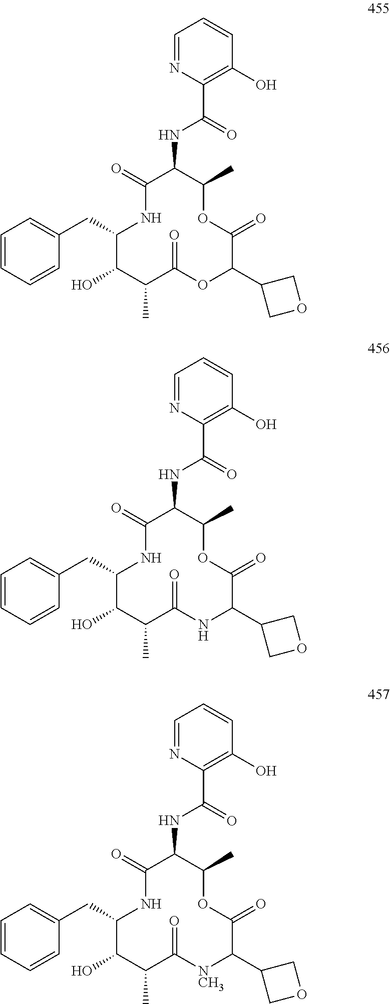 [SMILES: C[C@H]1OC(=O)C(C2COC2)OC(=O)[C@H](C)[C@H](O)[C@H](Cc2ccccc2)NC(=O)[C@H]1NC(=O)c1ncccc1O ; C[C@H]1OC(=O)C(C2COC2)NC(=O)[C@H](C)[C@H](O)[C@H](Cc2ccccc2)NC(=O)[C@H]1NC(=O)c1ncccc1O ; C[C@H]1OC(=O)C(C2COC2)N(C)C(=O)[C@H](C)[C@H](O)[C@H](Cc2ccccc2)NC(=O)[C@H]1NC(=O)c1ncccc1O]

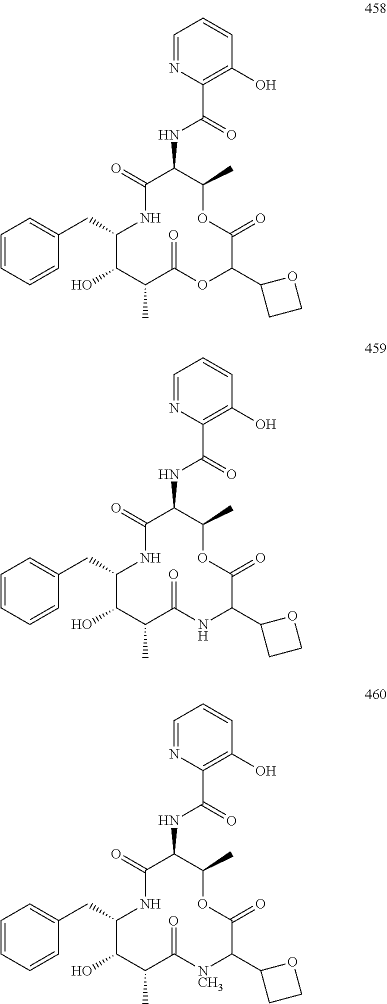 [SMILES: C[C@H]1OC(=O)C(C2CCO2)OC(=O)[C@H](C)[C@H](O)[C@H](Cc2ccccc2)NC(=O)[C@H]1NC(=O)c1ncccc1O ; C[C@H]1OC(=O)C(C2CCO2)NC(=O)[C@H](C)[C@H](O)[C@H](Cc2ccccc2)NC(=O)[C@H]1NC(=O)c1ncccc1O ; C[C@H]1OC(=O)C(C2CCO2)N(C)C(=O)[C@H](C)[C@H](O)[C@H](Cc2ccccc2)NC(=O)[C@H]1NC(=O)c1ncccc1O]

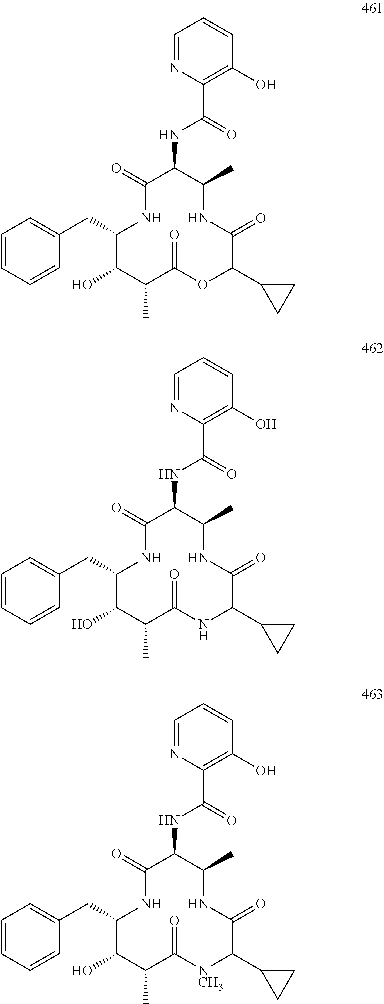 [SMILES: C[C@H]1NC(=O)C(C2CC2)OC(=O)[C@H](C)[C@H](O)[C@H](Cc2ccccc2)NC(=O)[C@H]1NC(=O)c1ncccc1O ; C[C@H]1NC(=O)C(C2CC2)NC(=O)[C@H](C)[C@H](O)[C@H](Cc2ccccc2)NC(=O)[C@H]1NC(=O)c1ncccc1O ; C[C@H]1NC(=O)C(C2CC2)N(C)C(=O)[C@H](C)[C@H](O)[C@H](Cc2ccccc2)NC(=O)[C@H]1NC(=O)c1ncccc1O]

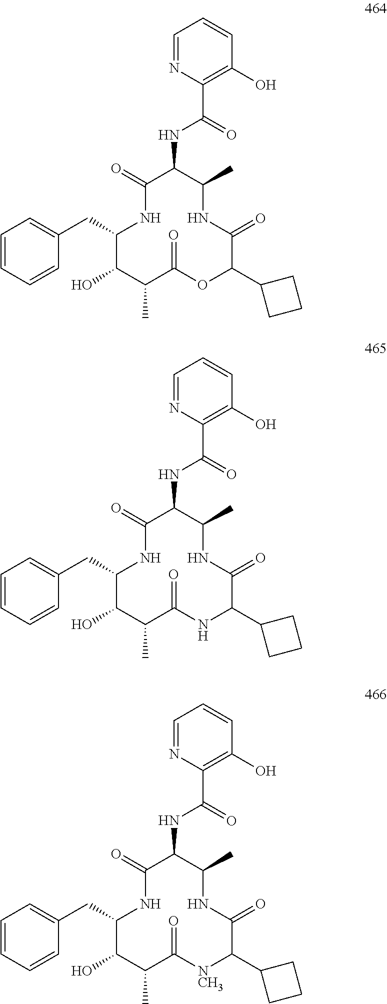 [SMILES: C[C@H]1NC(=O)C(C2CCC2)OC(=O)[C@H](C)[C@H](O)[C@H](Cc2ccccc2)NC(=O)[C@H]1NC(=O)c1ncccc1O ; C[C@H]1NC(=O)C(C2CCC2)NC(=O)[C@H](C)[C@H](O)[C@H](Cc2ccccc2)NC(=O)[C@H]1NC(=O)c1ncccc1O ; C[C@H]1NC(=O)C(C2CCC2)N(C)C(=O)[C@H](C)[C@H](O)[C@H](Cc2ccccc2)NC(=O)[C@H]1NC(=O)c1ncccc1O]

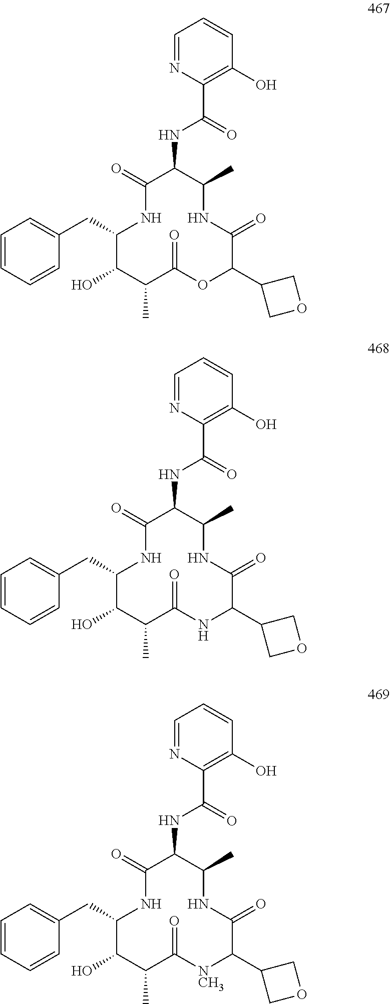 [SMILES: C[C@H]1NC(=O)C(C2COC2)OC(=O)[C@H](C)[C@H](O)[C@H](Cc2ccccc2)NC(=O)[C@H]1NC(=O)c1ncccc1O ; C[C@H]1NC(=O)C(C2COC2)NC(=O)[C@H](C)[C@H](O)[C@H](Cc2ccccc2)NC(=O)[C@H]1NC(=O)c1ncccc1O ; C[C@H]1NC(=O)C(C2COC2)N(C)C(=O)[C@H](C)[C@H](O)[C@H](Cc2ccccc2)NC(=O)[C@H]1NC(=O)c1ncccc1O]

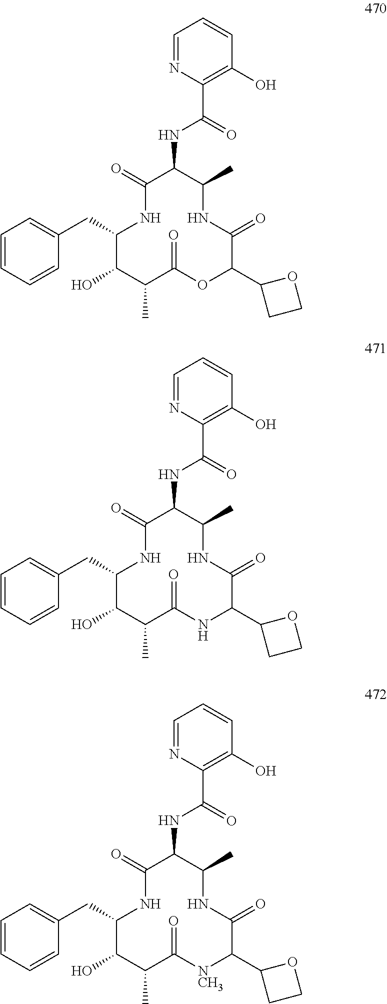 [SMILES: C[C@H]1NC(=O)C(C2CCO2)OC(=O)[C@H](C)[C@H](O)[C@H](Cc2ccccc2)NC(=O)[C@H]1NC(=O)c1ncccc1O ; C[C@H]1NC(=O)C(C2CCO2)NC(=O)[C@H](C)[C@H](O)[C@H](Cc2ccccc2)NC(=O)[C@H]1NC(=O)c1ncccc1O ; C[C@H]1NC(=O)C(C2CCO2)N(C)C(=O)[C@H](C)[C@H](O)[C@H](Cc2ccccc2)NC(=O)[C@H]1NC(=O)c1ncccc1O]

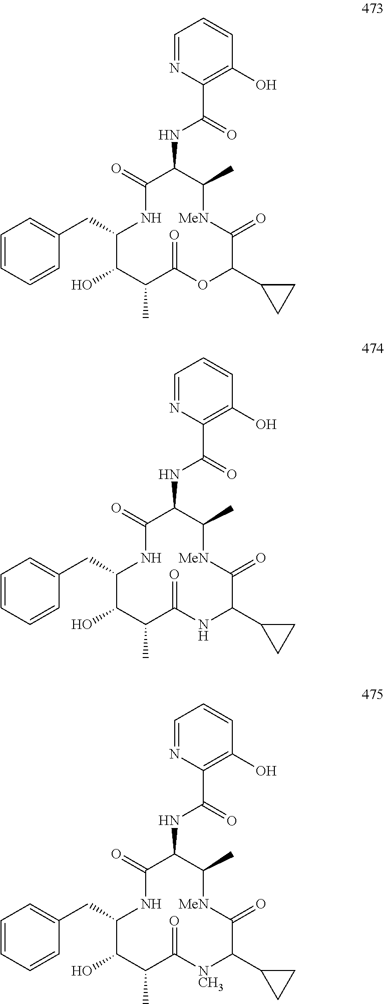 [SMILES: C[C@@H]1[C@H](NC(=O)c2ncccc2O)C(=O)N[C@@H](Cc2ccccc2)[C@@H](O)[C@@H](C)C(=O)OC(C2CC2)C(=O)N1C ; C[C@@H]1[C@H](NC(=O)c2ncccc2O)C(=O)N[C@@H](Cc2ccccc2)[C@@H](O)[C@@H](C)C(=O)NC(C2CC2)C(=O)N1C ; C[C@@H]1[C@H](NC(=O)c2ncccc2O)C(=O)N[C@@H](Cc2ccccc2)[C@@H](O)[C@@H](C)C(=O)N(C)C(C2CC2)C(=O)N1C]

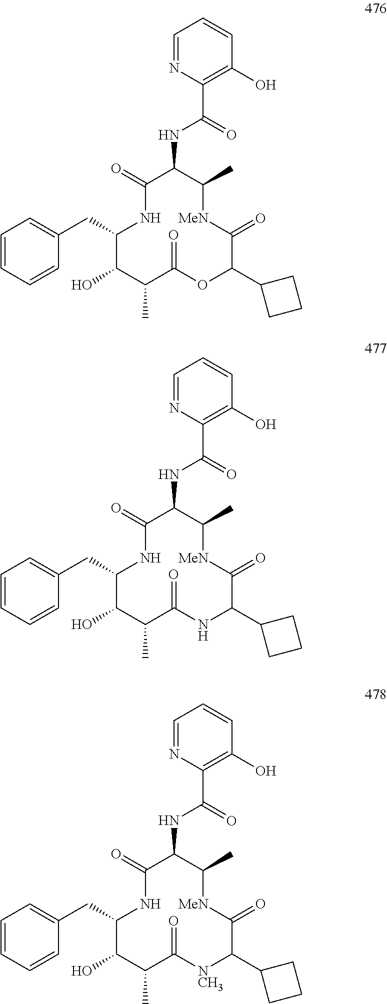 [SMILES: C[C@@H]1[C@H](NC(=O)c2ncccc2O)C(=O)N[C@@H](Cc2ccccc2)[C@@H](O)[C@@H](C)C(=O)OC(C2CCC2)C(=O)N1C ; C[C@@H]1[C@H](NC(=O)c2ncccc2O)C(=O)N[C@@H](Cc2ccccc2)[C@@H](O)[C@@H](C)C(=O)NC(C2CCC2)C(=O)N1C ; C[C@@H]1[C@H](NC(=O)c2ncccc2O)C(=O)N[C@@H](Cc2ccccc2)[C@@H](O)[C@@H](C)C(=O)N(C)C(C2CCC2)C(=O)N1C]

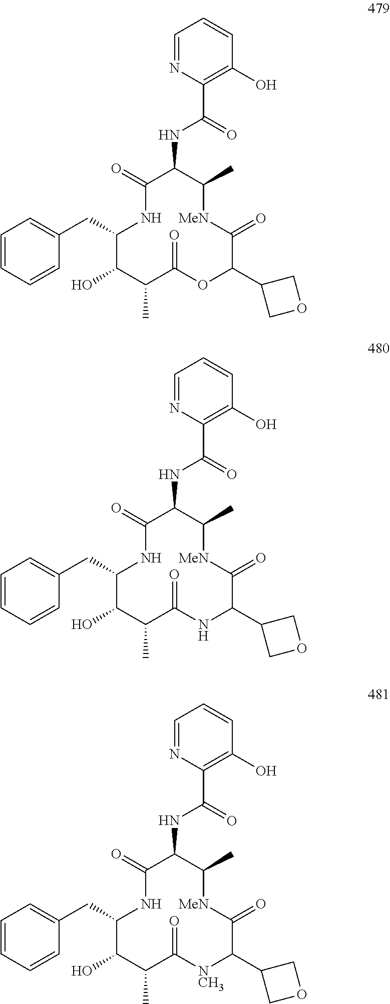 [SMILES: C[C@@H]1[C@H](NC(=O)c2ncccc2O)C(=O)N[C@@H](Cc2ccccc2)[C@@H](O)[C@@H](C)C(=O)OC(C2COC2)C(=O)N1C ; C[C@@H]1[C@H](NC(=O)c2ncccc2O)C(=O)N[C@@H](Cc2ccccc2)[C@@H](O)[C@@H](C)C(=O)NC(C2COC2)C(=O)N1C ; C[C@@H]1[C@H](NC(=O)c2ncccc2O)C(=O)N[C@@H](Cc2ccccc2)[C@@H](O)[C@@H](C)C(=O)N(C)C(C2COC2)C(=O)N1C]

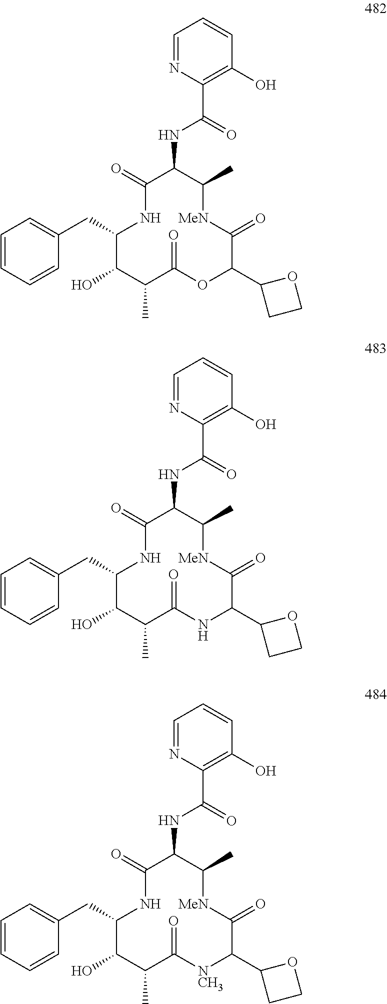 [SMILES: C[C@@H]1[C@H](NC(=O)c2ncccc2O)C(=O)N[C@@H](Cc2ccccc2)[C@@H](O)[C@@H](C)C(=O)OC(C2CCO2)C(=O)N1C ; C[C@@H]1[C@H](NC(=O)c2ncccc2O)C(=O)N[C@@H](Cc2ccccc2)[C@@H](O)[C@@H](C)C(=O)NC(C2CCO2)C(=O)N1C ; C[C@@H]1[C@H](NC(=O)c2ncccc2O)C(=O)N[C@@H](Cc2ccccc2)[C@@H](O)[C@@H](C)C(=O)N(C)C(C2CCO2)C(=O)N1C]

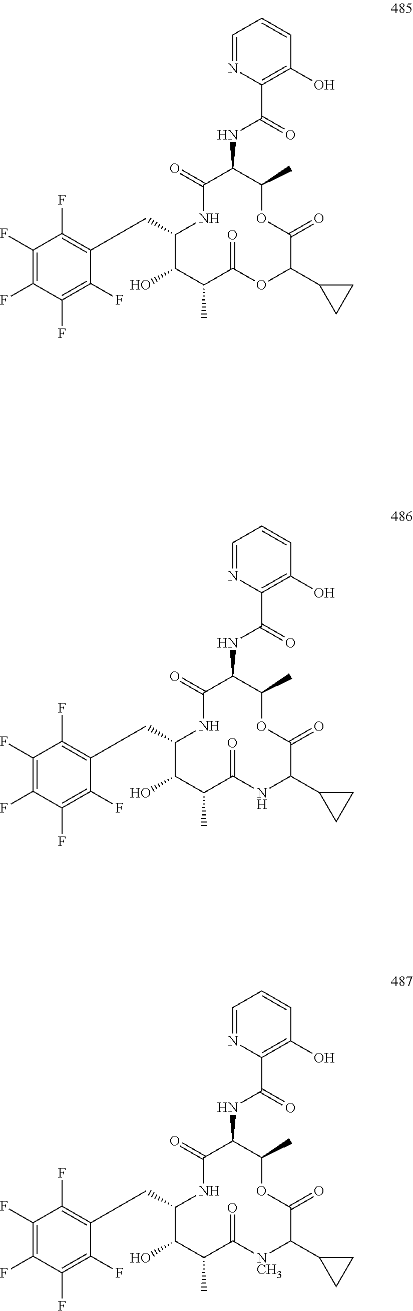 [SMILES: C[C@H]1OC(=O)C(C2CC2)OC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(C2CC2)NC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(C2CC2)N(C)C(=O)[C@H](C)[C@H](O)[C@H](Cc2c(F)c(F)c(F)c(F)c2F)NC(=O)[C@H]1NC(=O)c1ncccc1O]

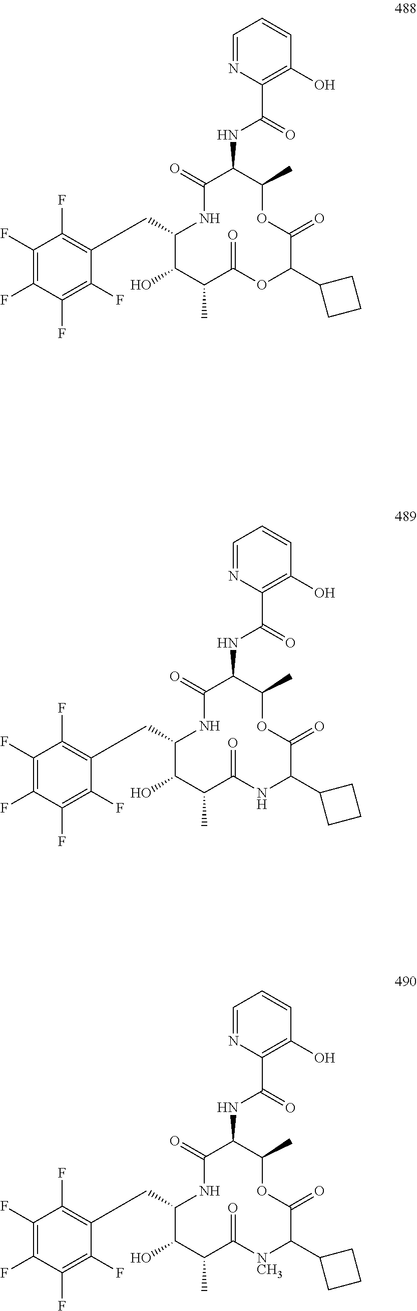 [SMILES: C[C@H]1OC(=O)C(C2CCC2)OC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(C2CCC2)NC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(C2CCC2)N(C)C(=O)[C@H](C)[C@H](O)[C@H](Cc2c(F)c(F)c(F)c(F)c2F)NC(=O)[C@H]1NC(=O)c1ncccc1O]

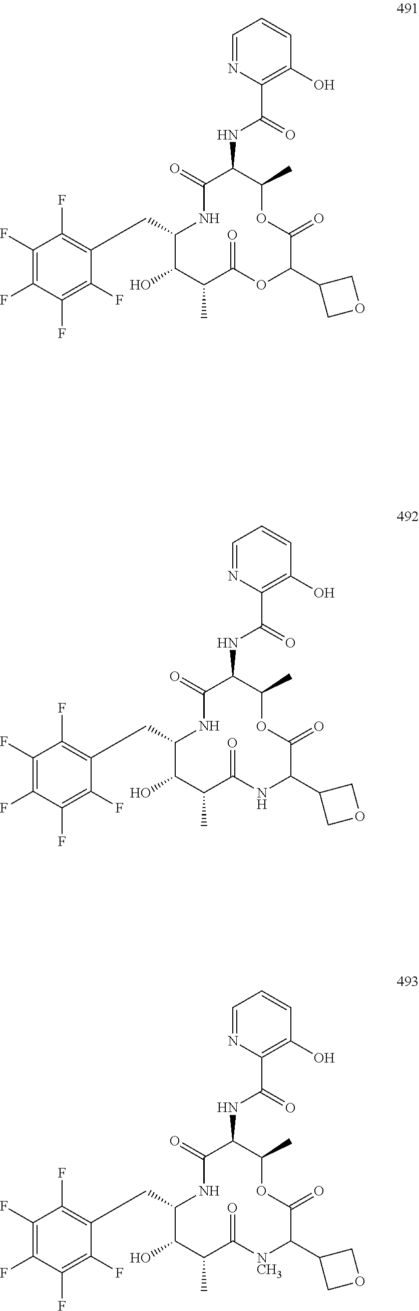 [SMILES: C[C@H]1OC(=O)C(C2COC2)OC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(C2COC2)NC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(C2COC2)N(C)C(=O)[C@H](C)[C@H](O)[C@H](Cc2c(F)c(F)c(F)c(F)c2F)NC(=O)[C@H]1NC(=O)c1ncccc1O]

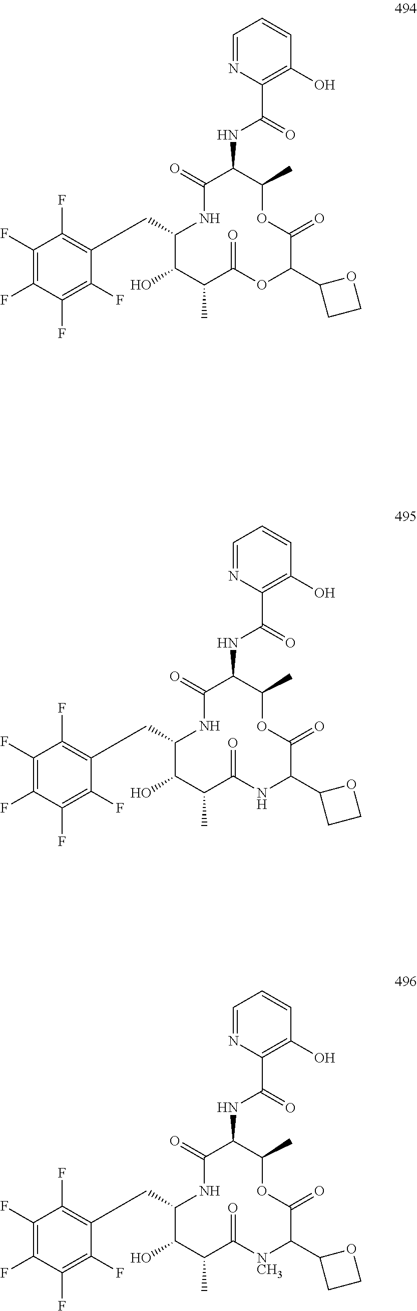 [SMILES: C[C@H]1OC(=O)C(C2CCO2)OC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(C2CCO2)NC(=O)[C@H](C)[C@H](O)[C@H](Cc2c(F)c(F)c(F)c(F)c2F)NC(=O)[C@H]1NC(=O)c1ncccc1O ; C[C@H]1OC(=O)C(C2CCO2)N(C)C(=O)[C@H](C)[C@H](O)[C@H](Cc2c(F)c(F)c(F)c(F)c2F)NC(=O)[C@H]1NC(=O)c1ncccc1O]

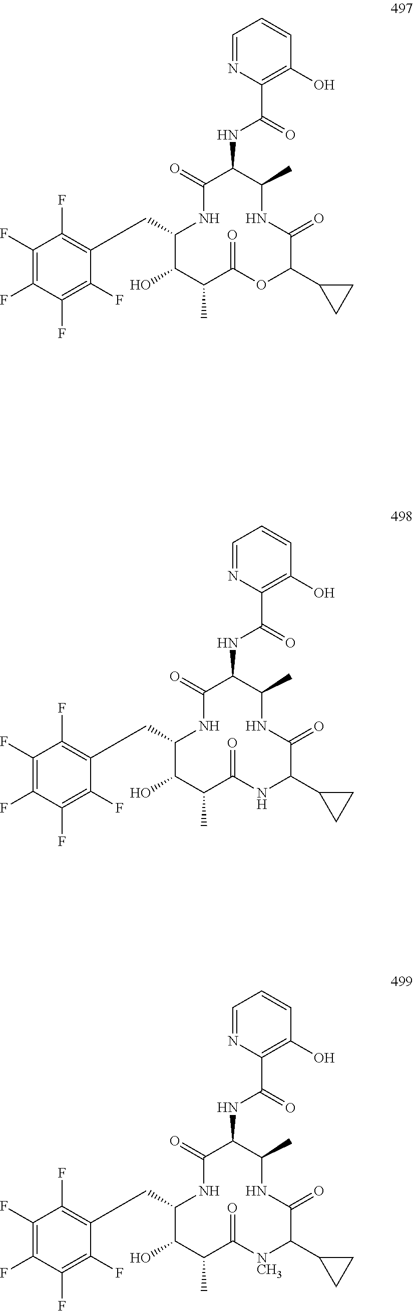 [SMILES: C[C@H]1NC(=O)C(C2CC2)OC(=O)[C@H](C)[C@H](O)[C@H](Cc2c(F)c(F)c(F)c(F)c2F)NC(=O)[C@H]1NC(=O)c1ncccc1O ; C[C@H]1NC(=O)C(C2CC2)NC(=O)[C@H](C)[C@H](O)[C@H](Cc2c(F)c(F)c(F)c(F)c2F)NC(=O)[C@H]1NC(=O)c1ncccc1O ; C[C@H]1NC(=O)C(C2CC2)N(C)C(=O)[C@H](C)[C@H](O)[C@H](Cc2c(F)c(F)c(F)c(F)c2F)NC(=O)[C@H]1NC(=O)c1ncccc1O]

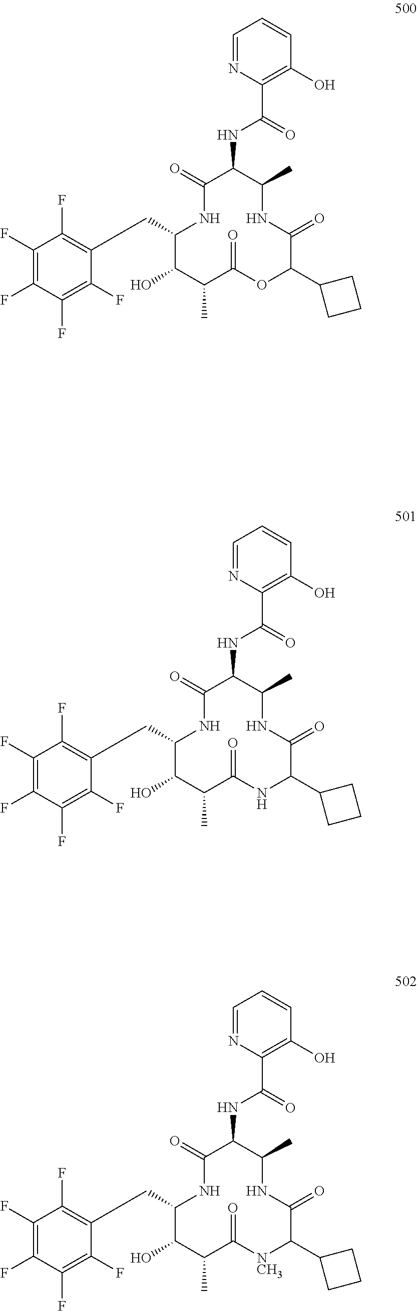 [SMILES: C[C@H]1NC(=O)C(C2CCC2)OC(=O)[C@H](C)[C@H](O)[C@H](Cc2c(F)c(F)c(F)c(F)c2F)NC(=O)[C@H]1NC(=O)c1ncccc1O ; C[C@H]1NC(=O)C(C2CCC2)NC(=O)[C@H](C)[C@H](O)[C@H](Cc2c(F)c(F)c(F)c(F)c2F)NC(=O)[C@H]1NC(=O)c1ncccc1O ; C[C@H]1NC(=O)C(C2CCC2)N(C)C(=O)[C@H](C)[C@H](O)[C@H](Cc2c(F)c(F)c(F)c(F)c2F)NC(=O)[C@H]1NC(=O)c1ncccc1O]

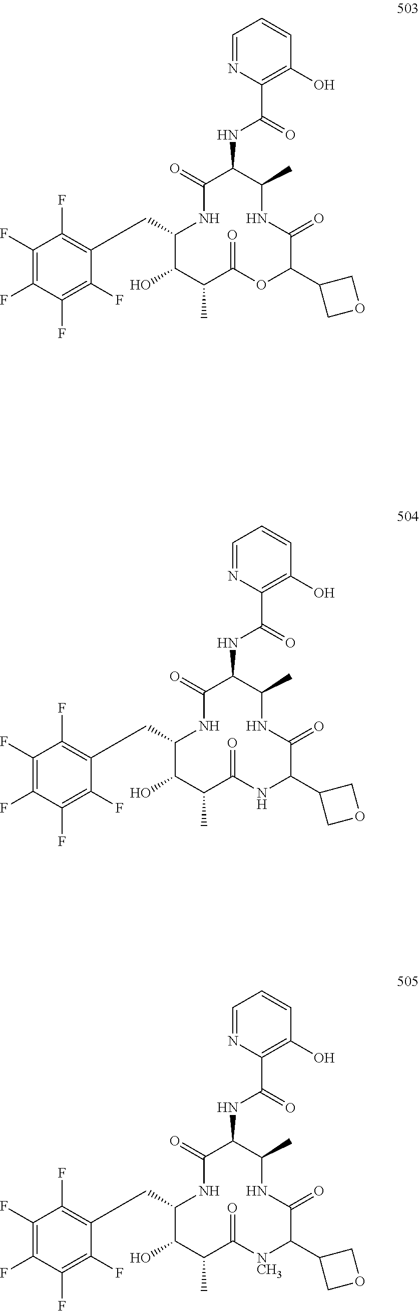 [SMILES: C[C@H]1NC(=O)C(C2COC2)OC(=O)[C@H](C)[C@H](O)[C@H](Cc2c(F)c(F)c(F)c(F)c2F)NC(=O)[C@H]1NC(=O)c1ncccc1O ; C[C@H]1NC(=O)C(C2COC2)NC(=O)[C@H](C)[C@H](O)[C@H](Cc2c(F)c(F)c(F)c(F)c2F)NC(=O)[C@H]1NC(=O)c1ncccc1O ; C[C@H]1NC(=O)C(C2COC2)N(C)C(=O)[C@H](C)[C@H](O)[C@H](Cc2c(F)c(F)c(F)c(F)c2F)NC(=O)[C@H]1NC(=O)c1ncccc1O]

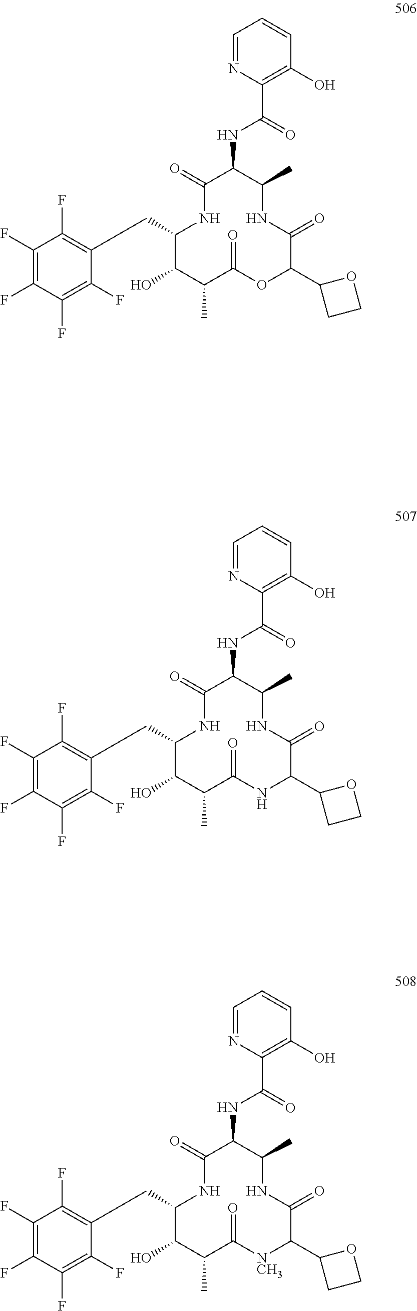 [SMILES: C[C@H]1NC(=O)C(C2CCO2)OC(=O)[C@H](C)[C@H](O)[C@H](Cc2c(F)c(F)c(F)c(F)c2F)NC(=O)[C@H]1NC(=O)c1ncccc1O ; C[C@H]1NC(=O)C(C2CCO2)NC(=O)[C@H](C)[C@H](O)[C@H](Cc2c(F)c(F)c(F)c(F)c2F)NC(=O)[C@H]1NC(=O)c1ncccc1O ; C[C@H]1NC(=O)C(C2CCO2)N(C)C(=O)[C@H](C)[C@H](O)[C@H](Cc2c(F)c(F)c(F)c(F)c2F)NC(=O)[C@H]1NC(=O)c1ncccc1O]

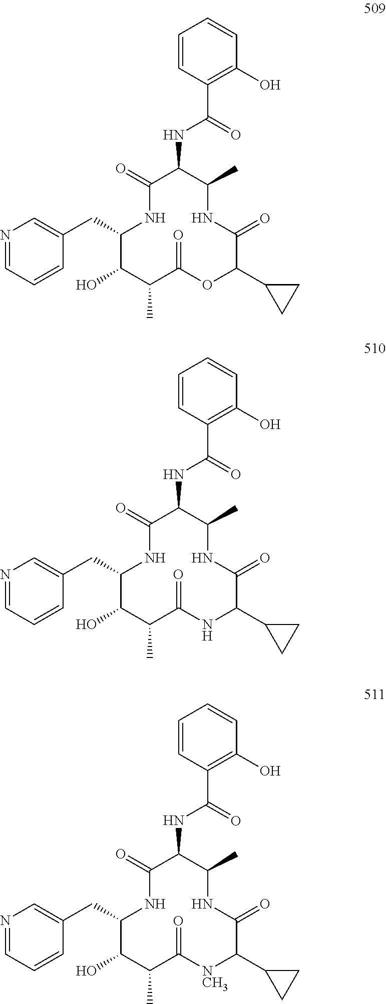 [SMILES: C[C@H]1NC(=O)C(C2CC2)OC(=O)[C@H](C)[C@H](O)[C@H](Cc2cccnc2)NC(=O)[C@H]1NC(=O)c1ccccc1O ; C[C@H]1NC(=O)C(C2CC2)NC(=O)[C@H](C)[C@H](O)[C@H](Cc2cccnc2)NC(=O)[C@H]1NC(=O)c1ccccc1O ; C[C@H]1NC(=O)C(C2CC2)N(C)C(=O)[C@H](C)[C@H](O)[C@H](Cc2cccnc2)NC(=O)[C@H]1NC(=O)c1ccccc1O]

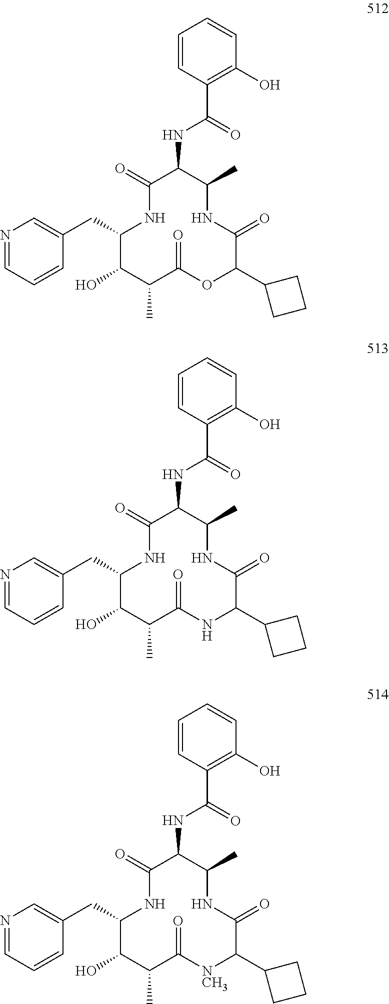 [SMILES: C[C@H]1NC(=O)C(C2CCC2)OC(=O)[C@H](C)[C@H](O)[C@H](Cc2cccnc2)NC(=O)[C@H]1NC(=O)c1ccccc1O ; C[C@H]1NC(=O)C(C2CCC2)NC(=O)[C@H](C)[C@H](O)[C@H](Cc2cccnc2)NC(=O)[C@H]1NC(=O)c1ccccc1O ; C[C@H]1NC(=O)C(C2CCC2)N(C)C(=O)[C@H](C)[C@H](O)[C@H](Cc2cccnc2)NC(=O)[C@H]1NC(=O)c1ccccc1O]

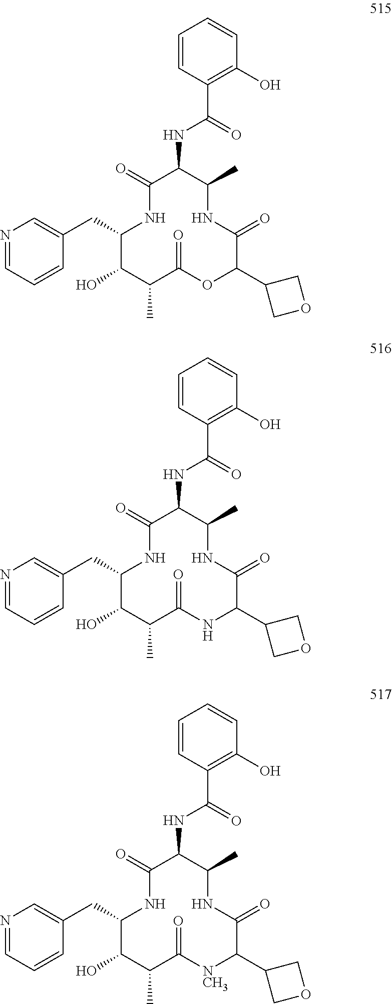 [SMILES: C[C@H]1NC(=O)C(C2COC2)OC(=O)[C@H](C)[C@H](O)[C@H](Cc2cccnc2)NC(=O)[C@H]1NC(=O)c1ccccc1O ; C[C@H]1NC(=O)C(C2COC2)NC(=O)[C@H](C)[C@H](O)[C@H](Cc2cccnc2)NC(=O)[C@H]1NC(=O)c1ccccc1O ; C[C@H]1NC(=O)C(C2COC2)N(C)C(=O)[C@H](C)[C@H](O)[C@H](Cc2cccnc2)NC(=O)[C@H]1NC(=O)c1ccccc1O]

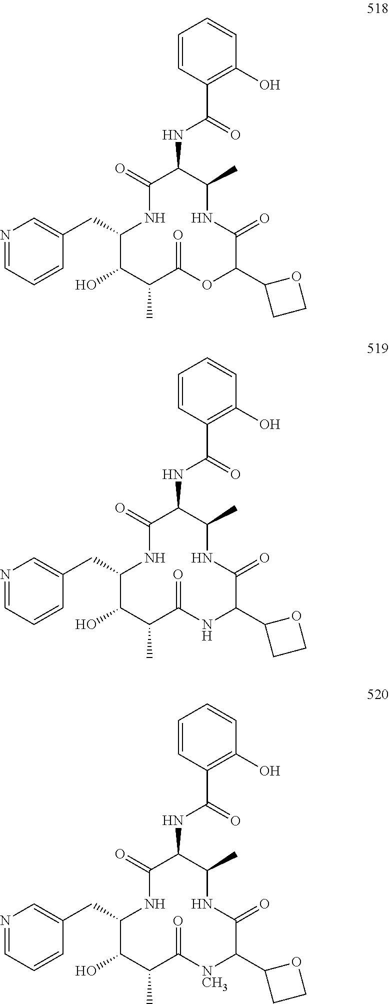 [SMILES: C[C@H]1NC(=O)C(C2CCO2)OC(=O)[C@H](C)[C@H](O)[C@H](Cc2cccnc2)NC(=O)[C@H]1NC(=O)c1ccccc1O ; C[C@H]1NC(=O)C(C2CCO2)NC(=O)[C@H](C)[C@H](O)[C@H](Cc2cccnc2)NC(=O)[C@H]1NC(=O)c1ccccc1O ; C[C@H]1NC(=O)C(C2CCO2)N(C)C(=O)[C@H](C)[C@H](O)[C@H](Cc2cccnc2)NC(=O)[C@H]1NC(=O)c1ccccc1O]

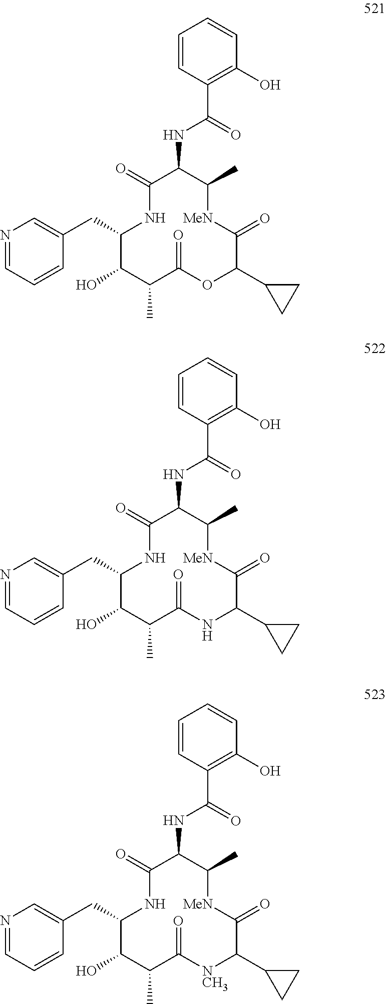 [SMILES: C[C@@H]1[C@H](NC(=O)c2ccccc2O)C(=O)N[C@@H](Cc2cccnc2)[C@@H](O)[C@@H](C)C(=O)OC(C2CC2)C(=O)N1C ; C[C@@H]1[C@H](NC(=O)c2ccccc2O)C(=O)N[C@@H](Cc2cccnc2)[C@@H](O)[C@@H](C)C(=O)NC(C2CC2)C(=O)N1C ; C[C@@H]1[C@H](NC(=O)c2ccccc2O)C(=O)N[C@@H](Cc2cccnc2)[C@@H](O)[C@@H](C)C(=O)N(C)C(C2CC2)C(=O)N1C]

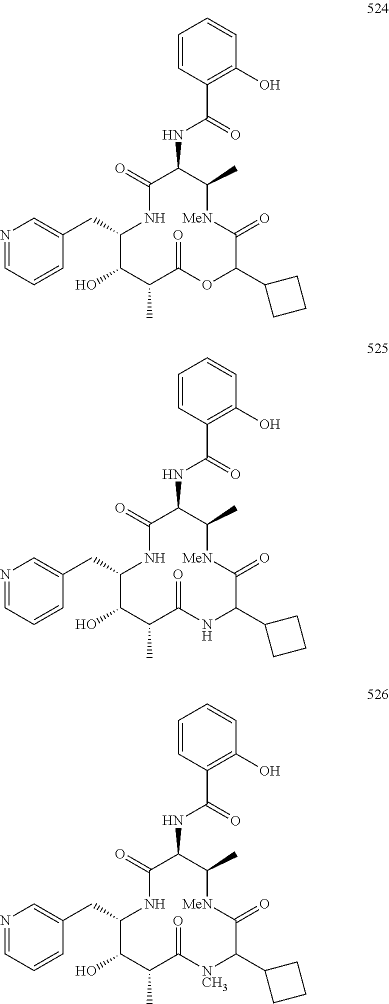 [SMILES: C[C@@H]1[C@H](NC(=O)c2ccccc2O)C(=O)N[C@@H](Cc2cccnc2)[C@@H](O)[C@@H](C)C(=O)OC(C2CCC2)C(=O)N1C ; C[C@@H]1[C@H](NC(=O)c2ccccc2O)C(=O)N[C@@H](Cc2cccnc2)[C@@H](O)[C@@H](C)C(=O)NC(C2CCC2)C(=O)N1C ; C[C@@H]1[C@H](NC(=O)c2ccccc2O)C(=O)N[C@@H](Cc2cccnc2)[C@@H](O)[C@@H](C)C(=O)N(C)C(C2CCC2)C(=O)N1C]

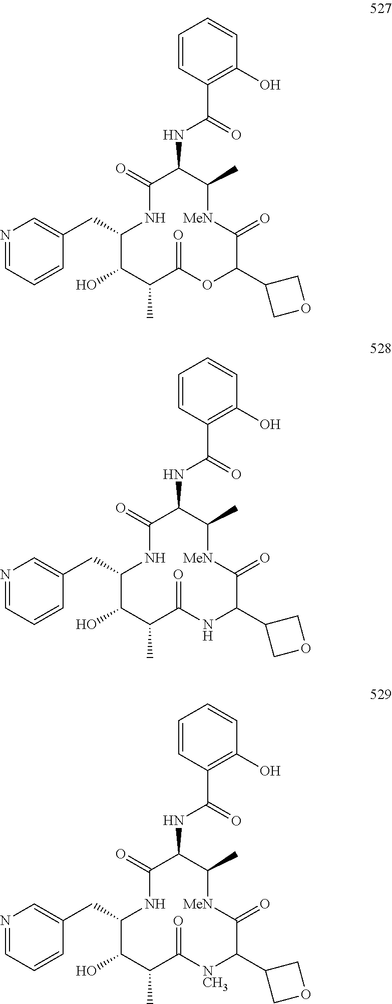 [SMILES: C[C@@H]1[C@H](NC(=O)c2ccccc2O)C(=O)N[C@@H](Cc2cccnc2)[C@@H](O)[C@@H](C)C(=O)OC(C2COC2)C(=O)N1C ; C[C@@H]1[C@H](NC(=O)c2ccccc2O)C(=O)N[C@@H](Cc2cccnc2)[C@@H](O)[C@@H](C)C(=O)NC(C2COC2)C(=O)N1C ; C[C@@H]1[C@H](NC(=O)c2ccccc2O)C(=O)N[C@@H](Cc2cccnc2)[C@@H](O)[C@@H](C)C(=O)N(C)C(C2COC2)C(=O)N1C]

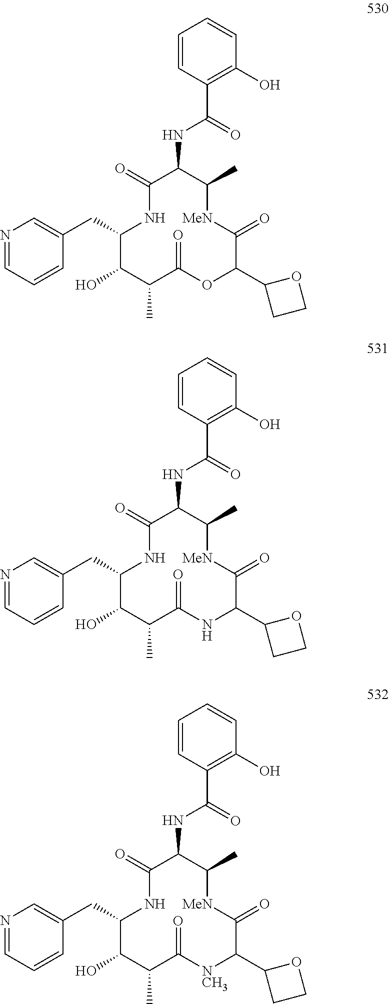 [SMILES: C[C@@H]1[C@H](NC(=O)c2ccccc2O)C(=O)N[C@@H](Cc2cccnc2)[C@@H](O)[C@@H](C)C(=O)OC(C2CCO2)C(=O)N1C ; C[C@@H]1[C@H](NC(=O)c2ccccc2O)C(=O)N[C@@H](Cc2cccnc2)[C@@H](O)[C@@H](C)C(=O)NC(C2CCO2)C(=O)N1C ; C[C@@H]1[C@H](NC(=O)c2ccccc2O)C(=O)N[C@@H](Cc2cccnc2)[C@@H](O)[C@@H](C)C(=O)N(C)C(C2CCO2)C(=O)N1C]

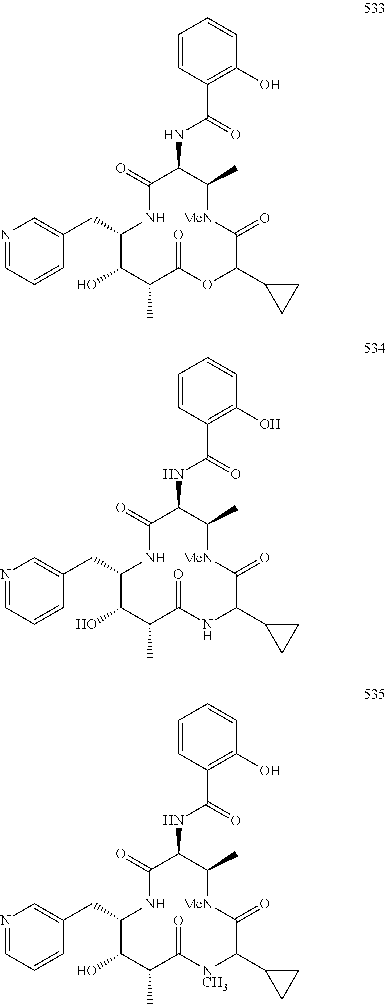 [SMILES: C[C@@H]1[C@H](NC(=O)c2ccccc2O)C(=O)N[C@@H](Cc2cccnc2)[C@@H](O)[C@@H](C)C(=O)OC(C2CC2)C(=O)N1C ; C[C@@H]1[C@H](NC(=O)c2ccccc2O)C(=O)N[C@@H](Cc2cccnc2)[C@@H](O)[C@@H](C)C(=O)NC(C2CC2)C(=O)N1C ; C[C@@H]1[C@H](NC(=O)c2ccccc2O)C(=O)N[C@@H](Cc2cccnc2)[C@@H](O)[C@@H](C)C(=O)N(C)C(C2CC2)C(=O)N1C]

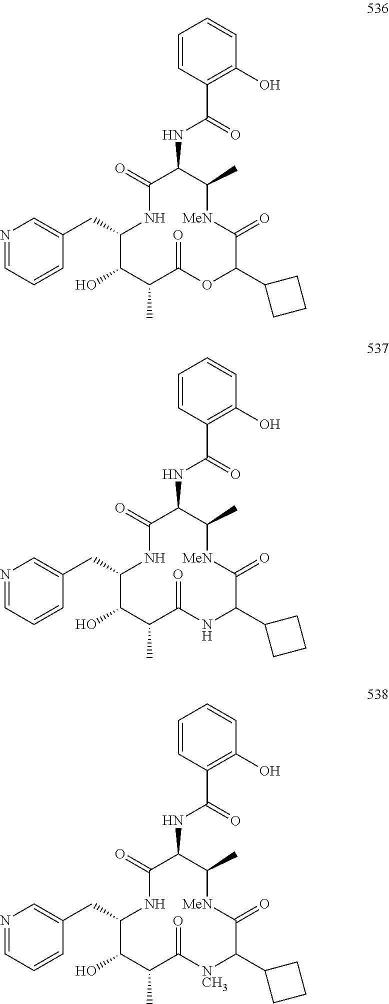 [SMILES: C[C@@H]1[C@H](NC(=O)c2ccccc2O)C(=O)N[C@@H](Cc2cccnc2)[C@@H](O)[C@@H](C)C(=O)OC(C2CCC2)C(=O)N1C ; C[C@@H]1[C@H](NC(=O)c2ccccc2O)C(=O)N[C@@H](Cc2cccnc2)[C@@H](O)[C@@H](C)C(=O)NC(C2CCC2)C(=O)N1C ; C[C@@H]1[C@H](NC(=O)c2ccccc2O)C(=O)N[C@@H](Cc2cccnc2)[C@@H](O)[C@@H](C)C(=O)N(C)C(C2CCC2)C(=O)N1C]

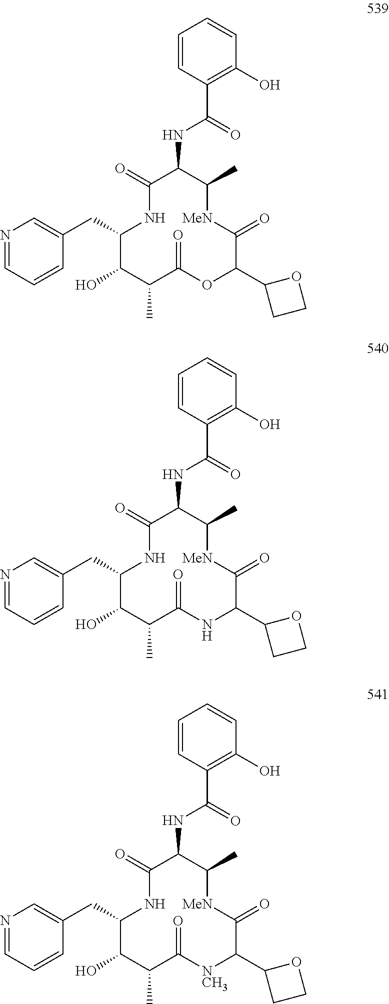 [SMILES: C[C@@H]1[C@H](NC(=O)c2ccccc2O)C(=O)N[C@@H](Cc2cccnc2)[C@@H](O)[C@@H](C)C(=O)OC(C2CCO2)C(=O)N1C ; C[C@@H]1[C@H](NC(=O)c2ccccc2O)C(=O)N[C@@H](Cc2cccnc2)[C@@H](O)[C@@H](C)C(=O)NC(C2CCO2)C(=O)N1C ; C[C@@H]1[C@H](NC(=O)c2ccccc2O)C(=O)N[C@@H](Cc2cccnc2)[C@@H](O)[C@@H](C)C(=O)N(C)C(C2CCO2)C(=O)N1C]

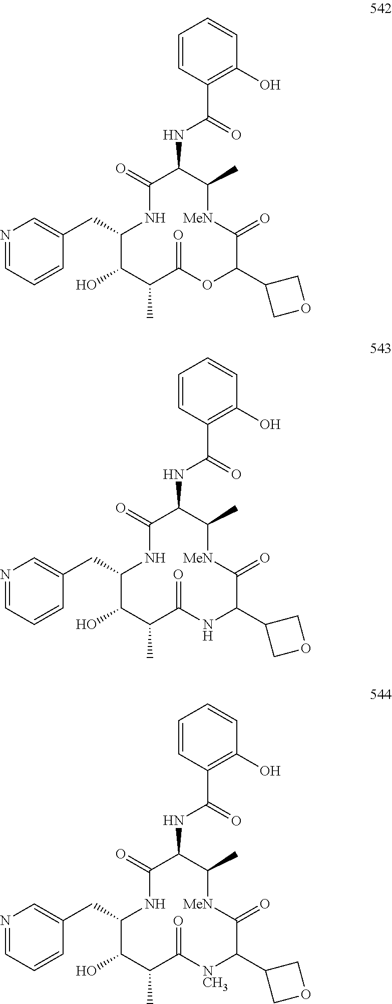 [SMILES: C[C@@H]1[C@H](NC(=O)c2ccccc2O)C(=O)N[C@@H](Cc2cccnc2)[C@@H](O)[C@@H](C)C(=O)OC(C2COC2)C(=O)N1C ; C[C@@H]1[C@H](NC(=O)c2ccccc2O)C(=O)N[C@@H](Cc2cccnc2)[C@@H](O)[C@@H](C)C(=O)NC(C2COC2)C(=O)N1C ; C[C@@H]1[C@H](NC(=O)c2ccccc2O)C(=O)N[C@@H](Cc2cccnc2)[C@@H](O)[C@@H](C)C(=O)N(C)C(C2COC2)C(=O)N1C]

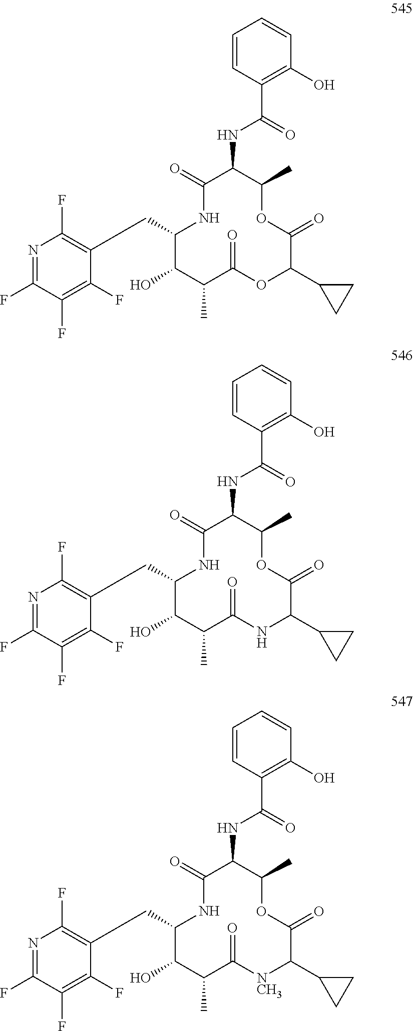 [SMILES: C[C@H]1OC(=O)C(C2CC2)OC(=O)[C@H](C)[C@H](O)[C@H](Cc2c(F)nc(F)c(F)c2F)NC(=O)[C@H]1NC(=O)c1ccccc1O ; C[C@H]1OC(=O)C(C2CC2)NC(=O)[C@H](C)[C@H](O)[C@H](Cc2c(F)nc(F)c(F)c2F)NC(=O)[C@H]1NC(=O)c1ccccc1O ; C[C@H]1OC(=O)C(C2CC2)N(C)C(=O)[C@H](C)[C@H](O)[C@H](Cc2c(F)nc(F)c(F)c2F)NC(=O)[C@H]1NC(=O)c1ccccc1O]

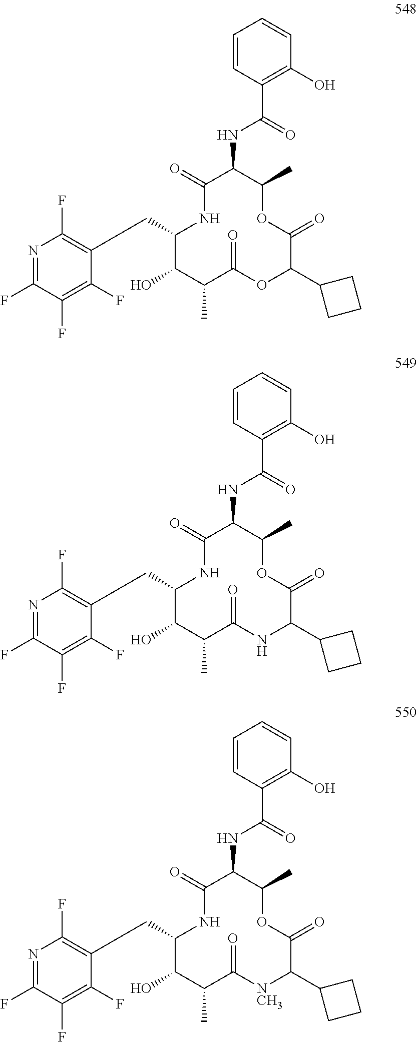 [SMILES: C[C@H]1OC(=O)C(C2CCC2)OC(=O)[C@H](C)[C@H](O)[C@H](Cc2c(F)nc(F)c(F)c2F)NC(=O)[C@H]1NC(=O)c1ccccc1O ; C[C@H]1OC(=O)C(C2CCC2)NC(=O)[C@H](C)[C@H](O)[C@H](Cc2c(F)nc(F)c(F)c2F)NC(=O)[C@H]1NC(=O)c1ccccc1O ; C[C@H]1OC(=O)C(C2CCC2)N(C)C(=O)[C@H](C)[C@H](O)[C@H](Cc2c(F)nc(F)c(F)c2F)NC(=O)[C@H]1NC(=O)c1ccccc1O]

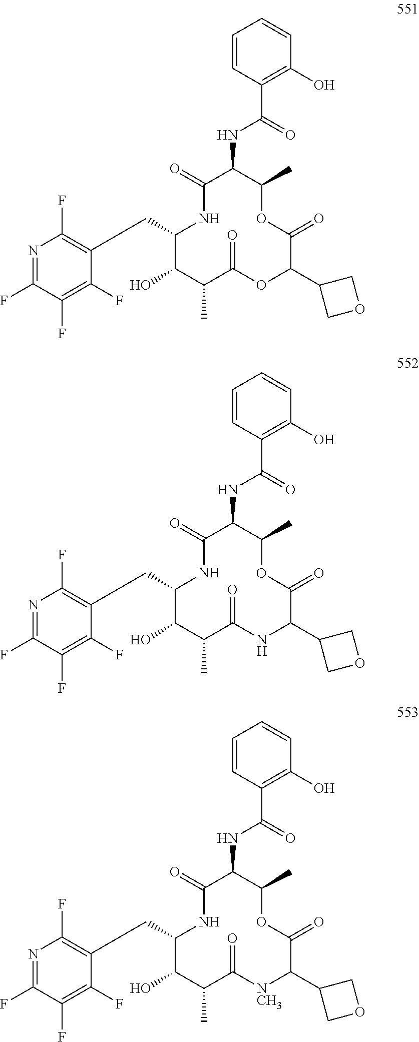 [SMILES: C[C@H]1OC(=O)C(C2COC2)OC(=O)[C@H](C)[C@H](O)[C@H](Cc2c(F)nc(F)c(F)c2F)NC(=O)[C@H]1NC(=O)c1ccccc1O ; C[C@H]1OC(=O)C(C2COC2)NC(=O)[C@H](C)[C@H](O)[C@H](Cc2c(F)nc(F)c(F)c2F)NC(=O)[C@H]1NC(=O)c1ccccc1O ; C[C@H]1OC(=O)C(C2COC2)N(C)C(=O)[C@H](C)[C@H](O)[C@H](Cc2c(F)nc(F)c(F)c2F)NC(=O)[C@H]1NC(=O)c1ccccc1O]

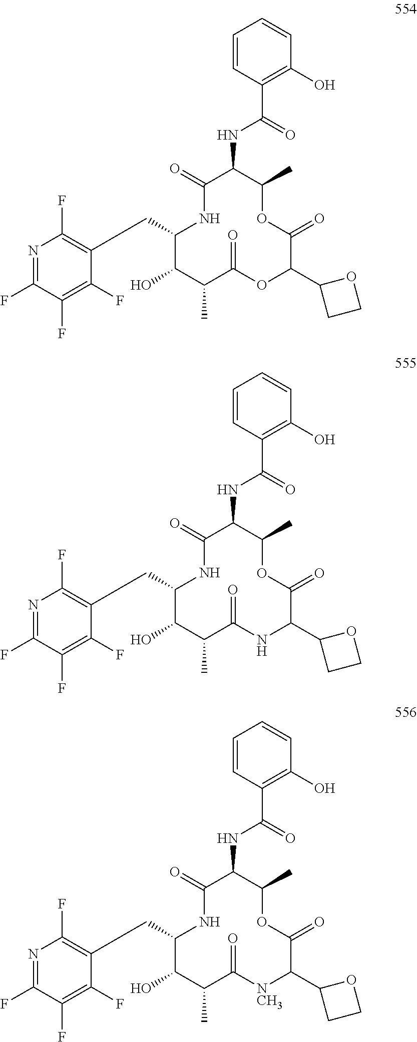 [SMILES: C[C@H]1OC(=O)C(C2CCO2)OC(=O)[C@H](C)[C@H](O)[C@H](Cc2c(F)nc(F)c(F)c2F)NC(=O)[C@H]1NC(=O)c1ccccc1O ; C[C@H]1OC(=O)C(C2CCO2)NC(=O)[C@H](C)[C@H](O)[C@H](Cc2c(F)nc(F)c(F)c2F)NC(=O)[C@H]1NC(=O)c1ccccc1O ; C[C@H]1OC(=O)C(C2CCO2)N(C)C(=O)[C@H](C)[C@H](O)[C@H](Cc2c(F)nc(F)c(F)c2F)NC(=O)[C@H]1NC(=O)c1ccccc1O]

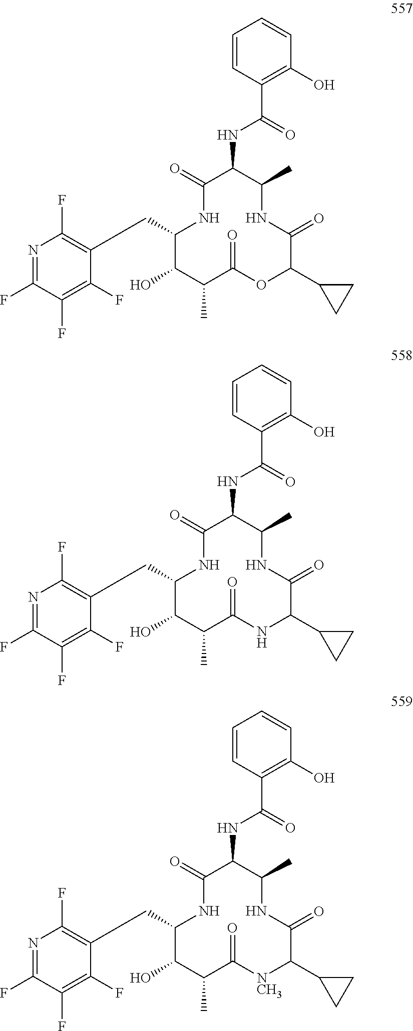 [SMILES: C[C@H]1NC(=O)C(C2CC2)OC(=O)[C@H](C)[C@H](O)[C@H](Cc2c(F)nc(F)c(F)c2F)NC(=O)[C@H]1NC(=O)c1ccccc1O ; C[C@H]1NC(=O)C(C2CC2)NC(=O)[C@H](C)[C@H](O)[C@H](Cc2c(F)nc(F)c(F)c2F)NC(=O)[C@H]1NC(=O)c1ccccc1O ; C[C@H]1NC(=O)C(C2CC2)N(C)C(=O)[C@H](C)[C@H](O)[C@H](Cc2c(F)nc(F)c(F)c2F)NC(=O)[C@H]1NC(=O)c1ccccc1O]

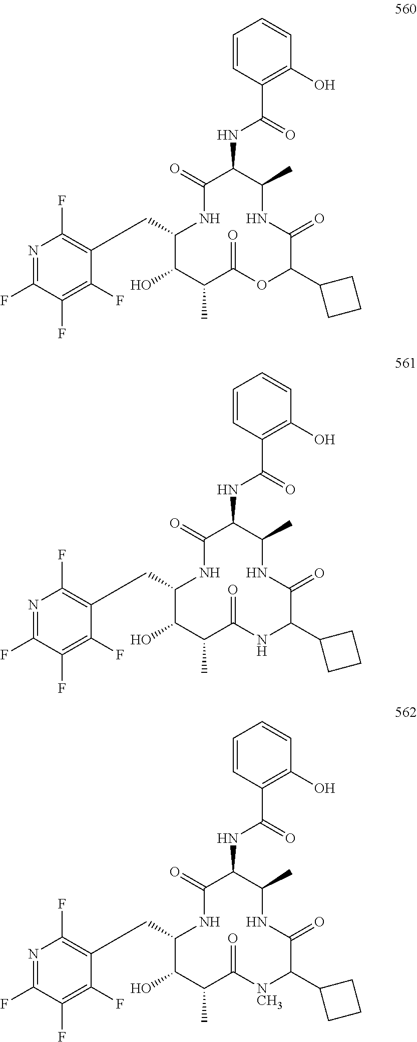 [SMILES: C[C@H]1NC(=O)C(C2CCC2)OC(=O)[C@H](C)[C@H](O)[C@H](Cc2c(F)nc(F)c(F)c2F)NC(=O)[C@H]1NC(=O)c1ccccc1O ; C[C@H]1NC(=O)C(C2CCC2)NC(=O)[C@H](C)[C@H](O)[C@H](Cc2c(F)nc(F)c(F)c2F)NC(=O)[C@H]1NC(=O)c1ccccc1O ; C[C@H]1NC(=O)C(C2CCC2)N(C)C(=O)[C@H](C)[C@H](O)[C@H](Cc2c(F)nc(F)c(F)c2F)NC(=O)[C@H]1NC(=O)c1ccccc1O]

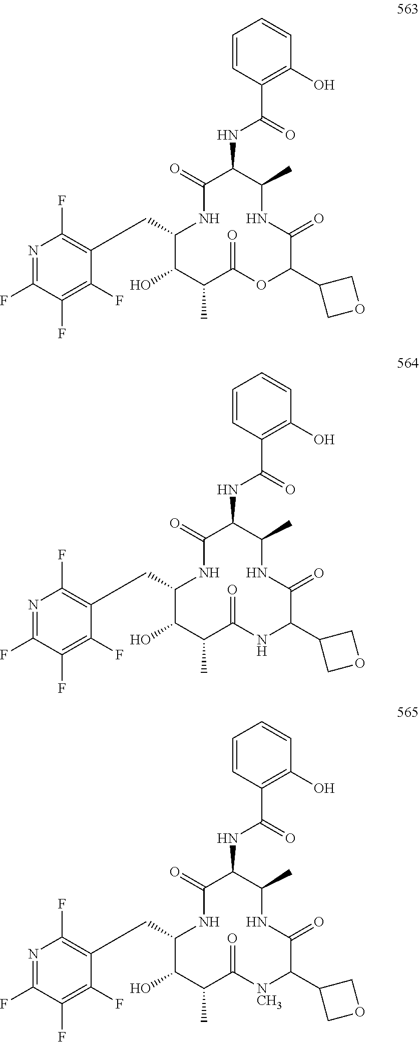 [SMILES: C[C@H]1NC(=O)C(C2COC2)OC(=O)[C@H](C)[C@H](O)[C@H](Cc2c(F)nc(F)c(F)c2F)NC(=O)[C@H]1NC(=O)c1ccccc1O ; C[C@H]1NC(=O)C(C2COC2)NC(=O)[C@H](C)[C@H](O)[C@H](Cc2c(F)nc(F)c(F)c2F)NC(=O)[C@H]1NC(=O)c1ccccc1O ; C[C@H]1NC(=O)C(C2COC2)N(C)C(=O)[C@H](C)[C@H](O)[C@H](Cc2c(F)nc(F)c(F)c2F)NC(=O)[C@H]1NC(=O)c1ccccc1O]

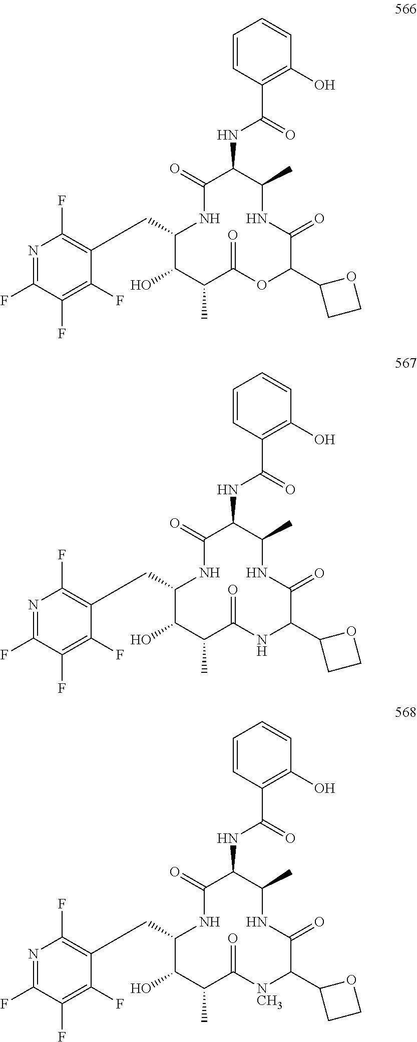 [SMILES: C[C@H]1NC(=O)C(C2CCO2)OC(=O)[C@H](C)[C@H](O)[C@H](Cc2c(F)nc(F)c(F)c2F)NC(=O)[C@H]1NC(=O)c1ccccc1O ; C[C@H]1NC(=O)C(C2CCO2)NC(=O)[C@H](C)[C@H](O)[C@H](Cc2c(F)nc(F)c(F)c2F)NC(=O)[C@H]1NC(=O)c1ccccc1O ; C[C@H]1NC(=O)C(C2CCO2)N(C)C(=O)[C@H](C)[C@H](O)[C@H](Cc2c(F)nc(F)c(F)c2F)NC(=O)[C@H]1NC(=O)c1ccccc1O]

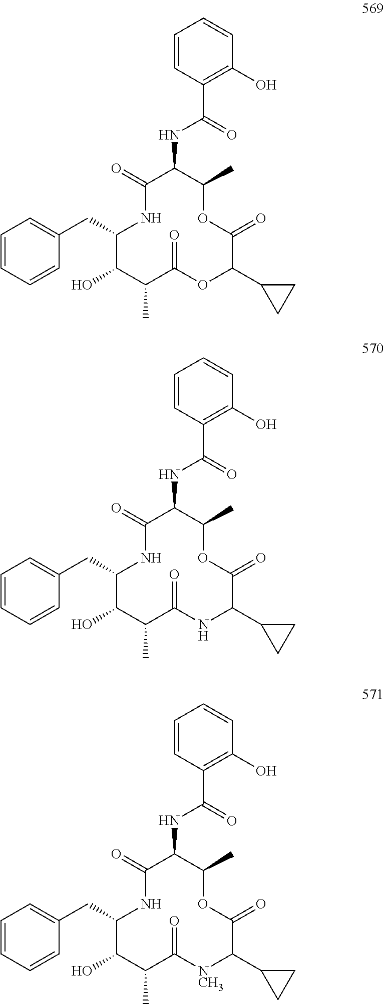 [SMILES: C[C@H]1OC(=O)C(C2CC2)OC(=O)[C@H](C)[C@H](O)[C@H](Cc2ccccc2)NC(=O)[C@H]1NC(=O)c1ccccc1O ; C[C@H]1OC(=O)C(C2CC2)NC(=O)[C@H](C)[C@H](O)[C@H](Cc2ccccc2)NC(=O)[C@H]1NC(=O)c1ccccc1O ; C[C@H]1OC(=O)C(C2CC2)N(C)C(=O)[C@H](C)[C@H](O)[C@H](Cc2ccccc2)NC(=O)[C@H]1NC(=O)c1ccccc1O]

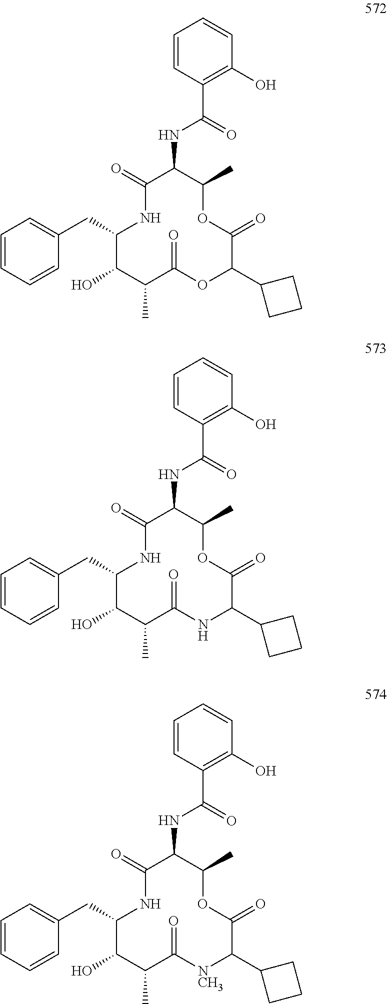 [SMILES: C[C@H]1OC(=O)C(C2CCC2)OC(=O)[C@H](C)[C@H](O)[C@H](Cc2ccccc2)NC(=O)[C@H]1NC(=O)c1ccccc1O ; C[C@H]1OC(=O)C(C2CCC2)NC(=O)[C@H](C)[C@H](O)[C@H](Cc2ccccc2)NC(=O)[C@H]1NC(=O)c1ccccc1O ; C[C@H]1OC(=O)C(C2CCC2)N(C)C(=O)[C@H](C)[C@H](O)[C@H](Cc2ccccc2)NC(=O)[C@H]1NC(=O)c1ccccc1O]

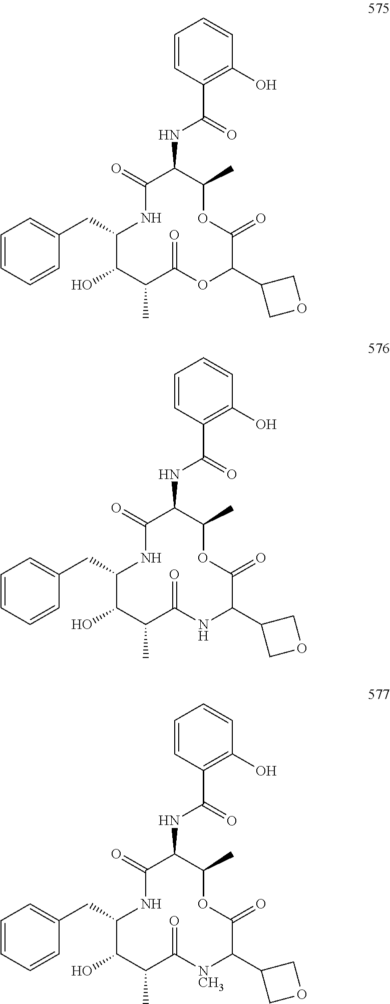 [SMILES: C[C@H]1OC(=O)C(C2COC2)OC(=O)[C@H](C)[C@H](O)[C@H](Cc2ccccc2)NC(=O)[C@H]1NC(=O)c1ccccc1O ; C[C@H]1OC(=O)C(C2COC2)NC(=O)[C@H](C)[C@H](O)[C@H](Cc2ccccc2)NC(=O)[C@H]1NC(=O)c1ccccc1O ; C[C@H]1OC(=O)C(C2COC2)N(C)C(=O)[C@H](C)[C@H](O)[C@H](Cc2ccccc2)NC(=O)[C@H]1NC(=O)c1ccccc1O]

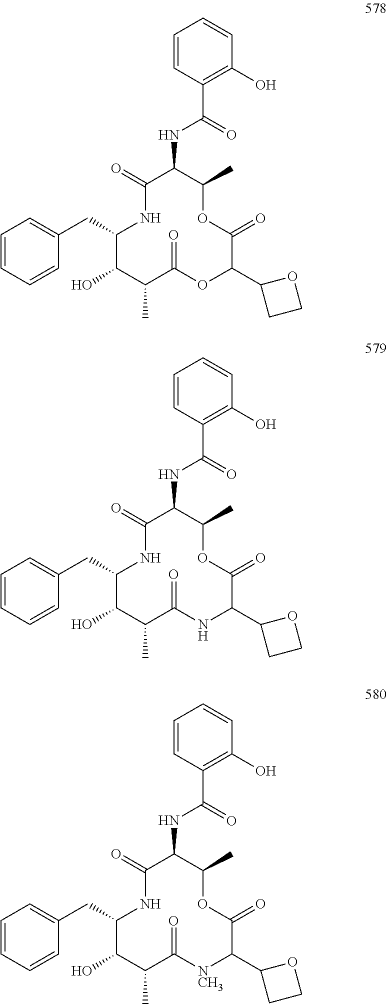 [SMILES: C[C@H]1OC(=O)C(C2CCO2)OC(=O)[C@H](C)[C@H](O)[C@H](Cc2ccccc2)NC(=O)[C@H]1NC(=O)c1ccccc1O ; C[C@H]1OC(=O)C(C2CCO2)NC(=O)[C@H](C)[C@H](O)[C@H](Cc2ccccc2)NC(=O)[C@H]1NC(=O)c1ccccc1O ; C[C@H]1OC(=O)C(C2CCO2)N(C)C(=O)[C@H](C)[C@H](O)[C@H](Cc2ccccc2)NC(=O)[C@H]1NC(=O)c1ccccc1O]

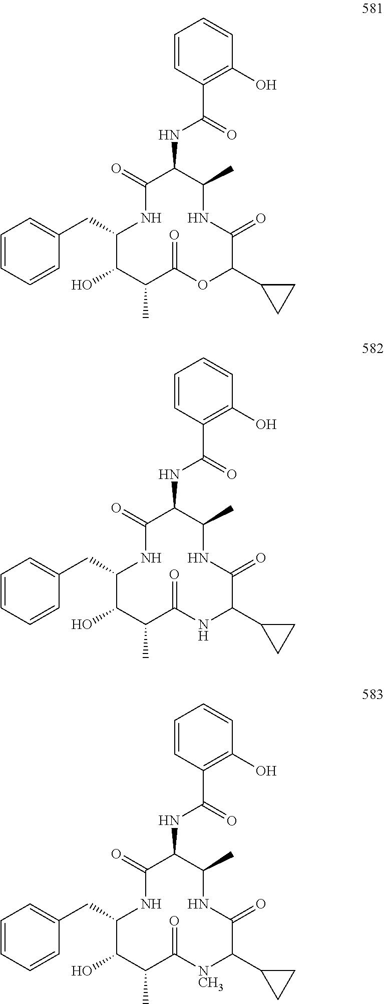 [SMILES: C[C@H]1NC(=O)C(C2CC2)OC(=O)[C@H](C)[C@H](O)[C@H](Cc2ccccc2)NC(=O)[C@H]1NC(=O)c1ccccc1O ; C[C@H]1NC(=O)C(C2CC2)NC(=O)[C@H](C)[C@H](O)[C@H](Cc2ccccc2)NC(=O)[C@H]1NC(=O)c1ccccc1O ; C[C@H]1NC(=O)C(C2CC2)N(C)C(=O)[C@H](C)[C@H](O)[C@H](Cc2ccccc2)NC(=O)[C@H]1NC(=O)c1ccccc1O]

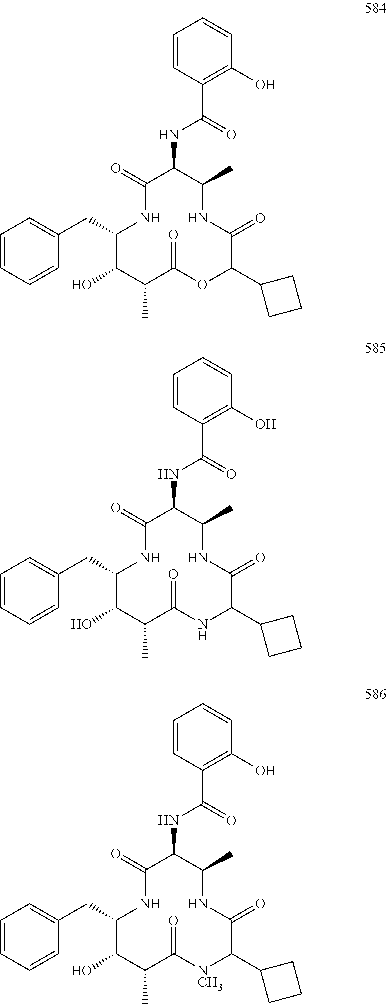 [SMILES: C[C@H]1NC(=O)C(C2CCC2)OC(=O)[C@H](C)[C@H](O)[C@H](Cc2ccccc2)NC(=O)[C@H]1NC(=O)c1ccccc1O ; C[C@H]1NC(=O)C(C2CCC2)NC(=O)[C@H](C)[C@H](O)[C@H](Cc2ccccc2)NC(=O)[C@H]1NC(=O)c1ccccc1O ; C[C@H]1NC(=O)C(C2CCC2)N(C)C(=O)[C@H](C)[C@H](O)[C@H](Cc2ccccc2)NC(=O)[C@H]1NC(=O)c1ccccc1O]

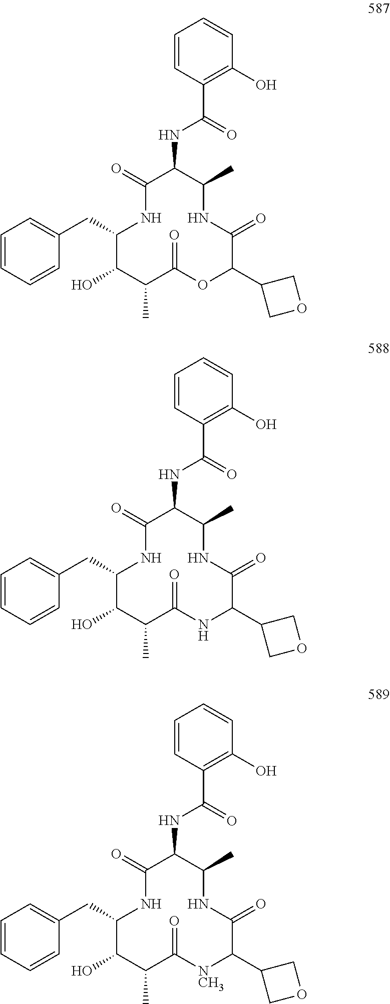 [SMILES: C[C@H]1NC(=O)C(C2COC2)OC(=O)[C@H](C)[C@H](O)[C@H](Cc2ccccc2)NC(=O)[C@H]1NC(=O)c1ccccc1O ; C[C@H]1NC(=O)C(C2COC2)NC(=O)[C@H](C)[C@H](O)[C@H](Cc2ccccc2)NC(=O)[C@H]1NC(=O)c1ccccc1O ; C[C@H]1NC(=O)C(C2COC2)N(C)C(=O)[C@H](C)[C@H](O)[C@H](Cc2ccccc2)NC(=O)[C@H]1NC(=O)c1ccccc1O]

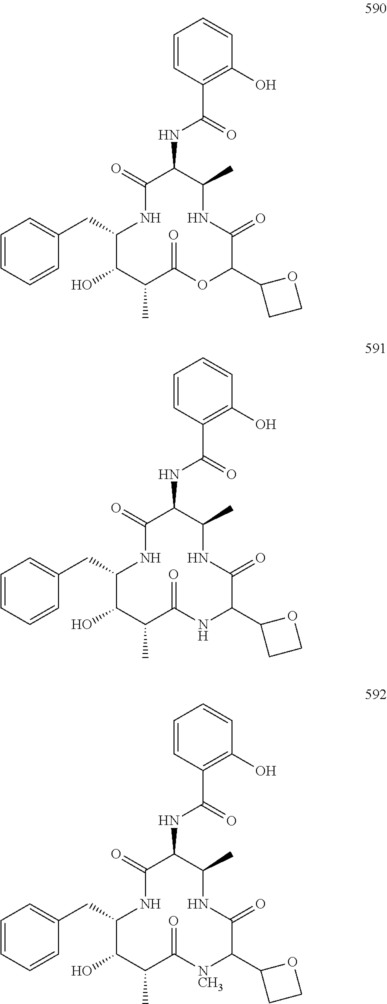 [SMILES: C[C@H]1NC(=O)C(C2CCO2)OC(=O)[C@H](C)[C@H](O)[C@H](Cc2ccccc2)NC(=O)[C@H]1NC(=O)c1ccccc1O ; C[C@H]1NC(=O)C(C2CCO2)NC(=O)[C@H](C)[C@H](O)[C@H](Cc2ccccc2)NC(=O)[C@H]1NC(=O)c1ccccc1O ; C[C@H]1NC(=O)C(C2CCO2)N(C)C(=O)[C@H](C)[C@H](O)[C@H](Cc2ccccc2)NC(=O)[C@H]1NC(=O)c1ccccc1O]

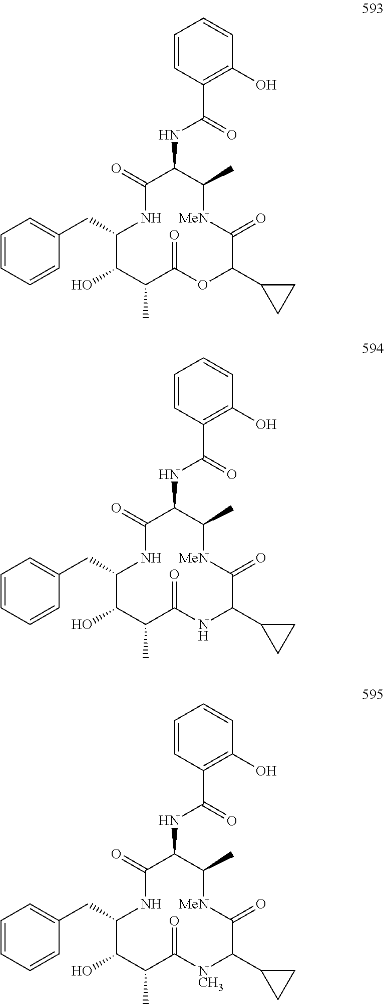 [SMILES: C[C@@H]1[C@H](NC(=O)c2ccccc2O)C(=O)N[C@@H](Cc2ccccc2)[C@@H](O)[C@@H](C)C(=O)OC(C2CC2)C(=O)N1C ; C[C@@H]1[C@H](NC(=O)c2ccccc2O)C(=O)N[C@@H](Cc2ccccc2)[C@@H](O)[C@@H](C)C(=O)NC(C2CC2)C(=O)N1C ; C[C@@H]1[C@H](NC(=O)c2ccccc2O)C(=O)N[C@@H](Cc2ccccc2)[C@@H](O)[C@@H](C)C(=O)N(C)C(C2CC2)C(=O)N1C]

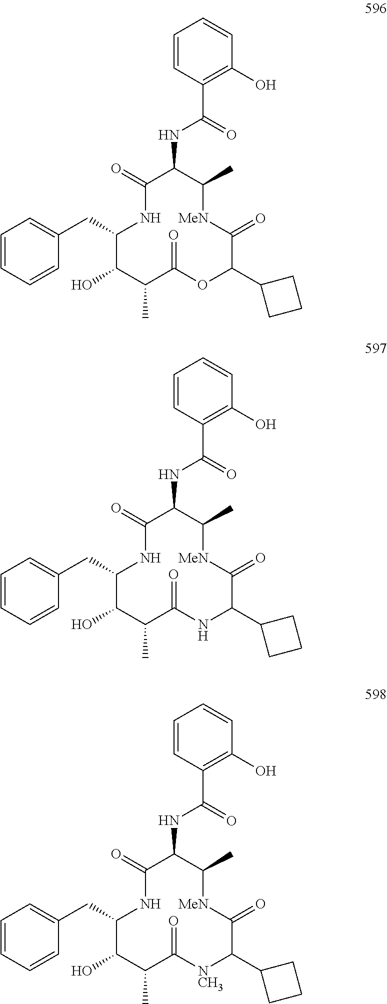 [SMILES: C[C@@H]1[C@H](NC(=O)c2ccccc2O)C(=O)N[C@@H](Cc2ccccc2)[C@@H](O)[C@@H](C)C(=O)OC(C2CCC2)C(=O)N1C ; C[C@@H]1[C@H](NC(=O)c2ccccc2O)C(=O)N[C@@H](Cc2ccccc2)[C@@H](O)[C@@H](C)C(=O)NC(C2CCC2)C(=O)N1C ; C[C@@H]1[C@H](NC(=O)c2ccccc2O)C(=O)N[C@@H](Cc2ccccc2)[C@@H](O)[C@@H](C)C(=O)N(C)C(C2CCC2)C(=O)N1C]

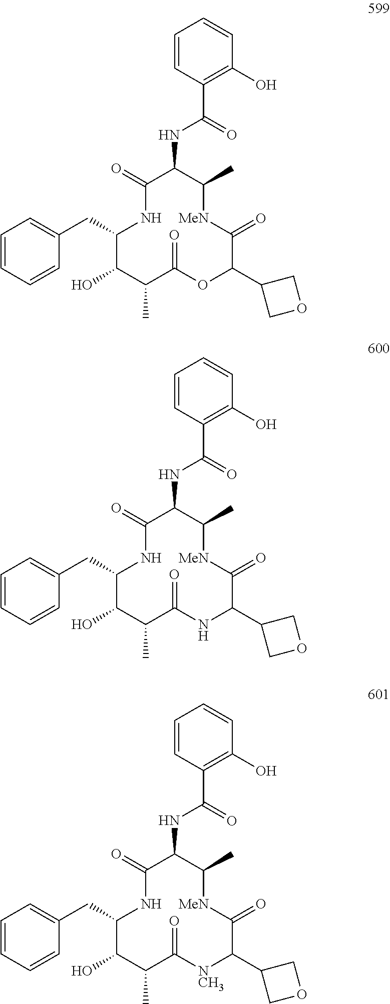 [SMILES: C[C@@H]1[C@H](NC(=O)c2ccccc2O)C(=O)N[C@@H](Cc2ccccc2)[C@@H](O)[C@@H](C)C(=O)OC(C2COC2)C(=O)N1C ; C[C@@H]1[C@H](NC(=O)c2ccccc2O)C(=O)N[C@@H](Cc2ccccc2)[C@@H](O)[C@@H](C)C(=O)NC(C2COC2)C(=O)N1C ; C[C@@H]1[C@H](NC(=O)c2ccccc2O)C(=O)N[C@@H](Cc2ccccc2)[C@@H](O)[C@@H](C)C(=O)N(C)C(C2COC2)C(=O)N1C]

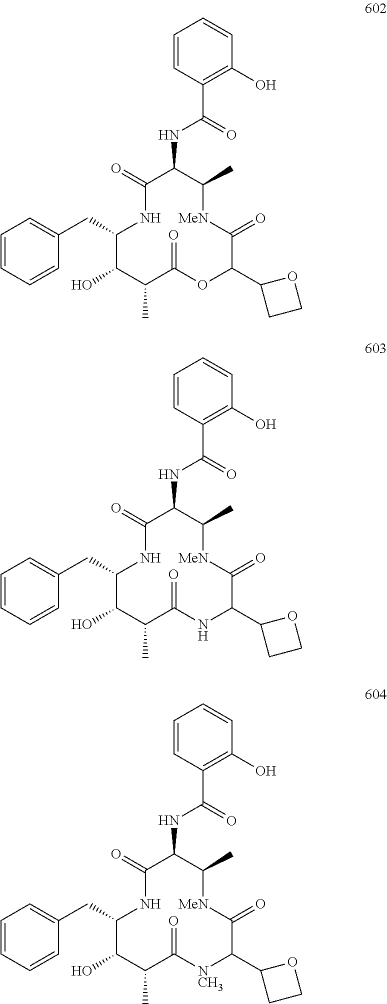 [SMILES: C[C@@H]1[C@H](NC(=O)c2ccccc2O)C(=O)N[C@@H](Cc2ccccc2)[C@@H](O)[C@@H](C)C(=O)OC(C2CCO2)C(=O)N1C ; C[C@@H]1[C@H](NC(=O)c2ccccc2O)C(=O)N[C@@H](Cc2ccccc2)[C@@H](O)[C@@H](C)C(=O)NC(C2CCO2)C(=O)N1C ; C[C@@H]1[C@H](NC(=O)c2ccccc2O)C(=O)N[C@@H](Cc2ccccc2)[C@@H](O)[C@@H](C)C(=O)N(C)C(C2CCO2)C(=O)N1C]

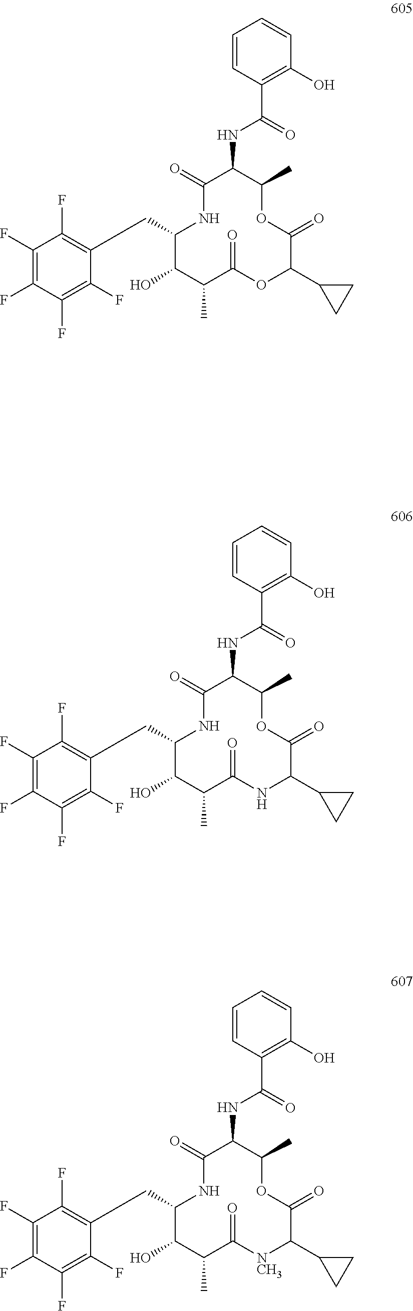 [SMILES: C[C@H]1OC(=O)C(C2CC2)OC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(C2CC2)NC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(C2CC2)N(C)C(=O)[C@H](C)[C@H](O)[C@H](Cc2c(F)c(F)c(F)c(F)c2F)NC(=O)[C@H]1NC(=O)c1ccccc1O]

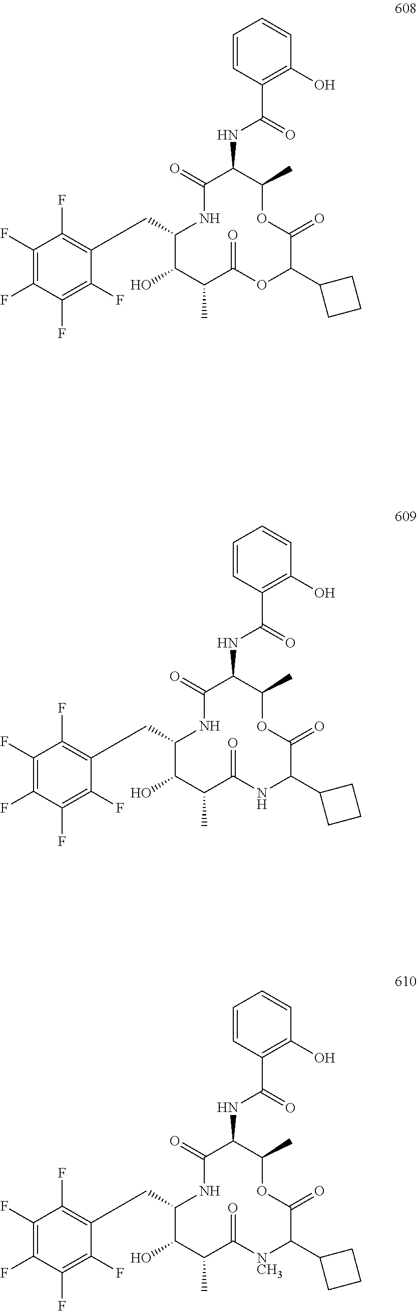 [SMILES: C[C@H]1OC(=O)C(C2CCC2)OC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(C2CCC2)NC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(C2CCC2)N(C)C(=O)[C@H](C)[C@H](O)[C@H](Cc2c(F)c(F)c(F)c(F)c2F)NC(=O)[C@H]1NC(=O)c1ccccc1O]

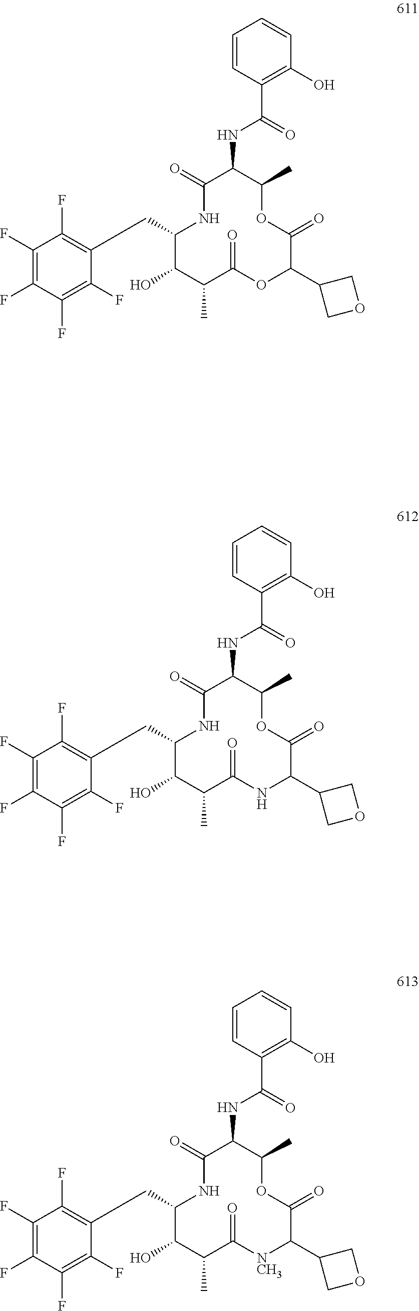 [SMILES: C[C@H]1OC(=O)C(C2COC2)OC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(C2COC2)NC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(C2COC2)N(C)C(=O)[C@H](C)[C@H](O)[C@H](Cc2c(F)c(F)c(F)c(F)c2F)NC(=O)[C@H]1NC(=O)c1ccccc1O]

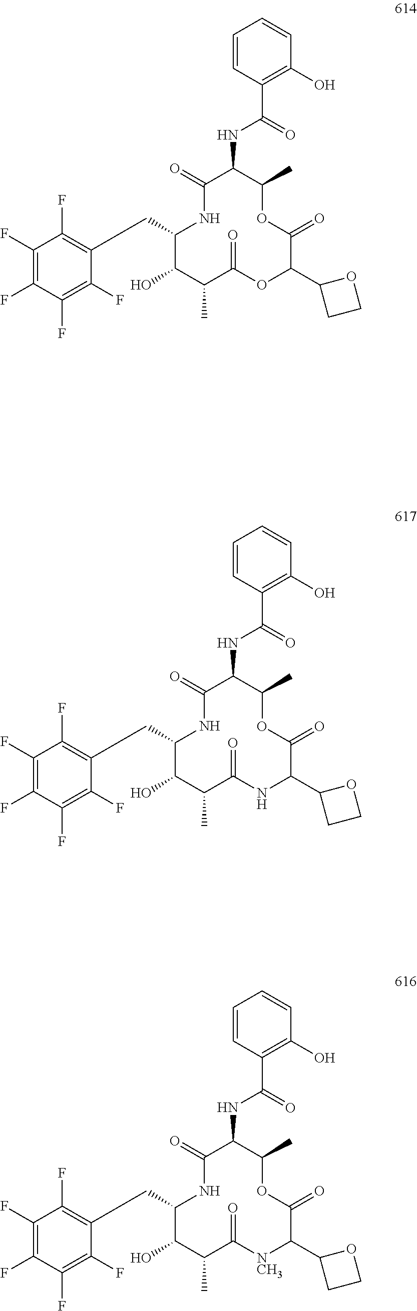 [SMILES: C[C@H]1OC(=O)C(C2CCO2)OC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(C2CCO2)NC(=O)[C@H](C)[C@H](O)[C@H](Cc2c(F)c(F)c(F)c(F)c2F)NC(=O)[C@H]1NC(=O)c1ccccc1O ; C[C@H]1OC(=O)C(C2CCO2)N(C)C(=O)[C@H](C)[C@H](O)[C@H](Cc2c(F)c(F)c(F)c(F)c2F)NC(=O)[C@H]1NC(=O)c1ccccc1O]

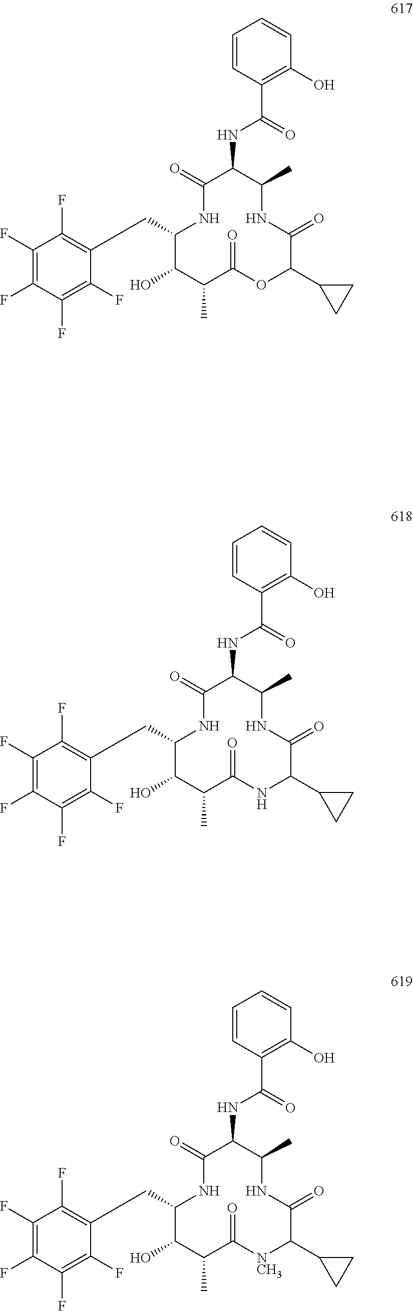 [SMILES: C[C@H]1NC(=O)C(C2CC2)OC(=O)[C@H](C)[C@H](O)[C@H](Cc2c(F)c(F)c(F)c(F)c2F)NC(=O)[C@H]1NC(=O)c1ccccc1O ; C[C@H]1NC(=O)C(C2CC2)NC(=O)[C@H](C)[C@H](O)[C@H](Cc2c(F)c(F)c(F)c(F)c2F)NC(=O)[C@H]1NC(=O)c1ccccc1O ; C[C@H]1NC(=O)C(C2CC2)N(C)C(=O)[C@H](C)[C@H](O)[C@H](Cc2c(F)c(F)c(F)c(F)c2F)NC(=O)[C@H]1NC(=O)c1ccccc1O]

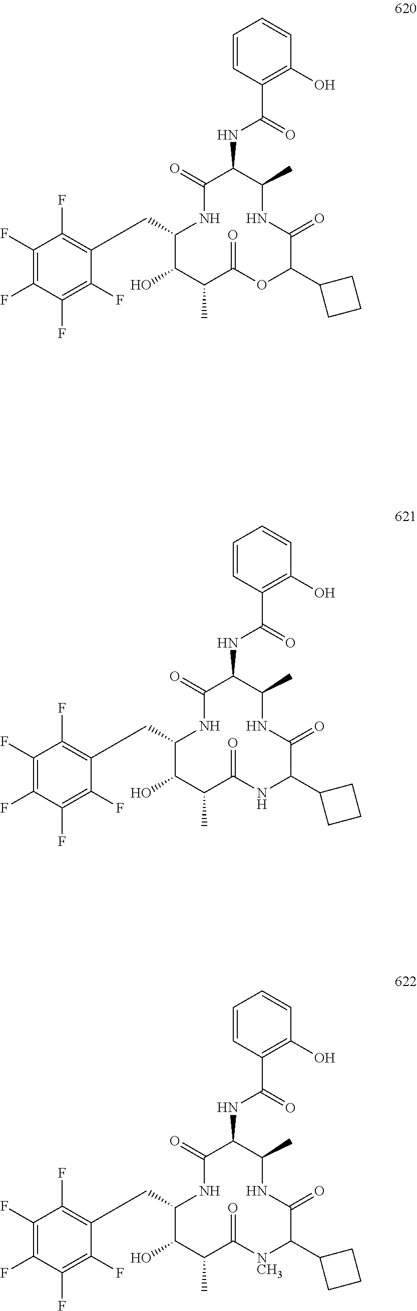 [SMILES: C[C@H]1NC(=O)C(C2CCC2)OC(=O)[C@H](C)[C@H](O)[C@H](Cc2c(F)c(F)c(F)c(F)c2F)NC(=O)[C@H]1NC(=O)c1ccccc1O ; C[C@H]1NC(=O)C(C2CCC2)NC(=O)[C@H](C)[C@H](O)[C@H](Cc2c(F)c(F)c(F)c(F)c2F)NC(=O)[C@H]1NC(=O)c1ccccc1O ; C[C@H]1NC(=O)C(C2CCC2)N(C)C(=O)[C@H](C)[C@H](O)[C@H](Cc2c(F)c(F)c(F)c(F)c2F)NC(=O)[C@H]1NC(=O)c1ccccc1O]

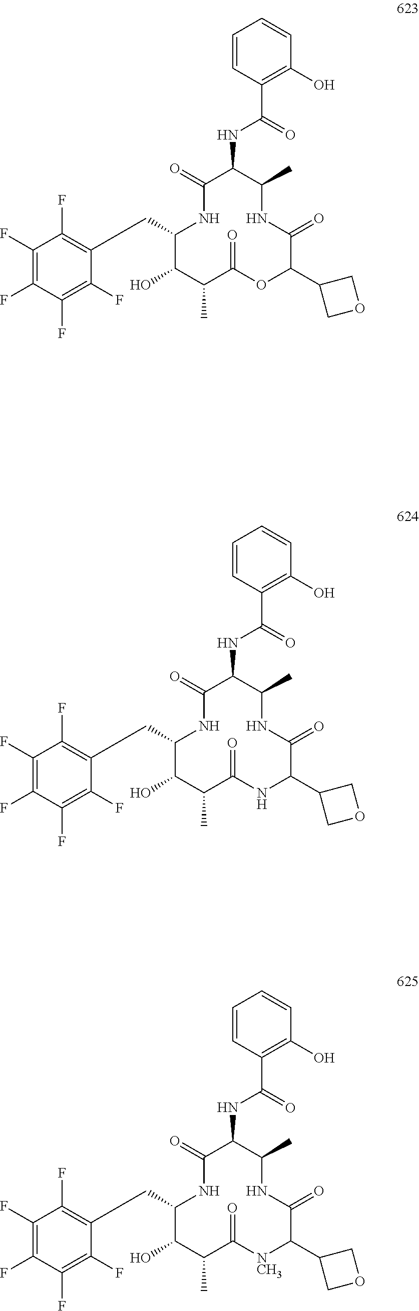 [SMILES: C[C@H]1NC(=O)C(C2COC2)OC(=O)[C@H](C)[C@H](O)[C@H](Cc2c(F)c(F)c(F)c(F)c2F)NC(=O)[C@H]1NC(=O)c1ccccc1O ; C[C@H]1NC(=O)C(C2COC2)NC(=O)[C@H](C)[C@H](O)[C@H](Cc2c(F)c(F)c(F)c(F)c2F)NC(=O)[C@H]1NC(=O)c1ccccc1O ; C[C@H]1NC(=O)C(C2COC2)N(C)C(=O)[C@H](C)[C@H](O)[C@H](Cc2c(F)c(F)c(F)c(F)c2F)NC(=O)[C@H]1NC(=O)c1ccccc1O]

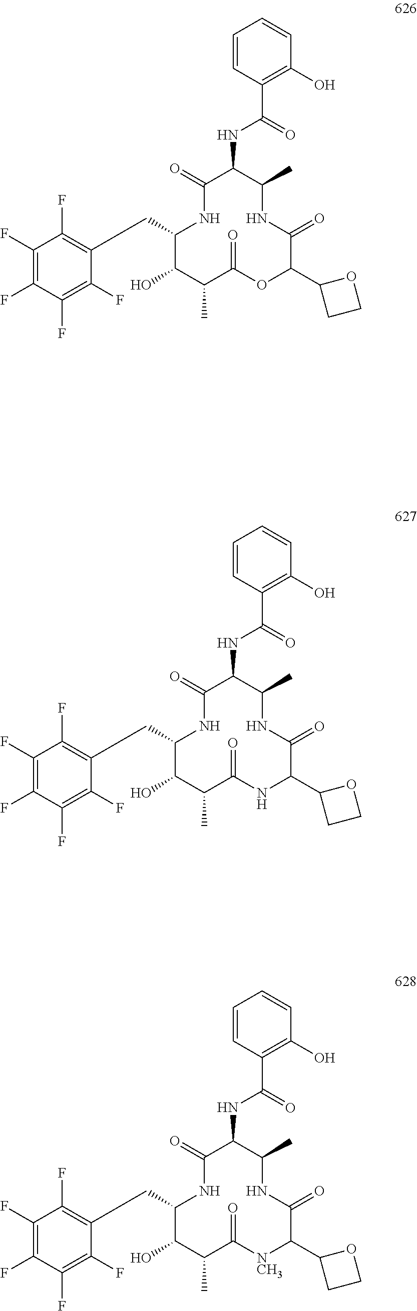 [SMILES: C[C@H]1NC(=O)C(C2CCO2)OC(=O)[C@H](C)[C@H](O)[C@H](Cc2c(F)c(F)c(F)c(F)c2F)NC(=O)[C@H]1NC(=O)c1ccccc1O ; C[C@H]1NC(=O)C(C2CCO2)NC(=O)[C@H](C)[C@H](O)[C@H](Cc2c(F)c(F)c(F)c(F)c2F)NC(=O)[C@H]1NC(=O)c1ccccc1O ; C[C@H]1NC(=O)C(C2CCO2)N(C)C(=O)[C@H](C)[C@H](O)[C@H](Cc2c(F)c(F)c(F)c(F)c2F)NC(=O)[C@H]1NC(=O)c1ccccc1O]

17. Compound according to claim 1, or a pharmaceutically acceptable salt thereof for use as a medicament.

18. Compound according to claim 1, or pharmaceutically acceptable salts thereof for use as a medicament for the treatment of bacterial infections.
